(19) United States
(12) Patent Application Publication
(10) Pub. No.: US 2018/0243298 A1
(43) Pub. Date: Aug. 30, 2018

(54) TRPV3 MODULATORS

(71) Applicant: AbbVie Inc., North Chicago, IL (US)

(72) Inventors: Lawrence Black, Libertyville, IL (US); Brian S. Brown, Evanston, IL (US); Bruce Clapham, Lindenhurst, IL (US); Marlon D. Cowart, Round Lake Beach, IL (US); Michael J. Dart, Highland Park, IL (US); Ramin Faghih, Lake Forest, IL (US); Jennifer Frost, Gurnee, IL (US); Arthur R. Gomtsyan, Vernon Hills, IL (US); Philip R. Kym, Libertyville, IL (US); Huaqing Liu, Buffalo Grove, IL (US); Robert G. Schmidt, Antioch, IL (US); Lei Shi, Gurnee, IL (US); Anurupa Shrestha, Gurnee, IL (US); Kevin Sippy, Antioch, IL (US)

(21) Appl. No.: 15/563,888

(22) PCT Filed: Mar. 30, 2016
(86) PCT No.: PCT/US2016/024940

§ 371 Date: Feb. 13, 2018

Related U.S. Application Data
(60) Provisional application No. 62/219,769, filed on Sep. 17, 2015.

(30) Foreign Application Priority Data
Apr. 2, 2015 (CN) PCT/CN2015/075758

(51) Int. Cl. A61K 31/506; A61P 17/06; A61P 17/04

(52) U.S. Cl. CPC A61K 31/506; A61P 17/04; A61P 17/06

(57) ABSTRACT

A compound of Formula (I) having efficacy for the treatment or prevention of conditions and disorders related to TRPV3 activity, e.g. pain, atopic dermatitis, eczema, itch or psoriasis, is disclosed. Compositions comprising such compounds and methods for treating conditions and disorders using such compounds and compositions are also presented.

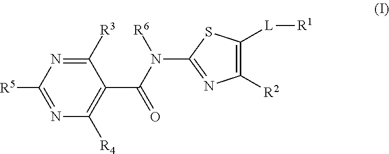

### CROSS REFERENCE TO RELATED APPLICATIONS

The present application claims the benefit of priority to International Application No. PCT/CN2015/075758, filed Apr. 2, 2015. The present application claims the benefit of priority to U.S. provisional Application Ser. No. 62/219,769, filed Sep. 17, 2015. The disclosures of the priority documents are hereby incorporated by reference as if set forth in their entirety.

### TECHNICAL FIELD

The present invention generally relates to compounds that are Transient Receptor Potential Vanilloid 3 (TRPV3) modulators, compositions comprising such compounds, and methods for treating conditions and disorders using such compounds and compositions, are disclosed herein.

### BACKGROUND OF THE INVENTION

A subset of the vanilloid channels (TRPV1-4) are referred to as thermoTRPs to reflect the observation that heat elicits channel opening across a continuum of temperatures with thresholds ranging from 25° C. to 52° C. (Caterina, M. J.; Rosen, T. A.; Tominaga, M.; Brake, A. J.; Julius, D., Nature 1999, 398, 436-441). TRPV3 characteristically responds to innocuous heat >31° C., exhibits exquisite sensitivity around the physiological temperature of humans, 37° C., and sensitizes dramatically following repetitive heating (Smith, G. D.; Gunthorpe, M. J.; Kelsell, R. E.; Hayes, P. D.; Reilly, P.; Facer, P.; Wright, J. E.; Jerman, J. C.; Walhin, J. P.; Ooi, L.; Egerton, J.; Charles, K. J.; Smart, D.; Randall, A. D.; Anand, P.; Davis, J. B., Nature 2002, 418, 186-190; Xu, H.; Ramsey, I. S.; Kotecha, S. A.; Moran, M. M.; Chong, J. A.; Lawson, D.; Ge, P.; Lilly, J.; Silos-Santiago, I.; Xie, Y.; DiStefano, P. S.; Curtis, R.; Clapham, D. E., Nature 2002, 418, 181-186; Peier, A. M.; Reeve, A. J.; Andersson, D. A.; Moqrich, A.; Earley, T. J.; Hergarden, A. C.; Story, G. M.; Colley, S.; Hogenesch, J. B.; McIntyre, P.; Bevan, S.; Patapoutian, A., Science 2002, 296, 2046-2049).

TRPV3 is a nonselective cation channel with permeability for calcium, but also to other cations, for example sodium. Multiple compounds that have been shown to activate TRPV3, include: monoterpenes, camphor (Peier, A. M. et al., 2002; Moqrich, A.; Hwang, S. W.; Earley, T. J.; Petrus, M. J.; Murray, A. N.; Spencer, K. S.; Andahazy, M.; Story, G. M.; Patapoutian, A., Science 2005, 307, 1468-1472; Xu, H.; Blair, N. T.; Clapham, D. E., J Neurosci. 2005, 25, 8924-8937), carvacrol, and thymol (Xu, H.; Delling, M.; Jun, J. C.; Clapham, D. E. Nat Neurosci. 2006, 9, 628-635; Vogt-Eisele, A. K.; Weber, K.; Sherkheli, M. A.; Vielhaber, G.; Panten, J.; Gisselmann, G.; Hatt, H., Br J Pharmacol. 2007, 151, 530-540; Story, G. M., Mol Cell Neurosci. 2006, 32, 335-343; Vogt-Eisele, A. K. et al., 2007); cinnamaldehyde (Macpherson, L. J. et al., 2006); incensole acetate (Moussaieff, A.; Rimmerman, N.; Bregman, T.; Straiker, A.; Felder, C. C.; Shoham, S.; Kashman, Y.; Huang, S. M.; Lee, H.; Shohami, E.; Mackie, K.; Caterina, M. J.; Walker, J. M.; Fride, E.; Mechoulam, R., FASEB J. 2008, 22, 3024-3034.); and vanilloid analogs, eugenol and ethyl vanillin (Hu, H. Z.; Gu, Q.; Wang, C.; Colton, C. K.; Tang, J.; Kinoshita-Kawada, M.; Lee, L. Y.; Wood, J. D.; Zhu, M. X., J Biol Chem. 2004, 279, 35741-35748; Vogt-Eisele, A. K. et al., 2007; Xu, H. et al., 2006). Though relatively weak (EC₅₀˜40 μM) and nonspecific across TRPs, 2-aminoethoxydiphenylborate (2-APB) and diphenylboronic anhydride (DPBA) have been widely and productively used to characterize key attributes of TRPV3 in cellular assays and electrophysiology (Hu, H. Z. et al., 2004; Chung, M. K.; Lee, H.; Mizuno, A.; Suzuki, M.; Caterina, M. J. J Neurosci. 2004, 24, 5177-5182; Chung, M. K.; Guiler, A. D.; Caterina, M. J., J Biol Chem. 2005, 280, 15928-15941). While heat and direct ligand binding are clearly central to TRPV3 pharmacology, accumulating evidence of potentiation by arachidonic acid, other unsaturated fatty acid derivatives (Hu, H. Z.; Xiao, R.; Wang, C.; Gao, N.; Colton, C. K.; Wood, J. D.; Zhu, M. X., J Cell Physiol. 2006, 208, 201-212), and nitric oxide (Aley, K. O.; McCarter, G.; Levine, J. D., J Neurosci. 1998, 18, 7008-7014; Yoshida, T.; Inoue, R.; Morii, T.; Takahashi, N.; Yamamoto, S.; Hara, Y.; Tominaga, M.; Shimizu, S.; Sato, Y.; Mori, Y., Nat Chem Biol. 2006, 2, 596-607) suggests that authentic activation involves stimulation of G protein-coupled receptors and downstream second messenger signal cascades (e.g., phospholipase C, protein kinase C) that mediate local inflammatory responses and nociceptor sensitization that could enhance TRPV3 function (Xu, H. et al., 2006) in a pathophysiological, as compared to basal state.

Evidence suggests that transcriptional regulation of the TRPV3 gene restricts its basal expression and is responsible for enhanced expression following nerve injury. Levels of TRPV3 mRNA recovered from rat L4 and L5 DRG neurons is elevated in the spinal nerve ligation model of neuropathic pain, as compared to uninjured rats (U.S. Pat. No. 7,396,910). Similar upregulation of TRPV3 has been observed in sensory neurons following peripheral nerve injury in humans (Facer, P.; Casula, M. A.; Smith, G. D.; Benham, C. D.; Chessell, I. P.; Bountra, C.; Sinisi, M.; Birch, R.; Anand, P., BMC Neurol. 2007, 7, 11-22; Smith G. D. et al., 2002).

One feature that distinguishes TRPV3 from the other thermoTRPs is its relatively prominent localization in skin (Peier, A. M. et al., 2002; Xu, H. et al., 2002). TRPV3 is also expressed in the dorsal root ganglia, trigeminal ganglia, spinal cord and brain (Xu, H. et al., 2002; Smith G. D. et al., 2002). Its distinctive tissue profile, with significant expression in keratinocytes proximal to nociceptive neurons (Chung, M. K.; Lee, H.; Caterina, M. J., J Biol Chem. 2003, 278, 32037-32046; Chung, M. K.; Lee, H.; Mizuno, A.; Suzuki, M.; Caterina, M. J. J Biol Chem. 2004, 279, 21569-21575; Peier, A. M. et al., 2002; Xu, H. et al., 2002) as well as upregulation of TRPV3 in disease states is consistent with a likely role of TRPV3 in pain (Caterina M J., Am J Physiol Regul Integr Comp Physiol. 2007, 292, R64-R76; Lee, H.; Caterina, M. J., Pflugers Arch. 2005, 451, 160-167; Giler, A. D.; Lee, H.; Iida, T.; Shimizu, I.; Tominaga, M.; Caterina, M., J Neurosci. 2002, 22, 6408-6414; Chung, M. K. et al., 2003; Chung, M. K.; Lee, H.; Mizuno, A.; Suzuki, M.; Caterina, M. J. J Biol Chem. 2004, 279, 21569-21575). In a keratinocyte cell line, stimulation of TRPV3 leads to release of inflammatory mediators including interleukin-1. Thus TRPV3 may also play an important role in regulating inflammation, itch (Steinhoff, M. and Biro, T. J. Invest. Dermatology, 2009, 129, 531-535) and pain that results from the release of inflammatory stimuli. In addition, localization of TRPV3 in non-neuronal tissues, especially skin, suggests also that pharmacological modulation of the channel may provide a therapy to treat diseases that impair the skin barrier (Montell, C. Cell, 2010, Apr. 16, 218-220) and have additional, as yet unidentified, benefit for disease states beyond pain. For example, TRPV3 has been confirmed to play a role in development of dry skin itch, various forms of dermatitis (Yoshioka T, Imura K, Asakawa M, Suzuki M, Oshima I, Hirasawa T, Sakata T, Horikawa T, Arimura A. J Invest Dermatol, 2009, 129, 714-22). such as, but not limited to, atopic dermatitis, as well as rosacea (Sulk M, Seeliger S, Aubert J, Schwab V D, Cevikbas F, Rivier M, Nowak P, Voegel J J, Buddenkotte J, Steinhoff M. J Invest Dermatol. 2012 132, 1253-62) and also hair growth disorders (Asakawa M, Yoshioka T, Matsutani T, Hikita I, Suzuki M, Oshima I, Tsukahara K, Arimura A, Horikawa T, Hirasawa T, Sakata T. J Invest Dermatol., 2006, 126, 2664-2672). Additionally, gain of function mutation in the TRPV3 gene was found in patients with Olmsted syndrome (Lin Z, Chen Q, Lee M, Cao X, Zhang J, Ma D, Chen L, Hu X, Wang H, Wang X, Zhang P, Liu X, Guan L, Tang Y, Yang H, Tu P, Bu D, Zhu X, Wang K, Li R, Yang Y. Am J. Hum Genet 2012, 90, 558-64), a rare congenital disorder characterized by alopecia, keratotic plaque formation and severe itching. Accordingly, compounds that can modulate one or more functions of TRPV3 can have various therapeutic utilities.

### SUMMARY

The present invention is directed to a compound of Formula (I):

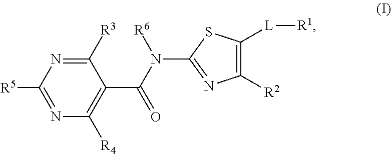

or a pharmaceutically acceptable salt thereof, wherein:

L is selected from the group consisting of a bond, —O—, —C(O)—, —C(O)O—, —C(O)N(R^{L}), —S—, —S(O)—, and —S(O)₂—;

R^{L} is selected from the group consisting of hydrogen and C₁-C₆-alkyl;

R¹ is selected from the group consisting of hydrogen, halogen, cyano, imino, C₁-C₁₀-alkyl, C₂-C₁₀-alkenyl, C₂-C₁₀-alkynyl, C₃-C₆-cycloalkyl, C₅-C₁₀-cycloalkenyl, aryl, 4- to 10-membered ring heterocyclyl, and 5- to 10-membered ring heteroaryl; wherein:
the imino may be unsubstituted or substituted with one or two substituents independently selected from the group consisting of hydroxyl, C₁-C₆-alkyl, C₁-C₆-alkoxy, aryl and 5- to 10-membered ring heteroaryl;
the R¹ C₁-C₁₀-alkyl may be unsubstituted or substituted with one, two, or three substituents independently selected from the group consisting of halogen, amino, cyano, C₃-C₆-cycloalkyl, aryl, 5- to 10-membered ring heteroaryl, 4- to 10-membered ring heterocyclyl, —OR¹⁰¹, —C(O)OR¹⁰², and ═NR¹⁰³;
   wherein the amino, C₃-C₆-cycloalkyl, aryl, 5- to 10-membered ring heteroaryl, 4- to 10-membered ring heterocyclyl substituents of the R¹ C₁-C₁₀-alkyl may be unsubstituted or substituted with one, two, or three substituents independently selected from the group consisting of halogen, C₁-C₆-alkyl, C₁-C₆-alkoxy, C₃-C₆-cycloalkyl, halo-C₁-C₆-alkyl, and 4- to 10-membered ring heterocyclyl;
   wherein R¹⁰¹ and R¹⁰² are independently selected from the group consisting of hydrogen, C₁-C₆-alkyl, halo-C₁-C₆-alkyl, C₃-C₆-cycloalkyl, halo-C₃-C₆-cycloalkyl, C₁-C₆-alkyl-C₃-C₆-cycloalkyl, aryl, haloaryl, and C₁-C₆-alkylaryl; and
   wherein R¹⁰³ is selected from the group consisting of hydrogen, hydroxy, C₁-C₆-alkyl, and C₁-C₆-alkoxy;
the R¹ C₂-C₁₀-alkenyl and the R¹ C₂-C₁₀-alkynyl may be unsubstituted or substituted with one, two, or three substituents independently selected from the group consisting of hydroxy, C₁-C₆-alkoxy, C₃-C₆-cycloalkyl, aryloxy, 5- to 10-membered ring heteroaryloxy, C₁-C₆-alkoxycarbonyl, alkylsilyl, and cyano-C₁-C₆-alkylcarbonylamino;
the R¹ C₃-C₆-cycloalkyl and the R¹ C₃-C₆-cycloalkenyl may be unsubstituted or substituted with one, two, or three substituents independently selected from the group consisting of halogen, cyano, oxo, OH, C₁-C₆-alkyl, halo-C₁-C₆-alkyl, C₁-C₆-alkoxy, halo-C₁-C₆-alkoxy, C₁-C₆-alkoxycarbonyl, C₁-C₆-alkyoxycarbonylamino, and halo-C₁-C₆-alkoxycarbonyl;
the R¹ aryl and the R¹ 5- to 10-membered ring heteroaryl may be unsubstituted or substituted with one, two, or three substituents independently selected from the group consisting of halogen, cyano, C₁-C₆-alkyl, C₁-C₆ alkoxy, hydroxy-C₁-C₆-alkyl, halo-C₁-C₆-alkyl, cyano-C₁-C₆-alkyl, C₁-C₆-alkoxy-C₁-C₆-alkyl, C₁-C₆-alkoxycarbonyl-C₁-C₆-alkyl, C₃-C₆-cycloalkyl, C₃-C₆-cycloalkyl-C₁-C₆-alkyl, C₅-C₁₀-Cycloalkenyl, aryl, aryl-C₁-C₆-alkyl, 4- to 10-membered ring heterocyclyl, 4- to 10-membered ring heterocyclyl-C₁-C₆-alkyl, 5- to 10-membered ring heteroaryl, 5- to 10-membered ring heteroaryl-C₁-C₆-alkyl, —OR¹⁰⁴, C(O)OR¹⁰⁵, —N(R¹⁰⁶)C(O)R¹⁰⁷, —N(R¹⁰⁸)C(O)NR¹⁰⁹R¹¹⁰, —S(O)₂R¹¹¹, —S(O)₂NR¹¹²R¹¹³, and —NS(O)₂NR¹¹²R¹¹³;
   wherein the C₃-C₆-cycloalkyl, C₃-C₆-cycloalkyl-C₁-C₆-alkyl, C₅-C₁₀-cycloalkenyl, aryl, aryl-C₁-C₆-alkyl, 4- to 10-membered ring heterocyclyl, 4- to 10-membered ring heterocyclyl-C₁-C₆-alkyl, 5- to 10-membered ring heteroaryl, and 5- to 10-membered ring heteroaryl-C₁-C₆-alkyl of the R¹ aryl and the R¹ 5- to 10-membered ring heteroaryl may be unsubstituted or substituted with one, two, or three substituents independently selected from the group consisting of halogen, cyano, C₁-C₆-alkyl, halo-C₁-C₆-alkyl, cyano-C₁-C₆-alkyl, and C₁-C₆-alkoxycarbonyl;
   wherein R¹⁰⁴, R¹⁰⁵, R¹⁰⁷, R¹⁰⁹, R¹¹⁰, R¹¹¹, R¹¹², and R¹¹³ are independently selected from the group consisting of hydrogen, C₁-C₆-alkyl, C₁-C₆-alkoxyC₁-C₆-alkyl, halo-C₁-C₆-alkyl, cyano-C₁-C₆-alkyl, C₃-C₆-cycloalkyl, halo-C₃-C₆-cycloalkyl, 4- to 10-membered ring heterocyclyl, 4- to 10-membered ring heterocyclyl-C₁-C₆-alkyl, aryl, haloaryl, and C₁-C₆-alkylaryl; and
   wherein R¹⁰⁶ and R¹⁰⁸ are independently selected from the group consisting of hydrogen and C₁-C₆-alkyl; and
the R¹ 4- to 10-membered ring heterocyclyl may be unsubstituted or substituted with one, two, or three substituents independently selected from the group consisting of halogen, OH, C₁-C₆-alkyl, halo-C₁-C₆-alkyl, C₁-C₆-alkoxycarbonyl, 4- to 10-membered ring heterocyclyl, and —S(O)₂R¹¹⁴; wherein R¹¹⁴ is selected from the group consisting of hydrogen and C₁-C₆-alkyl;

R² is selected from the group consisting of C₁-C₆-alkyl, C₂-C₆-alkenyl, C₃-C₆-cycloalkyl, aryl, 4- to 10-membered ring heterocyclyl, and 5- to 10-membered ring heteroaryl; wherein:
the R² C₁-C₆-alkyl or the C₂-C₆-alkenyl may be unsubstituted or substituted with one, two, or three substituents independently selected from the group consisting of halogen, hydroxy, cyano, C₁-C₆-alkoxy, halo-C₁-C₆-alkoxy, C₃-C₆-cycloalkyl, halo-C₃-C₆-cycloalkyl, C₃-C₆-cycloalkoxy, halo-C₃-C₆-cycloalkoxy, aryl, haloaryl, aryloxy, haloaryloxy, and C₁-C₆-alkylaryloxy;
the R² C₃-C₆-cycloalkyl may be unsubstituted or substituted with one, two, or three substituents independently selected from the group consisting of halogen, C₁-C₆-alkyl, and halo-C₁-C₆-alkyl;
the R² aryl and the R² 5- to 10-membered ring heteroaryl may be unsubstituted or substituted with one, two, or three substituents independently selected from the group consisting of halogen, hydroxy, cyano, C₁-C₆-alkyl, hydroxy-C₁-C₆-alkyl, halo-C₁-C₆-alkyl, cyano-C₁-C₆-alkyl, C₃-C₆-cycloalkyl, C₁-C₆-alkoxy, halo-C₁-C₆-alkoxy, —S(O)₂R²⁰¹, and —S(O)₂NR²⁰²R²⁰³; wherein R²⁰¹, R²⁰², and R²⁰³ are independently selected from the group consisting of hydrogen and C₁-C₆-alkyl; and
the R² 4- to 10-membered ring heterocyclyl may be unsubstituted or substituted with one, two, or three substituents independently selected from the group consisting of halogen, C₁-C₆-alkyl, and halo-C₁-C₆-alkyl;

R³ and R⁴ are independently selected from the group consisting of hydrogen, C₁-C₆-alkyl, C₁-C₆-alkoxy, C₁-C₆-alkylthio, and C₃-C₆-cycloalkyl, wherein the C₁-C₆-alkyl may be substituted with one, two, or three halogen;

R⁵ is selected from the group consisting of hydrogen, methyl, methoxy, and cyano; and

R⁶ is selected from the group consisting of hydrogen or —CH₂-phosphate.

The present invention further relates to a compound of Formula (I) wherein L is a bond and R¹ is pyridine substituted by cyano-C₁-C₆-alkyl.

The present invention further relates to pharmaceutical compositions comprising a therapeutically effective amount of a compound described herein or a pharmaceutically acceptable salt, solvate, salt of a solvate, or solvate of a salt thereof, in combination with a pharmaceutically acceptable carrier. The present invention further relates to pharmaceutical compositions comprising a therapeutically effective amount of a compound described herein or a pharmaceutically acceptable salt thereof, in combination with a pharmaceutically acceptable carrier. Such compositions can be administered in accordance with methods described herein, typically as part of a therapeutic regimen for treatment or prevention of conditions and disorders related to TRPV3 activity. More particularly, the methods are useful for treating conditions related to pain such as, but not limited to, itch, and pain such as, but not limited to, chronic pain, acute pain, neuropathic pain, nociceptive pain, osteoarthritic pain, inflammatory pain, fibromyalgia, post herpetic neuralgia, cancer pain (e.g. bone cancer pain), lower back pain, post operative pain, migraine, diabetic neuropathy, and eye pain, or combinations thereof. Accordingly, the present invention still further relates to pharmaceutical compositions for treating conditions related to pain such as, but not limited to, itch, and pain such as, but not limited to, chronic pain, acute pain, neuropathic pain, nociceptive pain, osteoarthritic pain, inflammatory pain, fibromyalgia, post herpetic neuralgia, cancer pain (e.g. bone cancer pain), lower back pain, post operative pain, migraine, diabetic neuropathy, and eye pain, or combinations thereof, the compositions comprising compounds, or pharmaceutically acceptable salts thereof, as described herein, optionally, in combination with a pharmaceutically acceptable carrier.

The present invention further relates to the uses of present compounds or pharmaceutically acceptable salts, solvates, or salts of solvates thereof, in the manufacture of medicaments for the treatment of the disease or conditions described above, alone or in combination with a pharmaceutically acceptable carrier, particularly for the treatment of itch or pain such as, but not limited to, chronic pain, acute pain, neuropathic pain, nociceptive pain, osteoarthritic pain, inflammatory pain, fibromyalgia, post herpetic neuralgia, cancer pain (e.g. bone cancer pain), lower back pain, post operative pain, migraine, diabetic neuropathy, and eye pain, or combinations thereof.

The compounds, compositions comprising the compounds, pharmaceutically acceptable salts, solvates, salts of the solvates, or solvates of the salts thereof, and methods for treating or preventing conditions and disorders by administering the compounds or compositions thereof, are further described herein.

These and other objectives are described further in the following paragraphs. These objectives should not be deemed to narrow the scope of the invention.

### DETAILED DESCRIPTION OF THE INVENTION

Compounds of Formula (I):

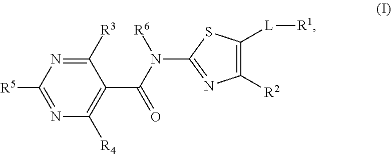

or a pharmaceutically acceptable salt thereof are disclosed, wherein L, R¹, R², R³, R⁴, R⁵, and R⁶ are as defined herein. Compositions comprising such compounds and methods for treating conditions and disorders using such compounds and compositions are also disclosed.

In various embodiments, compounds described herein may contain variables that occur more than one time in any substituent or in the compound described or any other formulae herein. Definition of a variable on each occurrence is independent of its definition at another occurrence. Further, combinations of variables are permissible only if such combinations result in stable compounds. Stable compounds are compounds that can be isolated from a reaction mixture.

### a. Definitions

It is noted that, as used in this specification and the intended claims, the singular form “a,” “an,” and “the” include plural referents unless the context clearly dictates otherwise. Thus, for example, reference to “a compound” includes a single compound as well as one or more of the same or different compounds, reference to “an optional pharmaceutically acceptable carrier” refers to a single optional pharmaceutically acceptable carrier as well as one or more pharmaceutically acceptable carriers, and the like.

As used in the specification and the appended claims, unless specified to the contrary, the following terms have the meaning indicated:

The term “alkenyl”, as used herein, means a straight or branched hydrocarbon chain containing from 2 to 10 carbons and containing at least one carbon-carbon double bond. In some embodiments, alkenyl may comprise a straight or branched hydrocarbon chain containing from 2 to 6 carbons and containing at least one carbon-carbon double bond. The term “C₂-C₄ alkenyl” means an alkenyl group containing 2-4 carbon atoms. Non-limiting examples of alkenyls include buta-2,3-dienyl, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenylene”, as used herein, means a divalent group derived from a straight or branched chain hydrocarbon of 2 to 4 carbon atoms and contains at least one carbon-carbon double. Representative examples of alkenylene include, but are not limited to, —CH═CH— and —CH₂CH═CH—.

The term “alkyl”, as used herein, means a straight or branched, saturated hydrocarbon chain containing from 1 to 10 carbon atoms. In some embodiments, an alkyl comprises a straight or branched, saturated hydrocarbon chain containing from 1 to 6 carbon atoms. The term “C_{X}-C_{y} alkyl” means a straight or branched chain, saturated hydrocarbon containing x to y carbon atoms. For example “C₁-C₆ alkyl” means a straight or branched chain, saturated hydrocarbon containing 1 to 6 carbon atoms. Examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 2-methylpentyl, 2,2-dimethylbutyl, 2-methylhexyl, 3-methylhexyl, 2,2-dimethylpentyl, 2,3-dimethylpentyl, n-heptyl, n-octyl, 2-methylheptyl, 3-methylheptyl, 2,3-dimethylheptyl, isooctyl, n-nonyl, and n-decyl.

The term “alkylene”, as used herein, means a divalent group derived from a straight or branched, saturated hydrocarbon chain of 1 to 10 carbon atoms, for example, of 1 to 6 carbon atoms. The term “C₁-C₆ alkylenyl”, as used herein, means a divalent group derived from a straight or branched, saturated hydrocarbon chain of 1 to 6 carbon atoms. Examples of an alkylene include, but are not limited to, —CH₂—, —C(H)(CH₃)—, —CH₂CH₂—, —CH₂CH₂CH₂—, —CH₂CH(CH₃)—, —CH₂CH₂CH₂CH₂—, —CH₂CH(CH₃)CH₂—, and —CH₂C(CH₃)₂CH₂—.

The term “alkoxy”, as used herein, means a straight or branched, saturated hydrocarbon chain containing from 1 to 6 carbon atoms and —O— terminating the hydrocarbon chain. The term “C_{X}-C_{y} alkoxy”, as used herein, means a straight or branched chain, saturated hydrocarbon containing x to y carbon atoms and —O— terminating the hydrocarbon chain. For example “C₁-C₆ alkoxy” means a straight or branched chain, saturated hydrocarbon containing 1 to 6 carbon atoms and —O— terminating the hydrocarbon chain. Examples of an alkoxy include, but are not limited to, methoxy, ethoxy, n-propoxy, iso-propoxy, n-butoxy, sec-butoxy, iso-butoxy, tert-butoxy, n-pentoxy, isopentoxy, neopentoxy, n-hexoxy, 2-methylpentoxy, and 2,2-dimethylbutoxy.

The term “alkynyl”, as used herein, means a straight or branched chain hydrocarbon group containing from 2 to 10 carbon atoms and containing at least one carbon-carbon triple bond. The term “C₂-C₄ alkynyl”, as used herein, means an alkynyl group containing from 2 to 4 carbon atoms. Representative examples of alkynyl include, but are not limited, to acetylenyl, 1-propynyl, 2-propynyl, 3-butynyl, 2-pentynyl, 1-butynyl, and 2-butynyl.

The term “aryl”, as used herein, means a phenyl or a bicyclic aryl. The bicyclic aryl may be naphthyl, or a phenyl fused to a monocyclic cycloalkyl, or a phenyl fused to a monocyclic cycloalkenyl. Non-limiting examples of the aryl groups include phenyl, dihydroindenyl (e.g. 2,3-dihydro-1H-inden-1-yl), indenyl, naphthyl, dihydronaphthalenyl, and tetrahydronaphthalenyl (e.g. 1,2,3,4-tetrahydronaphthalen-1-yl). The aryl groups can be unsubstituted or substituted, e.g., with alkyl, halo, haloalkyl, alkoxy, cyano, heterocyclo, etc., and the bicyclic aryl is attached to the parent molecular moiety through any substitutable carbon atom contained within the bicyclic ring system.

The term “cycloalkyl” or “cycloalkane”, as used herein, means a monocyclic or a bicyclic ring system. The term “monocyclic cycloalkyl”, as used herein, is a carbocyclic ring system containing three to eight carbon atoms, such as three to six carbon atoms, zero heteroatoms and zero double bonds. Examples of monocyclic ring systems include cyclopropyl, cyclobutyl, cyclopentyl, cyclohexyl, cycloheptyl, and cyclooctyl. The bicyclic cycloalkyl is a monocyclic cycloalkyl fused to a monocyclic cycloalkyl ring. The monocyclic or bicyclic cycloalkyl ring may contain one or two alkylene bridges, each consisting of one, two, three, or four carbon atoms, each linking two non-adjacent carbon atoms of the ring system. Non-limiting examples of such bridged cycloalkyl ring systems include bicyclo[3.1.1]heptane, bicyclo[2.2.1]heptane, bicyclo[2.2.2]octane, bicyclo[3.2.2]nonane, bicyclo[3.3.1]nonane, bicyclo[4.2.1]nonane, tricyclo[3.3.1.0^{3,7}]nonane (octahydro-2,5-methanopentalene or noradamantane), and tricyclo[3.3.1.1^{3,7}]decane (adamantane). The monocyclic and the bicyclic cycloalkyls can be unsubstituted or substituted, e.g., with alkyl, halo, haloalkyl, alkoxy, cyano, heterocyclo, etc., and are attached to the parent molecular moiety through any substitutable atom contained within the ring system.

The term “cycloalkenyl” or “cycloalkene”, as used herein, means a monocyclic or a bicyclic hydrocarbon ring system. The monocyclic cycloalkenyl has four, five, six, seven, eight, nine, or ten carbon atoms, e.g., C₄-C₁₀, or C₅-C₁₀ cycloalkenyl, and zero heteroatoms. The four-membered ring systems have one double bond, the five- or six-membered ring systems have one or two double bonds, and the seven- or eight-membered ring systems have one, two, or three double bonds. Representative examples of monocyclic cycloalkenyl groups include, but are not limited to, cyclobutenyl, cyclopentenyl, cyclohexenyl, cycloheptenyl, cyclooctenyl, cyclononenyl, cyclodecenyl. The bicyclic cycloalkenyl is a monocyclic cycloalkenyl fused to a monocyclic cycloalkyl group, or a monocyclic cycloalkenyl fused to a monocyclic cycloalkenyl group. The monocyclic or bicyclic cycloalkenyl ring may contain one or two alkylene bridges, each consisting of one, two, three, or four carbon atoms, each linking two non-adjacent carbon atoms of the ring system. Representative examples of the bicyclic cycloalkenyl groups include, but are not limited to, 4,5,6,7-tetrahydro-3aH-indene, octahydronaphthalenyl, and 1,6-dihydro-pentalene. The monocyclic and bicyclic cycloalkenyl can be unsubstituted or substituted, e.g., with alkyl, halo, haloalkyl, alkoxy, cyano, heterocyclo, etc., and are attached to the parent molecular moiety through any substitutable atom contained within the ring systems, and can be unsubstituted or substituted.

The term “halo” or “halogen”, as used herein, means Cl, Br, I, or F.

The term “haloalkyl”, as used herein, means an alkyl group, as defined herein, in which one, two, three, four, five or six hydrogen atoms are replaced by halogen. The term “C₁-C₆ haloalkyl”, as used herein, means a C₁-C₆ alkyl group, as defined herein, in which one, two, three, four, five, or six hydrogen atoms are replaced by halogen. The term “C₁-C₄ haloalkyl”, as used herein, means a C₁-C₄ alkyl group, as defined herein, in which one, two, three, four, five, or six hydrogen atoms are replaced by halogen. Representative examples of haloalkyl include, but are not limited to, chloromethyl, fluoromethyl, 2-fluoroethyl, 2,2,2-trifluoroethyl, trifluoromethyl, difluoromethyl, pentafluoroethyl, 2-chloro-3-fluoropentyl, trifluorobutyl (such as, but not limited to, 4,4,4-trifluorobutyl), and trifluoropropyl (such as, but not limited thereto, 3,3,3-trifluoropropyl).

The term “haloaryl”, as used herein, means a phenyl or bicyclic aryl in which one, two, three, four, five, six, seven, or eight hydrogen atoms are replaced by halogen. Non-limiting examples of the aryl groups include fluorophenyl, chlorophenyl, bromophenyl, iodophenyl, fluoro-, chloro-, bromo-, or iodo-dihydroindenyl (e.g. 2,3-dihydro-1H-inden-1-yl), fluoro-, chloro-, bromo-, or iodo-indenyl, fluoro-, chloro-, bromo-, or iodo-naphthyl, fluoro-, chloro-, bromo-, or iodo-dihydronaphthalenyl, and fluoro-, chloro-, bromo-, or iodo-tetrahydronaphthalenyl (e.g. 1,2,3,4-tetrahy dronaphthalen-1-yl).

The term “heterocycle” or “heterocyclic”, as used herein, means a monocyclic heterocycle or a bicyclic heterocycle. The monocyclic heterocycle, as used herein, is a three-, four-, five-, six-, seven-, eight-, nine-, or ten-membered ring containing at least one heteroatom independently selected from the group consisting of O, N, and S. The three-, four-, five-, six-, seven-, eight-, nine-, or ten-membered ring contains zero or one double bond, and one heteroatom selected from the group consisting of O, N, and S. The four-, five-, six-, seven-, eight-, nine-, or ten-membered heterocyclic ring contains zero or one double bond, and one heteroatom selected from the group consisting of O, N, and S. The five-membered ring contains zero or one double bond and one, two, or three heteroatoms selected from the group consisting of O, N, and S. The six-membered ring contains zero, one, or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Non-limiting examples of monocyclic heterocycles include azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle, as used herein, is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle. Non-limiting examples of bicyclic heterocycles include e.g. dihydrochromenyl (e.g. 3,4-dihydro-2H-chromen-4-yl), benzopyranyl, benzothiopyranyl, 2,3-dihydrobenzofuranyl, 2,3-dihydrobenzothienyl, and 2,3-dihydro-1H-indolyl. The monocyclic and the bicyclic heterocycles may contain an alkenylene bridge of two, three, or four carbon atoms, or one or two alkylene bridges of 1, 2, 3, or 4 carbon atoms, or combinations thereof, wherein each bridge links two non-adjacent atoms of the ring system. Non-limiting examples of such bridged heterocycles include octahydro-2,5-epoxypentalene, azabicyclo[2.2.1]heptyl (including 2-azabicyclo[2.2.1]hept-2-yl), hexahydro-2H-2,5-methanocyclopenta[b]furan, hexahydro-1H-1,4-methanocyclopenta[c]furan, aza-admantane (1-azatricyclo[3.3.1.1³° 7]decane), and oxa-adamantane (2-oxatricyclo[3.3.1.1³° 7]decane). In some embodiments, a 4- to 10-membered ring heterocyclyl may be selected from among 2,3-dihydrofuranyl, 2,5-dihydrofuranyl, tetrahydrofuranyl, 1,3-dioxolanyl, 1,3-dioxanyl, tetrahydro-2H-pyranyl, 3,4-dihydro-2H-pyranyl, 3,6-dihydro-2H-pyranyl, 2H-pyranyl, 4H-pyranyl, pyrrolidinyl, 2,3-dihydro-1H-pyrrolyl, 2,5-dihydro-1H-pyrrolyl, 4H-1,3-dioxinyl, 1,4-dioxanyl, 2,3-dihydro-1,4-dioxinyl, piperidinyl, 2-oxa-7-azaspiro[3.5]nonanyl, 1,2-dihydropyridinyl, 1,4-dihydropyridinyl, 2,3-dihydropyridinyl, 3,4-dihydropyridinyl, 1,2,3,6-tetrahydropyridinyl, isoxazolidinyl, oxazolidinyl, 2,3-dihydroisoxazolyl, 2,5-dihydroisoxazolyl, and morpholino, each of which may be substituted or unsubstituted. The monocyclic and the bicyclic heterocycles can be unsubstituted or substituted, e.g., with alkyl, halo, haloalkyl, alkoxy, cyano, heterocyclo, cycloalkyl, sulfonyl, etc., and are connected to the parent molecular moiety through any substitutable carbon atom or any substitutable nitrogen atom contained within the rings. The nitrogen and sulfur heteroatoms in the heterocycle rings may optionally be oxidized and the nitrogen atoms may optionally be quaternized.

The term “heteroaryl”, as used herein, means a monocyclic heteroaryl or a bicyclic heteroaryl. The heteroaryl may comprise 5- to 10-membered ring. The monocyclic heteroaryl is a five- or six-membered ring. The five-membered ring contains two double bonds. The five membered ring may contain one heteroatom selected from O or S; or one, two, three, or four nitrogen atoms and optionally one oxygen or one sulfur atom. The six-membered ring contains three double bonds and one, two, three or four nitrogen atoms. Representative examples of monocyclic heteroaryl include, but are not limited to, furanyl, imidazolyl, isoxazolyl, isothiazolyl, oxadiazolyl, 1,3-oxazolyl, pyridinyl, pyridazinyl, pyrimidinyl, pyrazinyl, pyrazolyl, pyrrolyl, tetrazolyl, thiadiazolyl, 1,3-thiazolyl, thienyl, triazolyl, and triazinyl. The bicyclic heteroaryl consists of a monocyclic heteroaryl fused to a phenyl, or a monocyclic heteroaryl fused to a monocyclic cycloalkyl, or a monocyclic heteroaryl fused to a monocyclic cycloalkenyl, or a monocyclic heteroaryl fused to a monocyclic heteroaryl, or a monocyclic heteroaryl fused to a monocyclic heterocycle. Non-limiting examples of bicyclic heteroaryl groups include benzofuranyl, benzothienyl, benzoxazolyl, benzimidazolyl, benzoxadiazolyl, 6,7-dihydro-5H-cyclopenta[b]pyridinyl (e.g. 6,7-dihydro-5H-cyclopenta[b]pyridin-7-yl), 6,7-dihydro-1,3-benzothiazolyl, imidazo[1,2-a]pyridinyl, indazolyl, indolyl, isoindolyl, isoquinolinyl, naphthyridinyl, pyridoimidazolyl, quinolinyl, thiazolo[5,4-b]pyridin-2-yl, thiazolo[5,4-d]pyrimidin-2-yl, and 5,6,7,8-tetrahydroquinolinyl (e.g. 5,6,7,8-tetrahydroquinolin-5-yl, 5,6,7,8-tetrahydroquinolin-8-yl). In some embodiments, 5- to 10-membered ring heteroaryl may be selected from among pyridinyl, pyrimidinyl, pyrazinyl, 1H-indolyl, 2H-indolyl, pyrazolyl, 1H-imidazolyl, oxazolyl, isoxazolyl, pyrazolyl, quinolinyl, isoquinolinyl, furo[3,2-b]pyridinyl, furo[4,3-b]pyridinyl, furo[5,4-b]pyridinyl, and benzo[c][1,2,5]oxadiazol-5-yl, each of which may be substituted or unsubstituted. The monocyclic and bicyclic heteroaryl groups can be substituted, e.g., with alkyl, halo, haloalkyl, alkoxy, cyano, heterocyclo, cycloalkyl, sulfonyl, etc., or unsubstituted and are connected to the parent molecular moiety through any substitutable carbon atom or any substitutable nitrogen atom contained within the ring systems.

The term “heteroatom”, as used herein, means a nitrogen, oxygen, or sulfur atom.

The term “oxo”, as used herein, means a ═O group.

The term “carbonyl”, as used herein, means a

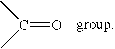

A “carbonyl” group may alternatively be disclosed as —C(O)—.

The term “carboxy” or “carboxyl”, as used herein, means a

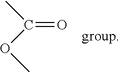

A “carboxy” or “carboxyl” group may alternatively be disclosed as —C(O)O—.

The term “hydroxy” or “hydroxyl”, as used herein, means a —OH group. In some embodiments, a hydroxy or hydroxyl group may be bonded to an alkyl thereby forming an hydroxyalkyl, such as, but not limited to hydroxymethyl, hydroxyethyl, etc.

The term “cyano”, as used herein, means a —C≡N group.

The term “imino”, as used herein, means a

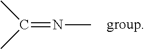

The imino may be bonded to one, two, or three groups, such as, but not limited to, alkyl, hydroxyl, alkoxy.

The term “thio”, as used herein, means a group comprising a —S— group.

The term “sulfonyl”, as used herein, means a group comprising a O group.

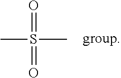

The term “phosphate”, as used herein, means a —PO₃H₂ group. One or both hydrogens in a phosphate may be replaced with cations, such as sodium or potassium.

“Treatment,” “treat,” or “treating”, as used herein, means to cure, reduce or to alleviate the existing symptoms of the subject being treated.

The term “subject”, as used herein, includes, but is not limited to, animals such as mammals, including, but not limited to, primates (e.g., humans), cows, sheep, goats, horses, dogs, cats, rabbits, rats, mice and the like. In preferred embodiments, the subject is a human.

### b. Compounds

Compounds of formula (I) have the following structure:

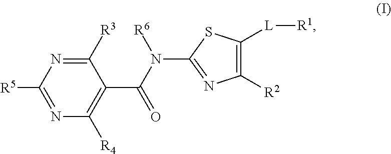

or a pharmaceutically acceptable salt thereof, wherein:

L is selected from the group consisting of a bond, —O—, —C(O)—, —C(O)O—, —C(O)N(R^{L})—, —S—, —S(O)—, and —S(O)₂—;

R^{L} is selected from the group consisting of hydrogen and C₁-C₆-alkyl;

R¹ is selected from the group consisting of hydrogen, halogen, cyano, imino, C₁-C₁₀-alkyl, C₂-C₁₀-alkenyl, C₂-C₁₀-alkynyl, C₃-C₆-cycloalkyl, C₅-C₁₀-cycloalkenyl, aryl, 4- to 10-membered ring heterocyclyl, and 5- to 10-membered ring heteroaryl; wherein:
the imino may be unsubstituted or substituted with one or two substituents independently selected from the group consisting of hydroxyl, C₁-C₆-alkyl, C₁-C₆-alkoxy, aryl and 5- to 10-membered ring heteroaryl;
the R¹ C₁-C₁₀-alkyl may be unsubstituted or substituted with one, two, or three substituents independently selected from the group consisting of halogen, amino, cyano, C₃-C₆-cycloalkyl, aryl, 5- to 10-membered ring heteroaryl, 4- to 10-membered ring heterocyclyl, —OR¹⁰¹, —C(O)OR¹⁰², and ═NR¹⁰³;
   wherein the amino, C₃-C₆-cycloalkyl, aryl, 5- to 10-membered ring heteroaryl, 4- to 10-membered ring heterocyclyl substituents of the R¹ C₁-C₁₀-alkyl may be unsubstituted or substituted with one, two, or three substituents independently selected from the group consisting of halogen, C₁-C₆-alkyl, C₁-C₆-alkoxy, C₃-C₆-cycloalkyl, halo-C₁-C₆-alkyl, and 4- to 10-membered ring heterocyclyl;
   wherein R¹⁰¹ and R¹⁰² are independently selected from the group consisting of hydrogen, C₁-C₆-alkyl, halo-C₁-C₆-alkyl, C₃-C₆-cycloalkyl, halo-C₃-C₆-cycloalkyl, C₁-C₆-alkyl-C₃-C₆-cycloalkyl, aryl, haloaryl, and C₁-C₆-alkylaryl; and
   wherein R¹⁰³ is selected from the group consisting of hydrogen, hydroxy, C₁-C₆-alkyl, and C₁-C₆-alkoxy;
the R¹ C₂-C₁₀-alkenyl and the R¹ C₂-C₁₀-alkynyl may be unsubstituted or substituted with one, two, or three substituents independently selected from the group consisting of hydroxy, C₁-C₆-alkoxy, C₃-C₆-cycloalkyl, aryloxy, 5- to 10-membered ring heteroaryloxy, C₁-C₆-alkoxycarbonyl, alkylsilyl, and cyano-C₁-C₆-alkylcarbonylamino;
the R¹ C₃-C₆-cycloalkyl and the R¹ C₃-C₆-cycloalkenyl may be unsubstituted or substituted with one, two, or three substituents independently selected from the group consisting of halogen, cyano, oxo, OH, C₁-C₆-alkyl, halo-C₁-C₆-alkyl, C₁-C₆-alkoxy, halo-C₁-C₆-alkoxy, C₁-C₆-alkoxycarbonyl, C₁-C₆-alkyoxycarbonylamino, and halo-C₁-C₆-alkoxycarbonyl;
the R¹ aryl and the R¹ 5- to 10-membered ring heteroaryl may be unsubstituted or substituted with one, two, or three substituents independently selected from the group consisting of halogen, cyano, C₁-C₆-alkyl, C₁-C₆ alkoxy, hydroxy-C₁-C₆-alkyl, halo-C₁-C₆-alkyl, cyano-C₁-C₆-alkyl, C₁-C₆-alkoxy-C₁-C₆-alkyl, C₁-C₆-alkoxycarbonyl-C₁-C₆-alkyl, C₃-C₆-cycloalkyl, C₃-C₆-cycloalkyl-C₁-C₆-alkyl, C₅-C₁₀-cycloalkenyl, aryl, aryl-C₁-C₆-alkyl, 4- to 10-membered ring heterocyclyl, 4- to 10-membered ring heterocyclyl-C₁-C₆-alkyl, 5- to 10-membered ring heteroaryl, 5- to 10-membered ring heteroaryl-C₁-C₆-alkyl, —OR¹⁰⁴, —C(O)OR¹⁰⁵, —N(R¹⁰⁶)C(O)R¹⁰⁷, —N(R¹⁰⁸)C(O)NR¹⁰⁹R¹¹⁰, —S(O)₂R¹¹¹, —S(O)₂NR¹¹²R¹¹³, and —NS(O)₂NR¹¹²R¹¹³;
   wherein the C₃-C₆-cycloalkyl, C₃-C₆-cycloalkyl-C₁-C₆-alkyl, C₅-C₁₀-cycloalkenyl, aryl, aryl-C₁-C₆-alkyl, 4- to 10-membered ring heterocyclyl, 4- to 10-membered ring heterocyclyl-C₁-C₆-alkyl, 5- to 10-membered ring heteroaryl, and 5- to 10-membered ring heteroaryl-C₁-C₆-alkyl of the R¹ aryl and the R¹ 5- to 10-membered ring heteroaryl may be unsubstituted or substituted with one, two, or three substituents independently selected from the group consisting of halogen, cyano, C₁-C₆-alkyl, halo-C₁-C₆-alkyl, cyano-C₁-C₆-alkyl, and C₁-C₆-alkoxycarbonyl;
   wherein R¹⁰⁴, R¹⁰⁵, R¹⁰⁷, R¹⁰⁹, R¹¹⁰, R¹¹¹, R¹¹², and R¹¹³ are independently selected from the group consisting of hydrogen, C₁-C₆-alkyl, C₁-C₆-alkoxyC₁-C₆-alkyl, halo-C₁-C₆-alkyl, cyano-C₁-C₆-alkyl, C₃-C₆-cycloalkyl, halo-C₃-C₆-cycloalkyl, 4- to 10-membered ring heterocyclyl, 4- to 10-membered ring heterocyclyl-C₁-C₆-alkyl, aryl, haloaryl, and C₁-C₆-alkylaryl; and
   wherein R¹⁰⁶ and R¹⁰⁸ are independently selected from the group consisting of hydrogen and C₁-C₆-alkyl; and
the R¹ 4- to 10-membered ring heterocyclyl may be unsubstituted or substituted with one, two, or three substituents independently selected from the group consisting of halogen, OH, C₁-C₆-alkyl, halo-C₁-C₆-alkyl, C₁-C₆-alkoxycarbonyl, 4- to 10-membered ring heterocyclyl, and —S(O)₂R¹¹⁴; wherein R¹¹⁴ is selected from the group consisting of hydrogen and C₁-C₆-alkyl;

R² is selected from the group consisting of C₁-C₆-alkyl, C₂-C₆-alkenyl, C₃-C₆-cycloalkyl, aryl, 4- to 10-membered ring heterocyclyl, and 5- to 10-membered ring heteroaryl; wherein:
the R² C₁-C₆-alkyl or the C₂-C₆-alkenyl may be unsubstituted or substituted with one, two, or three substituents independently selected from the group consisting of halogen, hydroxy, cyano, C₁-C₆-alkoxy, halo-C₁-C₆-alkoxy, C₃-C₆-cycloalkyl, halo-C₃-C₆-cycloalkyl, C₃-C₆-cycloalkoxy, halo-C₃-C₆-cycloalkoxy, aryl, haloaryl, aryloxy, haloaryloxy, and C₁-C₆-alkylaryloxy;
the R² C₃-C₆-cycloalkyl may be unsubstituted or substituted with one, two, or three substituents independently selected from the group consisting of halogen, C₁-C₆-alkyl, and halo-C₁-C₆-alkyl;
   the R² aryl and the R² 5- to 10-membered ring heteroaryl may be unsubstituted or substituted with one, two, or three substituents independently selected from the group consisting of halogen, hydroxy, cyano, C₁-C₆-alkyl, hydroxy-C₁-C₆-alkyl, halo-C₁-C₆-alkyl, cyano-C₁-C₆-alkyl, C₃-C₆-cycloalkyl, C₁-C₆-alkoxy, halo-C₁-C₆-alkoxy, —S(O)₂R²⁰¹, and —S(O)₂NR²⁰²R²⁰³; wherein R²⁰¹, R²⁰², and R²⁰³ are independently selected from the group consisting of hydrogen and C₁-C₆-alkyl; and
the R² 4- to 10-membered ring heterocyclyl may be unsubstituted or substituted with one, two, or three substituents independently selected from the group consisting of halogen, C₁-C₆-alkyl, and halo-C₁-C₆-alkyl;

R³ and R⁴ are independently selected from the group consisting of hydrogen, C₁-C₆-alkyl, C₁-C₆-alkoxy, C₁-C₆-alkylthio, and C₃-C₆-cycloalkyl, wherein the C₁-C₆-alkyl may be substituted with one, two, or three halogen;

R⁵ is selected from the group consisting of hydrogen, methyl, methoxy, and cyano; and

R⁶ is selected from the group consisting of hydrogen or —CH₂-phosphate.

In some embodiments, the R¹ comprises aryl or 5- to 10-membered ring heteroaryl selected from the group consisting of phenyl, pyridinyl, pyrimidinyl, pyrazinyl, 1H-indolyl, 2H-indolyl, pyrazolyl, 1H-imidazolyl, oxazolyl, isoxazolyl, pyrazolyl, quinolinyl, isoquinolinyl, furo[3,2-b]pyridinyl, furo[4,3-b]pyridinyl, furo[5,4-b]pyridinyl, and benzo[c][1,2,5]oxadiazol-5-yl, each of which may be substituted or unsubstituted.

In some embodiments, R¹ comprises C₃-C₆-cycloalkyl or C₃-C₆-cycloalkenyl selected from the group consisting of cyclopropyl, cyclobutyl, cyclobutenyl, cyclopropyl, cyclopropenyl, cyclohexyl, and cyclohexenyl, each of which may be substituted or unsubstituted.

In some embodiments, R¹ comprises 4- to 10-membered ring heterocyclyl selected from the group consisting of 2,3-dihydrofuranyl, 2,5-dihydrofuranyl, tetrahydrofuranyl, 1,3-dioxolanyl, 1,3-dioxanyl, tetrahydro-2H-pyranyl, 3,4-dihydro-2H-pyranyl, 3,6-dihydro-2H-pyranyl, 2H-pyranyl, 4H-pyranyl, pyrrolidinyl, 2,3-dihydro-1H-pyrrolyl, 2,5-dihydro-1H-pyrrolyl, 4H-1,3-dioxinyl, 1,4-dioxanyl, 2,3-dihydro-1,4-dioxinyl, piperidinyl, 2-oxa-7-azaspiro[3.5]nonanyl, 1,2-dihydropyridinyl, 1,4-dihydropyridinyl, 2,3-dihydropyridinyl, 3,4-dihydropyridinyl, 1,2,3,6-tetrahydropyridinyl, isoxazolidinyl, oxazolidinyl, 2,3-dihydroisoxazolyl, 2,5-dihydroisoxazolyl, and morpholino, each of which may be substituted or unsubstituted.

In some embodiments, R¹ is selected from the group consisting of hydrogen, methyl, ethyl, propyl, isopropyl, trifluoromethyl, ethenyl, ethynyl, propenyl, propynyl, t-butyl, butenyl, butynyl, cyano, iodo, chloro, fluoro, and bromo, each of which may be substituted or unsubstituted.

In some embodiments, R² is selected from the group consisting of methyl, trifluoromethyl, ethyl, n-propyl, isopropyl, tert-butyl, neopentyl, cyclopropylethyl, cyclopropyl, cyclobutyl, cyclopentyl, cyclohexyl, difluorocyclohexyl, cyclopropylvinyl, dimethylbutenyl, and fluorostyryl.

In some embodiments, R² is selected from the group consisting of phenyl, trifluoromethoxyphenyl, 4-fluoro-2-methoxyphenyl, para-fluorophenethyl, para-fluorophenoxyethyl, toylyoxymethyl, meta-trifluoromethylpyridinyl, para-trifluoromethylpyridinyl, para-fluoropyridinyl, para-trifluoromethoxypyridinyl, and para-difluoromethylpyridinyl.

In some embodiments, R⁵ and R⁶ are hydrogen.

In some embodiments, the compound of the present invention may have the following Formula (II-A):

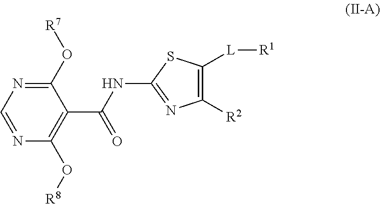

or a pharmaceutically acceptable salt thereof. In some embodiments, L, R¹, and R² of Formula (II-A) are as defined above in the context of Formula (I), and each may be substituted or unsubstituted as set forth above in the context of Formula (I). In some embodiments, R⁷ and R⁸ are independently selected from the group consisting of hydrogen, C₁-C₆-alkyl, and C₃-C₆-cycloalkyl. In some embodiments, R⁷ and R⁸ are both methyl. In some embodiments, R⁷ and R⁸ are both ethyl. In some embodiments, R⁷ is methyl, and R⁸ is ethyl.

In some embodiments of the compound of Formula (II-A), or the pharmaceutically acceptable salt thereof, R² is C₁-C₁₀ alkyl, which may be substituted or unsubstituted.

In some embodiments of the compound of Formula (II-A), or the pharmaceutically acceptable salt thereof, R² is C₁-C₁₀ alkyl substituted with C₃-C₆ cycloalkyl.

In some embodiments of the compound of Formula (II-A), or the pharmaceutically acceptable salt thereof, R² is C₂-C₁₀ alkenyl, which may be substituted or unsubstituted.

In some embodiments of the compound of Formula (II-A), or the pharmaceutically acceptable salt thereof, R² is C₂-C₁₀ alkenyl substituted with C₃-C₆ cycloalkyl.

In some embodiments of the compound of Formula (II-A), or the pharmaceutically acceptable salt thereof, R² is C₂-C₁₀ alkenyl substituted with aryl, which may be substituted or unsubstituted. In some embodiments, the aryl may be phenyl, substituted with one or two halo.

In some embodiments of the compound of Formula (II-A), or the pharmaceutically acceptable salt thereof, R² is C₃-C₆ cycloalkyl, which may be substituted or unsubstituted.

In some embodiments of the compound of Formula (II-A), or the pharmaceutically acceptable salt thereof, R² is C₃-C₆ cycloalkyl substituted one or two halo.

In some embodiments of the compound of Formula (II-A), or the pharmaceutically acceptable salt thereof, R² is aryl, which may be substituted or unsubstituted.

In some embodiments of the compound of Formula (II-A), or the pharmaceutically acceptable salt thereof, R² is phenyl substituted with one or two halo.

In some embodiments, the compound of the present invention may have the following Formula (II-B):

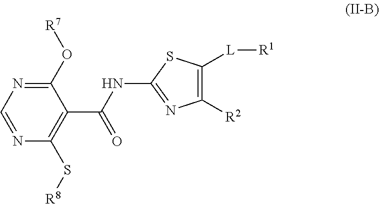

or a pharmaceutically acceptable salt thereof. In some embodiments, L, R¹, and R² of Formula (II-B) are as defined above in the context of Formula (I), and each may be substituted or unsubstituted as set forth above in the context of Formula (I). In some embodiments, R⁷ and R⁸ are independently selected from the group consisting of hydrogen, C₁-C₆-alkyl, and C₃-C₆-cycloalkyl. In some embodiments, R⁷ and R⁸ are both methyl. In some embodiments, R⁷ and R⁸ are both ethyl. In some embodiments, R⁷ is methyl, and R⁸ is ethyl.

In some embodiments of the compound of Formula (II-B), or the pharmaceutically acceptable salt thereof, R² is aryl, which may be substituted or unsubstituted.

In some embodiments of the compound of Formula (II-B), or the pharmaceutically acceptable salt thereof, R² is phenyl, which may be substituted with cyano.

In some embodiments, the compound of the present invention comprises a compound of Formula (I) in which R² is X, and X is selected from the group consisting of C(R¹⁰)₃, pyridine-C(R¹⁰)₃, and pyridine-O—C(R¹⁰)₃.

In some embodiments, the compound of the present invention comprises a compound of Formula (I) in which R² is X, the compound having the following Formula (III):

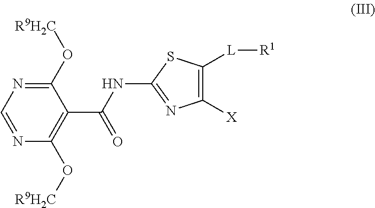

or a pharmaceutically acceptable salt thereof. In some embodiments, L and R¹ are as defined above in the context of Formula (I), and each may be substituted or unsubstituted as set forth above in the context of Formula (I). In some embodiments, X is either C(R¹⁰)₃, pyridine-C(R¹⁰)₃, or pyridine-O—C(R¹⁰)₃; wherein each R⁹ is independently —H or —CH₃; and each R¹⁰ is independently —H, —CH₃, or —F.

In some embodiments of the compound of Formula (III), or the pharmaceutically acceptable salt thereof, each R⁹ is —H and X is selected from the group consisting of —CF₃, pyridine-CF₃, and —C(CH₃)₃.

In some embodiments of the compound of Formula (III), or the pharmaceutically acceptable salt thereof, each R⁹ is —CH₃ and X is selected from the group consisting of —CF₃, pyridine-CF₃, and —C(CH₃)₃.

In some embodiments of the compound of Formula (III), or the pharmaceutically acceptable salt thereof, each R⁹ is —H, X is —CF₃, L is a bond, and R¹ is halo.

In some embodiments of the compound of Formula (III), or the pharmaceutically acceptable salt thereof, each R⁹ is —H, X is —CF₃, L is a bond, and R¹ is iodo.

In some embodiments of the compound of Formula (III), or the pharmaceutically acceptable salt thereof, each R⁹ is —H, X is —CF₃, L is a bond, and R¹ is chloro.

In some embodiments of the compound of Formula (III), or the pharmaceutically acceptable salt thereof, each R⁹ is —H, X is —CF₃, L is a bond, and R¹ is cyano.

In some embodiments of the compound of Formula (III), or the pharmaceutically acceptable salt thereof, each R⁹ is —H or —CH₃, X is —CF₃, L is a bond, and R¹ is C₁-C₁₀ alkyl, which may be substituted or unsubstituted. In some embodiments, R¹ is methyl. In some embodiments, R¹ is ethyl. In some embodiments, R¹ is n-propyl.

In some embodiments of the compound of Formula (III), or the pharmaceutically acceptable salt thereof, each R⁹ is —H or —CH₃, X is —CF₃, L is a bond, and R¹ is C₁-C₁₀ alkyl, which may be substituted or unsubstituted. In some embodiments, R¹ is a substituted methyl. In some embodiments, R¹ is a substituted ethyl. In some embodiments, R¹ is a substituted n-propyl.

In some embodiments of the compound of Formula (III), or the pharmaceutically acceptable salt thereof, each R⁹ is —H, X is —CF₃, L is a bond, and R¹ is C₁-C₁₀ alkyl, which may be substituted or unsubstituted. In some embodiments, R¹ is a substituted methyl. In some embodiments, R¹ is a substituted ethyl. In some embodiments, R¹ is a substituted n-propyl.

In some embodiments of the compound of Formula (III), or the pharmaceutically acceptable salt thereof, each R⁹ is —H or —CH₃, X is —CF₃, L is a bond, and R¹ is C₁-C₁₀ alkyl, substituted with OR¹⁰¹. In some embodiments, R¹⁰¹ is hydrogen. In some embodiments, R¹⁰¹ is C₁-C₆-alkyl.

In some embodiments of the compound of Formula (III), or the pharmaceutically acceptable salt thereof, each R⁹ is —H, X is —CF₃, L is a bond, and R¹ is C₁-C₁₀ alkyl, substituted with OR¹⁰¹. In some embodiments, R¹⁰¹ is hydrogen. In some embodiments, R¹⁰¹ is C₁-C₆-alkyl.

In some embodiments of the compound of Formula (III), or the pharmaceutically acceptable salt thereof, each R⁹ is —H or —CH₃, X is —CF₃, L is a bond, and R¹ is C₁-C₁₀ alkyl, substituted with OR¹⁰¹. In some embodiments, R¹⁰¹ is methyl.

In some embodiments of the compound of Formula (III), or the pharmaceutically acceptable salt thereof, each R⁹ is —H, X is —CF₃, L is a bond, and R¹ is C₁-C₁₀ alkyl, substituted with OR¹⁰¹. In some embodiments, R¹⁰¹ is methyl.

In some embodiments of the compound of Formula (III), or the pharmaceutically acceptable salt thereof, each R⁹ is —H, X is —CF₃, L is a bond, and R¹ is methyl, substituted with OR¹⁰¹. In some embodiments, R¹⁰¹ is methyl.

In some embodiments of the compound of Formula (III), or the pharmaceutically acceptable salt thereof, each R⁹ is —H or —CH₃, X is —CF₃, L is a bond, and R¹ is C₁-C₁₀ alkyl, substituted with amino. In some embodiments, the amino may be substituted with one or two C₁-C₆-alkyl. The C₁-C₆-alkyl may be, but not limited to, methyl, ethyl, n-propyl, isopropyl, n-butyl, sec-butyl, isobutyl, or tert-butyl.

In some embodiments of the compound of Formula (III), or the pharmaceutically acceptable salt thereof, each R⁹ is —H or —CH₃, X is —CF₃, L is a bond, and R¹ is C₁-C₁₀ alkyl, substituted with 4- to 10-membered ring heterocyclyl, which may be substituted or unsubstituted.

In some embodiments of the compound of Formula (III), or the pharmaceutically acceptable salt thereof, each R⁹ is —H or —CH₃, X is —CF₃, L is a bond, and R¹ is C₁-C₁₀ alkyl, substituted with 4- to 10-membered ring heterocyclyl that is further substituted with one or two halo. In some embodiments, the halo may be fluoro.

In some embodiments of the compound of Formula (III), or the pharmaceutically acceptable salt thereof, each R⁹ is —H or —CH₃, X is —CF₃, L is a bond, and R¹ is C₁-C₁₀ alkyl, substituted with 4- to 10-membered ring heterocyclyl that is further substituted with C₁-C₆ alkoxy.

In some embodiments of the compound of Formula (III), or the pharmaceutically acceptable salt thereof, each R⁹ is —H or —CH₃, X is —CF₃, L is a bond, and R¹ is C₁-C₁₀ alkyl, substituted with 4- to 10-membered ring heterocyclyl that is further substituted with 4- to 10-membered ring heterocyclyl.

In some embodiments of the compound of Formula (III), or the pharmaceutically acceptable salt thereof, each R⁹ is —H or —CH₃, X is —CF₃, L is a bond, and R¹ is C₁-C₁₀ alkyl, substituted with aryl. In some embodiments, aryl comprises phenyl.

In some embodiments of the compound of Formula (III), or the pharmaceutically acceptable salt thereof, each R⁹ is —H, X is —CF₃, L is a bond, and R¹ is C₂-C₁₀-alkenyl, which may be substituted or unsubstituted.

In some embodiments of the compound of Formula (III), or the pharmaceutically acceptable salt thereof, each R⁹ is —H, X is —CF₃, L is a bond, and R¹ is C₂-C₁₀-alkenyl, substituted with C₁-C₆ alkoxy.

In some embodiments of the compound of Formula (III), or the pharmaceutically acceptable salt thereof, each R⁹ is —H, X is —CF₃, L is a bond, and R¹ is C₂-C₁₀-alkynyl, which may be substituted or unsubstituted.

In some embodiments of the compound of Formula (III), or the pharmaceutically acceptable salt thereof, each R⁹ is —H, X is —CF₃, L is a bond, and R¹ is C₂-C₁₀-alkynyl, which may be substituted with C₃-C₆ cycloalkyl.

In some embodiments of the compound of Formula (III), or the pharmaceutically acceptable salt thereof, each R⁹ is —H, X is —CF₃, L is a bond, and R¹ is C₂-C₁₀-alkynyl, which may be substituted with C₁-C₆ alkoxy.

In some embodiments of the compound of Formula (III), or the pharmaceutically acceptable salt thereof, each R⁹ is —H, X is —CF₃, L is a bond, and R¹ is C₂-C₁₀-alkynyl, which may be substituted with hydroxyalkyl.

In some embodiments of the compound of Formula (III), or the pharmaceutically acceptable salt thereof, each R⁹ is —H, X is —CF₃, L is a bond, and R¹ is C₂-C₁₀-alkynyl, which may be substituted with alkylsilyl, such as trialkylsilyl.

In some embodiments of the compound of Formula (III), or the pharmaceutically acceptable salt thereof, each R⁹ is —H, X is —CF₃, L is a bond, and R¹ is aryl, which may be substituted or unsubstituted.

In some embodiments of the compound of Formula (III), or the pharmaceutically acceptable salt thereof, each R⁹ is —H, X is —CF₃, L is a bond, and R¹ is phenyl substituted with C₁-C₆ alkyl, which may be substituted or unsubstituted.

In some embodiments of the compound of Formula (III), or the pharmaceutically acceptable salt thereof, each R⁹ is —H, X is —CF₃, L is a bond, and R¹ is phenyl substituted with cyano.

In some embodiments of the compound of Formula (III), or the pharmaceutically acceptable salt thereof, each R⁹ is —H, X is —CF₃, L is a bond, and R¹ is phenyl substituted with halogen.

In some embodiments of the compound of Formula (III), or the pharmaceutically acceptable salt thereof, each R⁹ is —H, X is —CF₃, L is a bond, and R¹ is phenyl substituted with fluoro.

In some embodiments of the compound of Formula (III), or the pharmaceutically acceptable salt thereof, each R⁹ is —H, X is —CF₃, L is a bond, and R¹ is phenyl substituted with chloro.

In some embodiments of the compound of Formula (III), or the pharmaceutically acceptable salt thereof, each R⁹ is —H, X is —CF₃, L is a bond, and R¹ is phenyl substituted with bromo.

In some embodiments of the compound of Formula (III), or the pharmaceutically acceptable salt thereof, each R⁹ is —H, X is —CF₃, L is a bond, and R¹ is phenyl substituted with cyano.

In some embodiments of the compound of Formula (III), or the pharmaceutically acceptable salt thereof, each R⁹ is —H, X is —CF₃, L is a bond, and R¹ is phenyl substituted with cyano-C₁-C₆-alkyl.

In some embodiments of the compound of Formula (III), or the pharmaceutically acceptable salt thereof, each R⁹ is —H, X is —CF₃, L is a bond, and R¹ is phenyl substituted with —OR¹⁰⁴. In some embodiments, R¹⁰⁴ is C₁-C₆-alkyl. In some embodiments, R¹⁰⁴ is hydrogen.

In some embodiments of the compound of Formula (III), or the pharmaceutically acceptable salt thereof, each R⁹ is —H, X is —CF₃, L is a bond, and R¹ is phenyl substituted with —S(O)₂NR²R¹¹³. In some embodiments, each of R¹¹² and R¹¹³ are independently C₁-C₆-alkyl. In some embodiments, each of R¹¹² and R¹¹³ are independently C₁-C₆-alkoxyC₁-C₆-alkyl.

In some embodiments of the compound of Formula (III), or the pharmaceutically acceptable salt thereof, each R⁹ is —H, X is —CF₃, L is a bond, and R¹ is phenyl substituted with —NS(O)₂NR¹¹²R¹¹³. In some embodiments, each of R¹¹² and R¹¹³ are independently C₁-C₆-alkyl. In some embodiments, each of R¹¹² and R¹¹³ are independently C₁-C₆-alkoxyC₁-C₆-alkyl.

In some embodiments of the compound of Formula (III), or the pharmaceutically acceptable salt thereof, each R⁹ is —H, X is —CF₃, L is a bond, and R¹ is phenyl substituted with —S(O)₂R¹¹¹ and, R¹¹¹ is 4- to 10-membered ring heterocyclyl.

In some embodiments of the compound of Formula (III), or the pharmaceutically acceptable salt thereof, each R⁹ is —H, X is —CF₃, L is a bond, and R¹ is phenyl substituted with 5- to 10-membered ring heteroaryl-C₁-C₆-alkyl.

In some embodiments of the compound of Formula (III), or the pharmaceutically acceptable salt thereof, each R⁹ is —H, X is —CF₃, L is a bond, and R¹ is 5- to 10-membered ring heteroaryl, which may be substituted or unsubstituted.

In some embodiments of the compound of Formula (III), or the pharmaceutically acceptable salt thereof, each R⁹ is —H, X is —CF₃, L is a bond, and R¹ is 5- to 10-membered ring heteroaryloxy, which may be substituted or unsubstituted.

In some embodiments of the compound of Formula (III), or the pharmaceutically acceptable salt thereof, each R⁹ is —H, X is —CF₃, L is a bond, and R¹ is selected from the group consisting of pyridine, pyrazole, pyrimidine, quinoline, isoxazole, benzo[c][1,2,5]oxadiazole, and furo[3,2-b]pyridine, each of which may be substituted or unsubstituted.

In some embodiments of the compound of Formula (III), or the pharmaceutically acceptable salt thereof, each R⁹ is —H, X is —CF₃, L is a bond, and R¹ is pyridine substituted with C₁-C₆-alkyl.

In some embodiments of the compound of Formula (III), or the pharmaceutically acceptable salt thereof, each R⁹ is —H, X is —CF₃, L is a bond, and R¹ is pyridine substituted with C₁-C₄-alkoxy.

In some embodiments of the compound of Formula (III), or the pharmaceutically acceptable salt thereof, each R⁹ is —H, X is —CF₃, L is a bond, and R¹ is pyridine substituted with cyano-C₁-C₆-alkyl.

In some embodiments of the compound of Formula (III), or the pharmaceutically acceptable salt thereof, each R⁹ is —H, X is —CF₃, L is a bond, and R¹ is pyridine substituted with cyano.

In some embodiments of the compound of Formula (III), or the pharmaceutically acceptable salt thereof, each R⁹ is —H, X is —CF₃, L is a bond, and R¹ is pyridine substituted with cyano and at least one other substituent.

In some embodiments of the compound of Formula (III), or the pharmaceutically acceptable salt thereof, each R⁹ is —H, X is —CF₃, L is a bond, and R¹ is pyridine substituted with C₃-C₆-cycloalkyl, which may be substituted or unsubstituted. In some embodiments, the C₃-C₆-cycloalkyl may be substituted with cyano.

In some embodiments of the compound of Formula (III), or the pharmaceutically acceptable salt thereof, each R⁹ is —H, X is —CF₃, L is a bond, and R¹ is pyridine substituted with 4- to 10-membered ring heterocyclyl, which may be substituted or unsubstituted.

In some embodiments of the compound of Formula (III), or the pharmaceutically acceptable salt thereof, each R⁹ is —H, X is —CF₃, L is a bond, and R¹ is pyridine substituted with morpholine, which may be substituted or unsubstituted.

In some embodiments of the compound of Formula (III), or the pharmaceutically acceptable salt thereof, each R⁹ is —H, X is —CF₃, L is a bond, and R¹ is pyridine substituted with halo.

In some embodiments of the compound of Formula (III), or the pharmaceutically acceptable salt thereof, each R⁹ is —H, X is —CF₃, L is a bond, and R¹ is pyridine substituted with fluoro.

In some embodiments of the compound of Formula (III), or the pharmaceutically acceptable salt thereof, each R⁹ is —H, X is —CF₃, L is a bond, and R¹ is pyridine substituted with chloro.

In some embodiments of the compound of Formula (III), or the pharmaceutically acceptable salt thereof, each R⁹ is —H, X is —CF₃, L is a bond, and R¹ is pyridine substituted with halo-C₁-C₆-alkyl. In some embodiments, the halo-C₁-C₆-alkyl may be trifluoromethyl.

In some embodiments of the compound of Formula (III), or the pharmaceutically acceptable salt thereof, each R⁹ is —H, X is —CF₃, L is a bond, and R¹ is pyridine substituted with halo-C₁-C₆-alkoxy.

In some embodiments of the compound of Formula (III), or the pharmaceutically acceptable salt thereof, each R⁹ is —H, X is —CF₃, L is a bond, and R¹ is pyridine substituted with —OR¹⁰⁴. In some embodiments, R¹⁰⁴ is C₁-C₆-alkyl.

In some embodiments of the compound of Formula (III), or the pharmaceutically acceptable salt thereof, each R⁹ is —H, X is —CF₃, L is a bond, and R¹ is pyridine substituted with —OR¹⁰⁴. In some embodiments, R¹⁰⁴ is hydrogen.

In some embodiments of the compound of Formula (III), or the pharmaceutically acceptable salt thereof, each R⁹ is —H, X is —CF₃, L is a bond, and R¹ is pyridine substituted with —OR¹⁰⁴. In some embodiments, R¹⁰⁴ is C₁-C₆-alkyl substituted with 4- to 10-membered heterocyclyl, which may be substituted or unsubstituted.

In some embodiments of the compound of Formula (III), or the pharmaceutically acceptable salt thereof, each R⁹ is —H, X is —CF₃, L is a bond, and R¹ is pyridine substituted with cyano and with —OR⁴. In some embodiments, R¹⁰⁴ is C₁-C₆-alkyl substituted with 4- to 10-membered heterocyclyl, which may be substituted or unsubstituted.

In some embodiments of the compound of Formula (III), or the pharmaceutically acceptable salt thereof, each R⁹ is —H, X is —CF₃, L is a bond, and R¹ is pyridine substituted with cyano and with —OR¹⁰⁴. In some embodiments, R¹⁰⁴ is methyl substituted with 4- to 10-membered heterocyclyl, which may be substituted or unsubstituted.

In some embodiments of the compound of Formula (III), or the pharmaceutically acceptable salt thereof, each R⁹ is —H, X is —CF₃, L is a bond, and R¹ is pyridine substituted with cyano and with —OR¹⁰⁴. In some embodiments, R¹⁰⁴ is methyl substituted with oxetanyl.

In some embodiments of the compound of Formula (III), or the pharmaceutically acceptable salt thereof, each R⁹ is —H, X is —CF₃, L is a bond, and R¹ is pyrazole substituted with C₁-C₆-alkyl, which may be substituted or unsubstituted. In some embodiments, C₁-C₆-alkyl is substituted with C₃-C₆-cycloalkyl.

In some embodiments of the compound of Formula (III), or the pharmaceutically acceptable salt thereof, each R⁹ is —H, X is —CF₃, L is a bond, and R¹ is pyrazole substituted with halo. In some embodiments, the halo may be fluoro. In some embodiments, the halo may be chloro.

In some embodiments of the compound of Formula (III), or the pharmaceutically acceptable salt thereof, each R⁹ is —H, X is —CF₃, L is a bond, and R¹ is pyrazole substituted with halo-C₁-C₆-alkyl. In some embodiments, the halo-C₁-C₆-alkyl may be trifluoromethyl.

In some embodiments of the compound of Formula (III), or the pharmaceutically acceptable salt thereof, each R⁹ is —H, X is —CF₃, L is a bond, and R¹ is pyrazole substituted with C₃-C₆-cycloalkyl, which may be substituted or unsubstituted.

In some embodiments of the compound of Formula (III), or the pharmaceutically acceptable salt thereof, each R⁹ is —H, X is —CF₃, L is a bond, and R¹ is pyrimidine, substituted with halo. In some embodiments, the halo may be fluoro. In some embodiments, the halo may be chloro.

In some embodiments of the compound of Formula (III), or the pharmaceutically acceptable salt thereof, each R⁹ is —H, X is —CF₃, L is a bond, and R¹ is pyrimidine substituted with halo-C₁-C₆-alkyl. In some embodiments, the halo-C₁-C₆-alkyl may be trifluoromethyl.

In some embodiments of the compound of Formula (III), or the pharmaceutically acceptable salt thereof, each R⁹ is —H, X is —CF₃, L is a bond, and R¹ is pyrimidine substituted with C₃-C₆-cycloalkyl, which may be substituted or unsubstituted.

In some embodiments of the compound of Formula (III), or the pharmaceutically acceptable salt thereof, each R⁹ is —H, X is —CF₃, L is a bond, and R¹ is pyrimidine substituted with 4- to 10-membered heterocyclyl, which may be substituted or unsubstituted.

In some embodiments of the compound of Formula (III), or the pharmaceutically acceptable salt thereof, each R⁹ is —H, X is —CF₃, L is a bond, and R¹ is pyrimidine substituted with cyano.

In some embodiments of the compound of Formula (III), or the pharmaceutically acceptable salt thereof, each R⁹ is —H, X is —CF₃, L is a bond, and R¹ is pyrimidine substituted with C₁-C₆ alkoxy.

In some embodiments of the compound of Formula (III), or the pharmaceutically acceptable salt thereof, each R⁹ is —H, X is —CF₃, L is a bond, and R¹ is quinoline, which may be substituted or unsubstituted.

In some embodiments of the compound of Formula (III), or the pharmaceutically acceptable salt thereof, each R⁹ is —H, X is —CF₃, L is a bond, and R¹ is isoxazole, which may be substituted or unsubstituted.

In some embodiments of the compound of Formula (III), or the pharmaceutically acceptable salt thereof, each R⁹ is —H, X is —CF₃, L is a bond, and R¹ is benzo[c][1,2,5]oxadiazole, which may be substituted or unsubstituted.

In some embodiments of the compound of Formula (III), or the pharmaceutically acceptable salt thereof, each R⁹ is —H, X is —CF₃, L is a bond, and R¹ is furo[3,2-b]pyridine, which may be substituted or unsubstituted

In some embodiments of the compound of Formula (III), or the pharmaceutically acceptable salt thereof, each R⁹ is —H, X is —CF₃, L is a bond, and R¹ is C₃-C₆-cycloalkyl, which may be substituted or unsubstituted.

In some embodiments of the compound of Formula (III), or the pharmaceutically acceptable salt thereof, each R⁹ is —H, X is —CF₃, L is a bond, and R¹ is C₅-C₁₀-cycloalkenyl, which may be substituted or unsubstituted.

In some embodiments of the compound of Formula (III), or the pharmaceutically acceptable salt thereof, each R⁹ is —H, X is —CF₃, L is a bond, and R¹ is C₅-C₁₀-cycloalkenyl, substituted with cyano.

In some embodiments of the compound of Formula (III), or the pharmaceutically acceptable salt thereof, each R⁹ is —H, X is —CF₃, L is a bond, and R¹ is 4- to 10-membered ring heterocyclyl, which may be substituted or unsubstituted. In some embodiments, R¹ is 3,6-dihydro-2H-pyran. In some embodiments, R¹ is 3,4-dihydro-2H-pyran. In some embodiments, R¹ is 2,3-dihydrofuran. In some embodiments, R¹ is tetrahydrofuran. In some embodiments, R¹ is tetrahydro-2H-pyran. In some embodiments, R¹ is morpholine. In some embodiments, R¹ is 1,3-dioxolane. In some embodiments, R¹ is 1,2,3,6-tetrahydropyridine. In some embodiments, R¹ is 4,5-dihydroisoxazole.

In some embodiments of the compound of Formula (III), or the pharmaceutically acceptable salt thereof, each R⁹ is —H, X is —CF₃, L is a bond, and R¹ is substituted imino. In some embodiments, the imino is substituted with one or more of phenyl, pyridine, hydroxyl, C₁-C₆ alkoxy. The phenyl and pyridine may be substituted with one or two halo.

In some embodiments of the compound of Formula (III), or the pharmaceutically acceptable salt thereof, each R⁹ is —H, X is —CF₃, L is a bond, and R¹ is substituted sulfonyl.

In some embodiments of the compound of Formula (III), or the pharmaceutically acceptable salt thereof, each R⁹ is —H, X is —CF₃, L is a bond, and R¹ is substituted sulfinyl.

In some embodiments of the compound of Formula (III), or the pharmaceutically acceptable salt thereof, each R⁹ is —H, X is —CF₃, L is a bond, and R¹ is substituted thio.

In some embodiments of the compound of Formula (III), or the pharmaceutically acceptable salt thereof, each R⁹ is —H, X is —CF₃, L is carboxyl, i.e., —C(O)O—, and R¹ is C₁-C₁₀ alkyl.

In some embodiments of the compound of Formula (III), or the pharmaceutically acceptable salt thereof, each R⁹ is —H, X is —CF₃, L is —C(O)—, and R¹ is C₁-C₁₀ alkyl.

In some embodiments of the compound of Formula (III), or the pharmaceutically acceptable salt thereof, each R⁹ is —H, X is —CF₃, L is —C(O)—, and R¹ is hydrogen.

In some embodiments of the compound of Formula (III), or the pharmaceutically acceptable salt thereof, each R⁹ is —H, X is —CF₃, L is —C(O)—, and R¹ is aryl.

In some embodiments of the compound of Formula (III), or the pharmaceutically acceptable salt thereof, each R⁹ is —H, X is —CF₃, L is —C(O)—, and R¹ is 5- to 10-membered ring heteroaryl, which may be substituted or unsubstituted. In some embodiments, the 5- to 10-membered ring heteroaryl is substituted with one or two halo.

In some embodiments of the compound of Formula (III), or the pharmaceutically acceptable salt thereof, each R⁹ is —H, X is pyridine-CF₃ or pyridine-O—CF₃, L is a bond, and R¹ is hydrogen.

In some embodiments of the compound of Formula (III), or the pharmaceutically acceptable salt thereof, each R⁹ is —H, X is pyridine-CF₃, L is a bond, and R¹ is hydrogen.

In some embodiments of the compound of Formula (III), or the pharmaceutically acceptable salt thereof, each R⁹ is —H, X is pyridine-CF₃ or pyridine-O—CF₃, L is a bond, and R¹ is cyano.

In some embodiments of the compound of Formula (III), or the pharmaceutically acceptable salt thereof, each R⁹ is —H, X is pyridine-CF₃ or pyridine-O—CF₃, L is a bond, and R¹ is amino, which may be substituted or unsubstituted. In some embodiments, the amino is substituted with one or two C₁-C₆ alkyl.

In some embodiments of the compound of Formula (III), or the pharmaceutically acceptable salt thereof, each R⁹ is —H, X is pyridine-CF₃ or pyridine-O—CF₃, L is a bond, and R¹ is C₁-C₁₀ alkyl, which may be substituted or unsubstituted. In some embodiments, R¹ is C₁-C₁₀ alkyl substituted with hydroxyl. In some embodiments, R¹ is C₁-C₁₀ alkyl substituted with OR¹⁰¹. In some embodiments, R¹ is C₁-C₁₀ alkyl substituted with C₁-C₆-alkoxy.

In some embodiments of the compound of Formula (III), or the pharmaceutically acceptable salt thereof, each R⁹ is —H, X is pyridine-CF₃, L is a bond, and R¹ is C₁-C₁₀ alkyl, which may be substituted or unsubstituted. In some embodiments, R¹ is C₁-C₁₀ alkyl substituted with hydroxyl. In some embodiments, R¹ is C₁-C₁₀ alkyl substituted with OR¹⁰¹. In some embodiments, R¹ is C₁-C₁₀ alkyl substituted with C₁-C₆-alkoxy.

In some embodiments of the compound of Formula (III), or the pharmaceutically acceptable salt thereof, each R⁹ is —H, X is pyridine-CF₃ or pyridine-O—CF₃, L is a bond, and R¹ is substituted imino. In some embodiments, the imino may be substituted with C₁-C₆ alkyl or C₁-C₆-alkoxy.

In some embodiments of the compound of Formula (III), or the pharmaceutically acceptable salt thereof, each R⁹ is —H, X is pyridine-CF₃, L is a bond, and R¹ is substituted imino. In some embodiments, the imino may be substituted with C₁-C₆ alkyl or C₁-C₆-alkoxy.

In some embodiments of the compound of Formula (III), or the pharmaceutically acceptable salt thereof, each R⁹ is —H, X is pyridine-CF₃ or pyridine-O—CF₃, L is a bond, and R¹ is 4- to 10-membered ring heteroaryl, which may be substituted or unsubstituted.

In some embodiments of the compound of Formula (III), or the pharmaceutically acceptable salt thereof, each R⁹ is —H, X is pyridine-CF₃ or pyridine-O—CF₃, L is —C(O)O—, and R¹ is hydrogen.

In some embodiments of the compound of Formula (III), or the pharmaceutically acceptable salt thereof, each R⁹ is —H, X is pyridine-CF₃ or pyridine-O—CF₃, L is —C(O)—, and R¹ is C₁-C₁₀ alkyl, which may be substituted or unsubstituted.

In some embodiments of the compound of Formula (III), or the pharmaceutically acceptable salt thereof, each R⁹ is —H, X is pyridine-CF₃ or pyridine-O—CF₃, L is —C(O)—, and R¹ is hydrogen.

In some embodiments of the compound of Formula (III), or the pharmaceutically acceptable salt thereof, each R⁹ is —H, X is pyridine-CF₂H, L is a bond, and R¹ is hydrogen.

In some embodiments of the compound of Formula (III), or the pharmaceutically acceptable salt thereof, each R⁹ is —H, X is —C(CH₃)₃, L is a bond, and R¹ is halo.

In some embodiments of the compound of Formula (III), or the pharmaceutically acceptable salt thereof, each R⁹ is —H, X is —C(CH₃)₃, L is a bond, and R¹ is cyano.

In some embodiments of the compound of Formula (III), or the pharmaceutically acceptable salt thereof, each R⁹ is —H, X is —C(CH₃)₃, L is —C(O)O—, and R¹ is C₁-C₁₀ alkyl.

In some embodiments of the compound of Formula (III), or the pharmaceutically acceptable salt thereof, each R⁹ is —H, X is —C(CH₃)₃, L is —C(O)O—, and R¹ is hydrogen.

In some embodiments of the compound of Formula (III), or the pharmaceutically acceptable salt thereof, each R⁹ is —H, X is —C(CH₃)₃, L is —C(O)—, and R¹ is hydrogen.

In some embodiments of the compound of Formula (III), or the pharmaceutically acceptable salt thereof, each R⁹ is —H, X is —C(CH₃)₃, L is —C(O)—, and R¹ is amino, which may be substituted or unsubstituted. In some embodiments, the amino is substituted with one or two C₁-C₆ alkyl.

Exemplary compounds, and pharmaceutically acceptable salts thereof, include but are not limited to:
4,6-dimethoxy-N-[5-methyl-4-(trifluoromethyl)-1,3-thiazol-2-yl]pyrimidine-5-carboxamide;
N-[5-iodo-4-(trifluoromethyl)-1,3-thiazol-2-yl]-4,6-dimethoxypyrimidine-5-carboxamide;
4,6-dimethoxy-N-[4-(trifluoromethyl)-1,3-thiazol-2-yl]pyrimidine-5-carboxamide;
N-[5-(4-chlorophenyl)-4-(trifluoromethyl)-1,3-thiazol-2-yl]-4,6-dimethoxypyrimidine-5-carboxamide;
N-[5-(4-chlorophenyl)-4-(trifluoromethyl)-1,3-thiazol-2-yl]-4,6-diethoxypyrimidine-5-carboxamide;
N-[5-ethyl-4-(trifluoromethyl)-1,3-thiazol-2-yl]-4,6-dimethoxypyrimidine-5-carboxamide;
N-[5-cyano-4-(trifluoromethyl)-1,3-thiazol-2-yl]-4,6-diethoxypyrimidine-5-carboxamide;
N-[5-(6-fluoropyridin-3-yl)-4-(trifluoromethyl)-1,3-thiazol-2-yl]-4,6-dimethoxypyrimidine-5-carboxamide;
N-(4,5-dimethyl-1,3-thiazol-2-yl)-4,6-dimethoxypyrimidine-5-carboxamide;
4,6-dimethoxy-N-{4-(trifluoromethyl)-5-[(trimethylsilyl)ethynyl]-1,3-thiazol-2-yl}pyrimidine-5-carboxamide;
N-[5-ethynyl-4-(trifluoromethyl)-1,3-thiazol-2-yl]-4,6-dimethoxypyrimidine-5-carboxamide;
N-[5-(3-hydroxyprop-1-yn-1-yl)-4-(trifluoromethyl)-1,3-thiazol-2-yl]-4,6-dimethoxypyrimidine-5-carboxamide;
N-[5-(3-hydroxypropyl)-4-(trifluoromethyl)-1,3-thiazol-2-yl]-4,6-dimethoxypyrimidine-5-carboxamide;
N-[5-(cyclopropylethynyl)-4-(trifluoromethyl)-1,3-thiazol-2-yl]-4,6-dimethoxypyrimidine-5-carboxamide;
4,6-dimethoxy-N-[5-(3-methoxyprop-1-yn-1-yl)-4-(trifluoromethyl)-1,3-thiazol-2-yl]pyrimidine-5-carboxamide;
4,6-dimethoxy-N-[5-(3-methoxypropyl)-4-(trifluoromethyl)-1,3-thiazol-2-yl]pyrimidine-5-carboxamide;
4,6-dimethoxy-N-{5-[(1Z)-3-methoxyprop-1-en-1-yl]-4-(trifluoromethyl)-1,3-thiazol-2-yl}pyrimidine-5-carboxamide;
4,6-dimethoxy-N-{5-[(1E)-3-methoxyprop-1-en-1-yl]-4-(trifluoromethyl)-1,3-thiazol-2-yl}pyrimidine-5-carboxamide;
N-[5-(3-hydroxy-3-methylbut-1-yn-1-yl)-4-(trifluoromethyl)-1,3-thiazol-2-yl]-4,6-dimethoxypyrimidine-5-carboxamide;
N-[5-(3-hydroxy-3-methylbutyl)-4-(trifluoromethyl)-1,3-thiazol-2-yl]-4,6-dimethoxypyrimidine-5-carboxamide; ethyl 2-{[(4,6-dimethoxypyrimidin-5-yl)carbonyl]amino}-4-(trifluoromethyl)-1,3-thiazole-5-carboxylate;
N-[5-(2-hydroxyethyl)-4-(trifluoromethyl)-1,3-thiazol-2-yl]-4,6-dimethoxypyrimidine-5-carboxamide; 4,6-dimethoxy-N-{5-[6-(morpholin-4-yl)pyridin-3-yl]-4-(trifluoromethyl)-1,3-thiazol-2-yl}pyrimidine-5-carboxamide;
N-[5-(3-cyanopyridin-4-yl)-4-(trifluoromethyl)-1,3-thiazol-2-yl]-4,6-dimethoxypyrimidine-5-carboxamide;
N-[5-(2-cyano-3-fluorophenyl)-4-(trifluoromethyl)-1,3-thiazol-2-yl]-4,6-dimethoxypyrimidine-5-carboxamide; N-(5-ethyl-4-phenyl-1,3-thiazol-2-yl)-4,6-dimethoxypyrimidine-5-carboxamide;
N-[5-(5-fluoropyridin-3-yl)-4-(trifluoromethyl)-1,3-thiazol-2-yl]-4,6-dimethoxypyrimidine-5-carboxamide;
4,6-dimethoxy-N-[5-(pyrazin-2-yl)-4-(trifluoromethyl)-1,3-thiazol-2-yl]pyrimidine-5-carboxamide;
4,6-dimethoxy-N-{5-[5-(morpholin-4-yl)pyrazin-2-yl]-4-(trifluoromethyl)-1,3-thiazol-2-yl}pyrimidine-5-carboxamide;
4,6-dimethoxy-N-{4-(trifluoromethyl)-5-[6-(trifluoromethyl)pyridin-2-yl]-1,3-thiazol-2-yl}pyrimidine-5-carboxamide;
N-[5-(6-cyanopyridin-2-yl)-4-(trifluoromethyl)-1,3-thiazol-2-yl]-4,6-dimethoxypyrimidine-5-carboxamide;
4,6-dimethoxy-N-[5-(morpholin-4-yl)-4-(trifluoromethyl)-1,3-thiazol-2-yl]pyrimidine-5-carboxamide;
N-{5-[2-(2-cyanopropan-2-yl)pyridin-4-yl]-4-(trifluoromethyl)-1,3-thiazol-2-yl}-4,6-dimethoxypyrimidine-5-carboxamide;
4,6-dimethoxy-N-[5-propyl-4-(trifluoromethyl)-1,3-thiazol-2-yl]pyrimidine-5-carboxamide;
N-[5-(2,2-dimethyl-1,3-dioxolan-4-yl)-4-(trifluoromethyl)-1,3-thiazol-2-yl]-4,6-dimethoxypyrimidine-5-carboxamide;
4,6-dimethoxy-N-{5-[2-(morpholin-4-yl)pyrimidin-5-yl]-4-(trifluoromethyl)-1,3-thiazol-2-yl}pyrimidine-5-carboxamide;
N-[5-cyclopropyl-4-(trifluoromethyl)-1,3-thiazol-2-yl]-4,6-dimethoxypyrimidine-5-carboxamide;
N-[5-(3,6-dihydro-2H-pyran-4-yl)-4-(trifluoromethyl)-1,3-thiazol-2-yl]-4,6-dimethoxypyrimidine-5-carboxamide;
4,6-dimethoxy-N-[5-(tetrahydro-2H-pyran-4-yl)-4-(trifluoromethyl)-1,3-thiazol-2-yl]pyrimidine-5-carboxamide;
N-[5-(3-cyanophenyl)-4-(trifluoromethyl)-1,3-thiazol-2-yl]-4,6-dimethoxypyrimidine-5-carboxamide;
N-[5-(4-cyanophenyl)-4-(trifluoromethyl)-1,3-thiazol-2-yl]-4,6-dimethoxypyrimidine-5-carboxamide;
N-[5-(2-cyanopyridin-3-yl)-4-(trifluoromethyl)-1,3-thiazol-2-yl]-4,6-dimethoxypyrimidine-5-carboxamide;
N-[5-(4-cyano-2-methoxyphenyl)-4-(trifluoromethyl)-1,3-thiazol-2-yl]-4,6-dimethoxypyrimidine-5-carboxamide;
N-[5-(6-cyano-1H-indol-3-yl)-4-(trifluoromethyl)-1,3-thiazol-2-yl]-4,6-dimethoxypyrimidine-5-carboxamide;
4,6-dimethoxy-N-[5-(pyridin-2-yl)-4-(trifluoromethyl)-1,3-thiazol-2-yl]pyrimidine-5-carboxamide;
N-[5-(5-chloropyridin-2-yl)-4-(trifluoromethyl)-1,3-thiazol-2-yl]-4,6-dimethoxypyrimidine-5-carboxamide;
N-[5-formyl-4-(trifluoromethyl)-1,3-thiazol-2-yl]-4,6-dimethoxypyrimidine-5-carboxamide;
4,6-dimethoxy-N-{5-[(4-methoxypiperidin-1-yl)methyl]-4-(trifluoromethyl)-1,3-thiazol-2-yl}pyrimidine-5-carboxamide;
4,6-dimethoxy-N-[5-(pyrrolidin-1-ylmethyl)-4-(trifluoromethyl)-1,3-thiazol-2-yl]pyrimidine-5-carboxamide;
4,6-dimethoxy-N-[5-(morpholin-4-ylmethyl)-4-(trifluoromethyl)-1,3-thiazol-2-yl]pyrimidine-5-carboxamide;
N-{5-[(cyclopentylamino)methyl]-4-(trifluoromethyl)-1,3-thiazol-2-yl}-4,6-dimethoxypyrimidine-5-carboxamide;
4,6-dimethoxy-N-[5-{[(2-methylpropyl)amino]methyl}-4-(trifluoromethyl)-1,3-thiazol-2-yl]pyrimidine-5-carboxamide;
N-{5-[(4-fluoropiperidin-1-yl)methyl]-4-(trifluoromethyl)-1,3-thiazol-2-yl}-4,6-dimethoxypyrimidine-5-carboxamide;
4,6-dimethoxy-N-[5-{[methyl(2-methylpropyl)amino]methyl}-4-(trifluoromethyl)-1,3-thiazol-2-yl]pyrimidine-5-carboxamide;
N-[5-{[tert-butyl(methyl)amino]methyl}-4-(trifluoromethyl)-1,3-thiazol-2-yl]-4,6-dimethoxypyrimidine-5-carboxamide;
N-{5-[(tert-butylamino)methyl]-4-(trifluoromethyl)-1,3-thiazol-2-yl}-4,6-dimethoxypyrimidine-5-carboxamide;
N-[5-{[(3 S)-3-fluoropyrrolidin-1-yl]methyl}-4-(trifluoromethyl)-1,3-thiazol-2-yl]-4,6-dimethoxypyrimidine-5-carboxamide;
N-[5-{[(3R)-3-fluoropyrrolidin-1-yl]methyl}-4-(trifluoromethyl)-1,3-thiazol-2-yl]-4,6-dimethoxypyrimidine-5-carboxamide;
4,6-dimethoxy-N-[5-{[(3S)-3-methoxypyrrolidin-1-yl]methyl}-4-(trifluoromethyl)-1,3-thiazol-2-yl]pyrimidine-5-carboxamide;
4,6-dimethoxy-N-[5-{[(3R)-3-methoxypyrrolidin-1-yl]methyl}-4-(trifluoromethyl)-1,3-thiazol-2-yl]pyrimidine-5-carboxamide;
4,6-dimethoxy-N-[5-(2-oxa-7-azaspiro[3.5]non-7-ylmethyl)-4-(trifluoromethyl)-1,3-thiazol-2-yl]pyrimidine-5-carboxamide;
4,6-dimethoxy-N-[5-(2-oxa-6-azaspiro[3.5]non-6-ylmethyl)-4-(trifluoromethyl)-1,3-thiazol-2-yl]pyrimidine-5-carboxamide;
N-[5-chloro-4-(trifluoromethyl)-1,3-thiazol-2-yl]-4,6-dimethoxypyrimidine-5-carboxamide;
4,6-dimethoxy-N-[5-(2-methylpyridin-3-yl)-4-(trifluoromethyl)-1,3-thiazol-2-yl]pyrimidine-5-carboxamide;
4,6-dimethoxy-N-{5-[1-methyl-3-(trifluoromethyl)-1H-pyrazol-5-yl]-4-(trifluoromethyl)-1,3-thiazol-2-yl}pyrimidine-5-carboxamide;
N-[5-(2-cyanopyridin-4-yl)-4-(trifluoromethyl)-1,3-thiazol-2-yl]-4,6-dimethoxypyrimidine-5-carboxamide;
N-[5-(1,3-dimethyl-1H-pyrazol-4-yl)-4-(trifluoromethyl)-1,3-thiazol-2-yl]-4,6-dimethoxypyrimidine-5-carboxamide;
{[(4,6-dimethoxypyrimidin-5-yl)carbonyl][5-(1,3-dimethyl-1H-pyrazol-4-yl)-4-(trifluoromethyl)-1,3-thiazol-2-yl]amino}methyl dihydrogen phosphate;
N-[5-ethyl-4-(trifluoromethyl)-1,3-thiazol-2-yl]-4,6-dimethoxy-2-methylpyrimidine-5-carboxamide;
4-ethoxy-N-[5-ethyl-4-(trifluoromethyl)-1,3-thiazol-2-yl]-6-methoxypyrimidine-5-carboxamide;
4,6-diethoxy-N-[5-ethyl-4-(trifluoromethyl)-1,3-thiazol-2-yl]pyrimidine-5-carboxamide;
4,6-dimethoxy-N-(5-(pyridin-4-yloxy)-4-(trifluoromethyl)thiazol-2-yl)pyrimidine-5-carboxamide;
4,6-dimethoxy-N-{4-[5-(trifluoromethyl)pyridin-2-yl]-1,3-thiazol-2-yl}pyrimidine-5-carboxamide;
([(4,6-dimethoxypyrimidin-5-yl)carbonyl]{4-[5-(trifluoromethyl)pyridin-2-yl]-1,3-thiazol-2-yl}amino)methyl dihydrogen phosphate;
ethyl 2-{[(4,6-dimethoxypyrimidin-5-yl)carbonyl]amino}-4-[5-(trifluoromethyl)pyridin-2-yl]-1,3-thiazole-5-carboxylate;
N-{5-formyl-4-[5-(trifluoromethyl)pyridin-2-yl]-1,3-thiazol-2-yl}-4,6-dimethoxypyrimidine-5-carboxamide;
4,6-dimethoxy-N-{5-(morpholin-4-ylmethyl)-4-[5-(trifluoromethyl)pyridin-2-yl]-1,3-thiazol-2-yl}pyrimidine-5-carboxamide;
4,6-dimethoxy-N-{5-(pyrrolidin-1-ylmethy)-4-[5-(trifluoromethyl)pyridin-2-yl]-1,3-thiazol-2-yl}pyrimidine-5-carboxamide;
N-{5-(dimethylcarbamoyl)-4-[5-(trifluoromethyl)pyridin-2-yl]-1,3-thiazol-2-yl}-4-hydroxy-6-methoxypyrimidine-5-carboxamide;
N-{5-(hydroxymethyl)-4-[5-(trifluoromethyl)pyridin-2-yl]-1,3-thiazol-2-yl}-4,6-dimethoxypyrimidine-5-carboxamide;
([(4,6-dimethoxypyrimidin-5-yl)carbonyl]{5-(hydroxymethyl)-4-[5-(trifluoromethyl)pyridin-2-yl]-1,3-thiazol-2-yl}amino)methyl dihydrogen phosphate;
N-{5-(1-hydroxyethyl)-4-[4-(trifluoromethoxy)phenyl]-1,3-thiazol-2-yl}-4,6-dimethoxypyrimidine-5-carboxamide;
N-{5-[(1R*)-1-hydroxyethyl]-4-[5-(trifluoromethyl)pyridin-2-yl]-1,3-thiazol-2-yl}-4,6-dimethoxypyrimidine-5-carboxamide;
N-{5-[(1S*)-1-hydroxyethyl]-4-[5-(trifluoromethyl)pyridin-2-yl]-1,3-thiazol-2-yl}-4,6-dimethoxypyrimidine-5-carboxamide;
N-{5-(2-hydroxypropan-2-yl)-4-[5-(trifluoromethyl)pyridin-2-yl]-1,3-thiazol-2-yl}-4,6-dimethoxypyrimidine-5-carboxamide;
4,6-dimethoxy-N-{5-(methoxymethyl)-4-[5-(trifluoromethyl)pyridin-2-yl]-1,3-thiazol-2-yl}pyrimidine-5-carboxamide;
N-{5-acetyl-4-[5-(trifluoromethyl)pyridin-2-yl]-1,3-thiazol-2-yl}-4,6-dimethoxypyrimidine-5-carboxamide;
N-{5-[(1E)-N-hydroxyethanimidoyl]-4-[5-(trifluoromethyl)pyridin-2-yl]-1,3-thiazol-2-yl}-4,6-dimethoxypyrimidine-5-carboxamide;
4,6-dimethoxy-N-{5-[(1E)-N-methoxyethanimidoyl]-4-[5-(trifluoromethyl)pyridin-2-yl]-1,3-thiazol-2-yl}pyrimidine-5-carboxamide;
4,6-dimethoxy-N-{5-(2-methyl-1,3-dioxolan-2-yl)-4-[5-(trifluoromethyl)pyridin-2-yl]-1,3-thiazol-2-yl}pyrimidine-5-carboxamide;
N-[4-(5-fluoropyridin-2-yl)-1,3-thiazol-2-yl]-4,6-dimethoxypyrimidine-5-carboxamide;
4,6-dimethoxy-N-{4-[5-(trifluoromethoxy)pyridin-2-yl]-1,3-thiazol-2-yl}pyrimidine-5-carboxamide;
4,6-dimethoxy-N-{4-[4-(trifluoromethoxy)phenyl]-1,3-thiazol-2-yl}pyrimidine-5-carboxamide;
N-{4-[5-(difluoromethyl)pyridin-2-yl]-1,3-thiazol-2-yl}-4,6-dimethoxypyrimidine-5-carboxamide;
4,6-dimethoxy-N-{5-methyl-4-[5-(trifluoromethyl)pyridin-2-yl]-1,3-thiazol-2-yl}pyrimidine-5-carboxamide;
N-{5-(3-hydroxypropyl)-4-[5-(trifluoromethyl)pyridin-2-yl]-1,3-thiazol-2-yl}-4,6-dimethoxypyrimidine-5-carboxamide;
N-{5-cyano-4-[5-(trifluoromethyl)pyridin-2-yl]-1,3-thiazol-2-yl}-4,6-dimethoxypyrimidine-5-carboxamide;
N-(4-cyclopentyl-1,3-thiazol-2-yl)-4,6-dimethoxypyrimidine-5-carboxamide;
N-[4-(4,4-difluorocyclohexyl)-1,3-thiazol-2-yl]-4,6-dimethoxypyrimidine-5-carboxamide;
N-(4-tert-butyl-5-fluoro-1,3-thiazol-2-yl)-4,6-dimethoxypyrimidine-5-carboxamide;
N-(4-cyclobutyl-5-fluoro-1,3-thiazol-2-yl)-4,6-dimethoxypyrimidine-5-carboxamide;
N-(5-cyano-4-cyclobutyl-1,3-thiazol-2-yl)-4,6-dimethoxypyrimidine-5-carboxamide;
N-{4-[(E)-2-cyclopropylethenyl]-1,3-thiazol-2-yl}-4,6-dimethoxypyrimidine-5-carboxamide;
N-{4-[(1E)-3,3-dimethylbut-1-en-1-yl]-1,3-thiazol-2-yl}-4,6-dimethoxypyrimidine-5-carboxamide;
N-[4-(2-cyclopropylethyl)-1,3-thiazol-2-yl]-4,6-dimethoxypyrimidine-5-carboxamide;
N-{4-[(E)-2-(4-fluorophenyl)ethenyl]-1,3-thiazol-2-yl}-4,6-dimethoxypyrimidine-5-carboxamide;
N-{4-[2-(4-fluorophenyl)ethyl]-1,3-thiazol-2-yl}-4,6-dimethoxypyrimidine-5-carboxamide;
N-[4-(4-fluoro-2-methoxyphenyl)-1,3-thiazol-2-yl]-4,6-dimethoxypyrimidine-5-carboxamide;
4,6-dimethoxy-N-[5-(p-tolylsulfonyl)-4-[5-(trifluoromethyl)-2-pyridyl]-1,3-thiazol-2-yl]pyrimidine-5-carboxamide;
4,6-dimethoxy-N-[5-(p-tolylsulfonyl)-4-(trifluoromethyl)-1,3-thiazol-2-yl]pyrimidine-5-carboxamide;
N-[5-benzylsulfonyl-4-(trifluoromethyl)-1,3-thiazol-2-yl]-4,6-dimethoxy-pyrimidine-5-carboxamide;
4,6-dimethoxy-N-[5-methylsulfinyl-4-[5-(trifluoromethyl)-2-pyridyl]-1,3-thiazol-2-yl]pyrimidine-5-carboxamide;
4,6-dimethoxy-N-[5-methylsulfonyl-4-[5-(trifluoromethyl)-2-pyridyl]-1,3-thiazol-2-yl]pyrimidine-5-carboxamide;
4,6-dimethoxy-N-[5-phenylsulfanyl-4-(trifluoromethyl)-1,3-thiazol-2-yl]pyrimidine-5-carboxamide;
N-[5-(2-hydroxypropan-2-yl)-4-(trifluoromethyl)-1,3-thiazol-2-yl]-4,6-dimethoxypyrimidine-5-carboxamide;
N-[5-(4-hydroxytetrahydro-2H-pyran-4-yl)-4-(trifluoromethyl)-1,3-thiazol-2-yl]-4,6-dimethoxypyrimidine-5-carboxamide;
N-[5-(4-fluorotetrahydro-2H-pyran-4-yl)-4-(trifluoromethyl)-1,3-thiazol-2-yl]-4,6-dimethoxypyrimidine-5-carboxamide;
N-[5-(1-hydroxycyclobutyl)-4-(trifluoromethyl)-1,3-thiazol-2-yl]-4,6-dimethoxypyrimidine-5-carboxamide;
N-{5-[hydroxy(phenyl)methyl]-4-(trifluoromethyl)-1,3-thiazol-2-yl}-4,6-dimethoxypyrimidine-5-carboxamide;
N-[5-benzoyl-4-(trifluoromethyl)-1,3-thiazol-2-yl]-4,6-dimethoxypyrimidine-5-carboxamide;
N-[5-(1-hydroxy-1-phenylethyl)-4-(trifluoromethyl)-1,3-thiazol-2-yl]-4,6-dimethoxypyrimidine-5-carboxamide;
N-[5-(1-hydroxy-2,2-dimethylpropyl)-4-(trifluoromethyl)-1,3-thiazol-2-yl]-4,6-dimethoxypyrimidine-5-carboxamide;
N-[5-(2,2-dimethylpropanoyl)-4-(trifluoromethyl)-1,3-thiazol-2-yl]-4,6-dimethoxypyrimidine-5-carboxamide;
4,6-dimethoxy-N-{5-[(E)-(methoxyimino)(phenyl)methyl]-4-(trifluoromethyl)-1,3-thiazol-2-yl}pyrimidine-5-carboxamide;
N-{5-[(E)-(hydroxyimino)(phenyl)methyl]-4-(trifluoromethyl)-1,3-thiazol-2-yl}-4,6-dimethoxypyrimidine-5-carboxamide;
4,6-dimethoxy-N-[5-(pyridin-2-ylcarbonyl)-4-(trifluoromethyl)-1,3-thiazol-2-yl]pyrimidine-5-carboxamide;
N-{5-[(E)-(hydroxyimino)(pyridin-2-yl)methyl]-4-(trifluoromethyl)-1,3-thiazol-2-yl}-4,6-dimethoxypyrimidine-5-carboxamide;
N-{5-[1-hydroxy-1-(pyridin-2-yl)ethyl]-4-(trifluoromethyl)-1,3-thiazol-2-yl}-4,6-dimethoxypyrimidine-5-carboxamide;
N-{5-[(5-fluoropyridin-2-yl)(hydroxy)methyl]-4-(trifluoromethyl)-1,3-thiazol-2-yl}-4,6-dimethoxypyrimidine-5-carboxamide;
N-{5-[(5-fluoropyridin-2-yl)carbonyl]-4-(trifluoromethyl)-1,3-thiazol-2-yl}-4,6-dimethoxypyrimidine-5-carboxamide;
N-{5-[(E)-(5-fluoropyridin-2-yl)(hydroxyimino)methyl]-4-(trifluoromethyl)-1,3-thiazol-2-yl}-4,6-dimethoxypyrimidine-5-carboxamide;
N-[5-acetyl-4-(trifluoromethyl)-1,3-thiazol-2-yl]-4,6-dimethoxypyrimidine-5-carboxamide;
N-{5-[(1E)-N-hydroxyethanimidoyl]-4-(trifluoromethyl)-1,3-thiazol-2-yl}-4,6-dimethoxypyrimidine-5-carboxamide;
4,6-dimethoxy-N-{5-[(1E)-N-methoxyethanimidoyl]-4-(trifluoromethyl)-1,3-thiazol-2-yl}pyrimidine-5-carboxamide;
4,6-dimethoxy-N-{5-[(1Z)—N-methoxyethanimidoyl]-4-(trifluoromethyl)-1,3-thiazol-2-yl}pyrimidine-5-carboxamide;
4,6-dimethoxy-N-[5-(2-methyl-1,3-dioxolan-2-yl)-4-(trifluoromethyl)-1,3-thiazol-2-yl]pyrimidine-5-carboxamide;
N-[5-(1,3-dioxolan-2-yl)-4-(trifluoromethyl)-1,3-thiazol-2-yl]-4,6-dimethoxypyrimidine-5-carboxamide;
N-[5-(1,3-dioxan-2-yl)-4-(trifluoromethyl)-1,3-thiazol-2-yl]-4,6-dimethoxypyrimidine-5-carboxamide;
4,6-dimethoxy-N-[5-(1,3-oxazol-5-yl)-4-(trifluoromethyl)-1,3-thiazol-2-yl]pyrimidine-5-carboxamide;
4,6-dimethoxy-N-[5-(4-methyl-1,3-oxazol-5-yl)-4-(trifluoromethyl)-1,3-thiazol-2-yl]pyrimidine-5-carboxamide;
N-[5-(1-ethyl-1H-pyrazol-5-yl)-4-(trifluoromethyl)-1,3-thiazol-2-yl]-4,6-dimethoxypyrimidine-5-carboxamide;
N-[5-(4,5-dihydrofuran-2-yl)-4-(trifluoromethyl)-1,3-thiazol-2-yl]-4,6-dimethoxypyrimidine-5-carboxamide;
4,6-dimethoxy-N-[5-(tetrahydrofuran-2-yl)-4-(trifluoromethyl)-1,3-thiazol-2-yl]pyrimidine-5-carboxamide;
N-(5-(4,5-dihydrofuran-3-yl)-4-(trifluoromethyl)thiazol-2-yl)-4,6-dimethoxypyrimidine-5-carboxamide;
4,6-dimethoxy-N-[5-(tetrahydrofuran-3-yl)-4-(trifluoromethyl)-1,3-thiazol-2-yl]pyrimidine-5-carboxamide;
N-[5-(3,4-dihydro-2H-pyran-5-yl)-4-(trifluoromethyl)-1,3-thiazol-2-yl]-4,6-dimethoxypyrimidine-5-carboxamide;
4,6-dimethoxy-N-[5-(tetrahydro-2H-pyran-3-yl)-4-(trifluoromethyl)-1,3-thiazol-2-yl]pyrimidine-5-carboxamide;
4,6-dimethoxy-N-{5-[(3S*)-tetrahydro-2H-pyran-3-yl]-4-(trifluoromethyl)-1,3-thiazol-2-yl}pyrimidine-5-carboxamide;
4,6-dimethoxy-N-{5-[(3R)-tetrahydro-2H-pyran-3-yl]-4-(trifluoromethyl)-1,3-thiazol-2-yl}pyrimidine-5-carboxamide;
N-{5-[2-(1-cyanocyclopropyl)pyridin-4-yl]-4-(trifluoromethyl)-1,3-thiazol-2-yl}-4,6-dimethoxypyrimidine-5-carboxamide;
N-{5-[2-(cyanomethyl)pyridin-4-yl]-4-(trifluoromethyl)-1,3-thiazol-2-yl}-4,6-dimethoxypyrimidine-5-carboxamide;
N-[5-(6-fluoropyridin-2-yl)-4-(trifluoromethyl)-1,3-thiazol-2-yl]-4,6-dimethoxypyrimidine-5-carboxamide;
N-{5-[6-(3-cyanooxetan-3-yl)pyridin-2-yl]-4-(trifluoromethyl)-1,3-thiazol-2-yl}-4,6-dimethoxypyrimidine-5-carboxamide;
N-{5-[6-(cyanomethyl)pyridin-2-yl]-4-(trifluoromethyl)-1,3-thiazol-2-yl}-4,6-dimethoxypyrimidine-5-carboxamide;
4,6-dimethoxy-N-{5-[5-(propan-2-yloxy)pyridin-3-yl]-4-(trifluoromethyl)-1,3-thiazol-2-yl}pyrimidine-5-carboxamide;
N-{5-[5-(1-cyanocyclopropyl)pyridin-2-yl]-4-(trifluoromethyl)-1,3-thiazol-2-yl}-4,6-dimethoxypyrimidine-5-carboxamide;
N-{5-[5-(2-cyanopropan-2-yl)pyridin-2-yl]-4-(trifluoromethyl)-1,3-thiazol-2-yl}-4,6-dimethoxypyrimidine-5-carboxamide;
N-[5-(2-cyanophenyl)-4-(trifluoromethyl)-1,3-thiazol-2-yl]-4-methoxy-6-(methylsulfanyl)pyrimidine-5-carboxamide;
N-[5-(2-cyanophenyl)-4-(trifluoromethyl)-1,3-thiazol-2-yl]-4-(ethylsulfanyl)-6-methoxypyrimidine-5-carboxamide;
2-cyano-N-[5-iodo-4-(trifluoromethyl)-1,3-thiazol-2-yl]-4-methoxy-6-(trifluoromethyl)pyrimidine-5-carboxamide;
N-[5-(2-cyanopyrimidin-5-yl)-4-(trifluoromethyl)-1,3-thiazol-2-yl]-4,6-dimethoxypyrimidine-5-carboxamide;
N-[4,5-bis(trifluoromethyl)-1,3-thiazol-2-yl]-4,6-dimethoxypyrimidine-5-carboxamide;
N-[5-(4-cyanocyclohex-1-en-1-yl)-4-(trifluoromethyl)-1,3-thiazol-2-yl]-4,6-dimethoxypyrimidine-5-carboxamide;
4,6-dimethoxy-N-{5-[1-(methylsulfonyl)-1,2,3,6-tetrahydropyridin-4-yl]-4-(trifluoromethyl)-1,3-thiazol-2-yl}pyrimidine-5-carboxamide;
4,6-dimethoxy-N-{4-(trifluoromethyl)-5-[2-(trifluoromethyl)pyridin-4-yl]-1,3-thiazol-2-yl}pyrimidine-5-carboxamide;
4,6-dimethoxy-N-[5-(quinolin-4-yl)-4-(trifluoromethyl)-1,3-thiazol-2-yl]pyrimidine-5-carboxamide;
N-[5-(1-cyclopropyl-1H-pyrazol-4-yl)-4-(trifluoromethyl)-1,3-thiazol-2-yl]-4,6-dimethoxypyrimidine-5-carboxamide;
4,6-dimethoxy-N-[5-(1,2-oxazol-4-yl)-4-(trifluoromethyl)-1,3-thiazol-2-yl]pyrimidine-5-carboxamide;
4,6-dimethoxy-N-{5-[3-(propan-2-yl)-4,5-dihydro-1,2-oxazol-5-yl]-4-(trifluoromethyl)-1,3-thiazol-2-yl}pyrimidine-5-carboxamide;
4,6-dimethoxy-N-{5-[3-(propan-2-yl)-1,2-oxazol-5-yl]-4-(trifluoromethyl)-1,3-thiazol-2-yl}pyrimidine-5-carboxamide;
4,6-dimethoxy-N-[5-(1,2-oxazol-3-yl)-4-(trifluoromethyl)-1,3-thiazol-2-yl]pyrimidine-5-carboxamide;
N-{5-[4-(2-cyanopropan-2-yl)phenyl]-4-(trifluoromethyl)-1,3-thiazol-2-yl}-4,6-dimethoxypyrimidine-5-carboxamide;
4,6-dimethoxy-N-{5-[1-(propan-2-yl)-1H-pyrazol-4-yl]-4-(trifluoromethyl)-1,3-thiazol-2-yl}pyrimidine-5-carboxamide;
N-{5-[1-(cyclopropylmethyl)-1H-pyrazol-4-yl]-4-(trifluoromethyl)-1,3-thiazol-2-yl}-4,6-dimethoxypyrimidine-5-carboxamide;
4,6-dimethoxy-N-[5-(1-methyl-1H-pyrazol-5-yl)-4-(trifluoromethyl)-1,3-thiazol-2-yl]pyrimidine-5-carboxamide;
N-[5-(1-ethyl-1H-pyrazol-4-yl)-4-(trifluoromethyl)-1,3-thiazol-2-yl]-4,6-dimethoxypyrimidine-5-carboxamide;
N-{4-[2-(4-fluorophenoxy)ethyl]-1,3-thiazol-2-yl}-4,6-dimethoxypyrimidine-5-carboxamide;
4,6-dimethoxy-N-{4-[(2-methylphenoxy)methyl]-1,3-thiazol-2-yl}pyrimidine-5-carboxamide;
4,6-dimethoxy-N-{5-[1-(propan-2-yl)-1H-pyrazol-5-yl]-4-(trifluoromethyl)-1,3-thiazol-2-yl}pyrimidine-5-carboxamide;
4,6-dimethoxy-N-{5-[1-methyl-3-(propan-2-yl)-1H-pyrazol-4-yl]-4-(trifluoromethyl)-1,3-thiazol-2-yl}pyrimidine-5-carboxamide;
4,6-dimethoxy-N-{5-[2-(morpholin-4-yl)pyridin-4-yl]-4-(trifluoromethyl)-1,3-thiazol-2-yl}pyrimidine-5-carboxamide;
4,6-dimethoxy-N-[5-(2-methoxypyrimidin-5-yl)-4-(trifluoromethyl)-1,3-thiazol-2-yl]pyrimidine-5-carboxamide;
N-[5-(5-cyano-6-methoxypyridin-3-yl)-4-(trifluoromethyl)-1,3-thiazol-2-yl]-4,6-dimethoxypyrimidine-5-carboxamide;
4,6-dimethoxy-N-{5-[2-(morpholin-4-yl)pyridin-3-yl]-4-(trifluoromethyl)-1,3-thiazol-2-yl}pyrimidine-5-carboxamide;
4,6-dimethoxy-N-{5-[1-methy-3-(trifluoromethyl)-H-pyrazol-4-yl]-4-(trifluoromethyl)-1,3-thiazol-2-yl}pyrimidine-5-carboxamide;
N-[5-(furo[3,2-b]pyridin-2-yl)-4-(trifluoromethyl)-1,3-thiazol-2-yl]-4,6-dimethoxypyrimidine-5-carboxamide;
N-{5-[1-(cyclopropylmethyl)-1H-pyrazol-5-yl]-4-(trifluoromethyl)-1,3-thiazol-2-yl}-4,6-dimethoxypyrimidine-5-carboxamide;
N-(4-tert-butyl-5-cyano-1,3-thiazol-2-yl)-4,6-dimethoxypyrimidine-5-carboxamide;
N-[4-cyclopropyl-5-(4-fluorobenzyl)-1,3-thiazol-2-yl]-4,6-dimethoxypyrimidine-5-carboxamide;
N-[4-(2,2-dimethylpropyl)-1,3-thiazol-2-yl]-4,6-dimethoxypyrimidine-5-carboxamide;
N-(5-bromo-4-tert-butyl-1,3-thiazol-2-yl)-4,6-dimethoxypyrimidine-5-carboxamide;
ethyl 4-tert-butyl-2-{[(4,6-dimethoxypyrimidin-5-yl)carbonyl]amino}-1,3-thiazole-5-carboxylate;
4,6-dimethoxy-N-{5-[6-(2,2,2-trifluoroethoxy)pyridin-3-yl]-4-(trifluoromethyl)-1,3-thiazol-2-yl}pyrimidine-5-carboxamide;
4,6-dimethoxy-N-{5-[3-(morpholin-4-ylsulfonyl)phenyl]-4-(trifluoromethyl)-1,3-thiazol-2-yl}pyrimidine-5-carboxamide;
4,6-dimethoxy-N-{4-(trifluoromethyl)-5-[5-(trifluoromethyl)pyridin-3-yl]-1,3-thiazol-2-yl}pyrimidine-5-carboxamide;
4,6-dimethoxy-N-[5-{3-[(2-methoxyethyl)(methyl)sulfamoyl]phenyl}-4-(trifluoromethyl)-1,3-thiazol-2-yl]pyrimidine-5-carboxamide;
4,6-dimethoxy-N-{5-[4-(morpholin-4-ylmethyl)phenyl]-4-(trifluoromethyl)-1,3-thiazol-2-yl}pyrimidine-5-carboxamide;
N-[5-(2,1,3-benzoxadiazol-5-yl)-4-(trifluoromethyl)-1,3-thiazol-2-yl]-4,6-dimethoxypyrimidine-5-carboxamide;
N-[5-(1-ethyl-3-methyl-1H-pyrazol-4-yl)-4-(trifluoromethyl)-1,3-thiazol-2-yl]-4,6-dimethoxypyrimidine-5-carboxamide;
N-[5-{3-[(dimethylsulfamoyl)amino]phenyl}-4-(trifluoromethyl)-1,3-thiazol-2-yl]-4,6-dimethoxypyrimidine-5-carboxamide;
4,6-dimethoxy-N-{4-(trifluoromethyl)-5-[2-(trifluoromethyl)pyrimidin-5-yl]-1,3-thiazol-2-yl}pyrimidine-5-carboxamide;
N-[5-(2-cyclopropylpyrimidin-5-yl)-4-(trifluoromethyl)-1,3-thiazol-2-yl]-4,6-dimethoxypyrimidine-5-carboxamide; and
N-{5-[5-cyano-6-(morpholin-4-yl)pyridin-3-yl]-4-(trifluoromethyl)-1,3-thiazol-2-yl}-4,6-dimethoxypyrimidine-5-carboxamide.

More particularly, compounds, and pharmaceutically acceptable salts thereof, of the invention include but are not limited to:
N-{5-[2-(2-cyanopropan-2-yl)pyridin-4-yl]-4-(trifluoromethyl)-1,3-thiazol-2-yl}-4,6-dimethoxypyrimidine-5-carboxamide;
4,6-dimethoxy-N-[5-(2-methylpyridin-3-yl)-4-(trifluoromethyl)-1,3-thiazol-2-yl]pyrimidine-5-carboxamide;
N-{4-[5-(difluoromethyl)pyridin-2-yl]-1,3-thiazol-2-yl}-4,6-dimethoxypyrimidine-5-carboxamide;
4,6-dimethoxy-N-{5-methyl-4-[5-(trifluoromethyl)pyridin-2-yl]-1,3-thiazol-2-yl}pyrimidine-5-carboxamide;
4,6-dimethoxy-N-[5-(4-methyl-1,3-oxazol-5-yl)-4-(trifluoromethyl)-1,3-thiazol-2-yl]pyrimidine-5-carboxamide;
4,6-dimethoxy-N-[5-(2-methoxypyrimidin-5-yl)-4-(trifluoromethyl)-1,3-thiazol-2-yl]pyrimidine-5-carboxamide;
N-[5-(1-ethyl-3-methyl-1H-pyrazol-4-yl)-4-(trifluoromethyl)-1,3-thiazol-2-yl]-4,6-dimethoxypyrimidine-5-carboxamide;
4,6-dimethoxy-N-{4-(trifluoromethyl)-5-[2-(trifluoromethyl)pyrimidin-5-yl]-1,3-thiazol-2-yl}pyrimidine-5-carboxamide;
N-(5-(6-cyano-5-(oxetan-3-yl-methoxy)pyridine-3-yl)-4-(trifluoromethyl)thiazol-2-yl)-4,6-dimethoxypyrimidine-5-carboxamide; and
4,6-dimethoxy-N-(5-(methoxymethyl)-4-(trifluoromethyl)thiazol-2-yl)pyrimidine-5-carboxamide.

The present compounds may exist as stereoisomers wherein asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, Pure Appl. Chem., 1976, 45: 13-30.

Various stereoisomers of the present compounds and mixtures thereof are included within the scope of this application. Stereoisomers include enantiomers and diastereomers, and mixtures of enantiomers or diastereomers. Individual stereoisomers may be prepared synthetically from commercially available starting materials which contain asymmetric or chiral centers or by preparation of racemic mixtures followed by resolution which is well known to those of ordinary skill in the art. These methods of resolution are exemplified by: (1) attachment of a mixture of enantiomers to a chiral auxiliary, separation of the resulting mixture of diastereomers by recrystallization or chromatography and liberation of the optically pure product from the auxiliary, or (2) direct separation of the mixture of optical enantiomers on chiral chromatographic columns.

Geometric isomers may exist in the present compounds. Various geometric isomers and mixtures thereof resulting from the disposition of substituents around a carbon-carbon double bond, a carbon-nitrogen double bond, a cycloalkyl group, or a heterocycle group are contemplated. Substituents around a carbon-carbon double bond or a carbon-nitrogen bond are designated as being of Z or E configuration and substituents around a cycloalkyl or a heterocycle are designated as being of cis or trans configuration.

Compounds disclosed herein may exhibit the phenomenon of tautomerism.

Thus, the formulae drawings within this specification can represent only one of the possible tautomeric or stereoisomeric forms. It is to be understood that the invention encompasses any tautomeric or stereoisomeric form, and mixtures thereof, and is not to be limited merely to any one tautomeric or stereoisomeric form utilized within the naming of the compounds or formulae drawings.

Compounds of the invention can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to, ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention. In another embodiment, the isotope-labeled compounds may contain deuterium (²H), tritium (³H) or ¹⁴C isotopes. Isotope-labeled compounds of this invention can be prepared by the general methods well known to persons having ordinary skill in the art. Such isotope-labeled compounds can be conveniently prepared by carrying out the procedures disclosed in the Examples and

Schemes sections by substituting a readily available isotope-labeled reagent for a non-labeled reagent. In some instances, compounds may be treated with isotope-labeled reagents to exchange a normal atom with its isotope, for example, hydrogen for deuterium can be exchanged by the action of a deuteric acid such as D₂SO₄/D₂O. In addition to the above, relevant procedures and intermediates are disclosed, for instance, in Lizondo, J et al., Drugs Fut, 21(11), 1116 (1996); Brickner, S J et al., J Med Chem, 39(3), 673 (1996); Mallesham, B et al., Org Lett, 5(7), 963 (2003); PCT publications WO1997010223, WO2005099353, WO1995007271, WO2006008754; U.S. Pat. Nos. 7,538,189; 7,534,814; 7,531,685; 7,528,131; 7,521,421; 7,514,068; 7,511,013; and US Patent Application Publication Nos. 20090137457; 20090131485; 20090131363; 20090118238; 20090111840; 20090105338; 20090105307; 20090105147; 20090093422; 20090088416; and 20090082471, the methods of which are hereby incorporated by reference.

The isotope-labeled compounds of the invention may be used as standards to determine the effectiveness of TRPV3 modulators in binding assays. Isotope containing compounds have been used in pharmaceutical research to investigate the in vivo metabolic fate of the compounds by evaluation of the mechanism of action and metabolic pathway of the nonisotope-labeled parent compound (Blake et al. J. Pharm. Sci. 64, 3, 367-391 (1975)). Such metabolic studies are important in the design of safe, effective therapeutic drugs, either because the in vivo active compound administered to the patient or because the metabolites produced from the parent compound prove to be toxic or carcinogenic (Foster et al., Advances in Drug Research Vol. 14, pp. 2-36, Academic press, London, 1985; Kato et al., J. Labelled Comp. Radiopharmaceut., 36(10):927-932 (1995); Kushner et al., Can. J. Physiol. Pharmacol., 77, 79-88 (1999).

In addition, non-radioactive isotope containing drugs, such as deuterated drugs called “heavy drugs,” can be used for the treatment of diseases and conditions related to TRPV3 activity. Increasing the amount of an isotope present in a compound above its natural abundance is called enrichment. Examples of the amount of enrichment include from about 0.5, 1, 2, 3, 4, 5, 6, 7, 8, 9, 10, 12, 16, 21, 25, 29, 33, 37, 42, 46, 50, 54, 58, 63, 67, 71, 75, 79, 84, 88, 92, 96, to about 100 mol %. Replacement of up to about 15% of normal atoms with a heavy isotope has been effected and maintained for a period of days to weeks in mammals, including rodents and dogs, with minimal observed adverse effects (Czajka D M and Finkel A J, Ann. N.Y. Acad. Sci. 1960 84: 770; Thomson J F, Ann. New York Acad. Sci 1960 84: 736; Czakja D M et al., Am. J. Physiol. 1961 201: 357). Acute replacement of as high as 15%-23% in human fluids with deuterium was found not to cause toxicity (Blagojevic N et al. in “Dosimetry & Treatment Planning for Neutron Capture Therapy”, Zamenhof R, Solares G and Harling O Eds. 1994. Advanced Medical Publishing, Madison Wis. pp. 125-134; Diabetes Metab. 23: 251 (1997)).

Stable isotope labeling of a drug may alter its physico-chemical properties such as pKa and lipid solubility. These effects and alterations may affect the pharmacodynamic response of the drug molecule if the isotopic substitution affects a region involved in a ligand-receptor interaction. While some of the physical properties of a stable isotope-labeled molecule are different from those of the unlabeled one, the chemical and biological properties are the same, with one exception: because of the increased mass of the heavy isotope, any bond involving the heavy isotope and another atom will be stronger than the same bond between the light isotope and that atom. Accordingly, the incorporation of an isotope at a site of metabolism or enzymatic transformation will slow said reactions potentially altering the pharmacokinetic profile or efficacy relative to the non-isotopic compound.

### c. Biological Data

(i) In Vitro Methods-Calcium Flux Assays:

Experiments were conducted using the FLIPR TETRA®. On the day prior to the experiment, recombinant HEK293 cells that stably express human and mouse TRPV3 were removed from tissue culture flasks and plated in growth medium at 20,000 cells/well into black-walled clear-bottom 384-well BIOCOAT™ poly-D-lysine assay plates (BD Biosciences, Bedford, Mass.) using a MULTIDROP® dispenser (ThermoScientific, Waltham, Mass.). On the day of the experiment, growth medium was removed, and the no-wash FLIPR® Calcium-4 dye (λ_{EX}=470-495 nm, λ_{EM}=515-575 nm; Molecular Devices, Sunnyvale, Calif.) was added to each well using the MULTIDROP® dispenser. Cells were incubated for 90-120 minutes in the dark. The compounds were dissolved in DMSO to prepare a 10 mM stock solution. The intensity of the fluorescence was captured and digitally transferred to an interfaced PC. The peak increase in fluorescence over baseline, measured in relative fluorescence units, was calculated and expressed as the percentage of the maximal 2-APB (2-aminoethoxyldiphenyl borate) response (in the absence of compound). The concentration of 2-APB corresponds to its EC₈₀. IC₅₀ of the compounds for human TRPV3 are shown in Table 1 wherein “A” refers to an IC₅₀ value of less than 0.05 Kb μM, “B” refers to an IC₅₀ value in the range of 0.05 Kb μM to 0.1 Kb μM, “C” refers to an IC₅₀ value in the range of 0.1 Kb μM to 0.5 Kb μM, “D” refers to an IC₅₀ value in the range of 0.5 Kb M to 1.0 Kb μM, and “E” refers to an IC₅₀ value greater than 1.0 Kb μM. “NR” indicates that data were not reported.

TABLE 1

| Example # | IC₅₀ (μM) |
|---|---|
| 1 | B |
| 2 | A |
| 3 | C |
| 4 | B |
| 5 | D |
| 6 | A |
| 7 | D |
| 8 | B |
| 9 | E |
| 10 | A |
| 11 | A |
| 12 | C |
| 13 | E |
| 14 | A |
| 15 | A |
| 16 | A |
| 17 | A |
| 18 | A |
| 19 | B |
| 20 | D |
| 21 | A |
| 22 | E |
| 23 | C |
| 24 | C |
| 25 | A |
| 26 | B |
| 27 | B |
| 28 | D |
| 29 | D |
| 30 | A |
| 31 | A |
| 32 | C |
| 33 | B |
| 34 | A |
| 35 | C |
| 36 | B |
| 37 | A |
| 38 | A |
| 39 | B |
| 40 | A |
| 41 | A |
| 42 | A |
| 43 | A |
| 44 | C |
| 45 | C |
| 46 | A |
| 47 | E |
| 48 | C |
| 49 | D |
| 50 | E |
| 51 | C |
| 52 | B |
| 53 | A |
| 54 | A |
| 55 | C |
| 56 | E |
| 57 | C |
| 58 | B |
| 59 | D |
| 60 | D |
| 61 | C |
| 62 | B |
| 63 | A |
| 64 | A |
| 65 | A |
| 66 | B |
| 67 | B |
| 68 | NR |
| 69 | B |
| 70 | A |
| 71 | A |
| 72 | C |
| 73 | A |
| 74 | NR |
| 75 | C |
| 76 | C |
| 77 | A |
| 78 | C |
| 79 | E |
| 80 | B |
| 81 | NR |
| 82 | C |
| 83 | C |
| 84 | C |
| 85 | B |
| 86 | A |
| 87 | B |
| 88 | B |
| 89 | A |
| 90 | B |
| 91 | D |
| 92 | A |
| 93 | C |
| 94 | C |
| 95 | A |
| 96 | C |
| 97 | B |
| 98 | C |
| 99 | C |
| 100 | A |
| 101 | B |
| 102 | C |
| 103 | E |
| 104 | E |
| 105 | B |
| 106 | D |
| 107 | D |
| 108 | C |
| 109 | C |
| 110 | C |
| 111 | D |
| 112 | E |
| 113 | D |
| 114 | C |
| 115 | B |
| 116 | E |
| 117 | E |
| 118 | D |
| 119 | E |
| 120 | C |
| 121 | B |
| 122 | A |
| 123 | C |
| 124 | A |
| 125 | A |
| 126 | A |
| 127 | E |
| 128 | C |
| 129 | E |
| 130 | C |
| 131 | A |
| 132 | A |
| 133 | C |
| 134 | C |
| 135 | A |
| 136 | A |
| 137 | B |
| 138 | C |
| 139 | C |
| 140 | B |
| 141 | B |
| 142 | C |
| 143 | A |
| 144 | B |
| 145 | A |
| 146 | C |
| 147 | B |
| 148 | B |
| 149 | E |
| 150 | C |
| 151 | A |
| 152 | C |
| 153 | A |
| 154 | B |
| 155 | C |
| 156 | A |
| 157 | B |
| 158 | A |
| 159 | A |
| 160 | A |
| 161 | C |
| 162 | C |
| 163 | B |
| 164 | C |
| 165 | D |
| 166 | B |
| 167 | A |
| 168 | B |
| 169 | A |
| 170 | C |
| 171 | B |
| 172 | C |
| 173 | C |
| 174 | A |
| 175 | A |
| 176 | C |
| 177 | C |
| 178 | D |
| 179 | C |
| 180 | A |
| 181 | B |
| 182 | B |
| 183 | C |
| 184 | A |
| 185 | A |
| 186 | A |
| 187 | C |
| 188 | A |
| 189 | A |
| 190 | B |
| 191 | B |
| 192 | A |
| 193 | A |
| 194 | C |
| 195 | B |
| 196 | A |
| 197 | A |
| 198 | A |
| 199 | A |
| 200 | A |
| 201 | B |
| 202 | A |
| 203 | A |
| 204 | A |
| 205 | C |
| 206 | C |

(ii) In vivo Data The following protocol was used to evaluate the efficacy of a compound against contact hypersensitivity:

Animals

Young adult female BALB/c mice (18±2 g body weight at 6-8 weeks old) Charles River Laboratories) were maintained under pathogen-free conditions and housed in an animal room maintained t at temperature of 21±1° C. and a humidity of 43±5%. Induction of contact hypersensitivity (CHS) and ear thickness measurements for all animals were carried out under light anesthesia (3-5% isoflurane/oxygen) for 1-2 minutes.

### Induction of Th2-Type C-HS Responses to FITC

Contact hypersensitivity consists of the afferent or initiation sensitizing phase, and the efferent or elicitation phase. The latter phase occurs when epidermal cells encounter a particular antigen to which the epidermal cells have previously been exposed, and is characterized in rodents by localized swelling in the skin.

Fluorescein isothiocyanate (FITC) was purchased from Sigma-Aldrich and dissolved in 1:1 in acetone/dibutylphthalate prior to epicutaneous application. Mice were sensitized by painting epicutaneously 40 uL of 0.5% FITC on the abdomen ventral skin shaved one day prior to sensitization. Six days later, mice were challenged by painting epicutaneously 20 uL total volume of 0.5% FITC on the right ear (10 uL on either side). Control mice were treated with vehicle only. Ear swelling was measured 24 hours after challenge. Treatment groups were dosed orally at a dose volume of 10 mL/kg with compounds either BID (1 hour prior to FITC challenge, 8 hours after first dose and 2 hours prior to ear swelling measurement at 24 hours post FITC challenge) or QD (1 hour prior to FITC challenge and 2 hours prior to ear swelling measurement at 24 hours post FITC challenge). Inflammation was determined by the degree of ear swelling of the hapten exposed ear compared with the untreated ear with a Quick Mini thickness-gauge caliber (Mitutoyo, Japan). The change in ear thickness after allergen treatment can be used to calculate the percent suppression of contact hypersensitivity.

The level of compound-mediated suppression of ear skin thickness in FITC-challenged mice is indicated in Table 2 wherein “A” refers to a reduction in ear skin thickness of >50%, “B” refers to a reduction in ear skin thickness of 30-50% and “C” refers to a reduction in ear skin thickness of <30% relative to vehicle-treated animals. Compound dosages are also provided in Table 2.

TABLE 2

| | | FITC % |
|---|---|---|
| Example # | FITC dose (po) | effect |
| 27 | 30 mg/kg; bid | C |
| 31 | 30 mg/kg; qd | A |
| 33 | 30 mg/kg; qd | A |
| 40 | 30 mg/kg, bid | C |
| 41 | 30 mg/kg, bid | C |
| 42 | 30 mg/kg; bid | C |
| 48 | 30 mg/kg, bid | C |
| 52 | 10 mg/kg, qd | B |
| 53 | 10 mg/kg, bid | B |
| 58 | 10 mg/kg, qd | B |
| 64 | 30 mg/kg, qd | B |
| 65 | 30 mg/kg, bid | C |
| 66 | 100 mg/kg, bid | A |
| 74 | 100 mg/kg, bid | A |
| 169 | 30 mg/kg, bid | C |
| 196 | 30 mg/kg, bid | C |
| 199 | 30 mg/kg, bid | B |
| 202 | 30 mg/kg, bid | C |

### d. Methods of Using the Compounds

The data in Table 1 demonstrate that present compounds are modulators of TRPV3 receptors, and thus are useful in the treatment of diseases, conditions, and/or disorders modulated by TRPV3. The relationship between therapeutic effect and inhibition of TRPV3 has been shown in: WO2007/056124; Wissenbach, U. et al., Biology of the cell (2004), 96, 47-54; Nilius, B. et al., Physiol Rev (2007), 87, 165-217; Okuhara, D. Y. et al., Expert Opinion on Therapeutic Targets (2007), 11, 391-401; Hu, H. Z. et al., Journal of Cellular Physiology (2006), 208, 201-212.

One embodiment is therefore directed to a method for treating a disease, condition, and/or disorder modulated by TRPV3 in a subject in need thereof, said method comprising administering to the subject a therapeutically effective amount of a compound, or a pharmaceutically acceptable salt, solvate, salt of a solvate or solvate of a salt thereof, optionally with a pharmaceutically acceptable carrier.

Diseases, conditions, and/or disorders that are modulated by TRPV3 include, but are not limited to: migraine, arthralgia, cardiac pain arising from an ischemic myocardium, acute pain, chronic pain, nociceptive pain, neuropathic pain, post-operative pain, pain due to neuralgia (e.g., post-herpetic neuralgia, traumatic neuralgia, fibromyalgia, trigeminal neuralgia), pain due to diabetic neuropathy, dental pain and cancer pain, inflammatory pain conditions (e.g. arthritis and osteoarthritis).

Diseases, conditions, and/or disorders that are modulated by TRPV3 also include, but are not limited to: pain such as neuropathic pain, nociceptive pain, dental pain, HIV pain, cardiac pain arising from an ischemic myocardium, pain due to migraine, arthralgia, neuropathies, neurodegeneration, retinopathy, neurotic skin disorder, stroke, urinary bladder hypersensitiveness, urinary incontinence, vulvodynia, gastrointestinal disorders such as irritable bowel syndrome, gastro-esophageal reflux disease, enteritis, ileitis, stomach-duodenal ulcer, inflammatory bowel disease, Crohn's disease, celiac disease, an inflammatory disease such as pancreatitis, a respiratory disorder such as allergic and non-allergic rhinitis, asthma or chronic obstructive pulmonary disease, irritation of skin, eye or mucous membrane, atopic dermatitis, eczema itch, fervescence, muscle spasms, emesis, dyskinesias, depression, Huntington's disease, memory deficits, restricted brain function, amyotrophic lateral sclerosis (ALS), dementia, arthritis, osteoarthritis, diabetes, obesity, urticaria, actinic keratosis, keratocanthoma, alopecia, Meniere's disease, tinnitus, hyperacusis, anxiety disorders and benign prostate hyperplasia.

One embodiment provides methods for treating atopic dermatitis, eczema, sebhorreic eczema, itch, or psoriasis in a subject (including a human subject) in need of such treatment.

The methods comprise administering to the subject a therapeutically effective amount of a compound as described herein, or a pharmaceutically acceptable salt, solvate, salt of a solvate, or solvate of a salt thereof, optionally with a pharmaceutically acceptable carrier. The method further comprises administration of the present compound as a single dose. The method also comprises repeated or chronic administration of the present compound over a period of days, weeks, months, or longer. In certain embodiments, the method comprises administering to the subject a therapeutically effective amount of a compound as described herein, or a pharmaceutically acceptable salt, solvate, salt of a solvate, or solvate of a salt thereof, in combination with one or more additional agents appropriate for the particular disease, condition, or disorder being treated.

When combinations of a TRPV3 inhibitor and one or more additional compounds or agents are administered, the invention contemplates administration of the combination via the same route of administration or administration of one or more of the TRPV3 inhibitor and the one or more additional compounds or agents via differing routes of administration.

Another embodiment provides a method for increasing the therapeutic effectiveness or potency of compounds described herein by repeated or chronic administration over a period of days, weeks, or months.

Actual dosage levels of active ingredients in the pharmaceutical compositions can be varied so as to obtain an amount of the active compound(s) that is effective to achieve the desired therapeutic response for a particular patient, composition, and mode of administration. The selected dosage level will depend upon the activity of the particular compound, the route of administration, the duration of treatment, other drugs, compounds and/or materials used in combination with the particular compound employed, the age, sex, weight, condition, general health and prior medical history of the patient being treated, the severity of the condition being treated, and like factors well known in the medical arts.

A physician or veterinarian having ordinary skill in the art can readily determine and prescribe the effective amount of the pharmaceutical composition required. For example, the physician or veterinarian could start doses of the compounds employed in the pharmaceutical compositions at levels lower than required to achieve the desired therapeutic effect and gradually increase the dosage until the desired effect is achieved. In the treatment of certain medical conditions, repeated or chronic administration of the compounds may be required to achieve the desired therapeutic response. “Repeated or chronic administration” refers to the administration of the compounds daily (i.e., every day) or intermittently (i.e., not every day) over a period of days, weeks, months, or longer. In particular, the treatment of chronic painful conditions is anticipated to require such repeated or chronic administration of compounds described herein. The compounds may become more effective upon repeated or chronic administration such that the therapeutically effective doses on repeated or chronic administration may be lower than the therapeutically effective dose from a single administration.

Compounds can also be administered as a pharmaceutical composition comprising the compounds of interest, or pharmaceutically acceptable salts, solvates, or salts of solvates thereof, in combination with one or more pharmaceutically acceptable carriers. The phrase “therapeutically effective amount” of a compound means a sufficient amount of the compound to treat disorders, at a reasonable benefit/risk ratio applicable to any medical treatment. It will be understood, however, that the total daily usage of the compounds and compositions will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well-known in the medical arts. For example, it is well within the skill of the art to start doses of the compound at levels lower than required to achieve the desired therapeutic effect and to gradually increase the dosage until the desired effect is achieved.

If desired, the effective daily dose can be divided into multiple doses for purposes of administration. Consequently, multiple dose compositions may contain such amounts or submultiples thereof to make up the daily dose. It is understood that the effective daily dose may vary with the duration of the treatment.

The compounds may be administered alone, or in combination with one or more other compounds described herein, or in combination (i.e. co-administered) with one or more additional pharmaceutical agents. For example, one or more compounds, or pharmaceutically acceptable salts, solvates, salts of solvates, or solvates of salts thereof, may be administered in combination with one or more agents such as topical corticosteroids, vitamin D analogues, anthralin, topical retinoids, calcineurin inhibitors, salicylic acid, coal tar, or analgesics.

Where separate dosage formulations are used, the compounds and one or more additional pharmaceutical agents may be administered at essentially the same time (e.g., concurrently) or at separately staggered times (e.g., sequentially).

### e. Pharmaceutical Compositions

Further provided herein is a pharmaceutical composition that comprises a compound or a pharmaceutically acceptable salt, solvate, salt of a solvate, or solvate of a salt thereof, formulated together with a pharmaceutically acceptable carrier.

Another aspect provides pharmaceutical composition comprising a compound or a pharmaceutically acceptable salt, solvate, salt of a solvate, or solvate of a salt thereof, in combination with an analgesic (e.g. acetaminophen or opioid such as morphine or other related opioids), or in combination with a nonsteroidal anti-inflammatory drug (NSAID), or a combination thereof, formulated together with a pharmaceutically acceptable carrier.

The pharmaceutical compositions can be administered to humans and other mammals orally, rectally, parenterally, intracistemally, intravaginally, intraperitoneally, topically (as by powders, ointments or drops), bucally or as an oral or nasal spray. The term “parenterally”, as used herein, refers to modes of administration which include intravenous, intramuscular, intraperitoneal, intrasternal, subcutaneous and intraarticular injection, and infusion.

The term “pharmaceutically acceptable carrier”, as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols such as, but not limited to, propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol; and phosphate buffer solutions. Other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutical compositions for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include: water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form can be brought about by the inclusion of agents which delay absorption such as aluminum monostearate and gelatin.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or: a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such carriers as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned carriers.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which can be prepared by mixing the compounds of this invention with suitable non-irritating carriers or carriers such as cocoa butter, polyethylene glycol or a suppository wax which are solid at room temperature but liquid at body temperature and therefore melt in the rectum or vaginal cavity and release the active compound.

The present compounds can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

Dosage forms for topical administration include powders, sprays, ointments and inhalants. The active compound may be mixed under sterile conditions with a pharmaceutically acceptable carrier and any needed preservatives, buffers or propellants which may be required. Opthalmic formulations, eye ointments, powders and solutions are also contemplated as being within the scope of this invention.

The compounds can be used in the form of pharmaceutically acceptable salts derived from inorganic or organic acids. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio.

Pharmaceutically acceptable salts are well known in the art. For example, S. M. Berge et al. describe pharmaceutically acceptable salts in detail in (J. Pharmaceutical Sciences, 1977, 66: 1 et seq). The salts can be prepared in situ during the final isolation and purification of the compounds or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as, but not limited to: methyl, ethyl, propyl, and butyl chlorides, bromides and iodides; dialkyl sulfates like dimethyl, diethyl, dibutyl and diamyl sulfates; long chain halides such as, but not limited to: decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid and citric acid.

Basic addition salts can be prepared in situ during the final isolation and purification of the compounds by reacting a carboxylic acid-containing moiety with a suitable base such as, but not limited to: the hydroxide, carbonate or bicarbonate of a pharmaceutically acceptable metal cation or with ammonia or an organic primary, secondary or tertiary amine. Pharmaceutically acceptable salts include, but are not limited to cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

The compounds can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

### f. General Synthesis

Compounds described herein when prepared by synthetic processes or by metabolic processes are encompassed within the scope of this application. Preparation of the compounds by metabolic processes includes those occurring in the human or animal body (in vivo) or processes occurring in vitro.

The compounds can be prepared by a variety of processes well known for the preparation of compounds of this class. For example, the compounds described herein can be synthesized as shown in Schemes 1 through 6.

Abbreviations which have been used in the descriptions of the Schemes and the Examples that follow are: THF for tetrahydrofuran, DPPF for 1,1′-dis(diphenylphosphino)ferrocene, NIS for N-iodosuccinimide, NBS for N-bromosuccinimide, NCS for N-chlorosuccinimide, Et₂O for diethylether, AcOH for acetic acid, DMF for N,N-dimethylformamide, EtOAc or EA for ethyl acetate, MTBE for methyl tert-butyl ether, MeOH for methanol, MeCN for acetonitrile, DMSO for dimethyl sulfoxide, EtOH for ethanol, amphos for (di-tert-butyl(4-dimethylaminophenyl)phosphine), DBA for bis(dibenzylideneacetone), t-BuOH for tert-butanol, DCM for dichloromethane, m-CPBA for meta-chloroperoxybenzoic acid, MsCl for methanesulfonyl chloride, DME for dimethoxyethane, PEPPSI for pyridine enhanced precatalyst preparation stabilization and initiation catalyst, and IBX for 2-iodoxybenzoic acid.

Compounds of formula (I), formula (II-A), formula (II-B), and formula (III) can be prepared using general procedures as illustrated in Schemes 1 through 6.

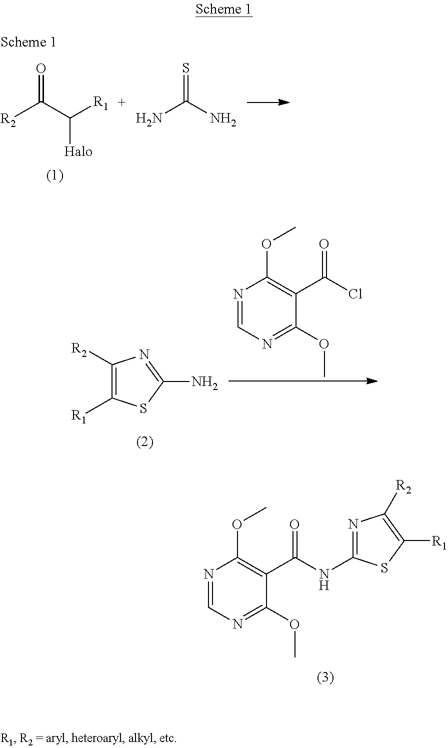

The substituted halo-ketone reactant (1) may be contacted with thiourea reactant to thereby prepare intermediate substituted thiazol-2-amine (2). The halo substituent on the halo-ketone reactant (1) may be fluoro, chloro, bromo, or iodo. The R₁ and R₂ substituents on reactants (1) and (2) are as defined herein. The cyclization reaction may be carried out in a solvent such as, but not limited to, methanol or ethanol, at room temperature or at an elevated temperature of at least about 40° C., such as between about 60° C. and about 70° C., optionally with stirring.

The intermediate substituted thiazol-2-amine (2) may be contacted with 4,6-dimethoxypyrimidine-5-carbonyl chloride reactant (the synthesis thereof is described in the examples) to thereby prepare compound (3). The coupling reaction may be carried out in a solvent such as, but not limited to, methylene chloride and/or DMF, at room temperature or at an elevated temperature of at least about 40° C., such as between about 60° C. and about 70° C., optionally with stirring.

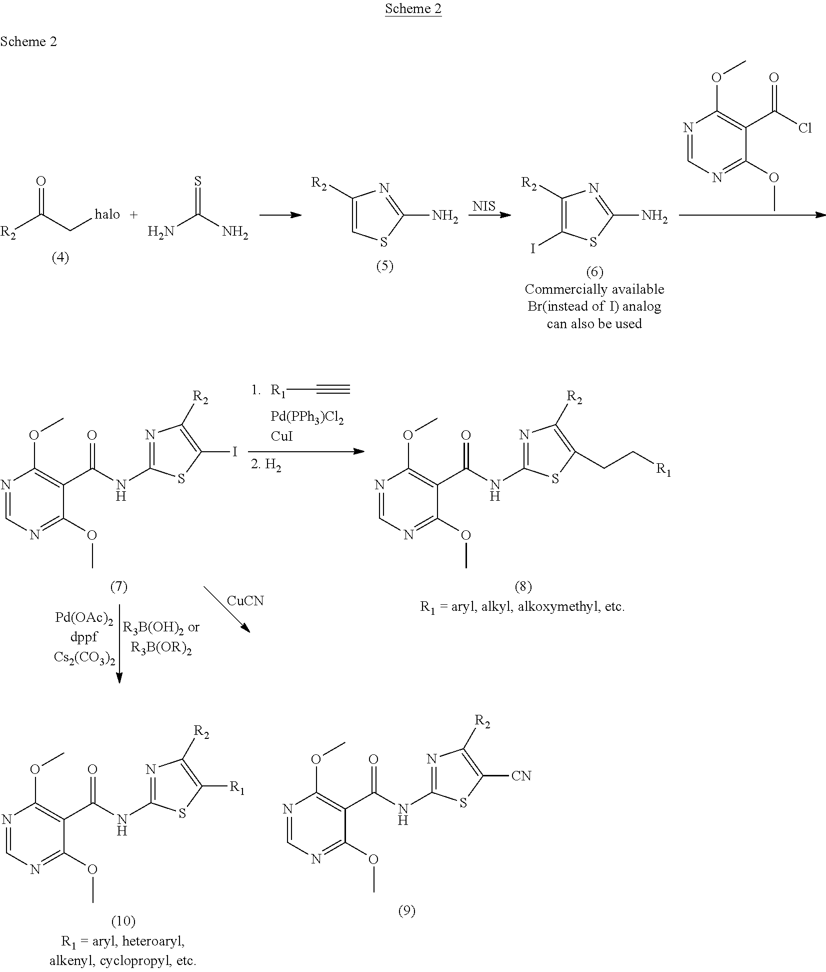

The substituted halo-ketone reactant (4) may be contacted with thiourea reactant to thereby prepare intermediate substituted thiazol-2-amine (5). The halo substituent on the halo-ketone reactant (4) may be chloro, bromo, or iodo. The R₁ and R₂ substituents on reactants (4) and (5) are as defined herein. The cyclization reaction may be carried out in a solvent such as, but not limited to, methanol or ethanol, at room temperature or at an elevated temperature of at least about 40° C., such as between about 60° C. and about 70° C., optionally with stirring.

The substituted intermediate substituted thiazol-2-amine (5) may be contacted with a reactant such as N-bromosuccinimide (NBS) or N-iodosuccinimide (NIS, shown in above Scheme 2) to thereby prepare bromo- or iodo-substituted intermediate (6). The halogenation reaction may be carried out in a solvent such as, but not limited to, methylene chloride and/or acetic acid, at room temperature or at an elevated temperature of at least about 40° C., such as between about 60° C. and about 70° C., optionally with stirring.

The bromo- or iodo-substituted intermediate (6) may be contacted with 4,6-dimethoxypyrimidine-5-carbonyl chloride reactant (the synthesis thereof is described in the examples) to thereby prepare compound (7). The coupling reaction may be carried out in a solvent such as, but not limited to, methylene chloride and/or DMF, at room temperature or at an elevated temperature of at least about 40° C., such as between about 60° C. and about 70° C., optionally with stirring.

Compound (7) may be further modified to prepare additional compounds (8), (9), and (10). Compound (7) may be reacted with a substituted acetylene in the presence of catalytic amounts of tetrakis(triphenylphosphine)palladium, Pd(PPh₃)Cl₂, and CuI to prepare an alkenyl intermediate (not pictured). The alkenyl intermediate may be hydrogenated in a hydrogen ambient atmosphere to thereby prepare the compound (8).

Alternatively, compound (7) may be reacted with copper cyanide to thereby prepare the compound (9). The reaction may be carried out in a solvent such as, but not limited to, methylene chloride and/or DMF, at room temperature or at an elevated temperature of at least about 40° C., such as between about 100° C. and about 150° C. The reaction may be catalyzed by microwave heating.

Alternatively, compound (7) may be contacted with a mixture of reactants including substituted boronic acid, cesium carbonate, and palladium acetate/1,1′-dis(diphenylphosphino)ferrocene to thereby prepare the compound (10). The coupling reaction may be carried out in a solvent such as, but not limited to, methylene chloride and/or DMF, at room temperature or at an elevated temperature of at least about 40° C., such as between about 70° C. and about 100° C., optionally with stirring.

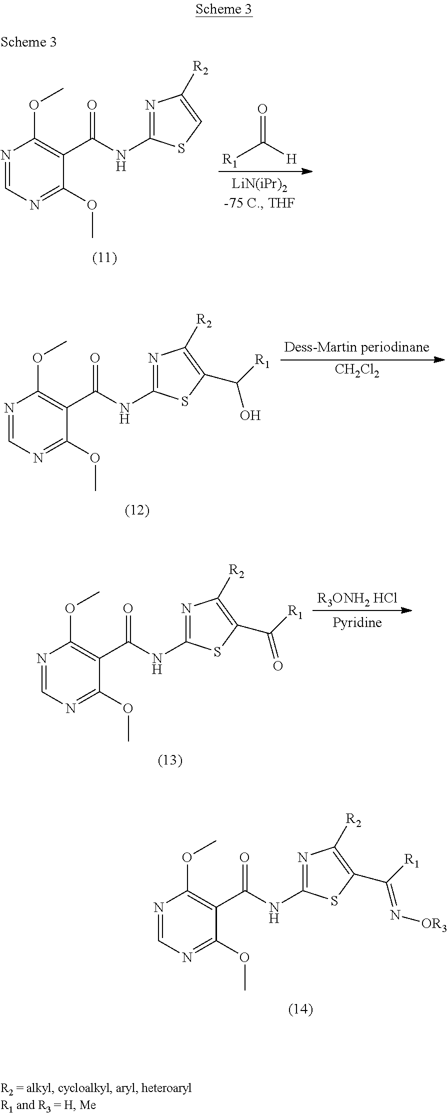

The compound (11) may be prepared according to the Examples. Compound (11) may be prepared, for example, by cyclizing substituted halo-ketone reactant (4) with thiourea reactant to thereby prepare intermediate substituted thiazol-2-amine (5), which is followed by contacting the intermediate prepared thereby with 4,6-dimethoxypyrimidine-5-carbonyl chloride reactant (the synthesis thereof is described in the examples). These reactions are described above in Scheme 1 and 2.

Compound (11) may be contacted with an aldehyde in the presence of a lithium catalyst to thereby prepare Compound (12). The coupling reaction may be carried out in a solvent such as, but not limited to, methylene chloride, DMF, and/or tetrahydrofuran, at low temperature, such as between about −75° C., optionally with stirring.

Compound (12) may be further contacted with the Dess-Martin periodinane reactant to thereby prepare Compound (13). The coupling reaction may be carried out in a solvent such as, but not limited to, methylene chloride, DMF, and/or tetrahydrofuran, at room temperature or at an elevated temperature of at least about 40° C., such as between about 70° C. and about 100° C., optionally with stirring.

Compound (13) may be further contacted with substituted hydroxylamine hydrochloride to prepare compound (14). The coupling reaction may be carried out in a solvent such as, but not limited to, pyridine, at room temperature or cooled in an ice bath, optionally with stirring.

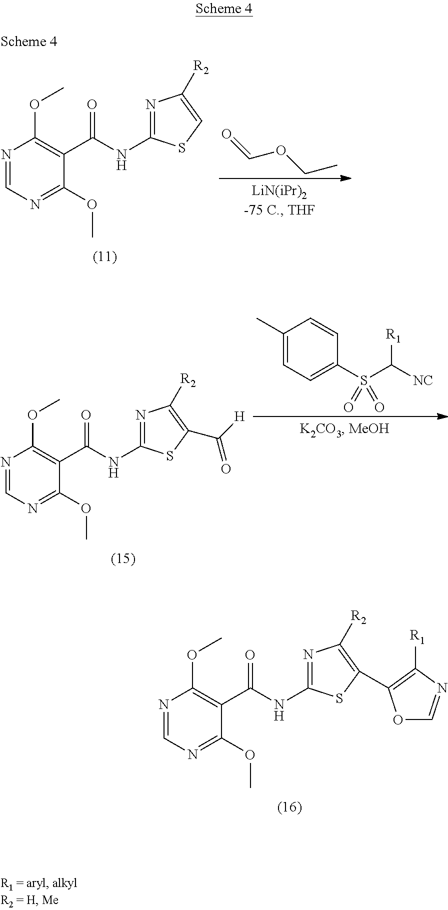

Compound (11) may be prepared according to the Examples. Compound (11) may be prepared, for example, by cyclizing substituted halo-ketone reactant (4) with thiourea reactant to thereby prepare intermediate substituted thiazol-2-amine (5), which is followed by contacting the intermediate prepared thereby with 4,6-dimethoxypyrimidine-5-carbonyl chloride reactant (the synthesis thereof is described in the examples). These reactions are described above in Scheme 1 and 2.

Compound (11) may be contacted with an aldehyde in the presence of a lithium catalyst to thereby prepare Compound (15). The coupling reaction may be carried out in a solvent such as, but not limited to, methylene chloride, DMF, and/or tetrahydrofuran, at low temperature, such as between about −75° C., optionally with stirring.

Compound (15) may be contacted with a substituted tosylmethyl isocyanide catalyst to thereby prepared Compound (16). The cyclization reaction may be carried out in the presence of base such as, but not limited to, potassium carbonate and in a solvent such as, but not limited to, methanol or ethanol, at room temperature or at an elevated temperature of at least about 40° C., such as between about 60° C. and about 70° C., optionally with stirring.

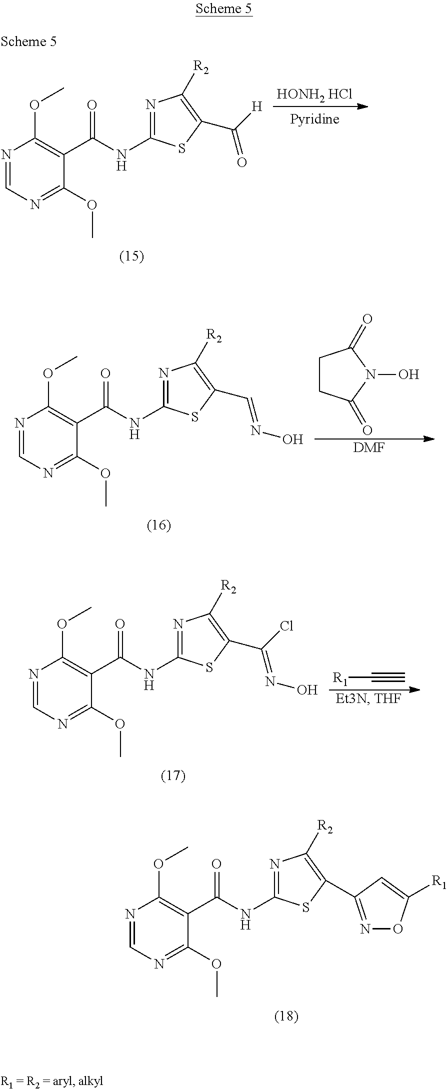

Compound (15) may be prepared according to the Examples and as described above in the context of Scheme 4. Compound (15) may be further contacted with hydroxylamine hydrochloride to prepare compound (16). The coupling reaction may be carried out in a solvent such as, but not limited to, pyridine, at room temperature or at an elevated temperature of at least about 40° C., such as between about 40° C. and about 60° C., optionally with stirring.

Compound (16) may be further contacted with N-chlorosuccinimide (NCS) to prepare Compound (17). Alternative halogenating agents include N-bromosuccinimide (NBS) or N-iodosuccinimide (NIS) to thereby prepare bromo- or iodo-substituted compounds. The halogenation reaction may be carried out in a solvent such as, but not limited to, methylene chloride, DMF, and/or tetrahydrofuran, at room temperature or at an elevated temperature of at least about 40° C., such as between about 60° C. and about 70° C., optionally with stirring.

Compound (17) may be further contacted with a substituted alkyne to prepare Compound (18). The cyclization reaction may be carried out in the presence of base such as, but not limited to, trimethylamine, and in a solvent such as, but not limited to, methylene chloride, DMF, and/or tetrahydrofuran, at room temperature or at an elevated temperature of at least about 40° C., such as between about 60° C. and about 70° C., optionally with stirring.

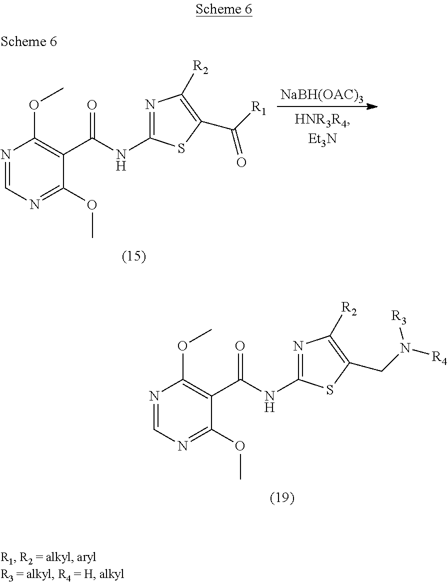

Compound (15) may be prepared according to the Examples and as described above in the context of Scheme 4. Compound (15) may be further contacted with substituted amine to prepare compound (19). The coupling reaction may be carried out in the presence of a reducing agent such as, but not limited to, sodium triacetoxyborohydride, and in a base such as, but not limited to, trimethylamine, at room temperature or at an elevated temperature of at least about 40° C., such as between about 60° C. and about 70° C., optionally with stirring.

It will be appreciated that the synthetic schemes and specific examples as illustrated in the Examples section are illustrative and are not to be read as limiting the scope of the invention as it is defined in the appended claims. All alternatives, modifications, and equivalents of the synthetic methods and specific examples are included within the scope of the claims.

Optimum reaction conditions and reaction times for each individual step may vary depending on the particular reactants employed and substituents present in the reactants used.

Unless otherwise specified, solvents, temperatures and other reaction conditions may be readily selected by one of ordinary skill in the art. Specific procedures are provided in the Examples section. Reactions may be worked up in the conventional manner, e.g. by eliminating the solvent from the residue and further purified according to methodologies generally known in the art such as, but not limited to: crystallization, distillation, extraction, trituration and chromatography. Unless otherwise described, the starting materials and reagents are either commercially available or may be prepared by one skilled in the art from commercially available materials using methods described in the chemical literature.

Routine experimentations, including appropriate manipulation of the reaction conditions, reagents and sequence of the synthetic route, protection of any chemical functionality that may not be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which may be found in T. Greene and P. Wuts, Protecting Groups in Organic Synthesis (3^{rd} ed.), John Wiley & Sons, NY (1999), which is incorporated herein by reference in its entirety. Synthesis of the compounds of the invention may be accomplished by methods analogous to those described in the synthetic schemes described hereinabove and in specific examples.

Starting materials, if not commercially available, may be prepared by procedures selected from standard organic chemical techniques, techniques that are analogous to the synthesis of known, structurally similar compounds, or techniques that are analogous to the above described schemes or the procedures described in the synthetic examples section.

When an optically active form of a compound of the invention is required, it may be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound of the invention is required, it may be obtained by carrying out one of the above procedures using a pure geometric isomer as a starting material, or by resolution of a mixture of the geometric isomers of the compound or intermediates using a standard procedure such as chromatographic separation.

### EXAMPLES

Generally, LCMS measurement were run on Agilent 1200 HPLC/6100 SQ System using the following conditions: Mobile Phase: A) Water (0.05% TFA), B) Acetonitrile (0.05% TFA); Gradient Phase: 5%-95% in 1.3 min; Flow rate: 1.6 mL/min; Column: XBridge, 2.5 min; Oven temp: 50° C.

### Example 1

### 4,6-dimethoxy-N-[5-methyl-4-(trifluoromethyl)-1,3-thiazol-2-yl]pyrimidine-5-carboxamide

### Example 1A

### 5-methyl-4-(trifluoromethyl)thiazol-2-amine

To a solution of thiourea (12.1 g, 159 mmol) in ethanol (120 mL) was added 3-bromo-1,1,1-trifluorobutan-2-one (32.5 g, 159 mmol) dropwise via syringe pump over 30 min. After the addition was complete, the mixture was stirred for 90 min at 60° C. and for an additional 3 h at 70° C. The reaction mixture was allowed to cool to ambient temperature and was concentrated under reduced pressure. The crude solids were washed with Et₂O and then dissolved in water. The aqueous solution was brought to a pH of 11 by addition of 1 N NaOH. Material was extracted from the aqueous layer with CH₂Cl₂ (3×50 mL). The combined organics were dried over anhydrous Na₂SO₄, filtered and concentrated under reduced pressure to give the desired Example 1A compound (22.0 g, 76% yield) which solidified upon concentration. MS (ESI) m/z 183 (M+H)⁺.

### Example 1B

### 4,6-dimethoxy-N-[5-methyl-4-(trifluoromethyl)-1,3-thiazol-2-yl]pyrimidine-5-carboxamide

The title compound was prepared as described in Example 2C, substituting Example 1A for Example 2B (25.3 mg, 67.8% yield). ¹H NMR (400 MHz, CDCL₃) δ 10.26 (s, 1H), 8.49 (s, 1H), 4.08 (s, 6H), 2.52 (d, J=2.1 Hz, 3H). MS (DCI/NH₃) m/z 349 (M+H)⁺.

### Example 2

### N-[5-iodo-4-(trifluoromethyl)-1,3-thiazol-2-yl]-4,6-dimethoxypyrimidine-5-carboxamide

### Example 2A

### 4,6-dimethoxypyrimidine-5-carboxylic acid

To a solution of 2,2,6,6-tetramethylpiperidine (25.6 ml, 152 mmol) in THF (1000 ml) at 0° C. was added a 2.5M hexane solution of n-butyllithium (60 ml, 150 mmol) dropwise. The mixture was stirred for 15 min, cooled to −78° C., and a solution of 4,6-dimethoxypyrimidine (16.4 g, 117 mmol) in THF (100 ml) was added dropwise. The mixture was stirred for 20 min, and CO₂ was bubbled into the reaction via cannula from a flask of warming dry ice. The mixture was stirred for 10 min, allowed to warm to ambient temperature, stirred for an additional 2 h, concentrated in vacuo, diluted with water, and washed with CH₂Cl₂. The aqueous layer was acidified to pH 1 with concentrated HCl, concentrated to ˜100 mL, filtered, washed with water (˜50 mL), and dried (55° C., vacuum oven) to give Example 2A (17.9 g, 83%), which was used without purification: MS (ESI) m/z 185.5 (M+H)

### Example 2B

### 5-iodo-4-(trifluoromethyl)thiazol-2-amine

Neat iodine monochloride (1.70 ml, 33.9 mmol) was added to a solution of 4-(trifluoromethyl)thiazol-2-amine (5.0 g, 29.6 mmol) (J. Hetrocycl. Chem., 1991, 28, 907-911) in CH₂Cl₂ (60 ml) and AcOH (15 ml) at 0 C, stirred for 1 h, allowed to warm to room temperature with stirring for 2 h. Additional iodine monochloride (0.34 ml, 6.79 mmol) was added, stirred for 2 h, and the resulting mixture was concentrated. The material was diluted with water, neutralized with solid Na₂CO₃, diluted with sat Na₂SO₃ (−8 mL), filtered, washed with water, and the solid dried to give crude Example 2B (8.3 g, 96%), which was used without purification. MS (ESI) m/z 295 (M+H)

### Example 2C

### N-[5-iodo-4-(trifluoromethyl)-1,3-thiazol-2-yl]-4,6-dimethoxypyrimidine-5-carboxamide

To a suspension of Example 2A (7.2 g, 39.2 mmol) in CH₂Cl₂ (100 mL) and catalytic DMF, oxalyl chloride (4.5 mL, 51.4 mmol) was added dropwise, and the mixture was stirred for 1 h. After the reaction mixture was concentrated in vacuo and dissolved in CH₂Cl₂ (100 mL), Example 2B (11.5 g, 39.3 mmol), triethylamine (7.5 mL, 53.8 mmol), and 4-dimethylaminopyridine (4.8 g, 39.6 mmol) were added, and mixture stirred overnight at ambient temperature. Reaction mixture was concentrated in vacuo to a solid, added 300 mL EtOAc, washed with 1N HCl (2×300 mL), sat NaHCO₃ (2×300 mL), brine and dried over Na₂SO₄. Solution was concentrated to an oil, diluted with 100 mL MTBE, filtered through a silica gel plug, rinsed with MTBE, and concentrated under reduced pressure to give desired Example 2C (16.0 g, 89% yield) as a yellow foam. ¹H NMR (400 MHz, DMSO-d₆) δ ppm 13.22 (s, 1H), 8.63 (s, 1H), 3.96 (s, 6H). MS (ESI) m/z 461 (M+H)⁺.

### Example 3

### 4,6-dimethoxy-N-[4-(trifluoromethyl)-1,3-thiazol-2-yl]pyrimidine-5-carboxamide

The title compound was prepared as described in Example 2C, substituting 4-(trifluoromethyl)thiazol-2-amine (J. Hetrocycl. Chem., 1991, 28, 907-911) for Example 2B (25.3 mg, 67.8% yield). ¹H NMR (400 MHz, DMSO-d₆) δ 12.98 (s, 1H), 8.63 (s, 1H), 8.04 (d, J=1.2 Hz, 1H), 3.96 (s, 6H). MS (ESI) m/z 335 (M+H)⁺.

### Example 4

### N-[5-(4-chlorophenyl)-4-(trifluoromethyl)-1,3-thiazol-2-yl]-4,6-dimethoxypyrimidine-5-carboxamide

### Example 4A

### 5-(4-chlorophenyl)-4-(trifluoromethyl)thiazol-2-amine

2 M Na₂CO₃ (0.8 g, 7.65 mmol) solution was added to a solution of Example 2B (0.9 g, 3.06 mmol), (4-chlorophenyl)boronic acid (574 mg, 3.67 mmol) and Pd(Ph₃P)₄ (0.11 g, 0.092 mmol) in dioxane (13.5 mL). The reaction was purged by N₂ and heated in a Biotage microwave at 100° C. for 2 hr. The reaction mixture was cooled to ambient temperature, diluted with saturated sodium chloride solution and extracted with ethyl acetate (3×200 mL). The combined organic layers were dried over Na₂SO₄, filtered through Buchner funnel and concentrated under reduced pressure. The residue was purified by silica gel chromatography (petroleum ether/ethyl acetate=5/1) to give the title compound (0.73 g, 85% yield) as a brown solid. 1H NMR (400 MHz, DMSO-d6) d ppm 7.41 (d, J=8.5 Hz, 2H), 7.50 (d, J=8.5 Hz, 2H), 7.55 (s, 2H). MS (ESI) m/z=279 (M+H)+.

### Example 4B

### N-[5-(4-chlorophenyl)-4-(trifluoromethyl)-1,3-thiazol-2-yl]-4,6-dimethoxypyrimidine-5-carboxamide

The title compound was prepared as described in Example 2C, substituting Example 4A for Example 2B (0.12 g 43%). ¹H NMR (400 MHz, DMSO-d₆) δ ppm 13.1 (s, 1H), 8.63 (s, 1H), 7.58 (m, 4H), 3.97 (s, 6H). MS (ESI) m/z 445 (M+H)⁺.

### Example 5

### N-[5-(4-chlorophenyl)-4-(trifluoromethyl)-1,3-thiazol-2-yl]-4,6-diethoxypyrimidine-5-carboxamide

### Example 5A

### Ethyl 4,6-diethoxypyrimidine-5-carboxylate

To anhydrous MeOH (30 ml) under nitrogen was added portionwise sodium (0.666 g, 29.0 mmol). The mixture was stirred until all metal had dissolved, and then a solution of methyl 4,6-dichloropyrimidine-5-carboxylate (2.0 g, 9.66 mmol) in 25 mL anhydrous MeOH was added dropwise. The mixture was stirred for one hour at ambient temperature, then refluxed for one hour, cooled to ambient temperature and concentrated under reduced pressure. The mixture was diluted with water (150 mL) and extracted three times with a 1:1 mixture of ethyl acetate:diethyl ether. Organic extracts were combined, washed once with brine, dried (MgSO₄), filtered and concentrated to give the title compound (2.09 g 90% yield). ¹H NMR (400 MHz, DMSO-d₆) δ ppm 8.53 (s, 1H), 4.42 (q, J=7.1, 7.1, 7.0 Hz, 4H), 4.28 (q, J=7.1, 7.1, 7.1 Hz, 2H), 1.28 (dt, J=9.5, 7.1, 7.1 Hz, 9H). MS (DCI/NH₃) m/z 241 (M+H)⁺.

### Example 5B

### 4,6-diethoxypyrimidine-5-carboxylic acid

Example 5A (2.09 g, 10.55 mmol) was stirred in THF (40 ml) and MeOH (10 ml), 1.0 M NaOH (84 ml, 84 mmol) was added, and the reaction mixture was heated at 50° C. for 3 h. The reaction mixture was concentrated under reduced pressure to one half of the original volume, chilled in an ice bath, acidified with 5M HCl to a pH ˜2, and the resulting white solid was filtered off and washed with ice cold water (product is water soluble). The product was dried overnight in vacuo to produce the title compound (0.72 g, 32% yield). ¹H NMR (400 MHz, DMSO-d₆) δ ppm 13.18 (s, 1H), 8.50 (s, 1H), 4.47-4.36 (m, 4H), 1.29 (t, J=7.0, 7.0 Hz, 6H). MS (DCI/NH₃) m/z 213 (M+H)⁺.

### Example 5C

### N-[5-(4-chlorophenyl)-4-(trifluoromethyl)-1,3-thiazol-2-yl]-4,6-diethoxypyrimidine-5-carboxamide

To a solution of Example 4A (0.1 g, 0.359 mmol) and Example 5A (0.08 g, 0.395 mmol) in dry pyridine (3 mL), phosphoryl trichloride (0.037 mL, 0.395 mmol) was added dropwise and the reaction was stirred at room temperature. After 4 h, the reaction was quenched with an addition of saturated solution of ammonium chloride and extracted with ethyl acetate. The organic phase was washed with water and brine, dried over magnesium sulfate, and filtered. The filtrate was concentrated under reduced pressure and the residue was purified by silica gel chromatography eluting with a gradient of 0-45% ethyl acetate in heptane to give 0.02 g (12%) of the title compound. 1H NMR (400 MHz, CDCl₃) δ ppm 9.8 (s, 1H), 8.6 (s, 1H), 7.58 (m, 4H), 4.4 (m, 4H), 1.3 (t, J=7 Hz, 6H). MS (ESI) m/z 473 (M+H)⁺.

### Example 6

### N-[5-ethyl-4-(trifluoromethyl)-1,3-thiazol-2-yl]-4,6-dimethoxypyrimidine-5-carboxamide

### Example 6A5-ethyl-4-(trifluoromethyl)thiazol-2-amine

The title compound was prepared as described in Example 1A, substituting 3-bromo-1,1,1-trifluoropentan-2-one for 3-bromo-1,1,1-trifluorobutan-2-one (1.4 g 54%). ¹H NMR (400 MHz, CDCl₃) δ ppm 3.9 (m, 2H), 1.2 (t, J=8 Hz, 3H). MS (ESI) m/z 197 (M+H)⁺.

### Example 6B

### N-[5-ethyl-4-(trifluoromethyl)-1,3-thiazol-2-yl]-4,6-dimethoxypyrimidine-5-carboxamide

The title compound was prepared as described in Example 2C, substituting Example 6A for Example 2B (0.2 g 38%). ¹H NMR (400 MHz, CDCl₃) δ ppm 10.0 (s, 1H), 8.5 (s, 1H), 4.1 (s, 6H), 2.9 (m, 2H), 1.3 (t, J=7 Hz, 3H). MS (ESI) m/z 363 (M+H)⁺.

### Example 7

### N-[5-cyano-4-(trifluoromethyl)-1,3-thiazol-2-yl]-4,6-diethoxypyrimidine-5-carboxamide

### Example 7A

### 4,6-diethoxy-N-(5-iodo-4-(trifluoromethyl)thiazol-2-yl)pyrimidine-5-carboxamide

The title compound was prepared as described in Example 2C substituting Example 5A for Example 2A (0.5 g 18%). ¹H NMR (400 MHz, CDCl₃) δ ppm 10.2 (s, 1H), 8.4 (s, 1H), 4.6 (dd, J=7 Hz, J=3 Hz, 4H), 1.4 (t, J=7 Hz, 6H). MS (ESI) m/z 489 (M+H)⁺.

### Example 7B

### N-[5-cyano-4-(trifluoromethyl)-1,3-thiazol-2-yl]-4,6-diethoxypyrimidine-5-carboxamide

To a solution of Example 7A (0.045 g, 0.092 mmol) in anhydrous DMF (3 mL), copper cyanide (9.91 mg, 0.111 mmol) was added and the reaction was irradiated in a microwave machine for 10 min at 150° C. After cooling, reaction was filtered, concentrated under reduced pressure and the residue was purified by silica gel chromatography eluting with a gradient of 0-35% ethyl acetate in heptane to give 0.012 g (31%) of title compound. ¹H NMR (400 MHz, CDCl₃) δ ppm 10.6 (s, 1H), 8.5 (s, 1H), 4.6 (m, 4H), 1.5 (t, J=7 Hz, 6H). MS (−ESI) m/z 386 (M−H).

### Example 8

### N-[5-(6-fluoropyridin-3-yl)-4-(trifluoromethyl)-1,3-thiazol-2-yl]-4,6-dimethoxypyrimidine-5-carboxamide

A vial was charged with Example 2C (0.5 g, 1.087 mmol), (6-fluoropyridin-3-yl)boronic acid (0.15 g, 1.09 mmol), cesium carbonate (2.2 mL, 2.17 mmol), PdCl₂(dppf).CH₂Cl₂ (0.089 g, 0.109 mmol) and anhydrous dioxane (15 mL). The vial was purged with nitrogen for 10 minutes and mixture was stirred overnight at 80° C. The mixture was cooled to room temperature, diluted with 100 mL of ethyl acetate, washed with water and brine, then dried over magnesium sulfate, filtered and concentrated under reduced pressure. Silica gel chromatography eluting with a gradient of 0-60% ethyl acetate in heptane gave the title compound (0.18 g, 39%). ¹H NMR (400 MHz, CDCl₃) δ ppm 10.2 (s, 1H), 8.5 (s, 1H), 8.3 (m, 1H), 7.9 (m, 1H), 7.0 (m, 1H) 4.16 (s, 6H). MS (ESI) m/z 430 (M+H)⁺.

### Example 9

### N-(4,5-dimethyl-1,3-thiazol-2-yl)-4,6-dimethoxypyrimidine-5-carboxamide

### Example 9A

### 4,5-dimethylthiazol-2-amine

The title compound was prepared as described in Example 1A, substituting 3-bromobutan-2-one for 3-bromo-1,1,1-trifluorobutan-2-one (0.2 g 57%). ¹H NMR (400 MHz, CDCl₃) δ ppm 2.2 (s, 3H), 2.1 (s, 3H). MS (APCI) m/z 129 (M+H)⁺.

### Example 9B

### N-(4,5-dimethyl-1,3-thiazol-2-yl)-4,6-dimethoxypyrimidine-5-carboxamide

The title compound was prepared as described in Example 2C, substituting Example 9A for Example 2B (0.04 g 44%). ¹H NMR (400 MHz, CDCl₃) δ ppm 10.0 (s, 1H), 8.5 (s, 1H), 4.0 (s, 6H), 2.3 (s, 3H), 2.2 (s, 3H). MS (ESI) m/z 295 (M+H)⁺.

### Example 10

### 4,6-dimethoxy-N-{4-(trifluoromethyl)-5-[(trimethylsilyl)ethynyl]-1,3-thiazol-2-yl}pyrimidine-5-carboxamide

A vial charged with Example 2C (0.2 g, 0.435 mmol), ethynyltrimethylsilane (0.061 mL, 0.435 mmol), cesium carbonate (0.212 g, 0.652 mmol) and anhydrous THF (5 mL) was purged with nitrogen, and copper(I) iodide (8.28 mg, 0.043 mmol) and PdCl₂(dppf).CH₂Cl₂ (0.035 g, 0.043 mmol) were added. The resulting mixture was stirred for 4 h at 60° C., cooled to room temperature, diluted with 50 mL of ethyl acetate and filtered over a pad of CELITE®. Organic phase was washed with water and brine, then dried over magnesium sulfate, filtered and concentrated under reduced pressure. The residue was purified by chromatography on silica gel eluting with a gradient of 0-30% EtOAc in hexanes to afford the title compound (0.06 g, 33%.). ¹H NMR (400 MHz, CDCl₃) δ ppm 10.2 (s, 1H), 8.5 (s, 1H), 4.1 (s, 6H), 0.27 (s, 9H). MS (ESI) m/z 431 (M+H)⁺.

### Example 11

### N-[5-ethynyl-4-(trifluoromethyl)-1,3-thiazol-2-yl]-4,6-dimethoxypyrimidine-5-carboxamide

To a solution of Example 10 (0.03 g, 0.070 mmol) in methanol (5 mL), potassium carbonate (0.029 g, 0.209 mmol) was added and the mixture stirred at room temperature for 1 h. Reaction was poured into ethyl acetate (50 mL) and washed with water and brine. Organic phase was dried over magnesium sulfate, filtered and concentrated under reduced pressure. The residue was purified by silica gel chromatography eluting with a gradient of 0-15% MeOH in CH₂Cl₂ to give the title compound (0.017 g, 68%.). ¹H NMR (400 MHz, CDCl₃) δ ppm 10.4 (s, 1H), 8.5 (s, 1H), 4.1 (s, 7H). MS (ESI) m/z 359 (M+H)⁺.

### Example 12

### N-[5-(3-hydroxyprop-1-yn-1-yl)-4-(trifluoromethyl)-1,3-thiazol-2-yl]-4,6-dimethoxypyrimidine-5-carboxamide

### Example 12A

### N-(5-(((tert-butyldimethylsilyl)oxy)ethynyl)-4-(trifluoromethyl)thiazol-2-yl)-4,6-dimethoxypyrimidine-5-carboxamide

The title compound was prepared as described in Example 10, substituting tertbutyldimethyl(2-propynyloxy)silane for ethynyltrimethylsilane (0.1 g, 35%). ¹H NMR (400 MHz, CDCl₃) δ ppm 10.2 (s, 1H), 8.5 (s, 1H), 4.6 (s, 2H), 4.1 (s, 6H), 0.9 (s, 9H), 0.2 (s, 6H). MS (ESI) m/z 503 (M+H)⁺.

### Example 12B

### N-[5-(3-hydroxyprop-1-yn-1-yl)-4-(trifluoromethyl)-1,3-thiazol-2-yl]-4,6-dimethoxypyrimidine-5-carboxamide

A solution of Example 12A (0.085 g, 0.169 mmol) in anhydrous THF (5 mL) was treated with tetrabutylammonium fluoride (0.254 mL, 0.254 mmol). After stirring for 2 h at room temperature, the reaction was diluted with 30 mL of CH₂Cl₂ and washed with water and brine. Organic phase was dried over magnesium sulfate, filtered and concentrated under reduced pressure. Residue was purified by silica gel chromatography eluting with a gradient of 0-60% EtOAc in heptanes to give the title compound (0.035 g, 54%). ¹H NMR (400 MHz, CD₃OD) δ ppm 8.5 (s, 1H), 4.4 (s, 2H) 4.0 (s, 6H). MS (ESI) m/z 389 (M+H)⁺.

### Example 13

### N-[5-(3-hydroxypropyl)-4-(trifluoromethyl)-1,3-thiazol-2-yl]-4,6-dimethoxypyrimidine-5-carboxamide

To a solution of Example 12B (0.06 g, 0.14 mmol) in THF (5 mL), Pd/C (5%, 0.025 g) was added and the mixture was stirred under an atmosphere of hydrogen for 16 h. Reaction was filtered and evaporated. The crude material was purified by silica gel chromatography eluting with a gradient of 0-15% methanol in CH₂Cl₂ to give the title compound (0.042 g, 77%). ¹H NMR (400 MHz, CDCl₃) δ ppm 10.1 (s, 1H), 8.5 (s, 1H), 4.1 (s, 6H), 3.7 (m, 2H), 3.0 (m, 2H), 1.9 (m, 2H). MS (ESI) m/z 393 (M+H)⁺.

### Example 14

### N-[5-(cyclopropylethynyl)-4-(trifluoromethyl)-1,3-thiazol-2-yl]-4,6-dimethoxypyrimidine-5-carboxamide

The title compound was prepared as described in Example 10, substituting ethynylcyclopropane for ethynyltrimethylsilane (0.07 g, 23%). ¹H NMR (400 MHz, CDCl₃) δ ppm 10.1 (s, 1H), 8.5 (s, 1H), 4.1 (s, 6H), 1.5 (m, 1H), 0.95 (m, 2H), 0.86 (m, 2H). MS (ESI) m/z 399 (M+H)⁺.

### Example 15

### 4,6-dimethoxy-N-[5-(3-methoxyprop-1-yn-1-yl)-4-(trifluoromethyl)-1,3-thiazol-2-yl]pyrimidine-5-carboxamide

The title compound was prepared as described in Example 10, substituting methyl propargyl ether for ethynyltrimethylsilane (1.7 g, 80%). ¹H NMR (400 MHz, DMSO-d₆) δ ppm 13.3 (s, 1H), 8.6 (s, 1H), 4.4 (s, 2H), 3.9 (s, 6H). MS (ESI) m/z 403 (M+H)⁺.

### Example 16

### 4,6-dimethoxy-N-[5-(3-methoxypropyl)-4-(trifluoromethyl)-1,3-thiazol-2-yl]pyrimidine-5-carboxamide

To a solution of Example 15 (0.705 g, 1.752 mmol) in THF (20 ml) in a 50 ml pressure bottle was added 5% Pd/C (0.15 g, 0.626 mmol), and the mixture was hydrogenated for 16 h at 30 psi at ambient temperature. The mixture was filtered and concentrated to an oil. The oil was taken up in a minimal amount of diethyl ether and triturated with heptane while vigorously stirring. The resulting solid was filtered off, washing with excess heptane to give the title compound (0.551 g, 77% yield) as a white solid. ¹H NMR (400 MHz, CDCl₃) δ 10.21 (s, 1H), 8.50 (s, 1H), 4.09 (s, 6H), 3.43 (t, J=6.1, 6.1 Hz, 2H), 3.35 (d, J=1.6 Hz, 3H), 3.01 (t, J=7.5, 7.5 Hz, 2H), 2.11-1.78 (m, 2H).. MS (DCI/NH₃) m/z 407 (M+H)⁺.

### Example 17

### 4,6-dimethoxy-N-{5-[(1Z)-3-methoxyprop-1-en-1-yl]-4-(trifluoromethyl)-1,3-thiazol-2-yl}pyrimidine-5-carboxamide

To a solution of Example 15 (0.162 g, 0.403 mmol) in THF (20 ml) in a 50 ml pressure bottle was added to 5% Pd/CaCO₃ (Lindlar) (0.016 g, 0.152 mmol), and the mixture was hydrogenated for 30 min at 30 psi at ambient temperature. After filtrating and concentrating, the crude material was purified by flash chromatography (4 g silica gel, 5-30% gradient of ethyl acetate in heptane) to provide the title compound (82 mg. 50% yield) as a white solid. ¹H NMR (501 MHz, CDCl₃) δ ppm 10.34 (s, 1H), 8.52 (s, 1H), 6.74 (dd, J=12.0, 1.8 Hz, 1H), 5.97 (dt, J=12.0, 6.0, 6.0 Hz, 1H), 4.27 (dd, J=6.0, 1.9 Hz, 2H), 4.12 (s, 6H), 3.41 (s, 3H). MS (DCI/NH₃) m/z 405 (M+H)⁺.

### Example 18

### 4,6-dimethoxy-N-{5-[(1E)-3-methoxyprop-1-en-1-yl]-4-(trifluoromethyl)-1,3-thiazol-2-yl}pyrimidine-5-carboxamide

A suspension of Example 2 (0.220 g, 0.478 mmol) in (E)-2-(3-methoxyprop-1-en-1-yl)-4,4,5,5-tetramethyl-1,3,2-dioxaborolane (0.101 ml, 0.478 mmol) and PdCl₂(dppf) (0.052 g, 0.072 mmol) was stirred in dioxane (5 ml) under a nitrogen atmosphere. After de-gassing, 1.0 M aqueous cesium carbonate (0.956 ml, 0.956 mmol) was added and stirred at ambient temperature for one hour, then heated at 80° C. overnight. The mixture was filtered through a bed of CELITE®, washed with excess ethyl acetate and the filtrate concentrated under reduced pressure. The crude material was purified by flash chromatography (12 g silica gel, 35-65% gradient of ethyl acetate in heptane) to provide the title compound (90 mg. 47% yield) as a light yellow solid. ¹H NMR (400 MHz, CDCl₃) δ ppm 10.16 (s, 1H), 8.51 (s, 1H), 6.77 (s, 1H), 6.20 (dt, J=15.8, 5.5, 5.5 Hz, 1H), 4.12 (s, 6H), 4.09 (d, J=5.4 Hz, 2H), 3.40 (s, 3H). MS (DCI/NH₃) m/z 405 (M+H)⁺.

### Example 19

### N-[5-(3-hydroxy-3-methylbut-1-yn-1-yl)-4-(trifluoromethyl)-1,3-thiazol-2-yl]-4,6-dimethoxypyrimidine-5-carboxamide

The title compound was prepared as described in Example 10, substituting 2-methylbut-3-yn-2-ol for ethynyltrimethylsilane (0.23 g, 85%). ¹H NMR (400 MHz, CDCl₃) δ ppm 10.1 (s, 1H), 8.5 (s, 1H), 4.1 (s, 6H), 1.6 (s, 6H). MS (ESI) m/z 417 (M+H)⁺.

### Example 20

### N-[5-(3-hydroxy-3-methylbutyl)-4-(trifluoromethyl)-1,3-thiazol-2-yl]-4,6-dimethoxypyrimidine-5-carboxamide

To a solution of Example 19 (0.2 g, 0.53 mmol) in methanol (10 mL), Pd(OH)₂/C (20%, 0.22 g) was added and the mixture was stirred under an atmosphere of hydrogen for 32 h. The mixture was filtered, concentrated under reduced pressure and the residue was purified by silica gel chromatography eluting with a gradient of 0-15% methanol in CH₂Cl₂ to give the title compound (0.1 g, 45%). ¹H NMR (400 MHz, CDCl₃) δ ppm 10.0 (s, 1H), 8.5 (s, 1H), 4.1 (s, 6H), 3.0 (m, 2H), 1.8 (m, 2H), 1.3 (s, 6H). MS (ESI) m/z 421 (M+H)⁺.

### Example 21

### ethyl 2-{[(4,6-dimethoxypyrimidin-5-yl)carbonyl]amino}-4-(trifluoromethyl)-1,3-thiazole-5-carboxylate

The title compound was prepared as described in Example 2C, substituting ethyl 2-amino-4-(trifluoromethyl)thiazole-5-carboxylate (Combi-Blocks) for Example 2B (2.6 g, 31%). ¹H NMR (400 MHz, CDCl₃) δ ppm 10.5 (s, 1H), 8.5 (s, 1H), 4.4 (m, 2H), 4.1 (s, 6H), 1.4 (t, J=8 Hz, 3H). MS (ESI) m/z 407 (M+H)⁺.

### Example 22

### N-[5-(2-hydroxyethyl)-4-(trifluoromethyl)-1,3-thiazol-2-yl]-4,6-dimethoxypyrimidine-5-carboxamide

### Example 22A

### 4,6-dimethoxy-N-(4-(trifluoromethyl)-5-vinylthiazol-2-yl)pyrimidine-5-carboxamide

The title compound was prepared as described in Example 8, substituting vinylboronic acid pinacol ester for (6-fluoropyridin-3-yl)boronic acid (1.3 g, 83%). ¹H NMR (400 MHz, CDCl₃) δ ppm 10.1 (s, 1H), 8.5 (s, 1H), 7.0 (m, 1H), 5.7 (dd, J=14 Hz, 1H), 5.4 (dd, J=8 Hz, 1H), 4.1 (s, 6H). MS (ESI) m/z 361 (M+H)⁺.

### Example 22B

### N-[5-(2-hydroxyethyl)-4-(trifluoromethyl)-1,3-thiazol-2-yl]-4,6-dimethoxypyrimidine-5-carboxamide

To a solution of Example 22A (0.2 g, 0.555 mmol) in anhydrous THF (5 mL), borane tetrahydrofuran complex (0.83 mL, 0.833 mmol) was added dropwise at room temperature. After 30 min of stirring an aqueous solution of sodium hydroxide (5.55 mL, 5.55 mmol) was added slowly followed by hydrogen peroxide solution (0.572 mL, 5.55 mmol). The mixture was stirred at room temperature for 2 h, then a saturated solution of sodium sulfite (1 mL) was added and extracted with EtOAc (50 mL). Organic phase was washed with water and brine, then dried over magnesium sulfate, filtered, concentrated under reduced pressure and the residue was purified by silica gel chromatography eluting with a gradient 0-50% of ethyl acetate in hexanes to give the title compound (0.06 g, 30%). ¹H NMR (400 MHz, CDCl₃) δ ppm 10.1 (s, 1H), 8.5 (s, 1H), 4.1 (s, 6H), 3.9 (m, 2H), 3.1 (m, 2H). m/z 379 (M+H)⁺.

### Example 23

### 4,6-dimethoxy-N-{5-[6-(morpholin-4-yl)pyridin-3-yl]-4-(trifluoromethyl)-1,3-thiazol-2-yl}pyrimidine-5-carboxamide

A solution of Example 8 (0.1 g, 0.233 mmol) in anhydrous DMF was treated with N-ethyl-N-isopropylpropan-2-amine (0.08 mL, 0.466 mmol) and morpholine (0.022 g, 0.26 mmol). The reaction was stirred for 12 h at 80° C., volatiles were removed under reduced pressure, and the residue was purified by silica gel chromatography eluting with a gradient 0-50% of EtOAc in hexanes to give the title compound (0.026 g, 26%). ¹H NMR (400 MHz, CDCl₃) δ ppm 10.3 (s, 1H), 8.5 (s, 1H), 8.3 (bs, 1H), 7.7 (m, 1H), 6.9 (m, 1H), 4.1 (s, 6H), 3.9 (m, 4H), 3.7 (m, 4H). m/z 497 (M+H)⁺.

### Example 24

### N-[5-(3-cyanopyridin-4-yl)-4-(trifluoromethyl)-1,3-thiazol-2-yl]-4,6-dimethoxypyrimidine-5-carboxamide

A sealed tube containing Example 2C (0.1 g, 0.217 mmol), 3-cyanopyridine-4-boronic acid pinacol ester (Alfa) (0.055 g, 0.239 mmol), potassium carbonate (0.060 g, 0.435 mmol), THF (2 mL), water (1 mL), and 1,1′-bis(di-tert-butylphosphino)ferrocene palladium dichloride (0.014 g, 0.022 mmol) was heated to 75° C. over 14 h. After cooling to ambient temperature, the mixture was diluted with EtOAc (50 mL) then washed with water and brine. Organic phase was dried over magnesium sulfate, filtered, concentrated under reduced pressure and the residue was purified by silica gel chromatography eluting with a gradient of 0-60% ethyl acetate in hexanes to give the title compound 0.024 g, 25%). ¹H NMR (400 MHz, CDCl₃) δ ppm 10.6 (s, 1H), 9.0 (bs, 1H), 8.9 (m, 1H), 8.5 (s, 1H), 7.5 (m, 1H), 4.1 (s, 6H). m/z 437 (M+H)⁺.

### Example 25

### N-[5-(2-cyano-3-fluorophenyl)-4-(trifluoromethyl)-1,3-thiazol-2-yl]-4,6-dimethoxypyrimidine-5-carboxamide

A vial charged with Example 2C (0.2 g, 0.435 mmol), (2-cyano-3-fluorophenyl)boronic acid (0.079 g, 0.478 mmol), dioxane (5 mL), cesium carbonate (0.283 g, 0.869 mmol) and tetrakis(triphenylphosphine)palladium (0) (0.050 g, 0.043 mmol) was heated to 75° C. over 14 h. After cooling to ambient temperature the mixture was filtered over CELITE®, and the solvent was removed under reduced pressure. The residue was purified by silica gel chromatography eluting with a gradient 0-55% of EtOAc in heptanes to give the title compound (0.04 g, 20%). ¹H NMR (400 MHz, CDCl₃) δ ppm 10.4 (s, 1H), 8.5 (s, 1H), 7.6 (m, 1H), 7.3 (m, 2H) 4.1 (s, 6H). m/z 454 (M+H)⁺.

### Example 26

### N-(5-ethyl-4-phenyl-1,3-thiazol-2-yl)-4,6-dimethoxypyrimidine-5-carboxamide

### Example 26A

### 4-ethyl-5-phenylthiazol-2-amine

The title compound was prepared as described in Example 1A, substituting 2-bromo-1-phenylbutan-1-one for 3-bromo-1,1,1-trifluorobutan-2-one (0.6 g, 62%). ¹H NMR (400 MHz, CDCl₃) δ ppm 7.4 (m, 5H). 2.8 (dd, J=8 Hz, J=3 Hz, 2H), 1.2 (t, J=8 Hz, 3H), MS (ESI) m/z 205 (M+H)⁺.

### Example 26B

### N-(5-ethyl-4-phenyl-1,3-thiazol-2-yl)-4,6-dimethoxypyrimidine-5-carboxamide

The title compound was prepared as described in Example 2C, substituting 26A for Example 2B (0.06 g, 33%). ¹H NMR (400 MHz, CDCl₃) δ ppm 10.5 (s, 1H), 8.5 (s, 1H), 7.5 (m, 5H), 4.0 (s, 6H), 2.9 (m, 2H), 1.3 (t, J=8 Hz, 3H). MS (ESI) m/z 371 (M+H)⁺.

### Example 27

### N-[5-(5-fluoropyridin-3-yl)-4-(trifluoromethyl)-1,3-thiazol-2-yl]-4,6-dimethoxypyrimidine-5-carboxamide

A microwave vial charged with Example 2C (0.2 g, 0.435 mmol), (5-fluoropyridin-3-yl)boronic acid (0.073 g, 0.522 mmol), Pd(amphos)Cl₂ (0.031 g, 0.043 mmol), potassium phosphate tribasic (0.652 mL, 1.304 mmol), and dioxane (3 mL) was purged with nitrogen for a few minutes and irradiated for 15 minutes at 150° C. in a Biotage microwave apparatus. After cooling to ambient temperature the mixture was diluted with 20 mL of EtOAc and filtered. The solvent was evaporated under reduced pressure and the residue was purified by silica gel chromatography eluting with a gradient 0-50% of EtOAc in heptanes to give the title compound (0.1 g, 54%). ¹H NMR (400 MHz, CDCl₃) δ ppm 10.8 (s, 1H), 8.5 (m, 3H), 7.5 (m, 1H), 4.2 (s, 6H). MS (ESI) m/z 430 (M+H)⁺.

### Example 28

### 4,6-dimethoxy-N-[5-(pyrazin-2-yl)-4-(trifluoromethyl)-1,3-thiazol-2-yl]pyrimidine-5-carboxamide

A high-pressure vial charged with Example 2C (0.2 g, 0.435 mmol), 2-(4,4,5,5-tetramethyl-1,3,2-dioxaborolan-2-yl)pyrazine (0.18 g, 0.869 mmol), cesium carbonate (0.28 g, 0.869 mmol), copper(I) chloride (0.043 g, 0.435 mmol), palladium (II) acetate (4.88 mg, 0.022 mmol), 1,1′-bis(di-tert-butylphosphino)ferrocene (0.012 g, 0.022 mmol), and anhydrous DMF (5 mL) was purged with nitrogen for few minutes, then the vial was heated at 100° C. for 16 h. After cooling to ambient temperature, the mixture was diluted with 20 mL of EtOAc and filtered over a pad of CELITE®. Organic phase was evaporated under reduced pressure and the residue was purified by silica gel chromatography eluting with a gradient 0-50% of ethyl acetate in heptane to give the title compound (0.025 g, 14%)¹H NMR (400 MHz, CDCl₃) δ ppm 10.8 (s, 1H), 8.9 (bs, 1H), 8.7 (s, 1H), 8.6 (bs, 1H) 8.5 (s, 1H), 4.1 (s, 6H). MS (ESI) m/z 413 (M+H)⁺.

### Example 29

### 4,6-dimethoxy-N-{5-[5-(morpholin-4-yl)pyrazin-2-yl]-4-(trifluoromethyl)-1,3-thiazol-2-yl}pyrimidine-5-carboxamide

The title compound was prepared as described in Example 28, substituting 4-(5-(4,4,5,5-tetramethyl-1,3,2-dioxaborolan-2-yl)pyrazin-2-yl)morpholine for 2-(4,4,5,5-tetramethyl-1,3,2-dioxaborolan-2-yl)pyrazine (0.02 g, 10%). ¹H NMR (400 MHz, CDCl₃) δ ppm 10.6 (s, 1H), 8.5 (s, 1H), 8.4 (s, 1H), 8.1 (s, 1H), 4.1 (s, 6H), 3.8 (m, 4H), 3.6 (m, 4H). MS (−ESI) m/z 496 (M−H).

### Example 30

### 4,6-dimethoxy-N-{4-(trifluoromethyl)-5-[6-(trifluoromethyl)pyridin-2-yl]-1,3-thiazol-2-yl}pyrimidine-5-carboxamide

The title compound was prepared as described in Example 28, substituting 2-(4,4,5,5-tetramethyl-1,3,2-dioxaborolan-2-yl)-6-(trifluoromethyl)pyridine for 2-(4,4,5,5-tetramethyl-1,3,2-dioxaborolan-2-yl)pyrazine (0.1 g, 53%). ¹H NMR (400 MHz, CDCl₃) δ ppm 10.4 (s, 1H), 8.5 (s, 1H), 7.9 (m, 1H), 7.8 (m, 1H), 7.6 (m, 1H) 4.1 (s, 6H). MS (ESI) m/z 480 (M+H)⁺.

### Example 31

### N-[5-(6-cyanopyridin-2-yl)-4-(trifluoromethyl)-1,3-thiazol-2-yl]-4,6-dimethoxypyrimidine-5-carboxamide

### Example 31A

### 5-bromo-4-(trifluoromethyl)thiazol-2-amine

The title compound was prepared as described in Example 2B, substituting N-bromosuccinimide (NBS) for iodine monochloride (50 g, 84%). ¹H NMR (400 MHz, DMSO-d₆) δ ppm 7.67 (s, 2H).

### Example 31B

### N-(5-bromo-4-(trifluoromethyl)thiazol-2-yl)-4,6-dimethoxypyrimidine-5-carboxamide

The title compound was prepared as described in Example 2C, substituting Example 31A for Example 2B (6.0 g, 55%). ¹H NMR (400 MHz, DMSO-d₆) δ ppm 13.29 (s, 1H), 8.64 (s, 1H), 3.97 (s, 6H). MS (DCI/NH₃) m/z 413 (M+H)⁺, 430 (M+NH₄)⁺.

### Example 31C

### N-[5-(6-cyanopyridin-2-yl)-4-(trifluoromethyl)-1,3-thiazol-2-yl]-4,6-dimethoxypyrimidine-5-carboxamide

A microwave flask charged with Example 31B (0.25 g, 0.605 mmol), 6-(4,4,5,5-tetramethyl-1,3,2-dioxaborolan-2-yl)picolinonitrile (COMBIPHOS) (0.153 g, 0.666 mmol), cesium carbonate (0.39 g, 1.210 mmol), PdCl₂(dppf)CH₂Cl₂ (0.049 g, 0.061 mmol), and anhydrous and degased dioxane (4 mL) was purged with nitrogen and the mixture was irradiated for 30 min at 100° C. in a Biotage microwave. After cooling to ambient temperature the mixture was diluted with 25 mL of EtOAc and filtered over a pad of CELITE®. Organic phase was evaporated under reduced pressure and the residue was purified by silica gel chromatography eluting with a gradient 0-30% of EtOAc in heptane to give the title compound (0.1 g, 46%). ¹H NMR (400 MHz, CDCl₃) δ ppm 10.3 (s, 1H), 8.5 (s, 1H), 7.9 (m, 2H), 7.6 (m, 1H), 4.1 (s, 6H). MS (ESI) m/z 437 (M+H)⁺.

### Example 32

### 4,6-dimethoxy-N-[5-(morpholin-4-yl)-4-(trifluoromethyl)-1,3-thiazol-2-yl]pyrimidine-5-carboxamide

A high-pressure vial charged with Example 2C (0.2 g, 0.435 mmol), isopropyl alcohol (3 mL), morpholine (0.056 mL, 0.652 mmol), copper(I) iodide (4.14 mg, 0.022 mmol), ethylene glycol (0.027 g, 0.435 mmol) and potassium phosphate tribasic (0.185 g, 0.869 mmol) was heated at 80° C. for 48 h. After cooling to ambient temperature, the mixture was filtered and the filtrate evaporated under reduced pressure. The residue was purified by silica gel chromatography eluting with a gradient 0-40% of EtOAc in hexanes to give the title compound (0.012 g, 7%). ¹H NMR (400 MHz, CDCl₃) δ ppm 10.1 (s, 1H), 8.5 (s, 1H), 4.1 (s, 6H), 3.7 (m, 4H), 3.5 (m, 4H). MS (−ESI) m/z 418 (M−H).

### Example 33

### N-{5-[2-(2-cyanopropan-2-yl)pyridin-4-yl]-4-(trifluoromethyl)-1,3-thiazol-2-yl}-4,6-dimethoxypyrimidine-5-carboxamide

To a 1 L round bottom flask was added 2-methyl-2-(4-(4,4,5,5-tetramethyl-1,3,2-dioxaborolan-2-yl)pyridin-2-yl)propanenitrile (17.0 g, 62.4 mmol) (PHARMABLOCK), Example 2C (27.4 g, 59.5 mmol), and potassium carbonate (12.3 g, 89 mmol). The solid was purged with nitrogen for 15 min, and then toluene (250 ml) and water (62.5 ml) were added under nitrogen atmosphere. Pd₂dba₃ (1.1 g, 1.2 mmol) and (1S,3R,5R,7S)-1,3,5,7-tetramethyl-8-phenyl-2,4,6-trioxa-8-phosphaadamantane (0.7 g, 2.4 mmol) were added and continuous nitrogen sparging applied while heating to 50° C. Sparging was then stopped and the reaction mixture was heated to 80° C. After heating overnight, 1-pyrrolidinecarbodithioic acid ammonium salt (0.49 g, 2.97 mmol) was added, and the mixture was stirred at ambient temperature for 1 h, filtered through CELITE® and washed with toluene (50 mL). The filtrate was diluted with toluene (50 mL) and washed with water (100 mL), brine (100 mL), then dried (Na₂SO₄), filtered and concentrated under reduced pressure. After adding silica (2× by weight) the mixture was stirred for 30 min, concentrated and loaded as a solid onto SiO₂ (330 g) and eluted with heptane/EA 0-40% over 90 min with 60 min hold to obtain a solid, which was recrystallized from ethyl acetate (2 vol) and heptane (10 vol). The solid was filtered off, washed with cold heptane (30 mL) and dried in a vacuum oven overnight at 50° C. to give the title compound (25.4 g, 89% yield). ¹H NMR (400 MHz, DMSO-d₆) δ 13.27 (s, 1H), 8.75 (d, J=5.1 Hz, 1H), 8.65 (s, 1H), 7.74 (s, 1H), 7.57 (dd, J=5.1, 1.7 Hz, 1H), 3.99 (s, 6H), 1.77 (s, 6H). MS (DCI) m/z 479 (M+H)⁺.

### Example 34

### 4,6-dimethoxy-N-[5-propyl-4-(trifluoromethyl)-1,3-thiazol-2-yl]pyrimidine-5-carboxamide

The title compound was obtained as a by-product during the preparation of Example 13 (0.01 g, 15%). ¹H NMR (400 MHz, CDCl₃) δ ppm 10.1 (s, 1H), 8.5 (s, 1H), 4.1 (s, 6H), 2.9 (m, 2H), 1.7 (m, 1H), 1.0 (t, J=7 Hz, 3H). MS (ESI) m/z 377 (M+H)⁺.

### Example 35

### N-[5-(2,2-dimethyl-1,3-dioxolan-4-yl)-4-(trifluoromethyl)-1,3-thiazol-2-yl]-4,6-dimethoxypyrimidine-5-carboxamide

### Example 35A

### N-(5-(1,2-dihydroxyethyl)-4-(trifluoromethyl)thiazol-2-yl)-4,6-dimethoxypyrimidine-5-carboxamide

To a solution of Example 22A (0.2 g, 0.555 mmol) in THF (3 mL) and water (0.3 mL) was added osmium(VIII) oxide (0.696 mL, 0.056 mmol) (solution in t-BuOH), 4-methylmorpholine 4-oxide (0.072 g, 0.611 mmol). The mixture was stirred at room temperature for 4 h. The mixture was quenched with an addition of aqueous solution of sodium thiosulfate (1 mL) and extracted with 10 mL of EtOAc. Organic phase was washed with water and brine, then dried over magnesium sulfate, filtered, concentrated under reduced pressure and the residue was purified by silica gel chromatography eluting with a gradient 0-5% of MeOH in CH₂Cl₂ to give the title compound (0.12 g, 54%). ¹H NMR (400 MHz, CDCl₃) δ ppm 10.0 (s, 1H), 8.5 (s, 1H), 4.6 (m, 1H) 4.1 (s, 6H), 4.0 (m, 1H), 3.7 (m, 1H). MS (ESI) m/z 395 (M+H)⁺.

### Example 35B

### N-[5-(2,2-dimethyl-1,3-dioxolan-4-yl)-4-(trifluoromethyl)-)-1,3-thiazol-2-yl]-4,6-dimethoxypyrimidine-5-carboxamide

A solution of Example 35A (0.1 g, 0.25 mmol), p-toluenesulfonic acid monohydrate (0.02 g, 0.1 mmol) and propan-2-one (1 mL) in benzene (10 mL) was heated to reflux with a Dean-Stark trap for 6 h. After cooling to ambient temperature, the volatiles were evaporated under reduced pressure and the residue was taken in 20 mL of CH₂Cl₂ and washed with a saturated solution of sodium bicarbonate, water and brine. Organic phase was dried over magnesium sulfate, filtered, concentrated under reduced pressure and the residue was purified by silica gel chromatography using a gradient 0-35% of EtOAc in hexanes to give the title compound (0.075 g, 68%). ¹H NMR (400 MHz, CDCl₃) δ ppm 10.0 (s, 1H), 8.5 (s, 1H), 5.5 (m, 1H), 4.3 (m, 1H) 4.1 (s, 6H), 3.8 (m, 1H), 1.45 (s, 6H). MS (ESI) m/z 435 (M+H)⁺.

### Example 36

### 4,6-dimethoxy-N-{5-[2-(morpholin-4-yl)pyrimidin-5-yl]-4-(trifluoromethyl)-1,3-thiazol-2-yl}pyrimidine-5-carboxamide

A suspension of Example 2C (0.200 g, 0.435 mmol), (2-morpholinopyrimidin-5-yl)boronic acid (0.118 g, 0.565 mmol) and PdCl₂(dppf) (0.048 g, 0.065 mmol) was stirred in dioxane (5 ml) under a nitrogen atmosphere. After de-gassing, 1.0 M aqueous cesium carbonate (0.87 ml, 0.87 mmol) was added and stirred at ambient temperature for one hour, then heated at 80° C. overnight. The mixture was filtered through a bed of CELITE®, washed with excess ethyl acetate and concentrated under reduced pressure. The crude material was purified by flash chromatography (12 g silica gel, 35-65% gradient of ethyl acetate in heptane) to provide the title compound (182 mg. 84% yield) as a light yellow solid. ¹H NMR (400 MHz, DMSO-d₆) δ ppm 13.09 (s, 1H), 8.63 (s, 1H), 8.51 (s, 2H), 3.97 (s, 6H), 3.83-3.75 (m, 4H), 3.73-3.64 (m, 4H). MS (DCI/NH₃) m/z 498 (M+H)⁺.

### Example 37

### N-[5-cyclopropyl-4-(trifluoromethyl)-1,3-thiazol-2-yl]-4,6-dimethoxypyrimidine-5-carboxamide

A suspension of Example 2C (2.000 g, 4.35 mmol), cyclopropylboronic acid (0.485 g, 5.65 mmol) and PdCl₂(dppf) (0.477 g, 0.652 mmol) was stirred in dioxane (10 ml) under a nitrogen atmosphere. After de-gassing, 1.0 M aqueous cesium carbonate (8.69 ml, 8.69 mmol) was added and stirred at ambient temperature for one hour, then heated at 80° C. overnight. The mixture was filtered through a bed of CELITE®, washed with excess ethyl acetate and concentrated under reduced pressure. The crude material was purified by preparative HPLC (Waters XBRIDGE™ C18 m OBD column, 50×100 mm, flow rate 90 mL/minute, 20-100% gradient of methanol in buffer (0.025 M aqueous ammonium bicarbonate, adjusted to pH 10 with ammonium hydroxide)) to provide the title compound (0.144 g. 8.9% yield) as a solid. ¹H NMR (400 MHz, DMSO-d₆) δ ppm 12.82 (s, 1H), 8.61 (s, 1H), 3.95 (s, 6H), 2.32-2.18 (m, 1H), 1.20-1.11 (m, 2H), 0.86-0.74 (m, 2H). MS (DCI/NH₃) m/z 375 (M+H)⁺.

### Example 38

### N-[5-(3,6-dihydro-2H-pyran-4-yl)-4-(trifluoromethyl)-1,3-thiazol-2-yl]-4,6-dimethoxypyrimidine-5-carboxamide

A suspension of Example 2C (1.00 g, 2.173 mmol), 2-(3,6-dihydro-2H-pyran-4-yl)-4,4,5,5-tetramethyl-1,3,2-dioxaborolane (0.577 g, 2.61 mmol) and PdCl₂(dppf) (0.239 g, 0.326 mmol) was stirred in dioxane (50 ml) under a nitrogen atmosphere. After de-gassing, 1.0 M aqueous cesium carbonate (3.26 ml, 3.26 mmol) was added and stirred at ambient temperature for one hour, then heated at 80° C. overnight. The mixture was filtered through a bed of CELITE®, washed with excess ethyl acetate and concentrated under reduced pressure. The crude material was purified by flash chromatograph (40 g silica gel, 0-10% gradient of ethyl acetate in dichloromethane) to provide the title compound (343 mg. 38% yield)¹H NMR (400 MHz, DMSO-d₆) δ ppm 12.95 (s, 1H), 8.61 (s, 1H), 6.12-6.09 (m, 1H), 4.19 (q, J=2.7, 2.6, 2.6 Hz, 2H), 3.94 (s, 6H), 3.79 (t, J=5.4, 5.4 Hz, 2H), 2.39-2.31 (m, 2H). MS (DCI/NH₃) m/z 417 (M+H)⁺.

### Example 39

### 4,6-dimethoxy-N-[5-(tetrahydro-2H-pyran-4-yl)-4-(trifluoromethyl)-1,3-thiazol-2-yl]pyrimidine-5-carboxamide

A mixture of ethanol (4.00 mL), Example 38 (174.9 mg, 0.420 mmol) and 20% Pd(OH)₂/C, wet (101.0 mg, 0.719 mmol) in a 20 mL pressure bottle was hydrogenated under 30 psi at 50° C. for 16 h. The mixture was filtered through a polypropylene membrane and concentrated to a foam, which was taken up in minimal amount of diethyl ether and triturated with heptane while vigorously stirring. The resulting solid was filtered off, washed with excess heptane to give the title compound (0.143 g, 73% yield) as a white solid. ¹H NMR (400 MHz, DMSO-d₆) δ ppm 12.88 (s, 1H), 8.62 (s, 1H), 3.95 (s, 8H), 3.52-3.35 (m, 3H), 1.85 (d, J=12.1 Hz, 2H), 1.69 (tt, J=12.0, 12.0, 6.2, 6.2 Hz, 2H). MS (DCI/NH₃) m/z 419 (M+H)⁺.

### Example 40

### N-[5-(3-cyanophenyl)-4-(trifluoromethyl)-1,3-thiazol-2-yl]-4,6-dimethoxypyrimidine-5-carboxamide

A suspension of Example 2 (0.300 g, 0.652 mmol), (3-cyanophenyl)boronic acid (0.125 g, 0.848 mmol) and PdCl₂(dppf)-CH₂Cl₂ (0.053 g, 0.065 mmol) was stirred in dioxane (5 ml) under a nitrogen atmosphere. After de-gassing, 1.0 M aqueous cesium carbonate (1.30 ml, 1.30 mmol) was added and stirred at ambient temperature for one hour, then heated at 80° C. overnight. The mixture was filtered through a bed of CELITE®, washed with excess ethyl acetate and concentrated under reduced pressure. The crude material was purified by flash chromatography (4 g silica gel, 30-50% gradient of ethyl acetate in heptane) to provide the desired product Example 40 (188 mg. 67% yield). ¹H NMR (400 MHz, CHCl₃-d) δ ppm 10.31 (s, 1H), 8.53 (s, 1H), 7.78-7.68 (m, 3H), 7.58 (t, J=7.8, 7.8 Hz, 1H), 4.14 (s, 6H). MS (DCI/NH₃) m/z 436 (M+H)⁺.

### Example 41

### N-[5-(4-cyanophenyl)-4-(trifluoromethyl)-1,3-thiazol-2-yl]-4,6-dimethoxypyrimidine-5-carboxamide

The title compound was prepared as described in Example 40, substituting (4-cyanophenyl)boronic acid for (3-cyanophenyl)boronic acid (0.25 g, 91% yield). ¹H NMR (400 MHz, CDCl₃) δ ppm 10.4 (s, 1H), 8.5 (s, 1H), 7.9 (m, 1H), 7.8 (m, 1H), 7.6 (m, 1H) 4.1 (s, 6H). MS (ESI) m/z 480 (M+H)⁺.

### Example 42

### N-[5-(2-cyanopyridin-3-yl)-4-(trifluoromethyl)-1,3-thiazol-2-yl]-4,6-dimethoxypyrimidine-5-carboxamide

The title compound was prepared as described in Example 40, substituting (2-cyanopyridin-3-yl)boronic acid (0.058 g, 0.391 mmol) for (3-cyanophenyl)boronic acid (12 mg, 8.4% yield). ¹H NMR (501 MHz, DMSO-d₆) δ ppm 13.34 (s, 1H), 8.87 (dd, J=4.8, 1.6 Hz, 1H), 8.64 (s, 1H), 8.26 (dd, J=8.0, 1.5 Hz, 1H), 7.89 (dd, J=8.0, 4.8 Hz, 1H), 3.97 (s, 6H). MS (DCI/NH₃) m/z 437 (M+H)⁺.

### Example 43

### N-[5-(4-cyano-2-methoxyphenyl)-4-(trifluoromethyl)-1,3-thiazol-2-yl]-4,6-dimethoxypyrimidine-5-carboxamide

A microwave vial charged with Example 2 (150 mg, 0.326 mmol), (4-cyano-2-methoxyphenyl)boronic acid (87 mg, 0.489 mmol), Pd(amphos)Cl₂ (23.08 mg, 0.033 mmol) and K₃PO₄ (208 mg, 0.978 mmol) was purged with nitrogen, dioxane (4.0 ml) and water (0.400 ml) were added, and the mixture was irradiated at 150° C. for 10 min in a Biotage microwave. The mixture was cooled to ambient temperature, filtered, diluted with brine, extracted three times with diethyl ether, and the combined extracts were washed with brine and concentrated under reduced pressure. The crude material was purified by flash chromatography (12 g silica gel, 0-40% gradient of ethyl acetate in heptane) to provide the title compound (69 mg. 46% yield). ¹H NMR (400 MHz, DMSO-d₆) δ ppm 13.13 (s, 1H), 8.62 (s, 1H), 7.66 (d, J=1.1 Hz, 1H), 7.61-7.51 (m, 2H), 3.96 (s, 6H), 3.86 (s, 3H). MS (DCI/NH₃) m/z 466 (M+H)⁺.

### Example 44

### N-[5-(6-cyano-1H-indol-3-yl)-4-(trifluoromethyl)-1,3-thiazol-2-yl]-4,6-dimethoxypyrimidine-5-carboxamide

The title compound was prepared as described in Example 43, substituting tert-butyl 6-cyano-3-(4,4,5,5-tetramethyl-1,3,2-dioxaborolan-2-yl)-1H-indole-1-carboxylate for (4-cyano-2-methoxyphenyl)boronic acid (32.7 mg, 21.1% yield). ¹H NMR (400 MHz, DMSO-d₆) δ ppm 13.04 (s, 1H), 12.23 (s, 1H), 8.62 (s, 1H), 8.00 (s, 1H), 7.95 (d, J=2.6 Hz, 1H), 7.71 (d, J=8.3 Hz, 1H), 7.47 (dd, J=8.3, 1.3 Hz, 1H), 3.97 (s, 6H). MS (DCI/NH₃) m/z 475 (M+H)⁺.

### Example 45

### 4,6-dimethoxy-N-[5-(pyridin-2-yl)-4-(trifluoromethyl)-1,3-thiazol-2-yl]pyrimidine-5-carboxamide

A suspension of Example 2C (0.100 g, 0.217 mmol), 2-(tributylstannyl)pyridine (0.091 ml, 0.239 mmol), Pd(Ph₃P)₄ (0.025 g, 0.022 mmol) and cesium fluoride (0.066 g, 0.435 mmol) was added to a sealable vial, purged with nitrogen, and anhydrous DMF (1.0 mL) and copper(I) iodide (4.14 mg, 0.022 mmol) were added. The reaction mixture was heated under nitrogen at 80° C. overnight, cooled to ambient temperature, filtered through a bed of CELITE®, washed with excess ethyl acetate, and concentrated under reduced pressure. The crude material was purified by flash chromatography (4 g silica gel, 0-30% gradient of ethyl acetate in heptane) to provide the title compound (9 mg. 10% yield). ¹H NMR (400 MHz, DMSO-d₆) δ ppm 13.10 (s, 1H), 8.72-8.67 (m, 1H), 8.63 (s, 1H), 7.98 (td, J=7.8, 7.8, 1.7 Hz, 1H), 7.72 (d, J=7.8 Hz, 1H), 7.48 (dd, J=7.2, 5.2 Hz, 1H), 3.97 (s, 6H). MS (DCI/NH₃) m/z 412 (M+H)⁺.

### Example 46

### N-[5-(5-chloropyridin-2-yl)-4-(trifluoromethyl)-1,3-thiazol-2-yl]-4,6-dimethoxypyrimidine-5-carboxamide

The title compound was prepared as described in Example 45, substituting 5-chloro-2-(tributylstannyl)pyridine for 2-(tributylstannyl)pyridine (7.5 mg, 7.8% yield). ¹H NMR (400 MHz, DMSO-d₆) δ ppm 13.16 (s, 1H), 8.77 (d, J=2.3 Hz, 1H), 8.64 (s, 1H), 8.12 (dd, J=8.5, 2.5 Hz, 1H), 7.74 (d, J=8.5 Hz, 1H), 3.97 (s, 6H). MS (DCI/NH₃) m/z 446 (M+H)⁺.

### Example 47

### N-[5-formyl-4-(trifluoromethyl)-1,3-thiazol-2-yl]-4,6-dimethoxypyrimidine-5-carboxamide

To a solution of diisopropylamine (2.6 mL, 18.24 mmol) in THF (30 mL) chilled to −75° C. was added 2.5 M nBuLi (7.4 mL, 18.50 mmol) in hexanes. The mixture was stirred at −5° C. for 30 min, then chilled to −75° C., and a solution of Example 3 (2.0 g, 5.98 mmol) in THF (10 mL) was then added dropwise, The yellow solution was stirred at −75° C. for 5 min. Ethyl formate (2.0 mL, 24.57 mmol) was added next and the resulting red solution was stirred at −75° C. for 1 h. The reaction was quenched with 200 mL of saturated solution of NH₄Cl, extracted with 200 mL of ethyl acetate, washed with brine, dried over Na₂SO₄, filtered, and concentrated under reduced pressure. The crude material was purified by flash chromatography (120 g silica gel, 0-50% gradient of ethyl acetate in heptane) to give the title compound (1.79 g, 82% yield). ¹H NMR (400 MHz, DMSO-d₆) δ 13.54 (s, 1H), 10.12 (d, J=1.0 Hz, 1H), 8.63 (s, 1H), 3.95 (s, 6H). MS (ESI) m/z 361 (M−H)⁺.

### Example 48

### 4,6-dimethoxy-N-{5-[(4-methoxypiperidin-1-yl)methyl]-4-(trifluoromethyl)-1,3-thiazol-2-yl}pyrimidine-5-carboxamide

A solution of Example 47 (0.2 g, 0.552 mmol) and 4-methoxypiperidine (0.127 g, 1.104 mmol) in anhydrous THF (5 mL) was stirred for ˜20 min at room temperature, Silicycle SILICABOND cyanoborohydride (loading 0.89 mmol/g, 1.2 g) and two drops of AcOH were added, and the mixture was stirred for 14 h. The mixture was diluted with EtOAc (30 mL) and filtered through CELITE®. The filtrate was concentrated under reduced pressure and the residue was purified by silica gel chromatography using a gradient 0-10% of methanol in CH₂Cl₂ to give the title compound (0.13 g, 53%). ¹H NMR (400 MHz, CDCl₃) δ ppm 10.0 (s, 1H), 8.5 (s, 1H), 4.15 (m, 1H), 4.1 (s, 6H) 4.1 (s, 6H), 3.7 (bs, 2H), 3.3 (s, 3H), 2.7 (m, 2H), 2.3 (m, 2H), 1.8 (m, 2H), 1.6 (m, 1H). MS (ESI) m/z 462 (M+H)⁺.

### Example 49

### 4,6-dimethoxy-N-[5-(pyrrolidin-1-ylmethyl)-4-(trifluoromethyl)-1,3-thiazol-2-yl]pyrimidine-5-carboxamide

A solution of Example 47 (0.030 g, 0.083 mmol) and pyrrolidine (0.012 g, 0.166 mmol) in THF (1.0 ml) and acetic acid (0.05 mL) was stirred at ambient temperature under nitrogen atmosphere, then SILICABOND cyanoborohydride (0.89 mmol/g) (0.186 mg, 0.166 mmol) was added and the mixture was stirred overnight, filtered, washed with ethyl acetate, and concentrated under reduced pressure. The crude material was purified by flash chromatography (4 g silica gel, 0-30% gradient of ethyl acetate in heptane) to provide the title compound (25 mg. 75% yield). ¹H NMR (400 MHz, DMSO-d₆) δ ppm 12.80 (s, 1H), 8.61 (s, 1H), 3.96 (s, 6H), 3.17 (d, J=5.2 Hz, 2H), 2.60-2.55 (m, 4H), 1.77-1.73 (m, 4H). MS (DCI/NH₃) m/z 418 (M+H)⁺.

### Example 50

### 4,6-dimethoxy-N-[5-(morpholin-4-ylmethyl)-4-(trifluoromethyl)-1,3-thiazol-2-yl]pyrimidine-5-carboxamide

The title compound was prepared as described in Example 49, substituting morpholine for pyrrolidine (12.1 mg, 33.6% yield). ¹H NMR (400 MHz, DMSO-d₆) δ ppm 12.69 (s, 1H), 8.62 (s, 1H), 3.95 (s, 6H), 3.80 (d, J=1.7 Hz, 2H), 3.65-3.57 (m, 4H), 2.64-2.57 (m, 4H). MS (DCI/NH₃) m/z 434 (M+H)⁺.

### Example 51

### N-{5-[(cyclopentylamino)methyl]-4-(trifluoromethyl)-1,3-thiazol-2-yl}-4,6-dimethoxypyrimidine-5-carboxamide

The title compound was prepared as described in Example 49, substituting cyclopentyl amine for pyrrolidine (42.0 mg, 44.1% yield). ¹H NMR (400 MHz, DMSO-d₆) δ ppm 13.17 (s, 1H), 9.31 (s, 1H), 8.64 (s, 1H), 4.51 (s, 2H), 3.96 (s, 6H), 3.66-3.55 (m, 1H), 2.08-1.97 (m, 2H), 1.76-1.52 (m, 6H). MS (DCI/NH₃) m/z 432 (M+H)⁺.

### Example 52

### 4,6-dimethoxy-N-[5-{[(2-methylpropyl)amino]methyl}-4-(trifluoromethyl)-1,3-thiazol-2-yl]pyrimidine-5-carboxamide

The title compound was prepared as described in Example 49, substituting isobutylamine for pyrrolidine (37.1 mg, 25.2% yield). ¹H NMR (400 MHz, DMSO-d₆) δ ppm 12.66 (s, 1H), 8.61 (s, 1H), 3.96 (s, 8H), 2.38 (d, J=6.7 Hz, 2H), 1.75-1.62 (m, 0H), 0.89 (d, J=6.6 Hz, 1H). MS (DCI/NH₃) m/z 420 (M+H)⁺.

### Example 53

### N-{5-[(4-fluoropiperidin-1-yl)methyl]-4-(trifluoromethyl)-1,3-thiazol-2-yl}-4,6-dimethoxypyrimidine-5-carboxamide

The title compound was prepared as described in Example 49, substituting 4-fluoropiperidine for pyrrolidine (85 mg, 13.7% yield). ¹H NMR (400 MHz, DMSO-d₆) δ ppm 12.64 (s, 1H), 8.62 (s, 1H), 4.73 (d, J=48.8 Hz, 1H), 3.96 (s, 6H), 3.81 (s, 2H), 2.67-2.58 (m, 2H), 2.50-2.43 (m, 2H), 1.97-1.69 (m, 4H). MS (DCI/NH₃) m/z 420 (M+H)⁺. MS (DCI/NH₃) m/z 450 (M+H)⁺.

### Example 54

### 4,6-dimethoxy-N-[5-{[methyl(2-methylpropyl)amino]methyl}-4-(trifluoromethyl)-1,3-thiazol-2-yl]pyrimidine-5-carboxamide

The title compound was prepared as described in Example 49, substituting N-2-dimethylpropan-1-amine for pyrrolidine (84 mg, 46.8% yield). ¹H NMR (500 MHz, DMSO-d₆) δ ppm 12.79 (s, 1H), 8.61 (s, 1H), 3.96 (s, 6H), 3.77 (s, 2H), 2.24-2.17 (m, 5H), 1.80 (dt, J=13.4, 6.7, 6.7 Hz, 1H), 0.96-0.84 (m, 6H). MS (DCI/NH₃) m/z 434 (M+H)⁺.

### Example 55

### N-[5-{[tert-butyl(methyl)amino]methyl}-4-(trifluoromethyl)-1,3-thiazol-2-yl]-4,6-dimethoxypyrimidine-5-carboxamide

The title compound was prepared as described in Example 49, substituting N-2-dimethylpropan-2-amine for pyrrolidine (24.5 mg, 36% yield). ¹H NMR (400 MHz, DMSO-d₆) δ ppm 13.26 (s, 1H), 8.64 (s, 1H), 3.96 (s, 8H), 2.70 (s, 3H), 1.43 (s, 9H). MS (DCI/NH₃) m/z 434 (M+H)⁺.

### Example 56

### N-{5-[(tert-butylamino)methyl]-4-(trifluoromethyl)-1,3-thiazol-2-yl}-4,6-dimethoxypyrimidine-5-carboxamide

The title compound was prepared as described in Example 49, substituting t-butylamine for pyrrolidine (10.8 mg, 16.3% yield). ¹H NMR (400 MHz, DMSO-d₆) δ ppm 13.18 (s, 1H), 9.08 (s, 1H), 8.64 (s, 1H), 4.50 (s, 2H), 3.96 (s, 6H), 1.37 (s, 9H). MS (DCI/NH₃) m/z 420 (M+H)⁺.

### Example 57

### N-[5-{[(3 S)-3-fluoropyrrolidin-1-yl]methyl}-4-(trifluoromethyl)-1,3-thiazol-2-yl]-4,6-dimethoxypyrimidine-5-carboxamide

The title compound was prepared as described in Example 49, substituting (S)-3-fluoropyrrolidine for pyrrolidine (7.1 mg, 28.3% yield). ¹H NMR (400 MHz, pyridine-d₅) δ ppm 14.53 (s, 1H), 8.64 (s, 1H), 5.15 (d, J=58.3 Hz, 1H), 4.01 (s, 2H), 3.86 (s, 6H), 2.99 (dd, J=26.5, 11.4 Hz, 1H), 2.89-2.73 (m, 2H), 2.51-2.43 (m, 1H), 2.08-1.93 (m, 2H). MS (DCI/NH₃) m/z 436 (M+H)⁺.

### Example 58

### N-[5-{[(3R)-3-fluoropyrrolidin-1-yl]methyl}-4-(trifluoromethyl)-1,3-thiazol-2-yl]-4,6-dimethoxypyrimidine-5-carboxamide

The title compound was prepared as described in Example 49, substituting (R)-3-fluoropyrrolidine for pyrrolidine (5.9 mg, 23.5% yield). ¹H NMR (400 MHz, pyridine-d₅) δ ppm 14.53 (s, 1H), 8.64 (s, 1H), 5.15 (d, J=58.3 Hz, 1H), 4.01 (s, 2H), 3.86 (s, 6H), 2.99 (dd, J=26.5, 11.4 Hz, 1H), 2.89-2.73 (m, 2H), 2.51-2.43 (m, 1H), 2.08-1.93 (m, 2H). MS (DCI/NH₃) m/z 436 (M+H)⁺.

### Example 59

### 4,6-dimethoxy-N-[5-{[(3S)-3-methoxypyrrolidin-1-yl]methyl}-4-(trifluoromethyl)-1,3-thiazol-2-yl]pyrimidine-5-carboxamide

The title compound was prepared as described in Example 49, substituting (S)-3-methoxypyrrolidine for pyrrolidine (12.8 mg, 39.9% yield). ¹H NMR (400 MHz, pyridine-d₅) δ ppm 14.49 (s, 1H), 8.64 (s, 1H), 5.15 (d, J=58.3 Hz, 1H), 4.00 (d, J=1.8 Hz, 2H), 3.86 (s, 6H), 3.18 (s, 3H), 2.92 (dd, J=10.0, 6.2 Hz, 1H), 2.74-2.67 (m, 2H), 2.60 (td, J=8.6, 8.2, 5.4 Hz, 1H), 1.99-1.91 (m, 1H), 1.86-1.77 (m, 1H). MS (DCI/NH₃) m/z 448 (M+H)⁺.

### Example 60

### 4,6-dimethoxy-N-[5-{[(3R)-3-methoxypyrrolidin-1-yl]methyl}-4-(trifluoromethyl)-1,3-thiazol-2-yl]pyrimidine-5-carboxamide

The title compound was prepared as described in Example 49, substituting (R)-3-methoxypyrrolidine for pyrrolidine (12.9 mg, 49.9% yield). ¹H NMR (400 MHz, pyridine-d₅) δ ppm 14.49 (s, 1H), 8.64 (s, 1H), 5.15 (d, J=58.3 Hz, 1H), 4.00 (d, J=1.8 Hz, 2H), 3.86 (s, 6H), 3.18 (s, 3H), 2.92 (dd, J=10.0, 6.2 Hz, 1H), 2.74-2.67 (m, 2H), 2.60 (td, J=8.6, 8.2, 5.4 Hz, 1H), 1.99-1.91 (m, 1H), 1.86-1.77 (m, 1H). MS (DCI/NH₃) m/z 448 (M+H)⁺.

### Example 61

### 4,6-dimethoxy-N-[5-(2-oxa-7-azaspiro[3.5]non-7-ylmethyl)-4-(trifluoromethyl)-1,3-thiazol-2-yl]pyrimidine-5-carboxamide

The title compound was prepared as described in Example 49, substituting 2-oxa-7-azaspiro[3.5]nonane for pyrrolidine (8.0 mg, 29.8% yield). ¹H NMR (400 MHz, pyridine-d₅) δ ppm 14.49 (s, 1H), 8.63 (s, 1H), 4.46 (d, J=5.8 Hz, 2H), 3.89-3.80 (m, 8H), 3.75 (d, J=5.8 Hz, 2H), 2.39-2.25 (m, 2H), 1.72-1.50 (m, 4H), 1.47-1.32 (m, 2H). MS (DCI/NH₃) m/z 474 (M+H)⁺.

### Example 62

### 4,6-dimethoxy-N-[5-(2-oxa-6-azaspiro[3.5]non-6-ylmethyl)-4-(trifluoromethyl)-1,3-thiazol-2-yl]pyrimidine-5-carboxamide

The title compound was prepared as described in Example 49, substituting 2-oxa-6-azaspiro[3.5]nonane for pyrrolidine (6.6 mg, 24.6% yield). ¹H NMR (400 MHz, pyridine-d₅) δ ppm 14.49 (s, 1H), 8.63 (s, 1H), 4.46 (d, J=5.8 Hz, 2H), 4.33 (d, J=5.8 Hz, 2H), 3.89-3.80 (m, 8H), 2.67-2.42 (m, 2H), 2.39-2.25 (m, 2H), 1.72-1.50 (m, 2H), 1.47-1.32 (m, 2H). MS (DCI/NH₃) m/z 474 (M+H)⁺.

### Example 63

### N-[5-chloro-4-(trifluoromethyl)-1,3-thiazol-2-yl]-4,6-dimethoxypyrimidine-5-carboxamide

To a solution of Example 3 (0.101 g, 0.302 mmol) in CHCl₃ (3 ml) was added PALAU'CHLOR (0.076 g, 0.363 mmol) (Aldrich), and the mixture was stirred at ambient temperate for 12 h under nitrogen. An additional amount of PALAU'CHLOR (0.076 g, 0.363 mmol) was added and stirring continued for 48 h. The solid was filtered off, the filtrate was washed with CHCl₃ and concentrated under reduced pressure. The crude material was purified by prep-HPLC on a Phenomenex LUNA C8(2) 5 μm 100 Å AXIA column (30 mm×75 mm) (A gradient of acetonitrile (A) and 0.1% trifluoroacetic acid in water (B) was used, at a flow rate of 50 mL/min (0-1.0 min 5% A, 1.0-8.5 min linear gradient 5-100% A, 8.5-11.5 min 100% A, 11.5-12.0 min linear gradient 95-5% A) to afford the title compound (42 mg, 38% yield). ¹H NMR (400 MHz, DMSO-d6) δ ppm 13.29 (s, 1H), 8.64 (s, 1H), 3.97 (s, 6H). MS (DCI/NH3) m/z 369 (M+H)+, 386 (M+NH4)+.

### Example 64

### 4,6-dimethoxy-N-[5-(2-methylpyridin-3-yl)-4-(trifluoromethyl)-1,3-thiazol-2-yl]pyrimidine-5-carboxamide

A nitrogen purged solution of THF (10 ml) and water (5 ml) was added to a mixture of Example 2C (2.0 g, 4.33 mmol), 2-methyl-3-(4,4,5,5-tetramethyl-1,3,2-dioxaborolan-2-yl)pyridine (1.1 g, 4.99 mmol), Cs₂CO₃ (2.8 g, 8.66 mmol), Pd₂dba₃ (0.12 g, 0.129 mmol), and (1S,3R,5R,7S)-1,3,5,7-tetramethyl-8-phenyl-2,4,6-trioxa-8-phosphaadamantane (0.15 g, 0.519 mmol) under N₂. The mixture was heated to 65° C. for 2 h, allowed to cool to ambient temperature, diluted with EtOAc, washed with water and brine, dried (Na₂SO₄), and subjected to chromatography on silica gel (20% EtOAc/CH₂Cl₂) to give the title compound (1.23 g, 67%): ¹H NMR (400 MHz, DMSO-d₆) δ 13.16 (s, 1H), 8.64 (s, 1H), 8.58 (dd, J=4.9, 1.7 Hz, 1H), 7.80 (dd, J=7.7, 1.7 Hz, 1H), 7.36 (dd, J=7.7, 4.9 Hz, 1H), 3.98 (s, 6H), 2.39 (s, 3H); MS (ESI) m/z 426 (M+H)⁺.

### Example 65

### 4,6-dimethoxy-N-{5-[1-methyl-3-(trifluoromethyl)-1H-pyrazol-5-yl]-4-(trifluoromethyl)-1,3-thiazol-2-yl}pyrimidine-5-carboxamide

DMF (15 mL) was added to a 20 mL Biotage microwave vial and dry nitrogen gas was bubbled through the liquid. The flask was charged with palladium(II) acetate (23.90 mg, 0.106 mmol) followed by 1,1′-bis(diphenylphosphino)ferrocene (122 mg, 0.213 mmol). This solution was stirred with nitrogen bubbling through it while the other reactants were weighed. Next, Example 2C (690 mg, 1.500 mmol) was added, followed by 1-methyl-5-(4,4,5,5-tetramethyl-1,3,2-dioxaborolan-2-yl)-3-(trifluoromethyl)-1H-pyrazole (CAS: 1025719-23-6, Combi-Blocks #PN-8567, 828 mg, 3.00 mmol). With nitrogen still bubbling through the reaction mixture, copper(I) chloride (156 mg, 1.500 mmol) and finally cesium carbonate (1954 mg, 6.00 mmol) were added. After the stirred reaction mixture had nitrogen bubbled through it for an additional 2 minutes, the vial was sealed with a crimp-cap septum. The reaction mixture was stirred at 100° C. under microwave irradiation for 126 minutes. The reaction mixture was diluted with water (200 mL) and extracted with EtOAc (2×50 mL). The combined organic layers were washed with brine (2×100 mL), dried (MgSO₄), and filtered. The filtrate was concentrated under reduced pressure to give a crude, dark olive-green oil that was purified by column chromatography on an Analogix IF-310 (Isco REDISEP GOLD 220 g, 85:15 to 70:30 heptane/EtOAc). Product fractions were combined and concentrated under reduced pressure to give a white foam (344 mg, 47.6% yield) that was repurified (Isco RediSep Gold 12 g, 100% DCM to 97:3 DCM/EtOAc). Product fractions from this second column were combined and concentrated under reduced pressure to give the title compound as a white solid (122.3 mg, 16.9% yield). ¹H NMR (Chloroform-d) δ: 10.45 (s, 1H), 8.54 (s, 1H), 6.68 (s, 1H), 4.15 (s, 6H), 3.86 (s, 3H). MS (DCI-NH₃) m/z=483 (M+H)⁺, m/z=500 (M+NH₄)⁺.

### Example 66

### N-[5-(2-cyanopyridin-4-yl)-4-(trifluoromethyl)-1,3-thiazol-2-yl]-4,6-dimethoxypyrimidine-5-carboxamide

A nitrogen purged solution of dioxane (2 mL) was added to a mixture of Example 31B (2.0 g, 4.79 mmol), 4-(4,4,5,5-tetramethyl-1,3,2-dioxaborolan-2-yl)picolinonitrile (1.2 g, 5.27 mmol) (ArkPharma), Cs₂CO₃ (3.1 g, 9.58 mmol), copper(I) chloride (0.47 g, 4.79 mmol), and 1,1′-bis(diphenylphosphino)ferrocene-palladium(II)dichloride dichloromethane complex (0.4 g, 0.479 mmol) under nitrogen. The mixture was stirred at 100° C. for 90 min, diluted with EtOAc (20 mL), washed with water (5 mL) and brine (5 mL), then dried (Na₂SO₄) and concentrated under reduced pressure. The residue was subjected to chromatography on SiO₂ eluting with 0-50% ethyl acetate over 90 min with 60 min hold to give the title compound (1.34 g, 64.1% yield). ¹H NMR (400 MHz, DMSO-d₆) δ 13.30 (s, 1H), 8.87 (d, J=5.1 Hz, 1H), 8.63 (s, 1H), 8.30-8.22 (m, 1H), 7.89 (dd, J=5.1, 1.8 Hz, 1H), 3.96 (s, 6H). MS (ESI) m/z 437 (M+H)⁺.

### Example 67

### N-[5-(1,3-dimethyl-1H-pyrazol-4-yl)-4-(trifluoromethyl)-1,3-thiazol-2-yl]-4,6-dimethoxypyrimidine-5-carboxamide

To a solution of Example 2C (2.0 g, 4.35 mmol) and 1,3-dimethyl-1 h-pyrazole-4-boronic acid pinacol ester (ArkPharma) (1.0 g, 4.35 mmol) in THF (10 mL) were added 1,1′-bis(di-tert-butylphosphino)ferrocene palladium dichloride (0.28 g, 0.435 mmol) and cesium carbonate (4.35 mL, 8.71 mmol) under nitrogen. The reaction mixture was stirred overnight at 60° C. and cooled to ambient temperature. Ethyl acetate (100 mL) was added, washed with water, and then the organic phase was dried (Na₂SO₄), filtered and concentrated under reduced pressure. The residue was purified on SiO₂ eluting with heptane/ethyl acetate 0-80% over 60 min with 60 min hold to give the title compound (0.39 g, 20.6% yield). ¹H NMR (400 MHz, DMSO-d6) δ 13.00 (s, 1H), 8.63 (s, 1H), 7.87 (s, 1H), 3.97 (s, 6H), 3.82 (s, 3H), 2.15 (s, 3H). MS (ESI) m/z 429 (M+H)⁺.

### Example 68

### {[(4,6-dimethoxypyrimidin-5-yl)carbonyl][5-(1,3-dimethyl-1H-pyrazol-4-yl)-4-(trifluoromethyl)-1,3-thiazol-2-yl]amino}methyl dihydrogen phosphate

### Example 68A

### di-tert-butyl ((N-(5-(1,3-dimethyl-1H-pyrazol-4-yl)-4-(trifluoromethyl)thiazol-2-yl)-4,6-dimethoxypyrimidine-5-carboxamido)methyl) phosphate

A mixture of Example 67 (2.0 g, 4.65 mmol), di-tert-butyl (chloromethyl) phosphate (1.6224 g, 6.27 mmol), potassium iodide (0.78 g, 4.68 mmol) and Cs₂CO₃ (3.03 g, 9.29 mmol) in N-methyl-2-pyrrolidone (12 ml) was stirred at 60° C. for 90 min, diluted with EtOAc, washed with water, saturated aqueous NaHCO₃, brine, and dried (Na₂SO₄) to give 4.15 g of yellow oil, which was subjected to chromatography on silica gel (10-15% EtOAc/CH₂Cl₂), precipitated with ˜1:1 Et₂O/heptane, and filtered to give the title compound (1.92 g, 63.5% yield): ¹H NMR (501 MHz, DMSO-d₆) δ 8.73 (s, 1H), 7.90 (s, 1H), 5.69 (d, J=5.0 Hz, 2H), 3.98 (s, 6H), 2.15 (s, 3H), 1.32 (s, 18H); MS (ESI) m/z 651 (M+H).

### Example 68B

### {[(4,6-dimethoxypyrimidin-5-yl)carbonyl][5-(1,3-dimethyl-1H-pyrazol-4-yl)-4-(trifluoromethyl)-1,3-thiazol-2-yl]amino}methyl dihydrogen phosphate

To a solution of sulfuric acid (2.75 ml, 51.6 mmol) in ethanol (20 ml) at 0° C. was added Example 68A (3.35 g, 5.14 mmol). The solution was allowed to warm, stirred for 3 hr, diluted with water (˜0.3 mL), concentrated to ˜half volume, diluted with water, filtered, and dried in vacuo to give the title compound (2.90 g, 101% yield, containing ˜4% impurity by ¹H NMR, and 3.2 wt % water): ¹H NMR (400 MHz, Methanol-d4) δ 8.56 (s, 1H), 7.80 (s, 1H), 5.86 (d, J=5.9 Hz, 2H), 4.04 (s, 6H), 3.91 (s, 3H), 2.25 (s, 3H); MS (ESI) m/z 537 (M−H).

### Example 69

### N-[5-ethyl-4-(trifluoromethyl)-1,3-thiazol-2-yl]-4,6-dimethoxy-2-methylpyrimidine-5-carboxamide

### Example 69A

To a solution of 2,2,6,6-tetramethylpiperidine (2.5 ml, 14.81 mmol) in THF (50 ml) at 0° C. was added a 2.5M hexane solution of butyllithium (5.93 ml, 14.81 mmol) dropwise. The mixture was stirred for 15 min, cooled to −78° C., and a solution of 4,6-dimethoxy-2-methylpyrimidine (1.63 g, 10.58 mmol) (Combi-Blocks) in THF (8 ml) was added dropwise. The mixture was stirred for 20 min, and CO₂ was bubbled into the reaction via cannula from a flask of dry ice. The mixture was stirred for 10 min, allowed to warm to room temperature, and then stirred for 45 additional minutes. The reaction mixture was concentrated in vacuo, the residue was diluted with water and 1N NaOH (7.5 mL), and extracted with EtOAc. The aqueous layer was acidified to pH-1 with 1N HCl, extracted with EtOAc, dried (Na₂SO₄), and concentrated under reduced pressure to give the title compound (1.31 g, 62.5% yield) as a white solid, which was used without purification. MS (DCI) m/z 198.9 (M+H).

### Example 69B

### N-[5-ethyl-4-(trifluoromethyl)-1,3-thiazol-2-yl]-4,6-dimethoxy-2-methylpyrimidine-5-carboxamide

The title compound was prepared as described in Example 2C, substituting Example 69A for Example 2A, and substituting Example 6A for Example 2B (46 mg, 6% yield). ¹H NMR (400 MHz, DMSO-d₆) δ 1.24 (t, J=10.8 Hz, 3H), 2.80-2.99 (m, 2H), 3.84 (s, 3H), 3.92 (s, 6H), 12.73 (s, 1H). MS (DCI) m/z 377 (M+H).

### Example 70

### 4-ethoxy-N-[5-ethyl-4-(trifluoromethyl)-1,3-thiazol-2-yl]-6-methoxypyrimidine-5-carboxamide

### Example 70A

### 4-chloro-N-(5-ethyl-4-(trifluoromethyl)thiazol-2-yl)-6-methoxypyrimidine-5-carboxamide

The title compound was prepared as described in Example 2C, substituting 4-chloro-6-methoxypyrimidine-5-carboxylic acid for Example 2A, and substituting Example 6A for Example 2B (410 mg, 13.2%). MS (DCI) m/z 383.9 (M+H).

### Example 70B

4-ethoxy-N-[5-ethyl-4-(trifluoromethyl)-1,3-thiazol-2-yl]-6-methoxypyrimidine-5-carboxamide

To a solution of Example 70A (40 mg, 0.109 mmol) in ethanol (1 mL) was added a solution of sodium ethoxide (25.5 μl, 0.109 mmol, 21 weight % in ethanol), and the mixture was stirred at room temperature for 48 h followed by stirring at 40° C. for 18 h. The reaction mixture was concentrated in vacuo and the residue was diluted with EtOAc and washed with brine. The organic layer was dried (MgSO₄), concentrated under reduced pressure and the residue was purified by preparative HPLC on a Phenomenex LUNA C8(2) 5 um 100 Å AXIA column (30 mm×75 mm). A gradient of acetonitrile (A) and 0.1% trifluoroacetic acid in water (B) was used, at a flow rate of 50 mL/min (0-1.0 min 5% A, 1.0-8.5 min linear gradient 5-100% A, 8.5-11.5 min 100% A, 11.5-12.0 min linear gradient 95-5% A) to obtain the title compound (3.5 mg, 7.84% yield). ¹H NMR (500 MHz, DMSO-d₆) δ 1.25 (m, 6H), 2.82-3.01 (m, 2H), 3.93 (s, 3H), 4.42 (q, J=7.0 Hz, 2H), 8.57 (s, 1H), 12.80 (s, 1H). MS (DCI) m/z 377 (M+H).

### Example 71

### 4,6-diethoxy-N-[5-ethyl-4-(trifluoromethyl)-1,3-thiazol-2-yl]pyrimidine-5-carboxamide

The title compound was obtained as a result of purification of the reaction mixture described in Example 70B (4.8 mg, 10.7% yield). ¹H NMR (400 MHz, DMSO-d₆) δ 1.27 (m, 9H), 2.94 (m, 2H), 4.43 (m, 4H), 8.56 (s, 1H), 12.78 (s, 1H). MS (DCI) m/z 391 (M+H).

### Example 72

### 4,6-dimethoxy-N-(5-(pyridin-4-yloxy)-4-(trifluoromethyl)thiazol-2-yl)pyrimidine-5-carboxamide

A mixture of Example 31A (0.032 g, 0.130 mmol), pyridin-4-ol (0.015 g, 0.157 mmol), and Cs₂CO₃ (0.063 g, 0.193 mmol) in acetone (0.45 ml) was stirred at ambient temperature for 30 min, and heated to 65° C. for 2 h. The mixture was diluted with water, extracted with EtOAc (2×), and the organic phases were washed with brine, dried (Na₂SO₄), and subjected to chromatography on silica gel (35% EtOAc/CH₂Cl₂) to give the title compound (0.0029 g, 8.57% yield): ¹H NMR (501 MHz, CDCl₃) δ 8.63-8.46 (m, 2H), 7.08-6.95 (m, 2H), 5.43 (s, 2H).

### Example 73

### 4,6-dimethoxy-N-{4-[5-(trifluoromethyl)pyridin-2-yl]-1,3-thiazol-2-yl}pyrimidine-5-carboxamide

### Example 73A

### 4-(5-(trifluoromethyl)pyridin-2-yl)thiazol-2-amine

To a solution of 2-iodo-5-trifluoromethylpyridine (25 g, 92 mmol) in THF (200 mL) at −10° C. was added isopropylmagnesium chloride lithium chloride complex (77 mL, 101 mmol) dropwise via syringe pump over 70 min. The internal temperature was maintained between 0 and −10° C. during the addition. The mixture was allowed to stir for 30 min at 0° C. after the addition was complete, then 2-chloro-N-methoxy-N-methylacetamide (16.4 g, 119 mmol) in THF (25 mL) was added dropwise via syringe pump over 40 min. The mixture was then allowed to warm to ambient temperature over 90 min and allowed to stir at ambient temperature for 20 min. Thiourea (9.8 g, 128 mmol) in methanol (100 mL) was added and the mixture was allowed to stir at ambient temperature for 16 h. The mixture was quenched with saturated aqueous NaHCO₃ (100 mL), partially concentrated under reduced pressure and extracted with CH₂Cl₂ (3×50 mL). The combined organics were dried over anhydrous Na₂SO₄, filtered, concentrated under reduced pressure and purified via column chromatography (SiO₂, 100% CH₂Cl₂ to 60% EtOAc/CH₂Cl₂) to give the title compound (13.1 g, 58% yield). MS (ESI⁺) m/z 246 [M+H]⁺.

### Example 73B

### 4,6-dimethoxy-N-(4-(5-(trifluoromethyl)pyridin-2-yl)thiazol-2-yl)pyrimidine-5-carboxamide

The title compound was prepared as described in Example 2C, substituting Example 73A for Example 2B (9.45 g, 80% yield). ¹H NMR (400 MHz, DMSO-d₆) δ ppm 12.77 (s, 1H), 9.00-8.95 (m, 1H), 8.61 (s, 1H), 8.29 (dd, J=8.6, 2.3 Hz, 1H), 8.13-8.07 (m, 2H), 3.96 (s, 6H); MS (ESI⁺) m/z 412 [M+H]+.

### Example 74

### ([(4,6-dimethoxypyrimidin-5-yl)carbonyl]{4-[5-(trifluoromethyl)pyridin-2-yl]-1,3-thiazol-2-yl}amino)methyl dihydrogen phosphate

### Example 74A

### di-tert-butyl ((4,6-dimethoxy-N-(4-(5-(trifluoromethyl)pyridin-2-yl)thiazol-2-yl)pyrimidine-5-carboxamido)methyl) phosphate

A mixture of Example 73 (18.41 g, 44.7 mmol), di-tert-butyl (chloromethyl) phosphate (15.5 g, 59.9 mmol), potassium iodide (7.48 g, 45.1 mmol) and Cs₂CO₃ (29.21 g, 90 mmol) in N-methyl-2-pyrrolidone (135 ml) was stirred at 60° C. for 2 h, diluted with EtOAc (150 mL), washed with water (150 mL), saturated NaHCO₃, and brine. The organic phases were dried (Na₂SO₄) to give 37 g of tan, gummy solid, which was crystallized with cyclopentyl methyl ether (42 mL), and filtered to give the title compound (23.55 g, 83%): ¹H NMR (400 MHz, DMSO-d₆) δ 9.01 (d, J=2.4 Hz, 1H), 8.72 (s, 1H), 8.37 (dd, J=8.3, 2.5 Hz, 1H), 8.27 (m, 2H), 5.87 (d, J=6.1 Hz, 2H), 3.99 (s, 6H), 1.28 (s, 18H); MS (ESI) m/z 634 (M+H).

### Example 74B

### ([(4,6-dimethoxypyrimidin-5-yl)carbonyl]{4-[5-(trifluoromethyl)pyridin-2-yl]-1,3-thiazol-2-yl}amino)methyl dihydrogen phosphate

A mixture of Example 74A (22.62 g, 35.7 mmol), and concentrated aqueous hydrogen chloride (14 ml, 168 mmol) in ethanol (105 ml) was stirred at 50° C. for 90 min. The mixture was diluted with water (˜75 mL), filtered, dissolved in 1N NaOH (108 mL), extracted with CH₂Cl₂ (25 mL), organic layer acidified with 1N HCl (109 mL) with vigorous stirring, then filtered and dried in a vacuum oven to give the title compound (16.78 g, 32.2 mmol, 90% yield): ¹H NMR (400 MHz, Methanol-d₄) δ 8.87 (d, J=2.3 Hz, 1H), 8.57 (s, 1H), 8.40 (d, J=8.5 Hz, 1H), 8.18 (dd, J=8.3, 2.4 Hz, 1H), 8.13 (s, 1H), 5.96 (d, J=6.1 Hz, 2H), 4.04 (s, 6H); MS (ESI) m/z 522 (M+H).

### Example 75

### ethyl 2-{[(4,6-dimethoxypyrimidin-5-yl)carbonyl]amino}-4-[5-(trifluoromethyl)pyridin-2-yl]-1,3-thiazole-5-carboxylate

To a solution of diisopropylamine (0.208 mL, 1.46 mmol) in THF (3.2 mL) at −78° C. (dry-ice/acetone bath) was added n-BuLi (2.5 M solution in hexane, 0.583 mL, 1.46 mmol). The dry-ice/acetone bath was removed and the reaction mixture was stirred for 10 minutes and then chilled to −78° C. A solution of the product of Example 73B (200 mg, 0.49 mmol) in THF (5.0 mL) chilled to 0° C. was added dropwise over a period of 1 minute. After stirring at −78° C. for 2 minutes, ethyl chloroformate (0.467 mL, 4.86 mmol) was added dropwise over a period of 1 minute. The reaction mixture was stirred at −78° C. for 10 minutes and saturated aqueous ammonium chloride (0.5 mL) was added in one portion. The reaction mixture was warmed to ambient temperature and partitioned between dichloromethane (2×50 mL), saturated aqueous ammonium chloride (10 mL) and water (50 mL). The organic layers were combined, dried over anhydrous sodium sulfate and concentrated under reduced pressure. The resulting residue was purified via column chromatography (SiO₂, 20% to 60% EtOAc in heptane) to give the title compound (194 mg, 83% yield). ¹H NMR (400 MHz, DMSO-d₆) δ ppm 13.17 (s, 1H), 9.08-9.01 (m, 1H), 8.64 (s, 1H), 8.34 (dd, J=8.3, 2.3 Hz, 1H), 7.96 (d, J=8.2 Hz, 1H), 4.19 (q, J=7.1 Hz, 2H), 3.97 (s, 6H), 1.16 (t, J=7.1 Hz, 3H); MS (ESI⁺) m/z 484 [M+H]⁺.

### Example 76

### N-{5-formyl-4-[5-(trifluoromethyl)pyridin-2-yl]-1,3-thiazol-2-yl}-4,6-dimethoxypyrimidine-5-carboxamide

The title compound was prepared as described in Example 75, substituting dimethylformamide for ethyl chloroformate (41 mg, 40% yield). ¹H NMR (400 MHz, DMSO-d₆) δ ppm 13.29 (s, 1H), 10.82 (s, 1H), 9.14 (d, J=2.4 Hz, 1H), 8.64 (s, 1H), 8.44 (dd, J=8.5, 2.5 Hz, 1H), 8.24 (d, J=8.3 Hz, 1H), 3.96 (s, 6H); MS (ESI⁺) m/z 440 [M+H]⁺.

### Example 77

### 4,6-dimethoxy-N-{5-(morpholin-4-ylmethyl)-4-[5-(trifluoromethyl)pyridin-2-yl]-1,3-thiazol-2-yl}pyrimidine-5-carboxamide

A solution of Example 76 (0.150 g, 0.341 mmol) and morpholine (0.090 ml, 1.024 mmol) in THF (10.0 ml) and acetic acid (0.53 ml) was stirred for 30 min at ambient temperature, then SILICABOND cyanoborohydride (0.89 mmol/g) (1.343 g, 1.195 mmol) was added. The mixture was stirred overnight at ambient temperature, filtered, concentrated under reduced pressure, and the residue was purified by flash chromatography (12 g silica gel, 0-10% gradient of methanol in dichloromethane) to provide the title compound (68 mg, 32% yield). ¹H NMR (400 MHz, DMSO-d₆) δ ppm 13.04 (s, 1H), 9.22 (s, 1H), 8.65 (s, 1H), 8.43 (dd, J=8.5, 1.9 Hz, 1H), 8.25 (d, J=8.4 Hz, 1H), 4.99 (s, 2H), 4.03-3.72 (m, 10H), 3.65-3.22 (m, 4H). MS (DCI/NH₃) m/z 511 (M+H)⁺

### Example 78

### 4,6-dimethoxy-N-{5-(pyrrolidin-1-ylmethyl)-4-[5-(trifluoromethyl)pyridin-2-yl]-1,3-thiazol-2-yl}pyrimidine-5-carboxamide

The title compound was prepared as described in Example 77, substituting pyrrolidine for morpholine (49.8 mg, 24% yield). 1H NMR (400 MHz, DMSO-d6) δ ppm 13.03 (s, 1H), 9.20 (s, 1H), 8.65 (s, 1H), 8.43 (d, J=8.5, 2.1 Hz, 1H), 8.24 (d, J=8.4 Hz, 1H), 4.99 (d, J=5.2 Hz, 2H), 3.98 (s, 6H), 3.73-3.61 (m, 2H), 3.36-3.23 (m, 2H), 2.17-2.04 (m, 2H), 2.02-1.91 (m, 2H). MS (DCI/NH3) m/z 495 (M+H)+.

### Example 79

### N-{5-(dimethylcarbamoyl)-4-[5-(trifluoromethyl)pyridin-2-yl]-1,3-thiazol-2-yl}-4-hydroxy-6-methoxypyrimidine-5-carboxamide

### Example 79A

### 2-(4,6-dimethoxypyrimidine-5-carboxamido)-4-(5-(trifluoromethyl)pyridin-2-yl)thiazazole-5-carboxylic acid

To a solution of the product of Example 75 (170 mg, 0.35 mmol) in ethanol (20 mL) was added aqueous sodium hydroxide (2.5 M, 20 mL). After stirring at ambient temperature for 1 hour, a solution of citric acid (10% aqueous) was slowly added until precipitate started to form. Additional citric acid solution (10% aqueous, 10 mL) was added and the resulting mixture was partitioned between dichloromethane (2×100 mL) and water (50 mL). The organic layers were combined, dried over anhydrous sodium sulfate, filtered and concentrated under reduced pressure to give the title compound (160 mg, 100% yield). MS (ESI⁺) m/z 456 [M+H]⁺.

### Example 79B

### N-{5-(dimethylcarbamoyl)-4-[5-(trifluoromethyl)pyridin-2-yl]-1,3-thiazol-2-yl}-4-hydroxy-6-methoxypyrimidine-5-carboxamide

The product of Example 79A (35 mg, 0.08 mmol) was stirred in dichloromethane (2.0 mL) at ambient temperature and 1 drop of DMF was added, followed by oxalyl chloride (2.0 M in dichloromethane, 0.077 mL, 0.154 mmol). After stirring for 5 minutes, dimethylamine (2.0 M in THF, 0.27 mL, 0.54 mmol) was added. The mixture was stirred at ambient temperature for 1 hour and then concentrated under reduced pressure. The resulting residue was taken up in methanol (2 mL), filtered through a glass microfiber frit and purified by preparative HPLC [Waters XBRIDGE™ C18 5 μm OBD column, 50×100 mm, flow rate 90 mL/minute, 5-100% gradient of acetonitrile in buffer (0.1% TFA)] to give the title compound (15 mg, 42% yield). ¹H NMR (400 MHz, DMSO-d₆) δ ppm 13.39 (s, 1H), 13.08 (s, 1H), 8.95-8.92 (m, 1H), 8.44 (s, 1H), 8.29 (dd, J=8.5, 2.4 Hz, 1H), 8.16 (d, J=8.4 Hz, 1H), 4.00 (s, 3H), 3.02 (s, 3H), 2.74 (s, 3H); MS (ESI⁺) m/z 469 [M+H]⁺.

### Example 80

### N-{5-(hydroxymethyl)-4-[5-(trifluoromethyl)pyridin-2-yl]-1,3-thiazol-2-yl}-4,6-dimethoxypyrimidine-5-carboxamide

To a solution of the product of Example 76 (200 mg, 0.46 mmol) in a solvent mixture of methanol (15 mL) and THF (15 mL) at 0° C. was added sodium borohydride (20 mg, 0.53 mmol) in one portion. After 10 minutes, water (0.5 mL) was added and the resulting mixture was partitioned between dichloromethane (2×100 mL) and saturated, aqueous sodium bicarbonate solution (100 mL). The organic layers were combined, dried over anhydrous sodium sulfate, and concentrated under reduced pressure. The resulting residue was purified via column chromatography (SiO₂, 20% to 100% EtOAc in heptane) to give the title compound (202 mg, 100% yield). ¹H NMR (400 MHz, DMSO-d₆) δ ppm 12.54 (s, 1H), 9.02-8.99 (m, 1H), 8.60 (s, 1H), 8.28 (dd, J=8.5, 2.4 Hz, 1H), 8.11 (d, J=8.4 Hz, 1H), 5.91 (t, J=5.5 Hz, 1H), 5.13 (d, J=5.5 Hz, 2H), 3.95 (s, 6H); MS (ESI⁻) m/z 440 [M−H]⁻.

### Example 81

### ([(4,6-dimethoxypyrimidin-5-yl)carbonyl]{5-(hydroxymethyl)-4-[5-(trifluoromethyl)pyridin-2-yl]-1,3-thiazol-2-yl}amino)methyl dihydrogen phosphate

### Example 81A

### di-tert-butyl ((N-(5-(hydroxymethyl)-4-(5-(trifluoromethyl)pyridin-2-yl)thiazol-2-yl)-4,6-dimethoxypyrimidine-5-carboxamido)methyl) phosphate

A mixture of Example 80 (0.1822 g, 0.413 mmol), di-tert-butyl (chloromethyl) phosphate (0.140 g, 0.541 mmol), potassium iodide (0.0720 g, 0.434 mmol), and Cs₂CO₃ (0.2658 g, 0.816 mmol) in NMP (0.83 ml) was stirred at 60° C. for 90 min, diluted with EtOAc, washed with water and brine, dried (Na₂SO₄), and subjected to chromatography (20% EtOAc/DCM) to give di-tert-butyl ((N-(5-(hydroxymethyl)-4-(5-(trifluoromethyl)pyridin-2-yl)thiazol-2-yl)-4,6-dimethoxypyrimidine-5-carboxamido)methyl) phosphate (0.1611 g, 0.243 mmol, 59%): ¹H NMR (400 MHz, DMSO-d₆) δ 9.03 (d, J=2.0 Hz, 1H), 8.72 (s, 1H), 8.41-8.22 (m, 2H), 6.03 (t, J=5.5 Hz, 1H), 5.83 (d, J=5.9 Hz, 2H), 5.18 (d, J=5.5 Hz, 2H), 3.98 (s, 6H), 1.28 (s, 18H); MS (ESI) m/z 664 (M+H).

### Example 81B

### ([(4,6-dimethoxypyrimidin-5-yl)carbonyl]{5-(hydroxymethyl)-4-[5-(trifluoromethyl)pyridin-2-yl]-1,3-thiazol-2-yl}amino)methyl dihydrogen phosphate

A mixture of Example 81A (0.061 g, 0.091 mmol), and 2,2,2-trifluoroacetic acid (28 μl, 0.366 mmol) in CH₂Cl₂ (0.30 ml) was stirred for 3 h. Additional 2,2,2-trifluoroacetic acid (28 μl, 0.366 mmol) was added, and the mixture was heated to 35° C. overnight, concentrated under reduced pressure, diluted with water, made more basic by adding 0.4 mL of 1N NaOH, washed with CH₂Cl₂, acidified with 1N HCl (0.45 mL), and extracted with CH₂Cl₂. The organic layer was dried (Na₂SO₄) and concentrated to give the title compound (0.036 g, 72%): ¹H NMR (400 MHz, DMSO-d₆/D₂O) δ 8.99 (s, 1H), 8.64 (s, 1H), 8.33 (d, J=8.4 Hz, 1H), 8.24 (dd, J=8.6, 2.3 Hz, 1H), 5.77 (d, J=5.1 Hz, 2H), 5.16 (s, 2H), 3.96 (s, 6H); MS (ESI) m/z 552 (M+H).

### Example 82

### N-{5-(1-hydroxyethyl)-4-[4-(trifluoromethoxy)phenyl]-1,3-thiazol-2-yl}-4,6-dimethoxypyrimidine-5-carboxamide

The title compound was prepared as described in Example 75, substituting acetaldehyde for ethyl chloroformate and Example 93B for Example 73B (116 mg, 61% yield). ¹H NMR (400 MHz, DMSO-d₆) δ ppm 12.53 (s, 1H), 8.60 (s, 1H), 7.88-7.66 (m, 2H), 7.53-7.36 (m, 2H), 5.72 (d, J=4.2 Hz, 1H), 5.08 (qd, J=6.3, 4.3 Hz, 1H), 3.96 (s, 6H), 1.48 (d, J=6.3 Hz, 3H); MS (ESI⁺) m/z 471 [M+H]⁺.

### Example 83

### N-{5-[(1R*)-1-hydroxyethyl]-4-[5-(trifluoromethyl)pyridin-2-yl]-1,3-thiazol-2-yl}-4,6-dimethoxypyrimidine-5-carboxamide

The individual enantiomers of the mixture of Example 87A were separated by preparative chiral supercritical fluid chromatography (CHIRALPAK® OJ-H 5 μm 21×250 mm column; flow rate 70 mL/minute; 20% CH₃OH in CO₂) to afford the title compound as the first-eluting enantiomer (110 mg, 44% yield). ¹H NMR (400 MHz, DMSO-d6) δ ppm 12.38 (br s, 1H), 9.05-9.01 (m, 1H), 8.62 (s, 1H), 8.30 (dd, J=8.6, 2.4 Hz, 1H), 8.13 (d, J=8.5 Hz, 1H), 5.98-5.86 (m, 2H), 3.96 (s, 6H), 1.47 (d, J=5.7 Hz, 3H); MS (ESI⁻) m/z 454 (M−H)⁻.

### Example 84

### N-{5-[(1S*)-1-hydroxyethyl]-4-[5-(trifluoromethyl)pyridin-2-yl]-1,3-thiazol-2-yl}-4,6-dimethoxypyrimidine-5-carboxamide

The individual enantiomers of the mixture of Example 87A were separated by preparative chiral supercritical fluid chromatography (CHIRALPAK® OJ-H 5 μm 21×250 mm column; flow rate 70 mL/minute; 20% CH₃OH in CO₂) to afford the title compound as the second-eluting enantiomer (120 mg, 47% yield). ¹H NMR (501 MHz, DMSO-d6) δ ppm 12.33 (br s, 1H), 9.04-8.99 (m, 1H), 8.60 (s, 1H), 8.28 (dd, J=8.5, 2.4 Hz, 1H), 8.12 (d, J=8.4 Hz, 1H), 5.97-5.87 (m, 2H), 3.95 (s, 6H), 1.46 (d, J=5.7 Hz, 3H); MS (ESI⁻) m/z 454 (M−H)⁻.

### Example 85

### N-{5-(2-hydroxypropan-2-yl)-4-[5-(trifluoromethyl)pyridin-2-yl]-1,3-thiazol-2-yl}-4,6-dimethoxypyrimidine-5-carboxamide

The title compound was prepared as described in Example 75, substituting anhydrous acetone for ethyl chloroformate (26 mg, 23% yield). ¹H NMR (400 MHz, DMSO-d₆) δ ppm 12.54 (s, 1H), 9.07 (d, J=2.3 Hz, 1H), 8.60 (s, 1H), 8.35 (dd, J=8.5, 2.4 Hz, 1H), 8.22-8.18 (m, 1H), 7.15 (s, 1H), 3.95 (s, 6H), 1.62 (s, 6H); MS (ESI⁺) m/z 451 [M+H]⁺.

### Example 86

### 4,6-dimethoxy-N-{5-(methoxymethyl)-4-[5-(trifluoromethyl)pyridin-2-yl]-1,3-thiazol-2-yl}pyrimidine-5-carboxamide

The title compound was prepared as described in Example 75, substituting chloromethyl methyl ether for ethyl chloroformate (11 mg, 8% yield). ¹H NMR (400 MHz, DMSO-d₆) δ ppm 12.64 (s, 1H), 9.03-9.00 (m, 1H), 8.61 (s, 1H), 8.29 (dd, J=8.6, 2.4 Hz, 1H), 8.15-8.11 (m, 1H), 5.13 (s, 2H), 3.95 (s, 6H), 3.43 (s, 3H); MS (ESI⁻) m/z 454 [M−H]⁻.

### Example 87

### N-{5-acetyl-4-[5-(trifluoromethyl)pyridin-2-yl]-1,3-thiazol-2-yl}-4,6-dimethoxypyrimidine-5-carboxamide

### Example 87A

### N-{5-[1-hydroxyethyl]-4-[5-(trifluoromethyl)pyridin-2-yl]-1,3-thiazol-2-yl}-4,6-dimethoxypyrimidine-5-carboxamide

A solution of diisopropylamine (0.64 mL, 4.49 mmol) in THF (5 mL) was chilled to −75° C., and 2.5M n-butyllithium (1.8 mL, 4.50 mmol) in hexanes was then added. The light yellow solution was stirred at −5° C. for 30 min, and then chilled to −75° C. To that solution was added a solution of Example 73 (0.610 g, 1.483 mmol) in THF (5 mL) chilled to −5° C. dropwise. Resulting solution was stirred at −75° C. for 5 min, 5.0M acetaldehyde (1.5 mL, 7.50 mmol) in THF was added dropwise and the mixture was then stirred at −75° C. for 20 min. The reaction was allowed to warm to ambient temperature, quenched with 100 mL saturated NH₄Cl, extracted with 100 mL EtOAc, washed with brine, dried over Na₂SO₄, concentrated to an oil and subjected to chromatography on a Grace REVELERIS® 40 g column with 0-50% 3:1 EtOAc:EtOH in heptane (40 mL/min) to obtain the title compound (0.46 g, 1.0 mmol, 68.0% yield) as a beige foam. ¹H NMR (400 MHz, DMSO-d₆) δ 12.51 (s, 1H), 9.02-8.96 (m, 1H), 8.58 (s, 1H), 8.26 (ddd, J=8.5, 2.4, 0.8 Hz, 1H), 8.09 (dt, J =8.5, 0.8 Hz, 1H), 5.94-5.83 (m, 2H), 3.93 (s, 6H), 1.43 (d, J=6.6 Hz, 3H). MS (ESI⁻) m/z 454.1 (M−H).

### Example 87B

### N-{5-acetyl-4-[5-(trifluoromethyl)pyridin-2-yl]-1,3-thiazol-2-yl}-4,6-dimethoxypyrimidine-5-carboxamide

Dess-Martin periodinane (0.656 g, 1.547 mmol) was added to a solution of Example 87A (0.459 g, 1.008 mmol) in CH₂Cl₂ (15 mL). The yellow mixture was stirred overnight at ambient temperature, 200 mL EtOAc was added, and the resulting mixture was washed twice with saturated NaHCO₃, washed with brine, dried over Na₂SO₄, concentrated under reduced pressure and then subjected to chromatography on Grace REVELERIS® 40 g column with 0-100% 3:1 EtOAc:EtOH in heptane (40 mL/min) to obtain the title compound (0.25 g, 54.1% yield) as a yellow solid. ¹H NMR (400 MHz, DMSO-d₆) δ 13.08 (s, 1H), 9.10-9.04 (m, 1H), 8.62 (s, 1H), 8.38 (dd, J=8.4, 2.3 Hz, 1H), 8.03 (d, J=8.3 Hz, 1H), 3.95 (s, 6H), 2.30 (s, 3H). MS (ESI⁻) m/z 452.1 (M−H).

### Example 88

### N-{5-[(1E)-N-hydroxyethanimidoyl]-4-[5-(trifluoromethyl)pyridin-2-yl]-1,3-thiazol-2-yl}-4,6-dimethoxypyrimidine-5-carboxamide

Example 87B (0.215 g, 0.474 mmol) and hydroxylamine hydrochloride (0.099 g, 1.423 mmol) were dissolved in pyridine (6 mL), and then heated overnight at 50° C. The mixture was dissolved in 100 mL EtOAc, washed with 1.5N HCl, washed with brine, dried over Na₂SO₄, concentrated under reduced pressure and the residue was subjected to chromatography on Grace REVELERIS® 40 g column with 0-100% 3:1 EtOAc:EtOH in heptane (40 mL/min) to obtain the title compound (0.20 g, 0.428 mmol, 90% yield) as an off-white solid. ¹H NMR (400 MHz, DMSO-d₆) δ 12.77 (s, 1H), 11.45 (s, 1H), 9.01-8.97 (m, 1H), 8.59 (s, 1H), 8.29 (dd, J=8.5, 2.6 Hz, 1H), 8.04 (d, J=8.3 Hz, 1H), 3.93 (s, 6H), 1.92 (s, 3H). MS (ESI⁻) m/z 467.1 (M−H).

### Example 89

### 4,6-dimethoxy-N-{5-[(1E)-N-methoxyethanimidoyl]-4-[5-(trifluoromethyl)pyridin-2-yl]-1,3-thiazol-2-yl}pyrimidine-5-carboxamide

The title compound was prepared as described in Example 88, substituting O-methylhydroxylamine hydrochloride for hydroxylamine hydrochloride (4 mg, 47%). ¹H NMR (400 MHz, CDCl₃) δ 10.08 (s, 1H), 8.90 (s, 1H), 8.50 (s, 1H), 7.96 (dd, J=3.2, 1.7 Hz, 1H), 4.30 (d, J=1.4 Hz, 0H), 4.12 (d, J=1.5 Hz, 4H), 3.98 (s, 1H), 3.83 (s, 1H), 2.18 (s, 1H), 2.08 (s, 1H), 1.26 (s, 5H), 0.87 (dt, J=12.5, 7.5 Hz, 4H). MS (DCI/NH₃) m/z 483 (M+H)⁺.

### Example 90

### 4,6-dimethoxy-N-{5-(2-methyl-1,3-dioxolan-2-yl)-4-[5-(trifluoromethyl)pyridin-2-yl]-1,3-thiazol-2-yl}pyrimidine-5-carboxamide

The title compound was prepared as described in Example 137, substituting Example 87B for Example 133B (35 mg, 76%). ¹H NMR (501 MHz, CDCl₃) δ 10.4 (s, 1H), 8.97 (d, J=2 Hz, 1H), 8.45 (s, 1H), 8.08 (d, J=10 Hz, 1H), 7.95 (dd, J=10 Hz, J=2 Hz, 1H), 4.05 (s, 6H), 4.2-3.9 (m, 4H),), 1.88 (s, 3H). MS (DCI/NH₃) m/z 498 (M+H)⁺.

### Example 91

### N-[4-(5-fluoropyridin-2-yl)-1,3-thiazol-2-yl]-4,6-dimethoxypyrimidine-5-carboxamide

### Example 91A

### 4-(5-fluoropyridin-2-yl)thiazol-2-amine

A mixture of 2-chloro-1-(5-fluoropyridin-2-yl)ethanone (Enovation, 0.37 g, 2.13 mmol) and thiourea (0.16 g, 2.13 mmol) in methanol (10 mL) was stirred at ambient temperature for 16 h. The mixture was concentrated under reduced pressure to give the title product (0.45 g) which was carried on without purification. MS (ESI⁺) m/z 196 [M+H]⁺.

### Example 91B

### N-[4-(5-fluoropyridin-2-yl)-1,3-thiazol-2-yl]-4,6-dimethoxypyrimidine-5-carboxamide

The title compound was prepared as described in Example 2C, substituting Example 91A for Example 2B (0.3 g, 41% yield). ¹H NMR (400 MHz, DMSO-d6) δ ppm 12.70 (s, 1H), 8.63-8.60 (m, 2H), 7.98 (dd, J=8.8, 4.6 Hz, 1H), 7.85 (s, 1H), 7.81 (td, J=8.8, 3.0 Hz, 1H), 3.96 (s, 6H); MS (ESI⁺) m/z 362 [M+H]⁺.

### Example 92

### 4,6-dimethoxy-N-{4-[5-(trifluoromethoxy)pyridin-2-yl]-1,3-thiazol-2-yl}pyrimidine-5-carboxamide

### Example 92A

### 2-chloro-1-(5-(trifluoromethoxy)pyridin-2-yl)ethanone

To a solution of 2-chloro-1-(5-(trifluoromethoxy)pyridin-2-yl)ethanone (Alchem Pharmtech, 0.10 g, 0.42 mmol) in methanol (3 mL) was added thiourea (0.033 g, 0.44 mmol). This mixture was allowed to stir at ambient temperature for 16 h, and then was concentrated under reduced pressure and purified via column chromatography (SiO₂, 1% EtOAc/heptanes to 40% EtOAc/heptanes) to give the title product (0.030 g, 28% yield). MS (ESI⁺) m/z 240 [M+H]⁺.

### Example 92B

### 4,6-dimethoxy-N-{4-[5-(trifluoromethoxy)pyridin-2-yl]-1,3-thiazol-2-yl}pyrimidine-5-carboxamide

The title compound was prepared as described in Example 2C, substituting Example 92A for Example 2B (20 mg, 41% yield). ¹H NMR (400 MHz, DMSO-d₆) δ ppm 12.75 (s, 1H), 8.72-8.68 (m, 1H), 8.62 (s, 1H), 8.07-8.02 (m, 1H), 8.02-7.92 (m, 2H), 3.96 (s, 6H); MS (ESI⁺) m/z 428 [M+H]⁺.

### Example 93

### 4,6-dimethoxy-N-{4-[4-(trifluoromethoxy)phenyl]-1,3-thiazol-2-yl}pyrimidine-5-carboxamide

### Example 93A

### 4-(4-(trifluoromethoxy)phenyl)thiazol-2-amine

A mixture of 2-bromo-4′-(trifluoromethoxy)acetophenone (1.67 g, 5.90 mmol) and thiourea (0.45 g, 5.90 mmol) in ethanol (15 mL) was stirred at ambient temperature for 68 h. The mixture was concentrated under reduced pressure and the solids were washed with Et₂O and then dissolved in H₂O (20 mL). The solution was brought to pH 11 by the addition of 10% NaOH (aq) and the resulting solids were isolated by filtration, washed with H₂O and dried under reduced pressure to give the title compound (1.41 g, 92% yield). MS (ESI⁺) m/z 261 [M+H]⁺.

### Example 93B

### 4,6-dimethoxy-N-{4-[4-(trifluoromethoxy)phenyl]-1,3-thiazol-2-yl}pyrimidine-5-carboxamide

The title compound was prepared as described in Example 2C, substituting Example 93A for Example 2B (0.57 g, 1.34 mmol, 40% yield). ¹H NMR (400 MHz, DMSO-d₆) δ ppm 12.70 (s, 1H), 8.62 (s, 1H), 8.07-7.96 (m, 2H), 7.79 (s, 1H), 7.43 (d, J=8.3 Hz, 2H), 3.96 (s, 6H); MS (ESI⁺) m/z 427 [M+H]⁺

### Example 94

### N-{4-[5-(difluoromethyl)pyridin-2-yl]-1,3-thiazol-2-yl}-4,6-dimethoxypyrimidine-5-carboxamide

### Example 94A

### 4-(5-(difluoromethyl)pyridin-2-yl)thiazol-2-amine

To a solution of 2-bromo-5-(difluoromethyl)pyridine (2.0 g, 9.62 mmol) in THF (25 mL) at −10° C. was added isopropylmagnesium chloride lithium chloride complex (8.14 mL, 10.6 mmol) dropwise over 20 min. The internal temperature was maintained between 0 and −10° C. during the addition. The mixture was allowed to stir for 30 min at 0° C. after the addition was completed, and then the 2-chloro-N-methoxy-N-methylacetamide (1.72 g, 12.5 mmol) in THF (25 mL) was added dropwise via syringe pump over 20 min. The mixture was then allowed to warm to ambient temperature over 90 min and then allowed to stir at ambient temperature for 20 min. Thiourea (1.03 g, 13.5 mmol) in methanol (15 mL) was added and the mixture was allowed to stir at ambient temperature for 16 h. The mixture was quenched with saturated aqueous NaHCO₃ (100 mL), partially concentrated under reduced pressure and extracted with CH₂Cl₂ (3×50 mL). The combined organics were dried over anhydrous Na₂SO₄, filtered, concentrated under reduced pressure and purified via column chromatography (SiO₂, 100% CH₂Cl₂ to 60% EtOAc/CH₂Cl₂) to give the title compound (0.10 g, 4.6% yield). MS (ESI⁺) m/z 228 [M+H]⁺.

### Example 94B

### N-{4-[5-(difluoromethyl)pyridin-2-yl]-1,3-thiazol-2-yl}-4,6-dimethoxypyrimidine-5-carboxamide

The title compound was prepared as described in Example 2C, substituting Example 94A for Example 2B (30 mg, 0.076 mmol, 12.4% yield). ¹H NMR (400 MHz, DMSO-d6) δ ppm 12.74 (s, 1H), 8.80 (d, J=2.1 Hz, 1H), 8.61 (s, 1H), 8.13-7.96 (m, 3H), 7.17 (t, J=55.4 Hz, 1H), 3.96 (s, 6H); MS (ESI⁺) m/z 394 [M+H]⁺.

### Example 95

### 4,6-dimethoxy-N-{5-methyl-4-[5-(trifluoromethyl)pyridin-2-yl]-1,3-thiazol-2-yl}pyrimidine-5-carboxamide

The title compound was prepared as described in Example 75, substituting methyl iodide for ethyl chloroformate (0.21 g, 81%). ¹H NMR (400 MHz, DMSO-d₆) δ ppm 12.61 (s, 1H), 9.03-9.00 (m, 1H), 8.62 (s, 1H), 8.28 (dd, J=8.5, 2.4 Hz, 1H), 8.13 (d, J=8.5 Hz, 1H), 3.97 (s, 6H), 2.81 (s, 3H); MS (ESI⁻) m/z 424 [M−H]⁻.

### Example 96

### N-{5-(3-hydroxypropyl)-4-[5-(trifluoromethyl)pyridin-2-yl]-1,3-thiazol-2-yl}-4,6-dimethoxypyrimidine-5-carboxamide

### Example 96A

### N-(5-iodo-4-(5-(trifluoromethyl)pyridin-2-yl)thiazol-2-yl)-4,6-dimethoxypyrimidine-5-carboxamide

The title compound was prepared as described in Example 75, substituting iodine (5.0 M in THF) for ethyl chloroformate (0.51 g, 103% yield). ¹H NMR (400 MHz, DMSO-d₆) δ ppm 13.02 (s, 1H), 9.07-9.05 (m, 1H), 8.63 (s, 1H), 8.33 (dd, J=8.5, 2.4 Hz, 1H), 8.16 (d, J=8.3 Hz, 1H), 3.97 (s, 6H); MS (ESI⁻) m/z 536 [M−H]⁻.

### Example 96B

### (E)-N-(5-(3-hydroxyprop-1-en-1-yl)-4-(5-(trifluoromethyl)pyridin-2-yl)thiazol-2-yl)-4,6-dimethoxypyrimidine-5-carboxamide

A microwave vial (10 mL) was charged with (trans)-3-trimethylsiloxy-1-propenylboronic acid pinacol ester (Alfa, 244 mg, 0.95 mmol), Example 96A (256 mg, 0.48 mmol), potassium carbonate (132 mg, 0.95 mmol), PdCl₂(dppf)-CH₂Cl₂ complex (39 mg, 0.048 mmol), dimethoxyethane (4.0 mL) and water (0.8 mL). The vial was sealed and heated at 122° C. for 30 minutes in the microwave reactor (Biotage PERSONALCHEMISTRY™). The reaction mixture was cooled to ambient temperature and partitioned between dichloromethane (2×50 mL) and aqueous sodium carbonate (1.0 M, 50 mL). The organic layers were combined, dried over anhydrous sodium sulfate, and concentrated under reduced pressure. The resulting residue was purified by preparative HPLC [Waters XBRIDGE™ C18 5 μm OBD column, 50×100 mm, flow rate 90 mL/minute, 5-100% gradient of MeOH in buffer (0.1% TFA)] to give the title compound (70 mg, 31%). ¹H NMR (400 MHz, DMSO-d₆) δ ppm 12.75 (s, 1H), 9.06-9.02 (m, 1H), 8.63 (s, 1H), 8.30 (dd, J=8.5, 2.4 Hz, 1H), 8.12 (d, J=8.4 Hz, 1H), 7.89 (dt, J=16.1, 1.8 Hz, 1H), 6.26 (dt, J=16.0, 5.0 Hz, 1H), 4.98 (s, 1H), 4.15 (dd, J=4.9, 1.9 Hz, 2H), 3.97 (s, 6H); MS (ESI⁻) m/z 466 [M−H]⁻.

### Example 96C

### N-{5-(3-hydroxypropyl)-4-[5-(trifluoromethyl)pyridin-2-yl]-1,3-thiazol-2-yl}-4,6-dimethoxypyrimidine-5-carboxamide

To a microwave vial (2 mL) was added the product of Example 96B (38 mg, 0.08 mmol), palladium on carbon (10 wt. % loading, 6.9 mg), ammonium formate (31 mg, 0.49 mmol) and ethanol (1.45 mL) in sequential order. The vial was sealed and heated at 150° C. for 8 minutes in the microwave reactor (Biotage PERSONALCHEMISTRY™). The reaction mixture was cooled to ambient temperature and filtered through a pack of CELITE®. The filter cake was further washed with additional ethanol (20 mL). The filtrate was concentrated in vacuo and the residue was purified via column chromatography (SiO₂, 20% to 100% EtOAc in dichloromethane) to give the title compound (20 mg, 52% yield). ¹H NMR (400 MHz, DMSO-d₆) δ ppm 12.62 (s, 1H), 9.02-9.00 (m, 1H), 8.62 (s, 1H), 8.29 (dd, J=8.5, 2.4 Hz, 1H), 8.12 (d, J=8.4 Hz, 1H), 4.55 (t, J=5.2 Hz, 1H), 3.97 (s, 6H), 3.53-3.45 (m, 2H), 3.41-3.34 (m, 2H), 1.90-1.79 (m, 2H); MS (ESI⁻) m/z 468 [M−H]⁻.

### Example 97

### N-{5-cyano-4-[5-(trifluoromethyl)pyridin-2-yl]-1,3-thiazol-2-yl}-4,6-dimethoxypyrimidine-5-carboxamide

To a sealed tube was added DMF (2.0 mL), Example 76 (88 mg, 0.200 mmol), hydroxylamine hydrochloride (15.3 mg, 0.22 mmol), triethylamine (31 μL, 0.22 mmol) and propylphosphonic anhydride (Aldrich, 50 wt. % in EtOAc, 129 μL) in sequential order. The tube was sealed and stirred at 100° C. for 1 hour. The reaction mixture was cooled to ambient temperature and partitioned between dichloromethane (2×50 mL) and saturated aqueous sodium bicarbonate (100 mL). The organic layers were combined and dried over anhydrous sodium sulfate and concentrated under reduced pressure. The resulting residue was purified by preparative HPLC [Waters XBRIDGE™ C18 5 μm OBD column, 30×100 mm, flow rate 40 mL/minute, 20-100% gradient of MeOH in buffer (0.025 M aqueous ammonium bicarbonate, adjusted to pH 10 with ammonium hydroxide)]. Fractions containing the desired product were combined and concentrated under reduced pressure. The resulting residue was further purified by preparative HPLC [Waters XBRIDGE™ C18 5 μm OBD column, 30×100 mm, flow rate 40 mL/minute, 10-100% gradient of MeOH in buffer (0.1% TFA)] to give the title compound (20 mg, 52% yield). ¹H NMR (400 MHz, DMSO-d₆) δ ppm 13.60 (s, 1H), 9.17-9.13 (m, 1H), 8.66 (s, 1H), 8.44 (dd, J=8.4, 2.3 Hz, 1H), 8.21 (d, J=8.3 Hz, 1H), 3.98 (s, 6H); MS (ESI⁺) m/z 437 [M+H]⁺.

### Example 98

### N-(4-cyclopentyl-1,3-thiazol-2-yl)-4,6-dimethoxypyrimidine-5-carboxamide

### Example 98A

### 4-cyclopentylthiazol-2-amine

A mixture of 2-bromo-1-cyclopentylethan-1-one (Enamine, 1 g, 4.97 mmol) and thiourea (0.38 g, 5.0 mmol) in ethanol (15 mL) was stirred at ambient temperature for 16 h. The mixture was concentrated under reduced pressure and the solids were dissolved in H₂O (70 mL). The solution was brought to pH 11 by an addition of 10% NaOH (aq) and the resulting solids were isolated by filtration, washed with H₂O, and dried under reduced pressure to give the title compound (0.67 g, 80% yield). MS (ESI⁺) m/z 169 [M+H]⁺.

### Example 98B

### N-(4-cyclopentyl-1,3-thiazol-2-yl)-4,6-dimethoxypyrimidine-5-carboxamide

The title compound was prepared as described in Example 2C, substituting Example 98A for Example 2B (0.4 g, 81% yield). ¹H NMR (400 MHz, DMSO-d₆) δ ppm 12.44 (s, 1H), 8.57 (s, 1H), 6.83 (s, 1H), 3.93 (s, 6H), 3.14-3.00 (m, 1H), 2.03-1.88 (m, 2H), 1.75-1.57 (m, 6H); MS (ESI⁺) m/z 335 [M+H]⁺.

### Example 99

### N-[4-(4,4-difluorocyclohexyl)-1,3-thiazol-2-yl]-4,6-dimethoxypyrimidine-5-carboxamide

### Example 99A

### 4-(4,4-difluorocyclohexyl)thiazol-2-amine

A mixture of 2-chloro-1-(4,4-difluorocyclohexyl)ethan-1-one (Ukrorgsyntez, 1 g, 5.09 mmol) and thiourea (0.39 g, 5.09 mmol) in ethanol (15 mL) was stirred at ambient temperature for 16 h. The mixture was concentrated under reduced pressure and the solids were dissolved in H₂O (70 mL). The solution was brought to pH 11 by addition of 10% NaOH (aq) and the resulting solids were isolated by filtration, washed with H₂O and dried under reduced pressure to give the title compound (1.06 g, 4.9 mmol, 95% yield). MS (ESI⁺) m/z 219 [M+H]⁺.

### Example 99B

### N-[4-(4,4-difluorocyclohexyl)-1,3-thiazol-2-yl]-4,6-dimethoxypyrimidine-5-carboxamide

The title compound was prepared as described in Example 2C, substituting Example 99A for Example 2B (1.3 g, 70% yield). ¹H NMR (400 MHz, DMSO-d₆) δ ppm 12.46 (s, 1H), 8.59 (s, 1H), 6.92 (s, 1H), 3.94 (s, 6H), 2.87-2.73 (m, 1H), 2.19-1.83 (m, 6H), 1.78-1.62 (m, 2H); MS (ESI⁺) m/z 385 [M+H]⁺.

### Example 100

### N-(4-tert-butyl-5-fluoro-1,3-thiazol-2-yl)-4,6-dimethoxypyrimidine-5-carboxamide

### Example 100A

### N-(4-(tert-butyl)thiazol-2-yl)-4,6-dimethoxypyrimidine-5-carboxamide

The title compound was prepared as described in Example 2C, substituting commercially available 4-(tert-butyl)thiazol-2-amine for Example 2B (1.85 g, 79% yield). MS (ESI⁺) m/z 323 [M+H]⁺.

### Example 100B

### N-(4-tert-butyl-5-fluoro-1,3-thiazol-2-yl)-4,6-dimethoxypyrimidine-5-carboxamide

To a solution of Example 100A (0.22 g, 0.68 mmol) in acetonitrile (12 mL) was added 1-chloromethyl-4-fluoro-1,4-diazoniabicyclo[2.2.2]octane bis(tetrafluoroborate) (0.24 g, 0.68 mmol) (Selectfluor). The mixture was allowed to stir for 16 h then was quenched with H₂O (5 mL) and diluted with CH₂Cl₂ (10 mL). The layers were separated and the aqueous layer was extracted with CH₂Cl₂ (3×5 mL). The combined organics were dried over anhydrous Na₂SO₄, filtered, concentrated under reduced pressure and purified via column chromatography (SiO₂, 100% CH₂Cl₂ to 40% EtOAc in CH₂Cl₂) then (SiO₂, 5% EtOAc/heptanes to 50% EtOAc/heptanes) to give the title compound (0.15 g, 20% yield). ¹H NMR (400 MHz, DMSO-d₆) δ ppm 12.54 (s, 1H), 8.59 (s, 1H), 3.94 (s, 6H), 1.30 (d, J=1.6 Hz, 9H); MS (ESI⁺) m/z 341 [M+H]⁺.

### Example 101

### N-(4-cyclobutyl-5-fluoro-1,3-thiazol-2-yl)-4,6-dimethoxypyrimidine-5-carboxamide

### Example 101A

### N-(4-cyclobutylthiazol-2-yl)-4,6-dimethoxypyrimidine-5-carboxamide

The title compound was prepared as described in Example 2C, substituting commercially available 4-cyclobutylthiazol-2-amine for Example 2B (1.65 g, 79% yield). MS (ESI⁺) m/z 321 [M+H]⁺.

### Example 101B

### N-(4-cyclobutyl-5-fluoro-1,3-thiazol-2-yl)-4,6-dimethoxypyrimidine-5-carboxamide

To a solution of Example 101A (0.14 g, 0.43 mmol) in acetonitrile (5 mL) was added 1-chloromethyl-4-fluoro-1,4-diazoniabicyclo[2.2.2]octanebis(tetrafluoroborate) (0.15 g, 0.43 mmol) (Selectfluor). The mixture was allowed to stir for 16 h then was quenched with H₂O (5 mL) and diluted with CH₂Cl₂ (10 mL). The layers were separated and the aqueous layer was extracted with CH₂Cl₂ (3×5 mL). The combined organics were dried over anhydrous Na₂SO₄, filtered, concentrated under reduced pressure and purified via column chromatography (SiO₂, 100% CH₂Cl₂ to 40% EtOAc in CH₂Cl₂) then (SiO₂, 5% EtOAc/heptanes to 50% EtOAc/heptanes) to give the title compound (25 mg, 17% yield). ¹H NMR (400 MHz, DMSO-d₆) δ ppm 12.64 (s, 1H), 8.60 (s, 1H), 3.94 (s, 6H), 3.67-3.53 (m, 1H), 2.23 (dd, J=10.4, 8.2 Hz, 4H), 2.07-1.91 (m, 1H), 1.89-1.77 (m, 1H); MS (ESI⁺) m/z 339 [M+H]⁺.

### Example 102

### N-(5-cyano-4-cyclobutyl-1,3-thiazol-2-yl)-4,6-dimethoxypyrimidine-5-carboxamide

### Example 102A

### N-(4-cyclobutyl-5-formylthiazol-2-yl)-4,6-dimethoxypyrimidine-5-carboxamide

To a solution of diisopropylamine (0.75 mL, 5.34 mmol) in THF (10 mL) at −78° C. (dry-ice/acetone bath) was added n-butyllithium (2.14 mL, 5.34 mmol) (2.5 M solution in hexanes). The dry-ice bath was removed and the mixture was allowed to stir for 10 min then the dry-ice bath was replaced. A solution of Example 101A (0.57 g, 1.78 mmol) in THF (7 mL) at 0° C. was added dropwise to the newly formed lithium diisopropylamine solution. The mixture was stirred for 2 min after the addition was complete, then N,N-dimethylformamide (2.07 mL, 26.7 mmol) was added dropwise over 10 min. The reaction mixture was allowed to stir at −78° C. for 10 min, and then the dry-ice acetone bath was replaced with an ice-water bath. The mixture was allowed to stir at 0° C. for 30 min, and then was quenched with saturated aqueous NaHCO₃ (10 mL). The ice-bath was removed and the mixture was diluted with CH₂Cl₂ (15 mL). The layers were separated and the aqueous layer was extracted with CH₂Cl₂ (3×7 mL). The combined organics were dried over anhydrous Na₂SO₄, filtered, and concentrated under reduced pressure. The mixture was purified via column chromatography (SiO₂, 100% CH₂Cl₂ to 40% EtOAc in CH₂Cl₂) to give the title compound (0.29 g, 47% yield). MS (ESI⁺) m/z 349 [M+H]⁺.

### Example 102B

### N-(5-cyano-4-cyclobutyl-1,3-thiazol-2-yl)-4,6-dimethoxypyrimidine-5-carboxamide

To a solution of Example 102A (0.29 g, 0.832 mmol) in DMF (5 mL) was added triethylamine (0.35 mL, 2.5 mmol) followed by hydroxylamine hydrochloride (0.038 mL, 0.92 mmol) and 2,4,6-tripropyl-1,3,5,2,4,6-trioxatriphosphinane 2,4,6-trioxide (>50% in EtOAc, 0.54 mL, 0.92 mmol). The mixture was warmed to 100° C. and was allowed to stir for 90 min. Starting material was determined to remain after subjecting a sample of the mixture to LC/MS so the mixture was cooled slightly (raised out of oil bath for 5 min) and additional hydroxylamine hydrochloride (0.038 mL, 0.92 mmol) and 2,4,6-tripropyl-1,3,5,2,4,6-trioxatriphosphinane 2,4,6-trioxide (0.54 mL, 0.92 mmol) were added. The mixture was then stirred at 100° C. for additional 1 h at which time no starting material was present as detected by LC/MS. The mixture was allowed to cool to ambient temperature, quenched with saturated aqueous NaHCO₃ (7 mL), and diluted with CH₂Cl₂ (7 mL). The layers were separated and the aqueous material was extracted with CH₂Cl₂ (3×5 mL). The combined organics were dried over anhydrous Na₂SO₄, filtered, concentrated under reduced pressure and purified via column chromatography (SiO₂, 100% CH₂Cl₂ to 40% EtOAc in CH₂Cl₂) to give the title compound (0.15 g, 0.43 mmol, 52% yield). ¹H NMR (400 MHz, DMSO-d₆) δ ppm 13.41 (s, 1H), 8.63 (s, 1H), 3.96 (s, 6H), 3.78 (p, J=8.6 Hz, 1H), 2.40-2.25 (m, 4H), 2.14-1.98 (m, 1H), 1.95-1.80 (m, 1H); MS (ESI⁺) m/z 346 [M+H]⁺.

### Example 103

### N-{4-[(E)-2-cyclopropylethenyl]-1,3-thiazol-2-yl}-4,6-dimethoxypyrimidine-5-carboxamide

### Example 103A

### N-(4-bromothiazol-2-yl)-4,6-dimethoxypyrimidine-5-carboxamide

The title compound was prepared as described in Example 2C, substituting 2-amino-4-bromothiazole (CombiPhos) for Example 2B (0.49 g, 51% yield). MS (ESI⁺) m/z 345/347 [M+H]⁺.

### Example 103B

### N-{4-[(E)-2-cyclopropylethenyl]-1,3-thiazol-2-yl}-4,6-dimethoxypyrimidine-5-carboxamide

To a sealed tube (5 mL) was introduced Example 103A (166 mg, 0.48 mmol), (trans)-2-cyclopropylvinylboronic acid pinacol ester (140 mg, 0.72 mmol), potassium carbonate (146 mg, 1.06 mmol), PdCl₂(dppf)-CH₂Cl₂ complex (39 mg, 0.048 mmol), dimethoxyethane (2.0 mL), and water (0.7 mL) in sequential order. The tube was sealed and heated at 100° C. for 18 hours. The reaction mixture was cooled to ambient temperature and partitioned between dichloromethane (2×50 mL) and water (50 mL). The organic layers were combined, dried over anhydrous sodium sulfate and concentrated under reduced pressure. The resulting residue was purified via column chromatography (SiO₂, 20-100% EtOAc in heptane) to give the title compound (100 mg, 63% yield). ¹H NMR (400 MHz, DMSO-d₆) δ ppm 12.45 (s, 1H), 8.59 (s, 1H), 6.94 (s, 1H), 6.43 (d, J=15.4 Hz, 1H), 5.89 (dd, J=15.4, 9.3 Hz, 1H), 3.94 (s, 6H), 1.60-1.49 (m, 1H), 0.84-0.74 (m, 2H), 0.50-0.41 (m, 2H); MS (ESI⁺) m/z 333 [M+H]⁺.

### Example 104

### N-{4-[(1E)-3,3-dimethylbut-1-en-1-yl]-1,3-thiazol-2-yl}-4,6-dimethoxypyrimidine-5-carboxamide

The title compound was prepared as described in Example 103B, substituting 3,3-dimethyl-1-butenylboronic acid (Matrix) for (trans)-2-cyclopropylvinylboronic acid pinacol ester (120 mg, 72% yield). ¹H NMR (400 MHz, DMSO-d6) δ ppm 12.48 (s, 1H), 8.59 (s, 1H), 7.03 (s, 1H), 6.45 (d, J=15.8 Hz, 1H), 6.27 (d, J=15.8 Hz, 1H), 3.95 (s, 6H), 1.08 (s, 9H); MS (ESI⁺) m/z 345/347 [M+H]⁺.

### Example 105

### N-[4-(2-cyclopropylethyl)-1,3-thiazol-2-yl]-4,6-dimethoxypyrimidine-5-carboxamide

Palladium on carbon (10 wt. %, wet support, 16 mg) was added to a solution of Example 104 (82 mg, 0.25 mmol) in ethanol (10 mL). The reaction mixture was hydrogenated at ambient temperature under 14 psi for 24 hours. The resulting mixture was filtered through a glass microfiber frit and purified by preparative HPLC [Waters XBRIDGE™ C18 5 μm OBD column, 30×100 mm, flow rate 35 mL/minute, 20-100% gradient of acetonitrile in buffer (0.025 M aqueous ammonium bicarbonate, adjusted to pH 10 with ammonium hydroxide)] to give the title compound (37 mg, 45% yield). ¹H NMR (400 MHz, DMSO-d₆) δ ppm 12.40 (s, 1H), 8.57 (s, 1H), 6.83 (s, 1H), 3.93 (s, 6H), 2.71-2.63 (m, 2H), 1.51 (q, J=7.2 Hz, 2H), 0.75-0.62 (m, 1H), 0.41-0.34 (m, 2H), 0.06-−0.04 (m, 2H); MS (ESI⁺) m/z 334 [M+H]⁺.

### Example 106

### N-{4-[(E)-2-(4-fluorophenyl)ethenyl]-1,3-thiazol-2-yl}-4,6-dimethoxypyrimidine-5-carboxamide

The title compound was prepared as described in Example 103B, substituting (trans)-2-(4-fluorophenyl)vinylboronic acid for (trans)-2-cyclopropylvinylboronic acid pinacol ester (0.12 g, 78% yield). ¹H NMR (400 MHz, DMSO-d6) δ ppm 12.61 (s, 1H), 8.61 (s, 1H), 7.64-7.58 (m, 2H), 7.26-7.13 (m, 5H), 3.96 (s, 6H); MS (ESI⁺) m/z 387 [M+H]⁺.

### Example 107

### N-{4-[2-(4-fluorophenyl)ethyl]-1,3-thiazol-2-yl}-4,6-dimethoxypyrimidine-5-carboxamide

Palladium on carbon (5 wt. %, wet support; 60 mg) was added to a solution of Example 106 (93 mg, 0.24 mmol) in MeOH (10 mL). The reaction mixture was hydrogenated at ambient temperature under 30 psi for 16 hours. The reaction mixture was filtered through a glass microfiber frit and concentrated under reduced pressure. The resulting residue was purified via column chromatography (SiO₂, 20-80% EtOAc in heptane) to give the title compound (55 mg, 59% yield). ¹H NMR (400 MHz, DMSO-d6) δ ppm 12.46 (s, 1H), 8.59 (s, 1H), 7.28-7.21 (m, 2H), 7.13-7.06 (m, 2H), 6.84 (s, 1H), 3.95 (s, 6H), 2.97-2.86 (m, 4H); MS (APCI) m/z 389 [M+H]⁺.

### Example 108

### N-[4-(4-fluoro-2-methoxyphenyl)-1,3-thiazol-2-yl]-4,6-dimethoxypyrimidine-5-carboxamide

The title compound was prepared as described in Example 103B, substituting (4-fluoro-2-methoxyphenyl)boronic acid for (trans)-2-cyclopropylvinylboronic acid pinacol ester (75 mg, 66% yield). ¹H NMR (400 MHz, DMSO-d6) δ ppm 12.56 (s, 1H), 8.60 (br s, 1H), 8.10-7.94 (m, 1H), 7.66 (s, 1H), 7.09-6.96 (m, 1H), 6.92-6.77 (m, 1H), 3.94 (br s, 9H); MS (ESI⁺) m/z 391 [M+H]⁺.

### Example 109

### 4,6-dimethoxy-N-[5-(p-tolylsulfonyl)-4-[5-(trifluoromethyl)-2-pyridyl]-1,3-thiazol-2-yl]pyrimidine-5-carboxamide

The title compound was prepared as described in Example 75, substituting p-toluenesulfonyl fluoride for ethyl chloroformate (33 mg, 40% yield). ¹H NMR (501 MHz, DMSO-d6) δ ppm 13.48 (s, 1H), 9.14-9.12 (m, 1H), 8.75 (s, 1H), 8.44 (dd, J=8.5, 2.4 Hz, 1H), 8.15 (d, J=8.3 Hz, 1H), 8.12-8.08 (m, 2H), 7.54 (d, J=8.2 Hz, 2H), 4.07 (s, 6H), 2.49 (s, 3H); MS (ESI⁺) m/z 566 [M+H]⁺.

### Example 110

### 4,6-dimethoxy-N-[5-(p-tolylsulfonyl)-4-(trifluoromethyl)-1,3-thiazol-2-yl]pyrimidine-5-carboxamide

A solution of diisopropylamine (0.64 mL, 4.49 mmol) in THF (5 mL) was chilled to −75° C., and 2.5M n-butyllithium (1.8 mL, 4.50 mmol) in hexanes was then added. The light yellow solution was stirred at −5° C. for 30 min, and then chilled to −75° C. To the chilled mixture was added a solution of Example 73 (0.610 g, 1.483 mmol) in THF (5 mL) chilled to −5° C. dropwise. The resulting solution was stirred at −75° C. for 5 min, and then a solution of p-toluenesulfonyl fluoride (0.54 g, 3.10 mmol) in THF was added dropwise, followed by stirring the mixture at −75° C. for 20 min. The reaction was allowed to warm to ambient temperature, quenched with 100 mL saturated NH₄Cl, extracted with 100 mL EtOAc, washed with brine, dried over Na₂SO₄, concentrated to an oil and subjected to chromatography on a Grace REVELERIS® 40 g column with 0-50% 3:1 EtOAc:EtOH in heptane (40 mL/min) to obtain the title compound (0.393 g, 0.805 mmol, 53.8% yield) as a yellow foam. ¹H NMR (400 MHz, DMSO-d₆) δ 13.70 (s, 1H), 8.65 (s, 1H), 7.90 (d, J=8.2 Hz, 2H), 7.51 (d, J=8.1 Hz, 2H), 3.96 (s, 6H), 2.43 (s, 3H). MS (ESI⁻) m/z 487.1 (M−H).

### Example 111

### N-[5-benzylsulfonyl-4-(trifluoromethyl)-1,3-thiazol-2-yl]-4,6-dimethoxy-pyrimidine-5-carboxamide

### Example 111A

### N-[5-benzylthio-4-(trifluoromethyl)-1,3-thiazol-2-yl]-4,6-dimethoxy-pyrimidine-5-carboxamide

The title compound was prepared as described in Example 110, substituting dibenzyldisulfide for p-toluenesulfonyl fluoride (0.466 g, 68.3% yield). ¹H NMR (400 MHz, DMSO-d₆) δ 13.04 (s, 1H), 8.60 (s, 1H), 7.34-7.23 (m, 5H), 4.21 (s, 2H), 3.93 (s, 6H). MS (ESI+) m/z 456.9 (M+H).

### Example 111B

### N-[5-benzylsulfonyl-4-(trifluoromethyl)-1,3-thiazol-2-yl]-4,6-dimethoxy-pyrimidine-5-carboxamide

m-CPBA (0.54 g, 2.392 mmol) was added to a solution of Example 111A (0.466 g, 1.021 mmol) in CH₂Cl₂ (10 mL), and then stirred overnight at ambient temperature. The mixture was injected directly on a Grace REVELERIS® 40 g column, and subjected to chromatography with 0-100% EtOAc in heptane (40 mL/min) to obtain the title compound (0.378 g, 76% yield) as a white foam. ¹H NMR (400 MHz, DMSO-d₆) δ 13.58 (s, 1H), 8.61 (s, 1H), 7.40-7.32 (m, 3H), 7.32-7.24 (m, 2H), 4.86 (s, 2H), 3.93 (s, 6H). MS (ESI+) m/z 488.9 (M+H).

### Example 112

### N-(5-(benzylsulfinyl)-4-(trifluoromethyl)thiazol-2-yl)-4,6-dimethoxypyrimidine-5-carboxamide

To a suspension of Example 111A (0.457 g, 1.001 mmol), 2,2,2-trifluoroacetamide (0.226 g, 2.002 mmol), magnesium oxide (0.161 g, 4.00 mmol), and rhodium(II) acetate dimer (0.022 g, 0.050 mmol) in CH₂Cl₂ (10 mL) was added iodobenzene diacetate (0.484 g, 1.502 mmol). The mixture was stirred at ambient temperature for 5 hours then filtered through a CELITE® plug. The filtrate was concentrated and purified by chromatography (0-100% ethyl acetate in heptane gradient) to provide the title compound (0.109 g, 0.231 mmol, 23% yield) as a solid. ¹H NMR (400 MHz, DMSO-d₆) δ 13.30 (s, 1H), 8.61 (s, 1H), 7.35-7.26 (m, 3H), 7.22-7.12 (m, 2H), 4.56 (d, J=12.6 Hz, 1H), 4.44 (d, J=12.6 Hz, 1H), 3.94 (s, 6H). MS (ESI+) m/z 472.9 (M+H).

### Example 113

### 4,6-dimethoxy-N-[5-methylsulfinyl-4-[5-(trifluoromethyl)-2-pyridyl]-1,3-thiazol-2-yl]pyrimidine-5-carboxamide

### Example 113A

### 4,6-dimethoxy-N-[5-methylthio-4-[5-(trifluoromethyl)-2-pyridyl]-1,3-thiazol-2-yl]pyrimidine-5-carboxamide

The title compound was prepared as described in Example 75, substituting S-methyl methanethiosulfonate for ethyl chloroformate (0.839 g, 124%). ¹H NMR (400 MHz, DMSO-d₆) δ 12.76 (s, 1H), 9.02-8.98 (m, 1H), 8.63 (s, 1H), 8.27 (dd, J=8.7, 2.4 Hz, 1H), 8.08 (d, J=8.5 Hz, 1H), 3.97 (s, 6H), 2.65 (s, 3H). MS (ESI⁻) m/z 456.0 (M−H).

### Example 113B

### 4,6-dimethoxy-N-[5-methylsulfinyl-4-[5-(trifluoromethyl)-2-pyridyl]-1,3-thiazol-2-yl]pyrimidine-5-carboxamide

m-CPBA (0.44 g, 1.959 mmol) was added to a solution of Example 113A (0.84 g, 1.834 mmol) in CH₂Cl₂ (10 mL), and then stirred at ambient temperature for 3 hr. The mixture was injected directly onto a Grace REVELERIS® 80 g column and eluted with 0-100% 3:1 EtOAc:EtOH in heptane (50 mL/min) to obtain the title compound (0.1512 g, 0.319 mmol, 21.5% yield) as a beige solid. ¹H NMR (400 MHz, DMSO-d₆) δ 13.02 (s, 1H), 9.07-9.01 (m, 1H), 8.61 (s, 1H), 8.34 (ddd, J=8.3, 2.3, 0.8 Hz, 1H), 8.12 (d, J=8.3 Hz, 1H), 3.94 (s, 6H), 3.06 (s, 3H). MS (ESI+) m/z 474.0 (M+H). Obtained Example 114 (0.0842 g, 0.172 mmol, 11.6% yield) as a beige solid. ¹H NMR (400 MHz, DMSO-d₆) δ 13.33 (s, 1H), 9.18-9.12 (m, 1H), 8.63 (s, 1H), 8.40 (dd, J=8.5, 2.2 Hz, 1H), 8.11 (d, J=8.3 Hz, 1H), 3.96 (s, 6H), 3.78 (s, 3H). MS (ESI+) m/z 489.9 (M+H).

### Example 114

### 4,6-dimethoxy-N-[5-methylsulfonyl-4-[5-(trifluoromethyl)-2-pyridyl]-1,3-thiazol-2-yl]pyrimidine-5-carboxamide

The title compound was obtained by purification of Example 113B (0.084 g, 11.6% yield). ¹H NMR (400 MHz, DMSO-d₆) δ 13.33 (s, 1H), 9.18-9.12 (m, 1H), 8.63 (s, 1H), 8.40 (dd, J=8.5, 2.2 Hz, 1H), 8.11 (d, J=8.3 Hz, 1H), 3.96 (s, 6H), 3.78 (s, 3H). MS (ESI+) m/z 489.9 (M+H).

### Example 115

### 4,6-dimethoxy-N-[5-phenylsulfanyl-4-(trifluoromethyl)-1,3-thiazol-2-yl]pyrimidine-5-carboxamide

A 2.5M hexane solution of n-butyllithium (0.30 ml, 0.750 mmol) was added to a solution of diisopropylamine (0.10 ml, 0.763 mmol) in THF (0.50 ml) at −78° C., stirred for 10 min, allowed to warm to 0° C. for 5 min, and cooled to −78° C. A solution of Example 3 (0.075 g, 0.225 mmol) in THF (0.30 ml) was added, stirred for 20 min, and added to a solution of PhSSPh (0.074 g, 0.339 mmol) in THF (0.30 ml) at −78° C. The mixture was stirred for 1 h, allowing to warm, diluted with EtOAc, washed with water, saturated aqueous NaHCO₃, and brine, then dried (Na₂SO₄), and subjected to chromatography on silica gel (5% EtOAc/CH₂Cl₂) to give the title compound (0.064 g, 64%): ¹H NMR (400 MHz, DMSO-d₆) δ 13.24 (s, 1H), 8.62 (s, 1H), 7.43 (d, J=4.1 Hz, 4H), 7.40-7.32 (m, 1H), 3.95 (s, 6H); MS (ESI) m/z 443 (M+H).

### Example 116

### N-[5-(2-hydroxypropan-2-yl)-4-(trifluoromethyl)-1,3-thiazol-2-yl]-4,6-dimethoxypyrimidine-5-carboxamide

The title compound was prepared as described in Example 110, substituting acetone for p-toluenesulfonyl fluoride (0.375 g, 63.9% yield). ¹H NMR (400 MHz, DMSO-d₆) δ 12.67 (s, 1H), 8.61 (s, 1H), 6.20 (s, 1H), 3.95 (s, 6H), 1.58 (s, 6H). MS (ESI+) m/z 393.0 (M+H).

### Example 117

### N-[5-(4-hydroxytetrahydro-2H-pyran-4-yl)-4-(trifluoromethyl)-1,3-thiazol-2-yl]-4,6-dimethoxypyrimidine-5-carboxamide

The title compound was prepared as described in Example 110, substituting tetrahydro-4H-pyran-4-one for p-toluenesulfonyl fluoride (0.519 g, 80% yield). ¹H NMR (400 MHz, DMSO-d₆) δ 12.74 (s, 1H), 8.61 (s, 1H), 6.24 (s, 1H), 3.95 (s, 6H), 3.78-3.66 (m, 4H), 2.16-2.04 (m, 2H), 1.76 (d, J=13.1 Hz, 2H). MS (ESI+) m/z 435.0 (M+H).

### Example 118

### N-[5-(4-fluorotetrahydro-2H-pyran-4-yl)-4-(trifluoromethyl)-1,3-thiazol-2-yl]-4,6-dimethoxypyrimidine-5-carboxamide

To a suspension of Example 117 (0.36 g, 0.836 mmol) in CH₂Cl₂ (15 mL) was added Deoxofluor (0.25 mL, 1.356 mmol) at −75° C. The mixture was stirred at −75° C. for 1 hr and at 0° C. for 1 hr. The reaction was quenched with 100 mL saturated NaHCO₃, extracted with 100 mL EtOAc, washed with brine, dried over Na₂SO₄, concentrated to a yellow oil, which was subjected to chromatography on a Grace REVELERIS® 40 g column with 0-50% 3:1 EtOAc:EtOH in heptane (40 mL/min) to obtain the title compound (0.32 g, 87% yield) as a white solid. ¹H NMR (400 MHz, DMSO-d₆) δ 13.07 (s, 1H), 8.62 (s, 1H), 3.95 (s, 6H), 3.92-3.83 (m, 2H), 3.72-3.62 (m, 2H), 2.36-2.08 (m, 4H). MS (ESI+) m/z 437.0 (M+H).

### Example 119

### N-[5-(1-hydroxycyclobutyl)-4-(trifluoromethyl)-1,3-thiazol-2-yl]-4,6-dimethoxypyrimidine-5-carboxamide

The title compound was prepared as described in Example 110, substituting cyclobutanone for p-toluenesulfonyl fluoride (0.2669 g, 44.1% yield). ¹H NMR (400 MHz, DMSO-d₆) δ 12.80 (s, 1H), 8.61 (s, 1H), 6.04 (s, 1H), 3.95 (s, 6H), 2.56-2.45 (m, 2H), 2.45-2.34 (m, 2H), 2.03-1.89 (m, 1H), 1.78-1.65 (m, 1H). MS (ESI+) m/z 405.0 (M+H).

### Example 120

### N-{5-[hydroxy(phenyl)methyl]-4-(trifluoromethyl)-1,3-thiazol-2-yl}-4,6-dimethoxypyrimidine-5-carboxamide

The title compound was prepared as described in Example 110, substituting benzaldehyde for p-toluenesulfonyl fluoride (0.591 g, 90% yield). ¹H NMR (400 MHz, DMSO-d₆) δ 12.85 (s, 1H), 8.61 (s, 1H), 7.42-7.35 (m, 4H), 7.35-7.27 (m, 1H), 6.75 (d, J=4.0 Hz, 1H), 6.17 (dd, J=3.9, 1.6 Hz, 1H), 3.94 (s, 6H). MS (ESI+) m/z 441.0 (M+H).

### Example 121

### N-[5-benzoyl-4-(trifluoromethyl)-1,3-thiazol-2-yl]-4,6-dimethoxypyrimidine-5-carboxamide

The title compound was prepared as described in Example 110, substituting benzoyl chloride for p-toluenesulfonyl fluoride (0.494 g, 75% yield). ¹H NMR (400 MHz, DMSO-d₆) δ 13.41 (s, 1H), 8.63 (s, 1H), 7.95-7.87 (m, 2H), 7.78-7.71 (m, 1H), 7.59 (t, J=7.8 Hz, 2H), 3.96 (s, 6H). MS (ESI+) m/z 439.0 (M+H).

### Example 122

### N-[5-(1-hydroxy-1-phenylethyl)-4-(trifluoromethyl)-1,3-thiazol-2-yl]-4,6-dimethoxypyrimidine-5-carboxamide

A 3.0M solution of methyl magnesium chloride (0.80 mL, 2.400 mmol) in THF was added dropwise to a solution of Example 121 (463 mg, 1.056 mmol) in THF (10 mL) chilled to −75° C. The mixture was allowed to slowly warm to 5° C. over 1.5 hr. An additional 3.0M methyl magnesium chloride (0.80 mL, 2.400 mmol) in THF was added and the resulting mixture was stirred for 4 hr at ambient temperature, and chilled to −75° C. A 1.6M solution of methyllithium (1.3 mL, 2.080 mmol) in diethyl ether was then added, and the reaction mixture was stirred overnight at ambient temperature. The reaction was quenched with 50 mL saturated NH₄Cl, extracted with 50 mL EtOAc, washed with brine, dried over Na₂SO₄, concentrated to an oil, which was subjected to chromatography on a Grace REVELERIS® 40 g column with 0-50% EtOAc in heptane (40 mL/min) to obtain the title compound (227 mg, 47.3% yield) as a white solid. ¹H NMR (400 MHz, DMSO-d₆) δ 12.75 (s, 1H), 8.60 (s, 1H), 7.41 (d, J=7.5 Hz, 2H), 7.37-7.29 (m, 2H), 7.29-7.23 (m, 1H), 6.58 (s, 1H), 3.95 (s, 6H), 1.95 (s, 3H). MS (ESI+) m/z 455.0 (M+H).

### Example 123

### N-[5-(1-hydroxy-2,2-dimethylpropyl)-4-(trifluoromethyl)-1,3-thiazol-2-yl]-4,6-dimethoxypyrimidine-5-carboxamide

The title compound was prepared as described in Example 110, substituting trimethylacetaldehyde for p-toluenesulfonyl fluoride (0.527 g, 84% yield). ¹H NMR (400 MHz, DMSO-d₆) δ 12.81 (s, 1H), 8.61 (s, 1H), 6.18 (d, J=4.1 Hz, 1H), 4.82 (d, J=4.2 Hz, 1H), 3.96 (s, 6H), 0.94 (s, 9H). MS (ESI+) m/z 421.0 (M+H).

### Example 124

### N-[5-(2,2-dimethylpropanoyl)-4-(trifluoromethyl)-1,3-thiazol-2-yl]-4,6-dimethoxypyrimidine-5-carboxamide

The title compound was prepared as described in Example 110, substituting pivaloyl chloride for p-toluenesulfonyl fluoride (0.568 g, 91% yield). ¹H NMR (400 MHz, DMSO-d₆) δ 13.34 (s, 1H), 8.64 (s, 1H), 3.97 (s, 6H), 1.25 (s, 9H). MS (ESI+) m/z 419.0 (M+H).

### Example 125

### 4,6-dimethoxy-N-{5-[(E)-(methoxyimino)(phenyl)methyl]-4-(trifluoromethyl)-1,3-thiazol-2-yl}pyrimidine-5-carboxamide

Example 121 (0.38 g, 0.876 mmol) and O-methylhydroxylamine hydrochloride (0.09 g, 1.05 mmol) were dissolved in pyridine (5 mL) and stirred overnight at ambient temperature. Additional O-methylhydroxylamine hydrochloride (0.088 g, 1.051 mmol) was added, and heated overnight at 60° C. Then 50 mL water was added, extracted with 50 mL EtOAc, washed with brine, dried over Na₂SO₄, concentrated under reduced pressure to an oil, which was subjected to chromatography on a Grace REVELERIS® 40 g column with 0-50% 3:1 EtOAc:EtOH in heptane (40 mL/min) to obtain the title compound (0.36 g, 0.772 mmol, 88% yield) as an off-white solid. ¹H NMR (400 MHz, DMSO-d₆) δ 13.30 (s, 1H), 8.64 (s, 1H), 7.57-7.51 (m, 2H), 7.51-7.41 (m, 3H), 4.00 (s, 3H), 3.97 (s, 6H). MS (ESI+) m/z 468.0 (M+H).

### Example 126

### N-{5-[(E)-(hydroxyimino)(phenyl)methyl]-4-(trifluoromethyl)-1,3-thiazol-2-yl}-4,6-dimethoxypyrimidine-5-carboxamide

The title compound was prepared as described in Example 125, substituting hydroxylamine hydrochloride for O-methylhydroxylamine hydrochloride (0.316 g, 85% yield). ¹H NMR (400 MHz, DMSO-d₆) δ 13.22 (s, 1H), 12.27 (s, 1H), 8.64 (s, 1H), 7.56-7.48 (m, 2H), 7.46-7.39 (m, 3H), 3.98 (s, 6H). MS (ESI+) m/z 454.0 (M+H).

### Example 127

### 4,6-dimethoxy-N-[5-(pyridin-2-ylcarbonyl)-4-(trifluoromethyl)-1,3-thiazol-2-yl]pyrimidine-5-carboxamide

### Example 127A

### N-{5-[hydroxy(pyridin-2-yl)methyl]-4-(trifluoromethyl)-1,3-thiazol-2-yl}-4,6-dimethoxypyrimidine-5-carboxamide

The title compound was prepared as described in Example 110, substituting 2-pyridinecarboxaldehyde for p-toluenesulfonyl fluoride (1.214 g, 92% yield). ¹H NMR (400 MHz, DMSO-d₆) δ 12.85 (s, 1H), 8.61 (s, 1H), 8.54-8.46 (m, 1H), 7.87 (td, J=7.8, 1.8 Hz, 1H), 7.62 (d, J=7.8 Hz, 1H), 7.33 (ddd, J=7.5, 4.8, 1.1 Hz, 1H), 6.88 (d, J=4.6 Hz, 1H), 6.21 (d, J=4.4 Hz, 1H), 3.94 (s, 6H). MS (ESI+) m/z 441.9 (M+H).

### Example 127B

### 4,6-dimethoxy-N-[5-(pyridin-2-ylcarbonyl)-4-(trifluoromethyl)-1,3-thiazol-2-yl]pyrimidine-5-carboxamide

The title compound was prepared as described in Example 87B, substituting Example 127A for Example 87A (0.543 g, 91% yield). ¹H NMR (400 MHz, DMSO-d₆) δ 13.34 (s, 1H), 8.89-8.83 (m, 1H), 8.65 (s, 1H), 8.22 (dt, J=7.8, 1.2 Hz, 1H), 8.16 (td, J=7.7, 1.7 Hz, 1H), 7.80 (ddd, J=7.5, 4.7, 1.3 Hz, 1H), 3.98 (s, 6H). MS (ESI+) m/z 439.9 (M+H).

### Example 128

### N-{5-[(E)-(hydroxyimino)(pyridin-2-yl)methyl]-4-(trifluoromethyl)-1,3-thiazol-2-yl}-4,6-dimethoxypyrimidine-5-carboxamide

The title compound was prepared as described in Example 125, substituting hydroxylamine hydrochloride for O-methylhydroxylamine hydrochloride, and substituting Example 127 for Example 121 (0.453 g, 87% yield). ¹H NMR (400 MHz, DMSO-d₆) δ 13.17 (s, 1H), 12.62 (s, 1H), 8.64 (s, 1H), 8.55-8.50 (m, 1H), 7.98 (dt, J=8.0, 1.2 Hz, 1H), 7.90 (td, J=7.8, 1.8 Hz, 1H), 7.43 (ddd, J=7.4, 4.8, 1.3 Hz, 1H), 3.98 (s, 6H). MS (ESI+) m/z 454.9 (M+H).

### Example 129

### N-{5-[1-hydroxy-1-(pyridin-2-yl)ethyl]-4-(trifluoromethyl)-1,3-thiazol-2-yl}-4,6-dimethoxypyrimidine-5-carboxamide

3.0M Methyl magnesium chloride (0.95 mL, 2.85 mmol) in THF was added dropwise to a solution of Example 127B (544 mg, 1.238 mmol) in THF (10 mL) chilled below 0° C. The mixture was stirred for 10 min, chilled to −75° C., and then 1.6M methyllithium (1.75 mL, 2.80 mmol) in diethyl ether was added. The resulting reaction mixture was stirred overnight at ambient temperature. The reaction was quenched with 100 mL saturated NH₄Cl, extracted with 100 mL EtOAc, washed with brine, dried over Na₂SO₄, concentrated under reduced pressure to an oil, which was subjected to chromatography on a Grace REVELERIS® 40 g column with 0-50% 3:1 EtOAc:EtOH in heptane (40 mL/min) to obtain the title compound (267 mg, 47.4% yield) as a yellow foam. ¹H NMR (400 MHz, DMSO-d₆) δ 12.76 (s, 1H), 8.61 (s, 1H), 8.49 (ddd, J=4.4, 1.9, 0.9 Hz, 1H), 7.81 (td, J=7.7, 1.8 Hz, 1H), 7.58 (dt, J=8.1, 1.1 Hz, 1H), 7.29 (ddd, J=7.5, 4.8, 1.1 Hz, 1H), 6.70 (s, 1H), 3.96 (s, 6H), 1.97 (s, 3H). MS (ESI⁻) m/z 454.1 (M−H).

### Example 130

### N-{5-[(5-fluoropyridin-2-yl)(hydroxy)methyl]-4-(trifluoromethyl)-1,3-thiazol-2-yl}-4,6-dimethoxypyrimidine-5-carboxamide

The title compound was prepared as described in Example 110, substituting 5-fluoro-2-formylpyridine for p-toluenesulfonyl fluoride (0.610 g, 89% yield). ¹H NMR (400 MHz, DMSO-d₆) δ 12.89 (s, 1H), 8.61 (s, 1H), 8.52 (d, J=2.9 Hz, 1H), 7.79 (td, J=8.7, 2.9 Hz, 1H), 7.70 (dd, J=8.7, 4.5 Hz, 1H), 6.99 (d, J=4.4 Hz, 1H), 6.23 (d, J=4.2 Hz, 1H), 3.94 (s, 6H). MS (ESI+) m/z 459.8 (M+H).

### Example 131

### N-{5-[(5-fluoropyridin-2-yl)carbonyl]-4-(trifluoromethyl)-1,3-thiazol-2-yl}-4,6-dimethoxypyrimidine-5-carboxamide

The title compound was prepared as described in Example 87B, substituting Example 130 for Example 87A (0.497 g, 89% yield). ¹H NMR (400 MHz, DMSO-d₆) δ 13.32 (s, 1H), 8.86 (d, J=2.8 Hz, 1H), 8.62 (s, 1H), 8.30 (dd, J=8.8, 4.6 Hz, 1H), 8.05 (td, J=8.7, 2.8 Hz, 1H), 3.96 (s, 6H). MS (ESI+) m/z 457.9 (M+H).

### Example 132

### N-{5-[(E)-(5-fluoropyridin-2-yl)(hydroxyimino)methyl]-4-(trifluoromethyl)-1,3-thiazol-2-yl}-4,6-dimethoxypyrimidine-5-carboxamide

The title compound was prepared as described in Example 125, substituting hydroxylamine hydrochloride for O-methylhydroxylamine hydrochloride, and substituting Example 131 for Example 121 (0.2485 g, 95% yield). ¹H NMR (400 MHz, DMSO-d₆) δ 13.14 (s, 1H), 12.63 (s, 1H), 8.62 (s, 1H), 8.52 (d, J=2.8 Hz, 1H), 8.03 (dd, J=8.9, 4.5 Hz, 1H), 7.82 (td, J=8.7, 2.9 Hz, 1H), 3.96 (s, 6H). MS (ESI+) m/z 473.0 (M+H).

### Example 133

### N-[5-acetyl-4-(trifluoromethyl)-1,3-thiazol-2-yl]-4,6-dimethoxypyrimidine-5-carboxamide

### Example 133A

### N-[5-(1-hydroxyethyl)-4-(trifluoromethyl)-1,3-thiazol-2-yl]-4,6-dimethoxypyrimidine-5-carboxamide

The title compound was prepared as described in Example 110, substituting acetaldehyde for p-toluenesulfonyl fluoride (0.55 g, 98% yield). ¹H NMR (501 MHz, DMSO-d₆) δ 12.77 (s, 1H), 8.59 (s, 1H), 6.11 (d, J=4.0 Hz, 1H), 5.24-5.15 (m, 1H), 3.93 (s, 6H), 1.41 (d, J=6.4 Hz, 3H). MS (ESI+) m/z 379.0 (M+H).

### Example 133B

### N-[5-acetyl-4-(trifluoromethyl)-1,3-thiazol-2-yl]-4,6-dimethoxypyrimidine-5-carboxamide

The title compound was prepared as described in Example 87B, substituting Example 133A for Example 87A (0.39 g, 79% yield). ¹H NMR (400 MHz, DMSO-d₆) δ 13.39 (s, 1H), 8.62 (s, 1H), 3.95 (s, 6H), 2.58 (s, 3H). MS (ESI+) m/z 376.9 (M+H).

### Example 134

### N-{5-[(1E)-N-hydroxyethanimidoyl]-4-(trifluoromethyl)-1,3-thiazol-2-yl}-4,6-dimethoxypyrimidine-5-carboxamide

The title compound was prepared as described in Example 125, substituting hydroxylamine hydrochloride for O-methylhydroxylamine hydrochloride, and substituting Example 133B for Example 121 (0.272 g, 79% yield). ¹H NMR (400 MHz, DMSO-d₆) δ 13.03 (s, 1H), 11.90 (s, 1H), 8.59 (s, 1H), 3.92 (s, 6H), 2.13 (s, 3H). MS (ESI+) m/z 391.9 (M+H).

### Example 135

### 4,6-dimethoxy-N-{5-[(1E)-N-methoxyethanimidoyl]-4-(trifluoromethyl)-1,3-thiazol-2-yl}pyrimidine-5-carboxamide

Example 133 (16.7 g, 44.2 mmol) and o-methylhydroxylamine hydrochloride (9.2 g, 111 mmol) were dissolved in pyridine (160 mL) by cooling with ice bath, and then the mixture was stirred overnight at ambient temperature. The mixture was dissolved in 500 mL EtOAc, washed with 1.5N HCl (200 mL), brine (200 mL), and dried over Na₂SO₄ and concentrated in vacuo. The residue was purified on SiO₂ eluting with CH₂C₁₂/ethyl acetate 0-20% over 80 min with 60 min hold to give a mixture of E and Z isomers. Separation of the isomers by supercritical fluid chromatography using a CHIRALPAK® OD-H (21×250 mm) column, eluting at 60 mL/min of CO₂ gave the title compound (6.8 g, 38.1% yield). ¹H NMR (400 MHz, DMSO-d₆) δ 13.16 (s, 1H), 8.63 (s, 1H), 3.97 (s, 6H), 3.81 (s, 3H), 2.20 (s, 3H). MS (ESI) m/z 406 (M+H)⁺.

### Example 136

### 4,6-dimethoxy-N-{5-[(1Z)—N-methoxyethanimidoyl]-4-(trifluoromethyl)-1,3-thiazol-2-yl}pyrimidine-5-carboxamide

Separation of isomers as described in Example 135 yielded the title compound (1.72 g, 9.6% yield). ¹H NMR (400 MHz, DMSO-d₆) δ 13.12 (s, 1H), 8.63 (s, 1H), 3.96 (d, J=7.1 Hz, 9H), 2.20 (s, J=1.3 Hz, 3H). MS (ESI) m/z 406 (M+H)⁺.

### Example 137

### 4,6-dimethoxy-N-[5-(2-methyl-1,3-dioxolan-2-yl)-4-(trifluoromethyl)-1,3-thiazol-2-yl]pyrimidine-5-carboxamide

To a suspension of Example 133B (0.35 g, 0.938 mmol) in benzene (80 mL) was added ethylene glycol (5.0 mL, 90 mmol) and 4-methylbenzenesulfonic acid hydrate (0.054 g, 0.284 mmol). The mixture was refluxed for 2 h with a Dean-Stark trap and cooled to ambient temperature. 100 mL EtOAc was added to the mixture, which was then washed twice with saturated NaHCO₃, once with brine, dried over Na₂SO₄, and concentrated under reduced pressure. The residue was subjected to chromatography on a Grace REVELERIS® 40 g column with 0-100% 3:1 EtOAc:EtOH in heptane (40 mL/min) to obtain the title compound (0.36 g, 0.76 mmol, 81% yield) as a white solid. ¹H NMR (400 MHz, DMSO-d₆) δ 12.91 (s, 1H), 8.60 (s, 1H), 4.08-4.02 (m, 2H), 3.93 (s, 6H), 3.90-3.85 (m, 2H), 1.72 (s, 3H). MS (ESI+) m/z 421.0 (M+H).

### Example 138

### N-[5-(1,3-dioxolan-2-yl)-4-(trifluoromethyl)-1,3-thiazol-2-yl]-4,6-dimethoxypyrimidine-5-carboxamide

The title compound was prepared as described in Example 137, substituting Example 47 for Example 133B (0.438 g, 87% yield). ¹H NMR (400 MHz, DMSO-d₆) δ 13.00 (s, 1H), 8.59 (s, 1H), 6.25 (q, J=1.6 Hz, 1H), 4.12-4.01 (m, 2H), 4.01-3.94 (m, 2H), 3.92 (s, 6H). MS (ESI+) m/z 407.0 (M+H).

### Example 139

### N-[5-(1,3-dioxan-2-yl)-4-(trifluoromethyl)-1,3-thiazol-2-yl]-4,6-dimethoxypyrimidine-5-carboxamide

The title compound was prepared as described in Example 137, substituting Example 47 for Example 133B, and substituting 1,3-propanediol for ethylene glycol (0.424 g, 79% yield). ¹H NMR (500 MHz, DMSO-d₆) δ 12.99 (s, 1H), 8.62 (s, 1H), 5.99 (d, J=1.5 Hz, 1H), 4.18-4.10 (m, 2H), 4.03-3.95 (m, 2H), 3.95 (s, 6H), 2.05-1.94 (m, 1H), 1.49-1.41 (m, 1H). MS (ESI⁻) m/z 419.1 (M−H).

### Example 140

### 4,6-dimethoxy-N-[5-(1,3-oxazol-5-yl)-4-(trifluoromethyl)-1,3-thiazol-2-yl]pyrimidine-5-carboxamide

A 20 mL vial charged with Example 47 (0.30 g, 0.828 mmol), tosylmethyl isocyanide (0.16 g, 0.828 mmol), potassium carbonate (0.229 g, 1.656 mmol) and MeOH (5 mL) was heated at 60° C. for 3 hr. After cooling to ambient temperature, EtOAc (100 mL) was added, washed with water and brine, dried over Na₂SO₄, and concentrated under reduced pressure. The resulting solid was subjected to chromatography on a Grace REVELERIS® 40 g column with 0-100% 3:1 EtOAc:EtOH in heptane (40 mL/min) to obtain the title compound (0.21 g, 62.2% yield) as an off-white solid. ¹H NMR (400 MHz, DMSO-d₆) δ 13.26 (s, 1H), 8.60 (s, 1H), 8.59 (s, 1H), 7.58 (s, 1H), 3.93 (s, 6H). MS (ESI+) m/z 402.0 (M+H).

### Example 141

### 4,6-dimethoxy-N-[5-(4-methyl-1,3-oxazol-5-yl)-4-(trifluoromethyl)-1,3-thiazol-2-yl]pyrimidine-5-carboxamide

The title compound was prepared as described in Example 140, substituting 1-methyl-1-tosylmethyl isocyanide for tosylmethyl isocyanide (0.1324 g, 38.5% yield). ¹H NMR (400 MHz, DMSO-d₆) δ 13.28 (s, 1H), 8.62 (s, 1H), 8.50 (s, 1H), 3.95 (s, 6H), 2.20 (s, 3H). MS (ESI+) m/z 416.1 (M+H).

### Example 142

### N-[5-(1-ethyl-1H-pyrazol-5-yl)-4-(trifluoromethyl)-1,3-thiazol-2-yl]-4,6-dimethoxypyrimidine-5-carboxamide

To a 5 mL microwave vial charged with Example 2C (0.250 g, 0.543 mmol), Pd(Ph₃P)₄ (0.063 g, 0.054 mmol), and cesium carbonate (0.354 g, 1.087 mmol) under nitrogen was added 1-ethyl-5-(4,4,5,5-tetramethyl-1,3,2-dioxaborolan-2-yl)-1H-pyrazole (0.157 g, 0.706 mmol) in dioxane (4.5 mL), and the mixture heated at 110° C. for 30 min. Water (100 mL) was added to the mixture, which was then extracted twice with 100 mL EtOAc, washed with brine, dried over Na₂SO₄, concentrated under reduced pressure and subjected to chromatography on a Grace REVELERIS® 40 g column, eluted with 0-50% EtOAc in CH₂Cl₂ (40 mL/min) to obtain the title compound (0.1 g, 21.3% yield) as a white foam. ¹H NMR (400 MHz, DMSO-d₆) δ 13.27 (s, 1H), 8.64 (s, 1H), 7.61 (d, J=1.9 Hz, 1H), 6.52 (d, J=1.9 Hz, 1H), 4.05 (q, J=7.2 Hz, 2H), 3.97 (s, 6H), 1.30 (t, J=7.2 Hz, 3H). MS (ESI+) m/z 429.1 (M+H).

### Example 143

### N-[5-(4,5-dihydrofuran-2-yl)-4-(trifluoromethyl)-1,3-thiazol-2-yl]-4,6-dimethoxypyrimidine-5-carboxamide

A mixture of Example 2C (50 mg, 0.109 mmol), 2-(4,5-dihydrofuran-2-yl)-4,4,5,5-tetramethyl-1,3,2-dioxaborolane (25.6 mg, 0.13 mmol), PdCl₂(dppf)-CH₂Cl₂ adduct (4.4 mg, 5.4 mol) and cesium carbonate (89 mg, 0.272 mmol) in dioxane was purged with nitrogen for 5 min. The purged mixture was heated to 110° C. for 2 hrs and then allowed to attain ambient temperature, filtered through a layer of CELITE®, and washed with ethyl acetate. The filtrate was washed with water, washed with brine, dried over MgSO₄, and concentrated under reduced pressure. The resulting residue was subjected to chromatography on a silica gel column eluting with 0-100% ethyl acetate in heptanes to provide the title product (24 mg, 55%). ¹H NMR (400 MHz, CDCl₃) δ 9.99 (s, 1H), 8.51 (s, 1H), 5.46 (s, 1H), 4.49 (t, J=9.5 Hz, 2H), 4.13 (s, 6H), 2.87 (td, J=9.6, 3.1 Hz, 2H). MS (DCI/NH₃) m/z 403 (M+H)⁺.

### Example 144

### 4,6-dimethoxy-N-[5-(tetrahydrofuran-2-yl)-4-(trifluoromethyl)-1,3-thiazol-2-yl]pyrimidine-5-carboxamide

A 100 mL three neck round bottom flask was charged with Example 143 (34 mg, 0.085 mmol), dihydroxypalladium (6 mg, 8.45 mmol) and methanol (10 mL). The mixture was hydrogenated at ambient temperature for 3 hrs. The flask was purged with nitrogen and the content was filtered through a layer of CELITE® and washed with ethyl acetate. The filtrate was concentrated under reduced pressure and the resulting residue was subjected to chromatography on silica gel eluting with 0-100% ethyl acetate in heptanes to provide the title product (10 mg, 30%). ¹H NMR (400 MHz, CDCl₃) δ 9.91 (s, 1H), 8.51 (s, 1H), 5.39 (s, 1H), 4.12 (s, 6H), 4.11 (m, 1H), 3.89 (dt, J=8.3, 6.9 Hz, 1H), 2.44 (dq, J=13.6, 7.2 Hz, 1H), 2.06 (ttd, J=18.8, 13.0, 6.9 Hz, 2H), 1.88 (dq, J=14.3, 6.8 Hz, 1H). MS (DCI/NH₃) m/z 405 (M+H)⁺.

### Example 145

The title compound was prepared as described in Example 143, substituting 2-(4,5-dihydrofuran-3-yl)-4,4,5,5-tetramethyl-1,3,2-dioxaborolane for 2-(4,5-dihydrofuran-2-yl)-4,4,5,5-tetramethyl-1,3,2-dioxaborolane (49 mg, 56%). ¹H NMR (400 MHz, Chloroform-d) δ 10.01 (s, 1H), 8.51 (s, 1H), 6.81 (s, 1H), 4.54 (t, J=9.6 Hz, 2H), 4.12 (s, 6H), 3.01 (t, J=9.6, 2H). MS (DCI/NH₃) m/z 403 (M+H)⁺.

### Example 146

### 4,6-dimethoxy-N-[5-(tetrahydrofuran-3-yl)-4-(trifluoromethyl)-1,3-thiazol-2-yl]pyrimidine-5-carboxamide

The title compound was prepared as described in Example 144, substituting Example 145 for Example 143 (23 mg, 47%). ¹H NMR (400 MHz, CDCl₃) δ 10.12 (s, 1H), 8.51 (s, 1H), 4.16-4.04 (m, 8H), 4.04-3.78 (m, 2H), 3.78-3.66 (m, 1H), 2.47 (dtd, J=13.3, 8.1, 5.3 Hz, 1H), 2.04 (ddd, J=12.5, 8.2, 6.4 Hz, 1H). MS (DCI/NH₃) m/z 405 (M+H)⁺.

### Example 147

### N-[5-(3,4-dihydro-2H-pyran-5-yl)-4-(trifluoromethyl)-1,3-thiazol-2-yl]-4,6-dimethoxypyrimidine-5-carboxamide

The title compound was prepared as described in Example 143, substituting 2-(3,4-dihydro-2H-pyran-5-yl)-4,4,5,5-tetramethyl-1,3,2-dioxaborolane for 2-(4,5-dihydrofuran-2-yl)-4,4,5,5-tetramethyl-1,3,2-dioxaborolane (8 mg, 11%). MS (DCI/NH₃) m/z 417 (M+H)⁺.

### Example 148

### 4,6-dimethoxy-N-[5-(tetrahydro-2H-pyran-3-yl)-4-(trifluoromethyl)-1,3-thiazol-2-yl]pyrimidine-5-carboxamide

The title compound was prepared as described in Example 144, substituting Example 147 for Example 143 (7 mg, 58%). ¹H NMR (400 MHz, CDCl₃) δ 10.04 (s, 1H), 8.51 (s, 1H), 4.11 (s, 6H), 4.01-3.93 (m, 2H), 3.58-3.35 (m, 2H), 1.77-1.68 (m, 2H), 2.11-1.65 (m, 1H), 0.87 (qd, J =10.0, 8.2, 3.5 Hz, 2H). MS (DCI/NH₃) m/z 419 (M+H)⁺.

### Example 149

### 4,6-dimethoxy-N-{5-[(3S*)-tetrahydro-2H-pyran-3-yl]-4-(trifluoromethyl)-1,3-thiazol-2-yl}pyrimidine-5-carboxamide

Example 149 was obtained by separating Example 148 via supercritical fluid chromatography using an AD-H prep chiral column. The earlier eluting component was the title compound (40 mg, 30%), and the later eluting component was Example 150. ¹H NMR (400 MHz, CDCl₃) δ 10.04 (s, 1H), 8.51 (s, 1H), 4.11 (s, 6H), 4.01-3.93 (m, 2H), 3.58-3.35 (m, 2H), 1.77-1.68 (m, 2H), 2.11-1.65 (m, 1H), 0.87 (qd, J=10.0, 8.2, 3.5 Hz, 2H). MS (DCI/NH₃) m/z 419 (M+H)⁺.

### Example 150

### 4,6-dimethoxy-N-{5-[(3R)-tetrahydro-2H-pyran-3-yl]-4-(trifluoromethyl)-1,3-thiazol-2-yl}pyrimidine-5-carboxamide

Example 150 was obtained by separating Example 148 via supercritical fluid chromatography using an AD-H prep chiral column. The earlier eluting component was Example 149, and the later eluting component was the title compound (38 mg, 28%). ¹H NMR (400 MHz, CDCl₃) δ 10.04 (s, 1H), 8.51 (s, 1H), 4.11 (s, 6H), 4.01-3.93 (m, 2H), 3.58-3.35 (m, 2H), 1.77-1.68 (m, 2H), 2.11-1.65 (m, 1H), 0.87 (qd, J=10.0, 8.2, 3.5 Hz, 2H). MS (DCI/NH₃) m/z 419 (M+H)⁺.

### Example 151

### N-{5-[2-(1-cyanocyclopropyl)pyridin-4-yl]-4-(trifluoromethyl)-1,3-thiazol-2-yl}-4,6-dimethoxypyrimidine-5-carboxamide

### Example 151A

### 4-bromo-2-(bromomethyl)pyridine

A solution of (4-bromopyridin-2-yl) methanol (3.0 g, 15.96 mmol) in THF (60 ml) was cooled to 0° C. and treated with methylsulfonyl chloride (2.487 ml, 31.9 mmol), followed by triethylamine (6.67 ml, 47.9 mmol). The mixture was stirred at ambient temperature for 30 min, diluted with dichloromethane and the content was then washed with water. The organic phase was dried and concentrated under reduced pressure to provide the crude mesylate intermediate. The crude mesylate intermediate was dissolved in acetone (30 ml) and lithium bromide (6.24 g, 71.8 mmol) was added to the resulting solution. After stirring at ambient temperature for 3 hrs, the mixture was filtered through a layer of CELITE® and evaporated under reduced pressure. The residue was diluted with dichloromethane, washed with H₂O, dried and evaporated under reduced pressure, and the resulting residue was subjected to chromatography on a silica gel column eluting with 0-20% ethyl acetate in heptanes to provide the title compound (3.65 g, 91%). MS (DCI/NH₃) m/z 251, 253 (M+H)⁺.

### Example 151B

### 4-bromo-2-(bromomethyl)pyridine

To a solution of Example 151A (2 g, 7.97 mmol) in ethanol (90 ml) and water (10 ml) was added KCN (0.779 g, 11.96 mmol) and the mixture stirred for 16 hrs. The mixture was concentrated under reduced pressure and the resulting residue was subjected to chromatography on a silica gel column to provide the title compound (1.3 g, 83%). MS (DCI/NH₃) m/z 199 (M+H)⁺.

### Example 151C

### 1-(4-bromopyridin-2-yl)cyclopropanecarbonitrile

To Example 151B (300 mg, 1.523 mmol) was added 1,2-dibromoethane (0.197 ml, 2.284 mmol) and 50% aq. solution of sodium hydroxide (10 ml, 1.523 mmol) followed by N-benzyl-N,N-diethylethanaminium chloride (347 mg, 1.523 mmol). The mixture was heated to 80° C. for 1 h and then cooled to ambient temperature. The majority of the NaOH solution was pipetted out, and the remaining mixture was diluted with ether and H₂O. The aqueous phase was extracted with additional ether, and the organic phases were combined, washed with brine, dried, concentrated under reduced pressure and the resulting residue was subjected to chromatography on a silica gel column eluting with 0-20% ethyl acetate in heptanes to provide the title compound (185 mg, 55%). MS (DCI/NH₃) m/z 223, 225 (M+H)⁺.

### Example 151D

### 1-(4-(4,4,5,5-tetramethyl-1,3,2-dioxaborolan-2-yl)pyridin-2-yl)cyclopropanecarbonitrile

A solution of Example 151C (180 mg, 0.807 mmol), bis(pinacolato)diboron (246 mg, 0.968 mmol), PdCl₂(dppf)-CH₂Cl₂ (33 mg, 0.04 mmol) and potassium acetate (198 mg, 2.017 mmol) was mixed in anhydrous toluene (10 mL) and purged with a stream of nitrogen for 5 minutes. The purged mixture was heated to 90° C. for 3 hrs, cooled to room temperature and filtered through a layer of CELITE®. The filtrate was concentrated under reduced pressure to provide the title compound (280 mg) which was used directly without further purification. MS (DCI/NH₃) m/z 271 (M+H)⁺.

### Example 151E

### 4,6-Dimethoxy-pyrimidine-5-carboxylic acid {5-[2-(1-cyano-cyclopropyl)-pyridin-4-yl]-4-trifluoromethyl-thiazol-2-yl}-amide

The title compound was prepared as described in Example 143, substituting Example 151D for 2-(4,5-dihydrofuran-2-yl)-4,4,5,5-tetramethyl-1,3,2-dioxaborolane (98 mg, 53%). ¹H NMR (400 MHz, CDCl₃) δ 10.27 (s, 1H), 8.59-8.48 (m, 2H), 7.77 (d, J=1.4 Hz, 1H), 7.26 (d, J=5.2 Hz, 1H), 4.16 (s, 6H), 2.02-1.72 (m, 4H). MS (DCI/NH₃) m/z 477 (M+H)⁺.

### Example 152

### N-{5-[2-(cyanomethyl)pyridin-4-yl]-4-(trifluoromethyl)-1,3-thiazol-2-yl}-4,6-dimethoxypyrimidine-5-carboxamide

### Example 152A

### 2-(4-(4,4,5,5-tetramethyl-1,3,2-dioxaborolan-2-yl)pyridin-2-yl)acetonitrile

The title compound was prepared as described in Example 151D, substituting Example 151B for Example 151C (250 mg, 100%). MS (DCI/NH₃) m/z 245 (M+H)⁺.

### Example 152B

### 4,6-dimethoxy-N-[5-(2-methyl-1,3-dioxolan-2-yl)-4-(trifluoromethyl)-1,3-thiazol-2-yl]pyrimidine-5-carboxamide

The title compound was prepared as described in Example 143, substituting Example 152A for 2-(4,5-dihydrofuran-2-yl)-4,4,5,5-tetramethyl-1,3,2-dioxaborolane (160 mg, 64%). ¹H NMR (400 MHz, CDCl₃) δ 10.30 (s, 1H), 8.66 (dd, J=5.2, 0.8 Hz, 1H), 8.54 (s, 1H), 7.51 (d, J=1.6 Hz, 1H), 7.37 (dd, J=5.2, 1.7 Hz, 1H), 4.16 (s, 6H), 4.02 (s, 2H). MS (DCI/NH₃) m/z 451 (M+H)⁺.

### Example 153

### N-[5-(6-fluoropyridin-2-yl)-4-(trifluoromethyl)-1,3-thiazol-2-yl]-4,6-dimethoxypyrimidine-5-carboxamide

A solution of Example 2C (100 mg, 0.217 mmol), 2-fluoro-6-(4,4,5,5-tetramethyl-1,3,2-dioxaborolan-2-yl)pyridine (72.7 mg, 0.326 mmol), PdCl₂(dppf)-CH₂Cl₂ (8.87 mg, 10.87 μmol), copper (I) chloride (21.51 mg, 0.217 mmol) and cesium carbonate (142 mg, 0.435 mmol) in dioxane (4 ml) was purged with N₂ for 5 minutes and heated to 90° C. for 16 hrs. The mixture was cooled to ambient temperature and filtered through a layer of CELITE®. The filtrate was diluted with ethyl acetate, washed with water and brine, dried, concentrated under reduced pressure and the resulting residue was subjected to chromatography on a silica gel column eluting with 0-40% ethyl acetate in heptane to provide the title compound (51 mg, 55%). ¹H NMR (501 MHz, CDCl₃) δ 10.11 (s, 1H), 8.52 (s, 1H), 7.86 (q, J=7.9 Hz, 1H), 7.55 (dd, J=7.5, 2.2 Hz, 1H), 6.94 (dd, J=8.2, 2.8 Hz, 1H), 4.15 (s, 6H). MS (DCI/NH₃) m/z 430 (M+H)⁺.

### Example 154

### N-{5-[6-(3-cyanooxetan-3-yl)pyridin-2-yl]-4-(trifluoromethyl)-1,3-thiazol-2-yl}-4,6-dimethoxypyrimidine-5-carboxamide

To a solution of oxetane-3-carbonitrile (19.35 mg, 0.211 mmol) in anhydrous THF (10 mL) cooled to −78° C. was added potassium hexamethyldisilazide (1M in THF, 0.349 μL) and the mixture stirred for 30 min in a dry ice bath. A solution of Example 153 (50 mg, 0.116 mmol) in THF (2 mL) was added to the reaction mixture. The dry ice bath was removed and the mixture was stirred at ambient temperature for 2 hrs, diluted with ether and quenched with a saturated solution of NH₄Cl. The organic phase was separated and the aqueous phase was washed with additional ether. The combined organics were washed with brine, dried, filtered, concentrated under reduced pressure and the resulting residue was subjected to chromatography on a silica gel column eluting with 0-40% ethyl acetate-heptane to provide the title compound (8 mg, 14%). ¹H NMR (400 MHz, CDCl₃) δ 10.15 (s, 1H), 8.54 (s, 1H), 7.88 (t, J=7.9 Hz, 1H), 7.71 (d, J=7.5 Hz, 1H), 7.59 (d, J=7.8 Hz, 1H), 5.27 (d, J=5.8 Hz, 1H), 5.17 (d, J=5.8 Hz, 2H), 4.17 (s, 6H). MS (DCI/NH₃) m/z 493 (M+H)⁺.

### Example 155

### N-{5-[6-(cyanomethyl)pyridin-2-yl]-4-(trifluoromethyl)-1,3-thiazol-2-yl}-4,6-dimethoxypyrimidine-5-carboxamide

The title compound was prepared as described in Example 154, substituting acetonitrile for oxetane-3-carbonitrile (10 mg, 19%). ¹H NMR (400 MHz, CDCl₃) δ 10.13 (s, 1H), 8.53 (s, 1H), 7.83 (t, J=7.8 Hz, 1H), 7.66 (d, J=8.0 Hz, 1H), 7.47 (d, J=7.7 Hz, 1H), 4.15 (s, 6H), 3.97 (s, 2H). MS (DCI/NH₃) m/z 451 (M+H)⁺.

### Example 156

### 4,6-dimethoxy-N-{5-[5-(propan-2-yloxy)pyridin-3-yl]-4-(trifluoromethyl)-1,3-thiazol-2-yl}pyrimidine-5-carboxamide

A solution of Example 2C (100 mg, 0.217 mmol), (5-isopropoxypyridin-3-yl)boronic acid (43.3 mg, 0.239 mmol), Pd₂dba₃ (9.95 mg, 10.87 μmol), (1S,3R,5R,7S)-1,3,5,7-tetramethyl-8-phenyl-2,4,6-trioxa-8-phosphaadamantane (6.35 mg, 0.022 mmol) and potassium carbonate (75 mg, 0.543 mmol) in toluene (8 ml) and 2-Propanol (2.000 ml) was purged with N₂ for 5 minutes and heated to 90° C. for 16 hrs. The mixture was then filtered through a layer of CELITE® and the filtrate was diluted with ethyl acetate, washed with water and brine, dried, concentrated under reduced pressure and the resulting residue was subjected to chromatography on a silica gel column eluting with 0-40% ethyl acetate—heptane to provide the title compound (10 mg, 10%). ¹H NMR (400 MHz, Chloroform-d) δ 10.13 (s, 1H), 8.53 (s, 1H), 8.30 (dd, J=21.9, 2.3 Hz, 2H), 4.61 (p, J=6.1 Hz, 1H), 4.16 (s, 6H), 1.39 (d, J=6.0 Hz, 6H). MS (DCI/NH₃) m/z 470 (M+H)⁺.

### Example 157

### N-{5-[5-(1-cyanocyclopropyl)pyridin-2-yl]-4-(trifluoromethyl)-1,3-thiazol-2-yl}-4,6-dimethoxypyrimidine-5-carboxamide

### Example 157A

### 2-(6-bromopyridin-3-yl)acetonitrile

A solution of (6-bromopyridin-3-yl)methanol (2 g, 10.64 mmol) in CH₂Cl₂ (30 ml) at 0° C. was treated with MsCl (1.658 ml, 21.27 mmol) followed by the addition of triethylamine (4.45 ml, 31.9 mmol). The mixture was stirred at ambient temperature for 30 min, diluted with dichloromethane and washed with water. The organic phase was dried and concentrated to provide the crude mesylate intermediate. The crude mesylate intermediate was dissolved in acetonitrile (30 mL) and H₂O (6 mL) and treated with KCN (1.385 g, 21.285 mmol). The mixture was heated to 50° C. for 16 hrs, then diluted using ethyl acetate (50 mL). The aqueous layer was extracted with additional ethyl acetate. The combined organics were dried over MgSO4, filtered, concentrated under reduced pressure and the residue was subjected to chromatography on a silica gel column eluting with 0-20% ethyl acetate—heptane to provide the title compound (2.03 g, 97%). MS (DCI/NH₃) m/z 197, 199 (M+H)⁺.

### Example 157B

### 1-(6-bromopyridin-3-yl)cyclopropanecarbonitrile

The title compound was prepared as described in Example 151C, substituting Example 157A for Example 151B (430 mg, 82%). MS (DCI/NH₃) m/z 223, 225 (M+H)⁺.

### Example 157C

### 1-(6-(4,4,5,5-tetramethyl-1,3,2-dioxaborolan-2-yl)pyridin-3-yl)cyclopropanecarbonitrile

The title compound was prepared as described in Example 151D, substituting Example 157B for Example 151C. The crude material (500 mg) was used directly without purification. MS (DCI/NH₃) m/z 271 (M+H)⁺.

### Example 157D

### N-{5-[5-(1-cyanocyclopropyl)pyridin-2-yl]-4-(trifluoromethyl)-1,3-thiazol-2-yl}-4,6-dimethoxypyrimidine-5-carboxamide

The title compound was prepared as described in Example 153, substituting Example 157C for 2-fluoro-6-(4,4,5,5-tetramethyl-1,3,2-dioxaborolan-2-yl)pyridine (5 mg, 4%). ¹H NMR (400 MHz, CDCl₃) δ 10.06 (s, 1H), 8.61-8.48 (m, 2H), 7.77-7.61 (m, 2H), 4.15 (s, 6H), 1.94-1.77 (m, 2H), 1.33-1.20 (m, 2H). MS (DCI/NH₃) m/z 477 (M+H)⁺.

### Example 158

### N-{5-[5-(2-cyanopropan-2-yl)pyridin-2-yl]-4-(trifluoromethyl)-1,3-thiazol-2-yl}-4,6-dimethoxypyrimidine-5-carboxamide

### Example 158A

### 2-(6-bromopyridin-3-yl)-2-methylpropanenitrile

A solution of Example 157 A (430 mg, 2.18 mmol) in DMF (10 ml) was cooled to 0° C. and was then treated with NaH (262 mg, 6.55 mmol). The mixture was stirred for 30 min, followed by the addition of iodomethane (0.682 ml, 10.91 mmol). The mixture was stirred at ambient temperature for 1 h, diluted with ether and quenched with H₂O. The aqueous layer was extracted with additional ether. The combined organics were dried over MgSO₄, filtered, concentrated under reduced pressure and the resulting residue was subjected to chromatography on a silica gel column eluting with 0-20% ethyl acetate-heptane to provide the title compound (455 mg, 93%). MS (DCI/NH₃) m/z 225, 227 (M+H)⁺.

### Example 158B

### 2-methyl-2-(6-(4,4,5,5-tetramethyl-1,3,2-dioxaborolan-2-yl)pyridin-3-yl)propanenitrile

The title compound was prepared as described in Example 151D, substituting Example 158A for Example 151C. The crude material (355 mg) was used directly without purification. MS (DCI/NH₃) m/z 273 (M+H)⁺.

### Example 158C

### 4,6-Dimethoxy-pyrimidine-5-carboxylic acid {5-[5-(cyano-dimethyl-methyl)-pyridin-2-yl]-4-trifluoromethyl-thiazol-2-yl}-amide

The title compound was prepared as described in Example 153, substituting Example 158B for 2-fluoro-6-(4,4,5,5-tetramethyl-1,3,2-dioxaborolan-2-yl)pyridine (10 mg, 6.4%). ¹H NMR (400 MHz, CDCl₃) δ 10.11 (s, 0H), 8.54 (s, 1H), 7.82 (t, J=7.9 Hz, 1H), 7.64 (dd, J=7.9, 3.1 Hz, 2H), 4.16 (s, 6H), 1.79 (s, 3H), 1.55 (s, 3H). MS (DCI/NH₃) m/z 479 (M+H)⁺.

### Example 159

### N-[5-(2-cyanophenyl)-4-(trifluoromethyl)-1,3-thiazol-2-yl]-4-methoxy-6-(methylsulfanyl)pyrimidine-5-carboxamide

### Example 159A

### 2-(2-amino-4-(trifluoromethyl)thiazol-5-yl)benzonitrile

A solution of Example 2B (5.0 g, 17.00 mmol) and (2-cyanophenyl)boronic acid (3.7 g, 25.5 mmol) in THF (50 mL) was degassed with dry nitrogen for 5 min. 1,1′-Bis(di-tert-butylphosphino)ferrocene palladium dichloride (1108 mg, 1.700 mmol) and 2 M aqueous cesium carbonate (17.00 mL, 34.0 mmol) were added and the reaction mixture was degassed with dry nitrogen for an additional 5 min. The reaction was stirred at 60° C. for 2 hours. After cooling to ambient temperature, the reaction mixture was diluted with EtOAc (˜100 mL), washed with water (20 mL), and then washed with brine (20 mL). The organic layer was dried (Na₂SO₄) and filtered. The filtrate was concentrated under reduced pressure and the residue was purified by silica gel chromatography, eluting with heptane/ethyl acetate 0-100% over 40 minutes with a 60 minute hold to give the title compound as a beige solid (2.40 g, 52.4% yield). MS (APCI+) m/z=311 (M+H+CH₃CN)⁺.

### Example 159B

### Ethyl 4,6-dichloropyrimidine-5-carboxylate

To a solution of 4,6-dichloropyrimidine-5-carboxylic acid (15.00 g, 77.70 mmol) and K₂CO₃ (21.50 g, 155.50 mmol) in DMF (150.00 ml) was added iodoethane (13.30 g, 85.50 mmol). The mixture was stirred for 1 h at 80° C. under N₂. The mixture was poured into water (1.50 L) and extracted with EA (3×300 ml). The organic layers were concentrated under reduced pressure to give the title compound (18.0 g, 89.0%) as a brown oil which was used directly without purification. ¹H NMR: (400 MHz, CDCl₃): δ 8.82 (s, 1H), 4.49 (q, J=7.2 Hz, 2H), 1.42 (t, J=7.0 Hz, 3H). LC-MS (ESI): m/z 221.10 (M+H)

### Example 159C

### Ethyl 4-chloro-6-methoxypyrimidine-5-carboxylate

To a solution of Example 159B (1.0 g, 4.52 mmol) in MeOH (40 mL) at 0° C. in an ice bath was added NaOMe (0.257 g, 4.75 mmol) portionwise. The mixture was stirred at 0° C. for 2 h. The mixture was then diluted with water (100 mL) and the aqueous layer was extracted with ethyl acetate (3×100 mL). The combined organic layers were dried over anhydrous sodium sulfate, filtered and concentrated under reduced pressure to give the title compound (0.9 g, 90%) as a yellow solid. LC-MS (ESI): m/z 217.1 (M+H)⁺

### Example 159D

### Ethyl 4-methoxy-6-(methylthio)pyrimidine-5-carboxylate

To a solution of Example 159C (1.2 g, 5.54 mmol) in THF (24 mL) was added K₂CO₃ (1.148 g, 8.31 mmol) and 20% sodium methanethiolate (3.88 g, 11.08 mmol) in water. The mixture was heated in a Biotage microwave at 100° C. for 2 h. The reaction mixture was diluted with water (100 mL) and the aqueous layer was extracted with ethyl acetate (3×100 mL). The combined organic layers were dried over anhydrous sodium sulfate, filtered through a Buchner funnel and concentrated. The residue was purified by silica gel chromatography eluting with 1.5% EA in PE to give the title compound (1.07 g, 85%) as a white solid. ¹H NMR: (400 MHz, CDCl₃): δ 8.58 (s, 1H), 4.41 (q, J=7.0 Hz, 2H), 4.01 (s, 3H), 2.55 (s, 3H), 1.40 (t, J=7.2 Hz, 3H). LC-MS (ESI) m/z 229.1 (M+H)⁺

### Example 159E

### 4-methoxy-6-(methylthio)pyrimidine-5-carboxylic acid

To a solution of Example 159D (1.15 g, 5.04 mmol) in MeOH (18 mL) was added 2 M NaOH (0.605 g, 15.11 mmol) in 7.56 mL of water. The mixture was stirred at room temperature for 16 h. The mixture was concentrated under reduced pressure, the residue was diluted with water and then acidified with 2M HCl to pH=2-4 at ice bath temperature. The aqueous layer was extracted with ether (3×60 mL). The combined organic layers were dried over anhydrous sodium sulfate, filtered through a Buchner funnel and concentrated to give the title compound (1.0 g, 98% yield) as a white solid. ¹H NMR: (400 MHz, DMSO-d₆): δ 13.63 (brs, 1H), 8.69 (s, 1H), 3.95 (s, 3H), 2.51 (s, 3H). LC-MS: m/z 201.0 (M+H)⁺

### Example 159F

### N-[5-(2-cyanophenyl)-4-(trifluoromethyl)-1,3-thiazol-2-yl]-4-methoxy-6-(methylsulfanyl)pyrimidine-5-carboxamide

The title compound was prepared as described in Example 2C, substituting Example 159E for Example 2A and substituting Example 159A for Example 2B (10.7 mg, 11.8% yield). ¹H NMR: (400 MHz, CDCl₃) δ 10.81 (s, 1H), 8.66 (s, 1H), 7.79 (d, J=7.6 Hz, 1H), 7.67 (t, J=7.6 Hz, 1H), 7.61-7.50 (m, 2H), 4.28 (s, 3H), 2.55 (s, 3H). MS (+ESI): m/z=452 (M+H)⁺.

### Example 160

### N-[5-(2-cyanophenyl)-4-(trifluoromethyl)-1,3-thiazol-2-yl]-4-(ethylsulfanyl)-6-methoxypyrimidine-5-carboxamide

The title compound was prepared as described in Example 2C, substituting Example 159E for Example 2A and substituting Example 159A for Example 2B. The reaction mixture was purified by column chromatography on an Analogix IF-310 (Isco REDISEP GOLD 12 g, 98:2 DCM/EtOAc). The product fractions were combined and concentrated under reduced pressure to give the title compound as a white solid (40.2 mg, 46.3% yield). ¹H NMR (400 MHz, Chloroform-d) δ 10.77 (s, 1H), 8.63 (s, 1H), 7.79 (dd, J=7.8, 1.3 Hz, 1H), 7.67 (td, J=7.6, 1.3 Hz, 1H), 7.60 7.51 (m, 2H), 4.27 (s, 3H), 3.20 (q, J=7.4 Hz, 2H), 1.37 (t, J=7.4 Hz, 3H). MS (+ESI) m/z=466 (M+H)⁺.

### Example 161

### 2-cyano-N-[5-iodo-4-(trifluoromethyl)-1,3-thiazol-2-yl]-4-methoxy-6-(trifluoromethyl)pyrimidine-5-carboxamide

### Example 161A

### N-(5-iodo-4-(trifluoromethyl)thiazol-2-yl)-4-methoxy-2-(methylthio)-6-(trifluoromethyl)pyrimidine-5-carboxamide

The title intermediate was prepared as described in Example 2C, substituting 4-methoxy-2-(methylsulfanyl)-6-(trifluoromethyl)-5-pyrimidinecarboxylic acid (Matrix Scientific) for Example 2A. The reaction mixture was purified by column chromatography on an Analogix IF-310 (Isco REDISEP 24 g, 80:20 DCM/heptane). The product fractions were combined and concentrated under reduced pressure to give the title compound as a white solid (271.5 mg, 49.9% yield). ¹H NMR (400 MHz, CDCl₃) δ 10.38 (s, 1H), 4.02 (s, 3H), 2.62 (s, 3H). MS (DCI-NH₃) m/z=545 (M+H)⁺, m/z=562 (M+NH₄)⁺.

### Example 161B

### N-(5-iodo-4-(trifluoromethyl)thiazol-2-yl)-4-methoxy-2-(methylsulfonyl)-6-(trifluoromethyl)pyrimidine-5-carboxamide

A stirred, ambient temperature solution of Example 161A (195 mg, 0.358 mmol) in dichloromethane (10 mL) was treated with solid m-chloroperbenzoic acid (308.4 mg, 1.340 mmol).

The reaction mixture was stirred at ambient temperature for 27 hours. The reaction mixture was washed with ice cold aqueous sodium bicarbonate, with saturated aqueous sodium thiosulfate, and then with brine. The organic layer was dried (MgSO₄) and filtered. The filtrate was concentrated under reduced pressure and the residue was purified by column chromatography on an Analogix IF-310 (Isco REDISEP GOLD 40 g, 80:20 to 60:40 heptane/EtOAc). The product fractions were combined and concentrated under reduced pressure to give the title compound as a white solid (51.3 mg, 24.9% yield). ¹H NMR (400 MHz, Methanol-d4) δ 4.26 (s, 3H), 3.45 (s, 3H). MS (+ESI) m/z=577 (M+H)⁺.

### Example 161C

### 2-cyano-N-[5-iodo-4-(trifluoromethyl)-1,3-thiazol-2-yl]-4-methoxy-6-(trifluoromethyl)pyrimidine-5-carboxamide

A solution of Example 161B (22 mg, 0.038 mmol) and tetrabutylammonium cyanide (11.9 mg, 0.042 mmol) in dichloromethane (1 mL) was stirred at ambient temperature for 16 hours. Volatiles were removed under reduced pressure and the residue was eluted through a Waters silica gel SEP PAK with 7:3 to 1:1 heptane/EtOAc. The filtrate was concentrated under reduced pressure to give a pale yellow semi-solid that was crystallized from Et₂O/heptane to provide the title compound as white crystals (18.0 mg, 90% yield). ¹H NMR (400 MHz, CDCl₃) δ 4.16 (s, 3H). MS (−ESI) m/z=522 (M−H)⁻.

### Example 162

### N-[5-(2-cyanopyrimidin-5-yl)-4-(trifluoromethyl)-1,3-thiazol-2-yl]-4,6-dimethoxypyrimidine-5-carboxamide

A 50 mL round bottom flask was charged with Example 2C (400 mg, 0.869 mmol), 5-(4,4,5,5-tetramethyl-1,3,2-dioxaborolan-2-yl)pyrimidine-2-carbonitrile (261 mg, 1.130 mmol), [1,1′-bis(diphenylphosphino)ferrocene]dichloropalladium(II) complex with DCM (71.0 mg, 0.087 mmol), and cesium carbonate (566 mg, 1.738 mmol). A magnetic stirring bar was added and the flask contents were placed under a dry nitrogen atmosphere. Dioxane (8 mL) was introduced via syringe, and then nitrogen was bubbled through the stirred reaction mixture for 5 minutes. The reaction mixture was heated to 75° C. and stirred at that temperature for 16 hours. After cooling to ambient temperature, the reaction mixture was diluted with EtOAc and filtered through a pad of diatomaceous earth. The filtrate was concentrated under reduced pressure to give a crude brown oil that was purified by column chromatography on an Analogix IF-310 (Isco REDISEP GOLD 40 g, 70:30 heptane/EtOAc). Product fractions were combined and concentrated under reduced pressure to give a solid that was repurified (Isco REDISEP GOLD 24 g, 80:20 heptane/EtOAc). The product fractions were combined and concentrated under reduced pressure to give a white solid that was crystallized from Et₂O/heptane to give the title compound (25.3 mg, 6.6% yield). ¹H NMR (400 MHz, CDCl₃) δ 10.37 (s, 1H), 8.94 (s, 2H), 8.55 (s, 1H), 4.19 (s, 6H). MS (DCI-NH₃) m/z=438 (M+H)⁺, m/z=455 (M+NH₄)+, m/z=472 (M+NH₄+NH₃)⁺.

### Example 163

### N-[4,5-bis(trifluoromethyl)-1,3-thiazol-2-yl]-4,6-dimethoxypyrimidine-5-carboxamide

A mixture of Example 2C (138 mg, 0.3 mmol) and (1,10-phenanthroline)(trifluoromethyl)-copper(I) (113 mg, 0.360 mmol) in DMF (3 mL) was stirred at 100° C. for 20 hours in a vial sealed with a Teflon pressure relief cap. The reaction mixture was diluted with water and extracted with EtOAc. The organic layer was washed with dilute aqueous ammonium hydroxide and then with brine. The organic layer was dried (Na₂SO₄) and filtered. The filtrate was concentrated under reduced pressure to give a yellow semi-solid that was purified by column chromatography on an Analogix IF-310 (Isco REDISEP GOLD 40 g, 100% DCM to 90:10 DCM/EtOAc). Fractions containing impure product were combined and concentrated under reduced pressure. The residue was column purified again (Isco REDISEP GOLD 12 g, 99:1 to 97:3 DCM/EtOAc). Product fractions from this second column were combined and concentrated under reduced pressure to give the title compound as a white solid (1 mg, 0.8% yield). ¹H NMR (CDCl₃) δ 10.35 (s, 1H), 8.55 (s, 1H), 4.17 (s, 6H). MS (DCI-NH₃) m/z=403 (M+H)⁺, m/z=420 (M+NH₄)+

### Example 164

### N-[5-(4-cyanocyclohex-1-en-1-yl)-4-(trifluoromethyl)-1,3-thiazol-2-yl]-4,6-dimethoxypyrimidine-5-carboxamide

A microwave vial was charged with Example 2C (0.05 g, 0.109 mmol), 4-(4,4,5,5-tetramethyl-1,3,2-dioxaborolan-2-yl)cyclohex-3-enecarbonitrile (0.028 g, 0.120 mmol)DME (2 ml), aqueous potassium carbonate (0.239 ml, 0.239 mmol) and PdCl₂(dppf)-CH₂Cl₂ adduct (8.87 mg, 10.87 μmol). The mixture was heated at 120° C. for 10 mins, evaporated under reduced pressure and the residue was chromatographed on silica gel eluting with 0-100% EA-heptane to give the title compound (0.02 g, 41.9% yield). ¹H NMR (400 MHz, CDCl₃) 10.06 (1H, s), 8.54 (1H, s), 5.94 (1H, s), 4.18 (6H, s), 2.97-2.87 (1H, m), 2.64-2.33 (4H, m), 2.18-2.13 (1H, m), 2.05-2.09 (1H, m); MS (ESI) m/z 440 (M+H)⁺.

### Example 165

### 4,6-dimethoxy-N-{5-[1-(methylsulfonyl)-1,2,3,6-tetrahydropyridin-4-yl]-4-(trifluoromethyl)-1,3-thiazol-2-yl}pyrimidine-5-carboxamide

The title compound was prepared as described in Example 164, substituting 1-(methylsulfonyl)-4-(4,4,5,5-tetramethyl-1,3,2-dioxaborolan-2-yl)-1,2,3,6-tetrahydropyridine for 4-(4,4,5,5-tetramethyl-1,3,2-dioxaborolan-2-yl)cyclohex-3-enecarbonitrile (0.02 g, 37.3% yield). ¹H NMR (400 MHz, CDCl₃) 10.04 (1H, s), 8.51 (1H, s), 6.00 (1H, s), 4.13 (6H, s), 3.97 (2H, m), 3.54 (2H, m), 2.87 (3H, s), 2.57 (2H, m); MS (ESI) m/z 394 (M+H)⁺.

### Example 166

### 4,6-dimethoxy-N-{4-(trifluoromethyl)-5-[2-(trifluoromethyl)pyridin-4-yl]-1,3-thiazol-2-yl}pyrimidine-5-carboxamide

The title compound was prepared as described in Example 164, substituting (2-(trifluoromethyl)pyridin-4-yl)boronic acid for 4-(4,4,5,5-tetramethyl-1,3,2-dioxaborolan-2-yl)cyclohex-3-enecarbonitrile (0.043 g, 83% yield). ¹H NMR (400 MHz, CDCl₃) 10.30 (1H, s), 8.82 (1H, d, J=5), 8.54 (1H, s), 7.78 (1H, s), 7.59 (1H, d, J=5), 4.17 (6H, s); MS (ESI) m/z 480 (M+H)⁺.

### Example 167

### 4,6-dimethoxy-N-[5-(quinolin-4-yl)-4-(trifluoromethyl)-1,3-thiazol-2-yl]pyrimidine-5-carboxamide

The title compound was prepared as described in Example 164, substituting 4-(4,4,5,5-tetramethyl-1,3,2-dioxaborolan-2-yl)quinoline for 4-(4,4,5,5-tetramethyl-1,3,2-dioxaborolan-2-yl)cyclohex-3-enecarbonitrile (0.061 g, 60.8% yield). ¹H NMR (400 MHz, CDCl₃) 10.24 (1H, s), 8.98 (1H, m), 8.54 (1H, s), 8.20 (1H, m), 7.78 (1H, m), 7.78 (2H, m), 7.57 (1H, m), 7.44 (1H, m) 4.17 (6H, s); MS (ESI) m/z 462 (M+H)⁺.

### Example 168

### N-[5-(1-cyclopropyl-1H-pyrazol-4-yl)-4-(trifluoromethyl)-1,3-thiazol-2-yl]-4,6-dimethoxypyrimidine-5-carboxamide

The title compound was prepared as described in Example 164, substituting 1-cyclopropyl-4-(4,4,5,5-tetramethyl-1,3,2-dioxaborolan-2-yl)-1H-pyrazole for 4-(4,4,5,5-tetramethyl-1,3,2-dioxaborolan-2-yl)cyclohex-3-enecarbonitrile (0.065 g, 67.9% yield). ¹H NMR (400 MHz, CDCl₃) 10.82 (1H, s), 8.52 (1H, s), 7.66 (1H, s), 7.63 (1H, s), 4.13 (6H, s), 3.64 (1H, m), 1.21-1.17 (2H, m) 1.10-1.05 (2H, m); MS (ESI) m/z 441 (M+H)⁺.

### Example 169

### 4,6-dimethoxy-N-[5-(1,2-oxazol-4-yl)-4-(trifluoromethyl)-1,3-thiazol-2-yl]pyrimidine-5-carboxamide

The title compound was prepared as described in Example 164, substituting 4-isoxazoleboronic acid pinacol ester for 4-(4,4,5,5-tetramethyl-1,3,2-dioxaborolan-2-yl)cyclohex-3-enecarbonitrile (0.013 g, 14.9% yield). ¹H NMR (400 MHz, CDCl₃) 10.19 (1H, s), 8.65 (1H, s), 8.53 (1H, s), 8.48 (1H, s), 4.17 (6H, s); MS (ESI) m/z 402 (M+H)⁺.

### Example 170

### 4,6-dimethoxy-N-{5-[3-(propan-2-yl)-4,5-dihydro-1,2-oxazol-5-yl]-4-(trifluoromethyl)-1,3-thiazol-2-yl}pyrimidine-5-carboxamide

To a solution of Example 22A (0.100 g, 0.278 mmol) and isobutyraldehyde oxime (0.024 g, 0.278 mmol) in CH₂Cl₂ (2 ml) was added sodium hypochlorite (0.7 ml, 0.694 mmol). The biphasic system was stirred vigorously overnight, the mixture was separated over water and CHCl₃, and the organic layer was concentrated under reduced pressure. The resulting residue was subjected to chromatography on silica gel eluting with 0-50% ethyl acetate-heptane to isolate the title compound (0.048 g, 38.8% yield)¹H NMR (400 MHz, CDCl₃) 9.88 (1H, s), 8.51 (1H, s), 6.01 (1H, ddd, J=11, 7, 2), 4.17 (6H, s), 3.48 (1H, dd, J=17, 11), 2.95 (1H, dd, J=17, 7), 2.76 (1H, p, J=7,7,7,7), 1.20 (6H, d, J=7); MS (ESI) m/z 446 (M+H)⁺.

### Example 171

### 4,6-dimethoxy-N-{5-[3-(propan-2-yl)-1,2-oxazol-5-yl]-4-(trifluoromethyl)-1,3-thiazol-2-yl}pyrimidine-5-carboxamide

A microwave vial was charged with Example 11 (0.05 g, 0.140 mmol), (Z)—N-hydroxyisobutyrimidoyl chloride (0.017 g, 0.140 mmol), CHCl₃ (1.5 ml) and THF (1.5 ml). The mixture was heated in a microwave at 140° C. for 15 mins, concentrated and then purified by prep HPLC to give the title compound (0.015 g, 24.24% yield). ¹H NMR (400 MHz, CDCl₃) 10.27 (1H, s), 8.54 (1H, s), 6.47 (1H, s), 4.16 (6H, s), 3.12 (1H, p, J=7,7,7,7), 1.35 6H, d, J=7); MS (ESI) m/z 444 (M+H)⁺.

### Example 172

### 4,6-dimethoxy-N-[5-(1,2-oxazol-3-yl)-4-(trifluoromethyl)-1,3-thiazol-2-yl]pyrimidine-5-carboxamide

### Example 172A

### 4,6-dimethoxy-N-(5-(3-oxoprop-1-yn-1-yl)-4-(trifluoromethyl)thiazol-2-yl)pyrimidine-5-carboxamide

To a solution of Example 12 (0.5 g, 1.288 mmol) in DMSO (8 ml) was added IBX (0.96 g, 1.545 mmol). The mixture was stirred for 4 hours, transferred to a 100 mL flask, and ˜80 mL of water was added followed by EtOAc. The mixture was filtered through CELITE®, rinsed with EtOAc, and the aqueous phase was extracted twice with EtOAc. The combined organics were washed 5 times with brine, dried over MgSO₄, concentrated under reduced pressure and subjected to chromatography on silica gel eluting with 0-50% EA-heptane to give the title compound (0.261 g, 52.5% yield) as a solid. MS (ESI) m/z 387 (M+H)⁺.

### Example 172B

### 4,6-dimethoxy-N-[5-(1,2-oxazol-3-yl)-4-(trifluoromethyl)-1,3-thiazol-2-yl]pyrimidine-5-carboxamide

To a solution of Example 172A (. 1 g, 0.259 mmol) in ethanol (2 ml) was added aqueous hydroxylamine (0.017 g, 0.259 mmol). The mixture was then heated at 120° C. for 10 mins in a microwave. The mixture was added to a flash chromatography column and eluted with an ethylacetate-heptane gradient to give the title compound (0.012 g, 11.55% yield). ¹H NMR (400 MHz, CDCl₃) 10.28 (1H, s), 8.53, (1H, s), 6.62 (1H, s), 4.17 (6H, s); MS (ESI) m/z 402 (M+H)⁺.

### Example 173

### N-{5-[4-(2-cyanopropan-2-yl)phenyl]-4-(trifluoromethyl)-1,3-thiazol-2-yl}-4,6-dimethoxypyrimidine-5-carboxamide

The title compound was prepared as described in Example 184, substituting 2-methyl-2-(4-(4,4,5,5-tetramethyl-1,3,2-dioxaborolan-2-yl)phenyl)propanenitrile for 2-methoxy-5-(4,4,5,5-tetramethyl-1,3,2-dioxaborolan-2-yl)nicotinonitrile (30.2 mg, 72.79%). ¹H NMR (400 MHz, DMSO-d₆) δ ppm 8.62 (s, 1H), 7.70-7.65 (m, 2H), 7.60 (d, J=8.6 Hz, 2H), 3.97 (s, 6H), 1.74 (s, 6H). MS (APCI⁺) m/z 477.9 (M+H)⁺.

### Example 174

### 4,6-dimethoxy-N-{5-[1-(propan-2-yl)-1H-pyrazol-4-yl]-4-(trifluoromethyl)-1,3-thiazol-2-yl}pyrimidine-5-carboxamide

The title compound was prepared as described in Example 184, substituting 1-isopropyl-4-(4,4,5,5-tetramethyl-1,3,2-dioxaborolan-2-yl)-1H-pyrazole for 2-methoxy-5-(4,4,5,5-tetramethyl-1,3,2-dioxaborolan-2-yl)nicotinonitrile (34.8 mg, 72.4%). ¹H NMR (400 MHz, DMSO-d₆) δ ppm 8.62 (s, 1H), 8.13 (s, 1H), 7.69 (s, 1H), 4.57 (h, J=6.7 Hz, 1H), 3.97 (s, 6H), 1.46 (d, J=6.6 Hz, 6H). MS (APCI⁺) m/z 442.9 (M+H)⁺.

### Example 175

### N-{5-[1-(cyclopropylmethyl)-1H-pyrazol-4-yl]-4-(trifluoromethyl)-1,3-thiazol-2-yl}-4,6-dimethoxypyrimidine-5-carboxamide

The title compound was prepared as described in Example 184, substituting 1-(cyclopropylmethyl)-4-(4,4,5,5-tetramethyl-1,3,2-dioxaborolan-2-yl)-1H-pyrazole for 2-methoxy-5-(4,4,5,5-tetramethyl-1,3,2-dioxaborolan-2-yl)nicotinonitrile (8.7 mg, 22.0%,). ¹H NMR (400 MHz, DMSO-d₆) δ ppm 8.62 (s, 1H), 8.14 (s, 1H), 7.70 (s, 1H), 4.03 (d, J=7.2 Hz, 2H), 3.97 (s, 6H), 1.28 (tt, J=7.6, 4.7 Hz, 1H), 0.60-0.51 (m, 2H), 0.43-0.36 (m, 2H). MS (APCI⁺) m/z 454.9 (M+H)⁺.

### Example 176

### 4,6-dimethoxy-N-[5-(1-methyl-1H-pyrazol-5-yl)-4-(trifluoromethyl)-1,3-thiazol-2-yl]pyrimidine-5-carboxamide

The title compound was prepared as described in Example 184, substituting 1-methyl-5-(4,4,5,5-tetramethyl-1,3,2-dioxaborolan-2-yl)-1H-pyrazole for 2-methoxy-5-(4,4,5,5-tetramethyl-1,3,2-dioxaborolan-2-yl)nicotinonitrile (12.3 mg, 27.3%). ¹H NMR (400 MHz, DMSO-d₆) δ ppm 8.62 (d, J=6.1 Hz, 1H), 7.59 (d, J=1.9 Hz, 1H), 6.56 (d, J=1.9 Hz, 1H), 3.97 (d, J=5.0 Hz, 9H). MS (APCI⁺) m/z 414.9 (M+H)⁺.

### Example 177

### N-[5-(1-ethyl-1H-pyrazol-4-yl)-4-(trifluoromethyl)-1,3-thiazol-2-yl]-4,6-dimethoxypyrimidine-5-carboxamide

The title compound was prepared as described in Example 184, substituting 1-ethyl-4-(4,4,5,5-tetramethyl-1,3,2-dioxaborolan-2-yl)-1H-pyrazole for 2-methoxy-5-(4,4,5,5-tetramethyl-1,3,2-dioxaborolan-2-yl)nicotinonitrile (35.4 mg, 76.0%,). ¹H NMR (400 MHz, DMSO-d₆) δ ppm 8.62 (s, 1H), 8.12 (s, 1H), 7.69 (s, 1H), 4.20 (q, J=7.3 Hz, 2H), 3.97 (s, 6H), 1.41 (t, J=7.3 Hz, 3H). MS (APCI⁺) m/z 428.9 (M+H)⁺.

### Example 178

### N-{4-[2-(4-fluorophenoxy)ethyl]-1,3-thiazol-2-yl}-4,6-dimethoxypyrimidine-5-carboxamide

### Example 178A

### 4,6-dimethoxypyrimidine-5-carbonyl chloride

To a solution of Example 2A (15.0 g, 81 mmol) in CH₂Cl₂ (320 ml) and N,N-dimethylformamide (0.16 ml, 2.1 mmol) in CH₂Cl₂ (320 mL) was added (COCl)₂ (9.25 ml, 106 mmol) slowly over −30 min, the mixture was stirred for 3 h, and then concentrated to give the title compound (16.67 g), which was used without purification: ¹H NMR (400 MHz, CDCl₃) δ 8.48 (s, 1H), 4.07 (s, 6H)

### Example 178B

### N-{4-[2-(4-fluorophenoxy)ethyl]-1,3-thiazol-2-yl}-4,6-dimethoxypyrimidine-5-carboxamide

To a solution of Example 178A (40 mg, 0.19 mmol) in dichloromethane (0.5 mL) was added a solution of 4-(2-(4-fluorophenoxy)ethyl)thiazol-2-amine (45 mg, 19 mmol) in CH₂Cl₂ (0.5 ml) followed by an addition of neat triethyl amine (35 ul, 25 mmol) and dimethyl amino pyridine (23 mg, 0.18 mmol). The reaction mixture was allowed to stir at room temperature for 2 hours. The residues were dried, dissolved in 1:1 DMSO/MeOH and purified by reverse phase HPLC (TFA method). The samples were purified by preparative HPLC on a Phenomenex LUNA C8(2) 5 um 100 Å AXIA column (30 mm×150 mm). A gradient of MeCN (A) and 0.1% trifluoroacetic acid in water (B) was used at a flow rate of 50 mL/min (0-0.5 min 5% A, 0.5-8.5 min linear gradient 5-100% A, 8.7-10.7 min 100% A, 10.7-11.0 min linear gradient 100-5% A) to elute the title compound (12.5 mg 12% yield). ¹H NMR (400 MHz, DMSO-d₆) δ 8.58 (s, 1H), 7.14-7.05 (m, 2H), 7.00 (s, 1H), 6.98-6.91 (m, 2H), 4.24 (t, J=6.5 Hz, 2H), 3.94 (s, 6H), 3.07 (t, J=6.4 Hz, 2H). MS (ESI) m/z 405 (M+H)⁺.

### Example 179

### 4,6-dimethoxy-N-{4-[(2-methylphenoxy)methyl]-1,3-thiazol-2-yl}pyrimidine-5-carboxamide

The title compound was prepared as described in Example 178B, substituting 4-((o-tolyloxy)methyl)thiazol-2-amine for 4-(2-(4-fluorophenoxy)ethyl)thiazol-2-amine (19.6 mg 20% yield). ¹H NMR (400 MHz, DMSO-d₆) δ 8.60 (s, 1H), 7.29 (s, 1H), 7.16 (ddd, J=7.2, 5.8, 1.9 Hz, 2H), 7.11-7.00 (m, 1H), 6.87 (td, J=7.3, 1.1 Hz, 1H), 5.08 (s, 2H), 3.95 (s, 6H), 2.18 (s, 3H). MS (ESI) m/z 387 (M+H)⁺.

### Example 180

### 4,6-dimethoxy-N-{5-[1-(propan-2-yl)-1H-pyrazol-5-yl]-4-(trifluoromethyl)-1,3-thiazol-2-yl}pyrimidine-5-carboxamide

The title compound was prepared as described in Example 184, substituting 1-isopropyl-5-(4,4,5,5-tetramethyl-1,3,2-dioxaborolan-2-yl)-1H-pyrazole for 2-methoxy-5-(4,4,5,5-tetramethyl-1,3,2-dioxaborolan-2-yl)nicotinonitrile (17 mg, 41.5%,). ¹H NMR (400 MHz, DMSO-d₆) δ ppm 8.63 (s, 1H), 7.64 (d, J=1.7 Hz, 1H), 6.51 (d, J=1.9 Hz, 1H), 4.37 (p, J=6.5 Hz, 1H), 3.98 (s, 6H), 1.36 (d, J=6.5 Hz, 6H). MS (APCI⁺) m/z 442.9 (M+H)⁺.

### Example 181

### 4,6-dimethoxy-N-{5-[1-methyl-3-(propan-2-yl)-1H-pyrazol-4-yl]-4-(trifluoromethyl)-1,3-thiazol-2-yl}pyrimidine-5-carboxamide

The title compound was prepared as described in Example 184, substituting 3-isopropyl-1-methyl-4-(4,4,5,5-tetramethyl-1,3,2-dioxaborolan-2-yl)-1H-pyrazole for 2-methoxy-5-(4,4,5,5-tetramethyl-1,3,2-dioxaborolan-2-yl)nicotinonitrile (14.2 mg, 33.6%,). ¹H NMR (400 MHz, DMSO-d₆) δ ppm 8.62 (s, 1H), 7.81 (s, 1H), 3.97 (s, 6H), 3.83 (s, 3H), 2.91 (p, J=6.9 Hz, 1H), 1.16 (d, J=6.9 Hz, 7H). MS (APCI⁺) m/z 456.9 (M+H)⁺

### Example 182

### 4,6-dimethoxy-N-{5-[2-(morpholin-4-yl)pyridin-4-yl]-4-(trifluoromethyl)-1,3-thiazol-2-yl}pyrimidine-5-carboxamide

The title compound was prepared as described in Example 184, substituting 2-(piperidin-1-yl)-4-(4,4,5,5-tetramethyl-1,3,2-dioxaborolan-2-yl)pyridine for 2-methoxy-5-(4,4,5,5-tetramethyl-1,3,2-dioxaborolan-2-yl)nicotinonitrile (37.8 mg, 61.2%,). ¹H NMR (400 MHz, DMSO-d₆) δ ppm 8.63 (s, 1H), 8.19 (d, J=5.6 Hz, 1H), 7.11 (s, 1H), 6.89 (d, J=5.5 Hz, 1H), 3.97 (s, 6H), 3.79-3.75 (m, 2H), 3.74-3.68 (m, 2H), 3.56 (t, J=4.7 Hz, 4H). MS (APCI⁺) m/z 496.9 (M+H)⁺.

### Example 183

### 4,6-dimethoxy-N-[5-(2-methoxypyrimidin-5-yl)-4-(trifluoromethyl)-1,3-thiazol-2-yl]pyrimidine-5-carboxamide

The title compound was prepared as described in Example 184, substituting 2-methoxy-5-(4,4,5,5-tetramethyl-1,3,2-dioxaborolan-2-yl)pyrimidine for 2-methoxy-5-(4,4,5,5-tetramethyl-1,3,2-dioxaborolan-2-yl)nicotinonitrile (25.4 mg 50.4%,). ¹H NMR (400 MHz, DMSO-d₆) δ ppm 8.63 (s, 1H), 8.60 (d, J=2.4 Hz, 1H), 8.47 (d, J=2.4 Hz, 1H), 4.08 (s, 3H), 3.97 (s, 6H). MS (APCI⁺) m/z 467.0 (M+H)⁺.

### Example 184

### N-[5-(5-cyano-6-methoxypyridin-3-yl)-4-(trifluoromethyl)-1,3-thiazol-2-yl]-4,6-dimethoxypyrimidine-5-carboxamide

A microwave vial was charged with a stirring bar, a solution of Example 1B (40 mg, 0.09 mmol) in dioxane (1.0 mL), a solution of 2-methoxy-5-(4,4,5,5-tetramethyl-1,3,2-dioxaborolan-2-yl)nicotinonitrile (30.0 mg, 0.13 mmol), in dioxane (217 μL), [1,1′-bis(diphenylphosphino)ferrocene]dichloropalladium(II) complex with dichloromethane (7.10 mg, 0.009 mmol), and 261 μL of 1M aqueous solution of cesium carbonate (85.0 mg, 0.27 mmol). The vial was capped and placed to heat in the Biotage Microwave Optimizer at 120° C. for 20 minutes.

The mixture was then filtered and concentrated to dryness. The residue was dissolved in 1:1 DMSO/MeOH and purified by reverse phase HPLC on 2 coupled C8 5 um 100 Å columns (30 mm×75 mm each). A gradient of acetonitrile (A) and 0.1% trifluoroacetic acid in water (B) was used, at a flow rate of 50 mL/min (0-0.5 min 5% A, 0.5-8.5 min linear gradient 05-100% A, 8.7-10.7 min 100% A, 10.7-11 min linear gradient 100-05% A) to isolate the title compound (30.6 mg, 50.36%). ¹H NMR (400 MHz, DMSO-d₆) δ ppm 8.63 (s, 1H), 8.60 (d, J=2.4 Hz, 1H), 8.47 (d, J=2.4 Hz, 1H), 4.08 (s, 3H), 3.97 (s, 6H). MS (APCI⁺) m/z 467.0 (M+H)⁺.

### Example 185

### 4,6-dimethoxy-N-{5-[2-(morpholin-4-yl)pyridin-3-yl]-4-(trifluoromethyl)-1,3-thiazol-2-yl}pyrimidine-5-carboxamide

The title compound was prepared as described in Example 184, substituting 4-(3-(4,4,5,5-tetramethyl-1,3,2-dioxaborolan-2-yl)pyridin-2-yl)morpholine for 2-methoxy-5-(4,4,5,5-tetramethyl-1,3,2-dioxaborolan-2-yl)nicotinonitrile (30.6 mg, 57.7%,). ¹H NMR (400 MHz, DMSO-d₆) δ ppm 8.63 (s, 1H), 8.35 (dd, J=4.9, 1.8 Hz, 1H), 7.69 (dd, J=7.6, 1.8 Hz, 1H), 7.11 (dd, J=7.5, 4.9 Hz, 1H), 3.98 (s, 6H), 3.60 (dd, J=5.7, 3.6 Hz, 4H), 3.10-3.05 (m, 4H). MS (APCI⁺) m/z 497.0 (M+H)⁺.

### Example 186

### 4,6-dimethoxy-N-{5-[1-methyl-3-(trifluoromethyl)-1H-pyrazol-4-yl]-4-(trifluoromethyl)-1,3-thiazol-2-yl}pyrimidine-5-carboxamide

The title compound was prepared as described in Example 184, substituting 1-methyl-4-(4,4,5,5-tetramethyl-1,3,2-dioxaborolan-2-yl)-3-(trifluoromethyl)-1H-pyrazole for 2-methoxy-5-(4,4,5,5-tetramethyl-1,3,2-dioxaborolan-2-yl)nicotinonitrile (9.7 mg, 23.2%,). ¹H NMR (400 MHz, DMSO-d₆) δ ppm 8.66-8.59 (m, 1H), 8.22 (s, 1H), 4.02-3.93 (m, 9H). MS (APCI⁺) m/z 483.0 (M+H)⁺.

### Example 187

### N-[5-(furo[3,2-b]pyridin-2-yl)-4-(trifluoromethyl)-1,3-thiazol-2-yl]-4,6-dimethoxypyrimidine-5-carboxamide

To a solution of Example 2C (26 mg, 0.06 mmol) in DME (1.5 mL) in a 2 mL microwave vial was added 2-(4,4,5,5-tetramethyl-1,3,2-dioxaborolan-2-yl)furo[3,2-b]pyridine (29 mg, 0.12 mmol), followed by PEPPSI (15 mg, 0.02 mmol) and cesium carbonate (55 mg, 0.18 mmol). The reaction vessel was sealed and heated to 100° C. for 20 min. The reaction mixture was the filtered and the resulting products were collected and concentrated to dryness under reduced pressure. The residues were dissolved in 1:1 DMSO/MeOH and purified by reverse phase HPLC on 2 coupled C8 5 um 100 Å columns (30 mm×75 mm each). A gradient of acetonitrile (A) and 0.1% trifluoroacetic acid in water (B) was used, at a flow rate of 50 mL/min (0-0.5 min 5% A, 0.5-8.5 min linear gradient 05-100% A, 8.7-10.7 min 100% A, 10.7-11 min linear gradient 100-05% A) to obtain the title compound (4.1 mg, 13.1% yield)¹H NMR (400 MHz, DMSO-d₆) δ 8.64 (s, 1H), 8.61 (dd, J=4.8, 1.3 Hz, 1H), 8.15 (d, J=8.4 Hz, 1H), 7.50 (s, 1H), 7.48 (dd, J=8.4, 4.7 Hz, 1H), 3.98 (s, 6H). MS (APCI+) m/z 451.8 (M+H).

### Example 188

### N-{5-[1-(cyclopropylmethyl)-1H-pyrazol-5-yl]-4-(trifluoromethyl)-1,3-thiazol-2-yl}-4,6-dimethoxypyrimidine-5-carboxamide

To a solution of Example 2C (26 mg, 0.06 mmol) in DME (1.5 mL) in a 2 mL microwave vial was added 1-(cyclopropylmethyl)-5-(4,4,5,5-tetramethyl-1,3,2-dioxaborolan-2-yl)-1H-pyrazole (30 mg, 0.12 mmol), followed by PEPPSI (15 mg, 0.02 mmol) and cesium carbonate (55 mg, 0.18 mmol). The reaction vessel was sealed and heated to 100° C. for 20 min. The reaction mixture was filtered and the resulting products were collected and concentrated to dryness under reduced pressure. The residues were dissolved in 1:1 DMSO/MeOH and purified by reverse phase HPLC (a gradient of acetonitrile (A) and 0.1% trifluoroacetic acid in water (B) was used, at a flow rate of 50 mL/min (0-0.5 min 5% A, 0.5-8.5 min linear gradient 05-100% A, 8.7-10.7 min 100% A, 10.7-11 min linear gradient 100-05% A) to obtain the title compound (8.4 mg, 33.3% yield). ¹H NMR (400 MHz, DMSO-d₆) δ 8.63 (s, 1H), 7.62 (d, J=1.8 Hz, 1H), 6.55 (d, J=1.8 Hz, 1H), 3.98 (s, 6H), 3.91 (d, J=7.0 Hz, 2H), 1.18-1.07 (m, 1H), 0.51-0.44 (m, 2H), 0.26-0.19 (m, 2H). MS (APCI+) m/z 454.9 (M+H).

### Example 189

### N-(4-tert-butyl-5-cyano-1,3-thiazol-2-yl)-4,6-dimethoxypyrimidine-5-carboxamide

The title compound was prepared as described in Example 178B, substituting 2-amino-4-(tert-butyl)thiazole-5-carbonitrile for 4-(2-(4-fluorophenoxy)ethyl)thiazol-2-amine (37.7 mg, 44% yield). ¹H NMR (400 MHz, DMSO-d₆) δ 8.58 (s, 1H), 3.96 (s, 6H), 1.42 (s, 9H). MS (ESI) m/z 348 (M+H)⁺.

### Example 190

### N-[4-cyclopropyl-5-(4-fluorobenzyl)-1,3-thiazol-2-yl]-4,6-dimethoxypyrimidine-5-carboxamide

The title compound was prepared as described in Example 178B, substituting 4-cyclopropyl-5-(4-fluorobenzyl)thiazol-2-amine for 4-(2-(4-fluorophenoxy)ethyl)thiazol-2-amine (13.6 mg, 14% yield). ¹H NMR (400 MHz, DMSO-d₆) δ 8.55 (s, 1H), 7.38-7.26 (m, 2H), 7.22-7.08 (m, 2H), 4.15 (s, 2H), 3.91 (s, 6H), 2.11 (td, J=8.3, 4.3 Hz, 1H), 0.96-0.71 (m, 4H). MS (ESI) m/z 415 (M+H)⁺.

### Example 191

### N-[4-(2,2-dimethylpropyl)-1,3-thiazol-2-yl]-4,6-dimethoxypyrimidine-5-carboxamide

The title compound was prepared as described in Example 178B, substituting 4-neopentylthiazol-2-amine for 4-(2-(4-fluorophenoxy)ethyl)thiazol-2-amine (27 mg, 34% yield). ¹H NMR (400 MHz, DMSO-d₆) δ 8.58 (s, 1H), 6.85 (s, 1H), 3.94 (s, 6H), 0.92 (s, 9H). MS (ESI) m/z 337 (M+H)⁺.

### Example 192

### N-(5-bromo-4-tert-butyl-1,3-thiazol-2-yl)-4,6-dimethoxypyrimidine-5-carboxamide

The title compound was prepared as described in Example 178B, substituting 5-bromo-4-(tert-butyl)thiazol-2-amin for 4-(2-(4-fluorophenoxy)ethyl)thiazol-2-amine (10.7 mg, 10% yield). ¹H NMR (400 MHz, DMSO-d₆) δ 8.58 (s, 1H), 3.95 (s, 6H), 1.40 (s, 9H). MS (ESI) m/z 402 (M+H)⁺.

### Example 193

### ethyl 4-tert-butyl-2-{[(4,6-dimethoxypyrimidin-5-yl)carbonyl]amino}-1,3-thiazole-5-carboxylate

The title compound was prepared as described in Example 178B, substituting ethyl 2-amino-4-(tert-butyl)thiazole-5-carboxylate for 4-(2-(4-fluorophenoxy)ethyl)thiazol-2-amine (25.7 mg, 26% yield). ¹H NMR (400 MHz, DMSO-d₆) δ 4.27 (q, J=7.1 Hz, 2H), 3.95 (s, 6H), 1.42 (s, 9H), 1.30 (t, J=7.1 Hz, 3H). MS (ESI) m/z 395 (M+H)⁺.

### Example 194

### 4,6-dimethoxy-N-{5-[6-(2,2,2-trifluoroethoxy)pyridin-3-yl]-4-(trifluoromethyl)-1,3-thiazol-2-yl}pyrimidine-5-carboxamide

To a solution of Example 2C (40 mg, 0.09 mmol) in dioxane (0.5 mL) was added a solution of 5-(4,4,5,5-tetramethyl-1,3,2-dioxaborolan-2-yl)-2-(2,2,2-trifluoroethoxy)pyridine (32 mg, 0.10 mmol) in dioxane (0.5 ml) followed by a 1M solution of cesium carbonate (260 μl, 0.26 mmol) and Pd(dppf)dicholoromethane complex (7 mg, 0.1 mol %, 0.008 mmol). The reaction mixture was then heated in a Biotage microwave unit at 120° C. for 20 minutes. The residue was filtered through CELITE® and the filtrate was dried, dissolved in 1:1 DMSO/MeOH and purified by reverse phase HPLC (TFA method). The samples were purified by preparative HPLC on a Phenomenex LUNA C₈(2) 5 um 100 Å AXIA column (30 mm×150 mm). A gradient of ACN (A) and 0.1% trifluoroacetic acid in water (B) was used at a flow rate of 50 mL/min (0-0.5 min 5% A, 0.5-8.5 min linear gradient 5-100% A, 8.7-10.7 min 100% A, 10.7-11.0 min linear gradient 100-5% A) to obtain the title compound (12.7 mg, 24% yield). ¹H NMR (400 MHz, DMSO-d₆) δ 8.62 (s, 1H), 8.35 (d, J=2.4 Hz, 1H), 7.97 (dd, J=8.6, 2.5 Hz, 1H), 7.15 (d, J=8.5 Hz, 1H), 5.06 (q, J=9.1 Hz, 2H), 3.97 (s, 6H). MS (ESI) m/z 510 (M+H)⁺.

### Example 195

### 4,6-dimethoxy-N-{5-[3-(morpholin-4-ylsulfonyl)phenyl]-4-(trifluoromethyl)-1,3-thiazol-2-yl}pyrimidine-5-carboxamide

The title compound was prepared as described in Example 194, substituting (3 (morpholinosulfonyl)phenyl)boronic acid for 5-(4,4,5,5 tetramethyl-1,3,2-dioxaborolan-2-yl)-2-(2,2,2-trifluoroethoxy)pyridine (9.8 mg, 20% yield). ¹H NMR (400 MHz, DMSO-d₆) δ 8.58 (s, 1H), 7.97-7.77 (m, 4H), 3.97 (s, 6H), 2.98-2.87 (m, 4H). MS (ESI) m/z 560 (M+H)⁺.

### Example 196

### 4,6-dimethoxy-N-{4-(trifluoromethyl)-5-[5-(trifluoromethyl)pyridin-3-yl]-1,3-thiazol-2-yl}pyrimidine-5-carboxamide

The title compound was prepared as described in Example 194, substituting (5-(trifluoromethyl)pyridin-3-yl)boronic acid for 5-(4,4,5,5-tetramethyl-1,3,2-dioxaborolan-2-yl)-2-(2,2,2-trifluoroethoxy)pyridine (14.7 mg, 29% yield). ¹H NMR (400 MHz, DMSO-d₆) δ 9.26-9.07 (m, 1H), 9.02 (d, J=2.0 Hz, 1H), 8.63 (s, 1H), 8.52-8.35 (m, 1H), 3.98 (s, 6H). MS (ESI) m/z 480 (M+H)⁺.

### Example 197

### 4,6-dimethoxy-N-[5-{3-[(2-methoxyethyl)(methyl)sulfamoyl]phenyl}-4-(trifluoromethyl)-1,3-thiazol-2-yl]pyrimidine-5-carboxamide

The title compound was prepared as described in Example 194, substituting N-(2-methoxyethyl)-N-methyl-3-(4,4,5,5-tetramethyl-1,3,2-dioxaborolan-2-yl)benzenesulfonamide for 5-(4,4,5,5-tetramethyl-1,3,2-dioxaborolan-2-yl)-2-(2,2,2-trifluoroethoxy)pyridine (19.6 mg, 40% yield). ¹H NMR (400 MHz, DMSO-d₆) δ 8.63 (s, 1H), 7.97-7.73 (m, 3H), 3.96 (d, J=7.7 Hz, 6H), 3.45 (t, J=5.4 Hz, 2H), 3.26-3.13 (m, 5H), 2.77 (s, 3H). MS (ESI) m/z 561 (M+H)⁺.

### Example 198

### 4,6-dimethoxy-N-{5-[4-(morpholin-4-ylmethyl)phenyl]-4-(trifluoromethyl)-1,3-thiazol-2-yl}pyrimidine-5-carboxamide

The title compound was prepared as described in Example 194, substituting 4-(4-(4,4,5,5-tetramethyl-1,3,2-dioxaborolan-2-yl)benzyl)morpholine for 5-(4,4,5,5-tetramethyl-1,3,2-dioxaborolan-2-yl)-2-(2,2,2-trifluoroethoxy)pyridine (5.5 mg, 10% yield). ¹H NMR (400 MHz, DMSO-d₆) δ 8.63 (s, 1H), 7.47 (d, J=1.8 Hz, 4H), 3.97 (s, 6H), 3.60 (t, J=4.6 Hz, 4H), 3.54 (s, 2H), 2.39 (t, J=4.6 Hz, 4H). MS (ESI) m/z 510 (M+H)⁺.

### Example 199

### N-[5-(2,1,3-benzoxadiazol-5-yl)-4-(trifluoromethyl)-1,3-thiazol-2-yl]-4,6-dimethoxypyrimidine-5-carboxamide

The title compound was prepared as described in Example 194, substituting benzo[c][1,2,5]oxadiazol-5-ylboronic acid for 5-(4,4,5,5-tetramethyl-1,3,2-dioxaborolan-2-yl)-2-(2,2,2-trifluoroethoxy)pyridine (4 mg, 11% yield). ¹H NMR (400 MHz, DMSO-d₆) δ 8.61 (s, 1H), 8.28 (s, 1H), 8.18 (d, J=9.3 Hz, 1H), 7.68 (d, J=9.4 Hz, 1H), 3.97 (s, 6H). MS (ESI) m/z 453 (M+H)⁺.

### Example 200

### N-[5-(1-ethyl-3-methyl-1H-pyrazol-4-yl)-4-(trifluoromethyl)-1,3-thiazol-2-yl]-4,6-dimethoxypyrimidine-5-carboxamide

The title compound was prepared as described in Example 194, substituting 1-ethyl-3-methyl-4-(4,4,5,5-tetramethyl-1,3,2-dioxaborolan-2-yl)-1H-pyrazole for 5-(4,4,5,5-tetramethyl-1,3,2-dioxaborolan-2-yl)-2-(2,2,2-trifluoroethoxy)pyridine (17.1 mg, 45% yield). ¹H NMR (400 MHz, DMSO-d₆) δ 8.61 (s, 1H), 7.89 (s, 1H), 4.11 (q, J=7.2 Hz, 2H), 3.97 (s, 6H), 2.16 (s, 3H), 1.38 (t, J=7.3 Hz, 3H). MS (ESI) m/z 443 (M+H)⁺.

### Example 201

### N-[5-{3-[(dimethylsulfamoyl)amino]phenyl}-4-(trifluoromethyl)-1,3-thiazol-2-yl]-4,6-dimethoxypyrimidine-5-carboxamide

The title compound was prepared as described in Example 194, substituting (3-((N,N-dimethylsulfamoyl)amino)phenyl)boronic acid for 5-(4,4,5,5-tetramethyl-1,3,2-dioxaborolan-2-yl)-2-(2,2,2-trifluoroethoxy)pyridine (25.5 mg, 55% yield). ¹H NMR (400 MHz, DMSO-d₆) δ 8.62 (s, 1H), 7.46 (t, J=7.9 Hz, 1H), 7.38-7.29 (m, 2H), 7.21 (d, J=7.7 Hz, 1H), 3.97 (s, 6H), 2.73 (s, 6H). MS (ESI) m/z 533 (M+H)⁺.

### Example 202

### 4,6-dimethoxy-N-{4-(trifluoromethyl)-5-[2-(trifluoromethyl)pyrimidin-5-yl]-1,3-thiazol-2-yl}pyrimidine-5-carboxamide

The title compound was prepared as described in Example 194, substituting (2-(trifluoromethyl)pyrimidin-5-yl)boronic acid for 5-(4,4,5,5-tetramethyl-1,3,2-dioxaborolan-2-yl)-2-(2,2,2-trifluoroethoxy)pyridine (6 mg, 14% yield). H NMR (400 MHz, DMSO-d₆) δ 9.24 (s, 2H), 8.62 (s, 1H), 3.97 (s, 6H). MS (ESI) m/z 481 (M+H)⁺.

### Example 203

### N-[5-(2-cyclopropylpyrimidin-5-yl)-4-(trifluoromethyl)-1,3-thiazol-2-yl]-4,6-dimethoxypyrimidine-5-carboxamide

The title compound was prepared as described in Example 194, substituting 2-cyclopropyl-5-(4,4,5,5-tetramethyl-1,3,2-dioxaborolan-2-yl)pyrimidine for 5-(4,4,5,5-tetramethyl-1,3,2-dioxaborolan-2-yl)-2-(2,2,2-trifluoroethoxy)pyridine (9.4 mg, 24% yield). ¹H NMR (400 MHz, DMSO-d₆) δ 8.77 (s, 2H), 8.62 (s, 1H), 3.97 (s, 6H), 2.30 (tt, J=8.1, 4.7 Hz, 1H), 1.36-1.12 (m, 2H), 1.09 (dt, J=4.6, 3.0 Hz, 2H). MS (ESI) m/z 453 (M+H)⁺.

### Example 204

### N-{5-[5-cyano-6-(morpholin-4-yl)pyridin-3-yl]-4-(trifluoromethyl)-1,3-thiazol-2-yl}-4,6-dimethoxypyrimidine-5-carboxamide

The title compound was prepared as described in Example 194, substituting 2-morpholino-5-(4,4,5,5-tetramethyl-1,3,2-dioxaborolan-2-yl)nicotinonitrile for 5-(4,4,5,5-tetramethyl-1,3,2-dioxaborolan-2-yl)-2-(2,2,2-trifluoroethoxy)pyridine (8.9 mg, 20% yield). ¹H NMR (400 MHz, DMSO-d₆) δ 8.62 (s, 1H), 8.49 (d, J=2.5 Hz, 1H), 8.23 (d, J=2.4 Hz, 1H), 3.97 (s, 6H), 3.76 (s, 8H). MS (ESI) m/z 523 (M+H)⁺.

### Example 205

### N-(5-(6-cyano-5-(oxetan-3-yl-methoxy)pyridine-3-yl)-4-(trifluoromethyl)thiazol-2-yl)-4,6-dimethoxypyrimidine-5-carboxamide

### Example 205A

### N-(5-(6-cyano-5-fluoropyridin-3-yl)-4-(trifluoromethyl)thiazol-2-yl)-4,6-dimethoxypyrimidine-5-carboxamide

A microwave vial charged with Example 2 (0.2 g, 0.435 mmol), 3-fluoro-5-(4,4,5,5-tetramethyl-1,3,2-dioxaborolan-2-yl)picolinonitrile (Ark Pharm, 0.13 g, 0.522 mmol), Pd(amphos)Cl₂ (0.031 g, 0.043 mmol), potassium phosphate tribasic (0.652 mL, 1.304 mmol), and dioxane (3 mL) was purged with nitrogen for a few minutes, then irradiated for 15 minutes at 150° C. in a Biotage microwave apparatus. After cooling to ambient temperature the mixture was diluted with 20 mL of EtOAc and filtered. The solvent was evaporated under reduced pressure and the residue was purified by silica gel chromatography eluting with a gradient 0-60% of EtOAc in heptanes to give the title compound (0.09 g, 46%). ¹H NMR (400 MHz, CDCl₃) δ ppm 10.59 (s, 1H), 8.65 (bs, 1H), 8.56 (s, 1H), 7.72 (dd, J=5.1, 1.7 Hz, 1H) 4.15 (s, 6H). MS (ESI) m/z 455 (M+H)⁺.

### Example 205B

### N-(5-(6-cyano-5-(oxetan-3-yl-methoxy)pyridine-3-yl)-4-(trifluoromethyl)thiazol-2-yl)-4,6-dimethoxypyrimidine-5-carboxamide

A round bottom flask under nitrogen charged with Example 205A (1.9 g, 4.18 mmol), oxetan-3-ylmethanol (PharmaBlock, 0.553 g, 6.27 mmol) and anhydrous THF (15 mL) was cooled to −10° C. (ice-salt bath). After few minutes at that temperature potassium bis(trimethylsilyl)amide (1M solution in THF, 6.27 mL, 6.27 mmol) was added via a syringe and the solution was stirred gradually warming the reaction mixture to ambient temperature. After 1 h the reaction was quenched with addition of 35 mL of a saturated solution of ammonium chloride and extracted with EtOAc. The organic phase was dried over magnesium sulfate, filtered and evaporated to give a yellow solid. Silica gel chromatography using a gradient of heptane-EtOAc 0-70% EtOAc over 60 minutes gave the title compound (1.43 g, 65%) as a white solid. ¹H NMR (400 MHz, CDCl₃) δ ppm 10.35 (s, 1H), 8.58 (s, 1H), 8.41 (s, 1H), 7.44 (s, 1H), 4.95 (m, 2H), 4.52 (m, 2H), 4.38 (m, 2H), 4.18 (s, 6H), 3.55 (m 1H). MS (ESI) m/z 523 (M+H)⁺.

### Example 206

### 4,6-dimethoxy-N-(5-(methoxymethyl)-4-(trifluoromethyl)thiazol-2-yl)pyrimidine-5-carboxamide

Methanol (1.5 mL, 37 mmol), triethylsilane (4.4 mL, 27.54 mmol), and triethylsilyl trifluoromethanesulfonate (0.6 mL, 2.78 mmol) were added to a yellow solution of Example 47 (3.00 g, 8.28 mmol) in nitromethane (50 mL) and the reaction mixture stirred for 72 h at ambient temperature. The reaction mixture was concentrated to a yellow solid and chromatographed on a Grace Reveleris 120 g column, eluted with 0-100% EtOAc in Heptane (50 mL/min) to obtained the title compound (2.14 g, 68.4% yield) as a white solid. ¹H NMR (501 MHz, DMSO-d₆) δ 12.91 (s, 1H), 8.60 (s, 1H), 4.72 (q, J=1.7 Hz, 2H), 3.94 (s, 6H), 3.35 (s, 3H). MS (ESI+) m/z 378.9 (M+H).

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

What is claimed is:

1. A compound of Formula (I): 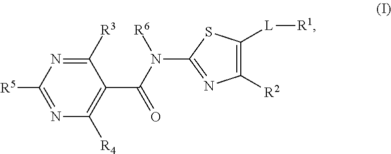
or a pharmaceutically acceptable salt thereof, wherein:
L is selected from the group consisting of a bond, —O—, —C(O)—, —C(O)O—, —C(O)N(R^{L})—, —S—, —S(O)—, and —S(O)₂—;
R^{L} is selected from the group consisting of hydrogen and C₁-C₆-alkyl;
R¹ is selected from the group consisting of hydrogen, halogen, cyano, imino, C₁-C₁₀-alkyl, C₂-C₁₀-alkenyl, C₂-C₁₀-alkynyl, C₃-C₆-cycloalkyl, C₅-C₁₀-cycloalkenyl, aryl, 4- to 10-membered ring heterocyclyl, and 5- to 10-membered ring heteroaryl; wherein:
the imino may be unsubstituted or substituted with one or two substituents independently selected from the group consisting of hydroxyl, C₁-C₆-alkyl, C₁-C₆-alkoxy, aryl and 5- to 10-membered ring heteroaryl;
the R¹ C₁-C₁₀-alkyl may be unsubstituted or substituted with one, two, or three substituents independently selected from the group consisting of halogen, amino, cyano, C₃-C₆-cycloalkyl, aryl, 5- to 10-membered ring heteroaryl, 4- to 10-membered ring heterocyclyl, —OR¹⁰¹, —C(O)OR¹⁰², and ═NR¹⁰³;
wherein the amino, C₃-C₆-cycloalkyl, aryl, 5- to 10-membered ring heteroaryl, 4- to 10-membered ring heterocyclyl substituents of the R¹ C₁-C₁₀-alkyl may be unsubstituted or substituted with one, two, or three substituents independently selected from the group consisting of halogen, C₁-C₆-alkyl, C₁-C₆-alkoxy, C₃-C₆-cycloalkyl, halo-C₁-C₆-alkyl, and 4- to 10-membered ring heterocyclyl;
wherein R¹⁰¹ and R¹⁰² are independently selected from the group consisting of hydrogen, C₁-C₆-alkyl, halo-C₁-C₆-alkyl, C₃-C₆-cycloalkyl, halo-C₃-C₆-cycloalkyl, C₁-C₆-alkyl-C₃-C₆-cycloalkyl, aryl, haloaryl, and C₁-C₆-alkylaryl; and
wherein R¹⁰³ is selected from the group consisting of hydrogen, hydroxy, C₁-C₆-alkyl, and C₁-C₆-alkoxy;
the R¹ C₂-C₁₀-alkenyl and the R¹ C₂-C₁₀-alkynyl may be unsubstituted or substituted with one, two, or three substituents independently selected from the group consisting of hydroxy, C₁-C₆-alkoxy, C₃-C₆-cycloalkyl, aryloxy, 5- to 10-membered ring heteroaryloxy, C₁-C₆-alkoxycarbonyl, alkylsilyl, and cyano-C₁-C₆-alkylcarbonylamino;
the R¹ C₃-C₆-cycloalkyl and the R¹ C₃-C₆-cycloalkenyl may be unsubstituted or substituted with one, two, or three substituents independently selected from the group consisting of halogen, cyano, oxo, OH, C₁-C₆-alkyl, halo-C₁-C₆-alkyl, C₁-C₆-alkoxy, halo-C₁-C₆-alkoxy, C₁-C₆-alkoxycarbonyl, C₁-C₆-alkyoxycarbonylamino, and halo-C₁-C₆-alkoxycarbonyl;
the R¹ aryl and the R¹ 5- to 10-membered ring heteroaryl may be unsubstituted or substituted with one, two, or three substituents independently selected from the group consisting of halogen, cyano, C₁-C₆-alkyl, C₁-C₆ alkoxy, hydroxy-C₁-C₆-alkyl, halo-C₁-C₆-alkyl, cyano-C₁-C₆-alkyl, C₁-C₆-alkoxy-C₁-C₆-alkyl, C₁-C₆-alkoxycarbonyl-C₁-C₆-alkyl, C₃-C₆-cycloalkyl, C₃-C₆-cycloalkyl-C₁-C₆-alkyl, C₅-C₁₀-cycloalkenyl, aryl, aryl-C₁-C₆-alkyl, 4- to 10-membered ring heterocyclyl, 4- to 10-membered ring heterocyclyl-C₁-C₆-alkyl, 5- to 10-membered ring heteroaryl, 5- to 10-membered ring heteroaryl-C₁-C₆-alkyl, —OR¹⁰⁴, C(O)OR¹⁰⁵, —N(R¹⁰⁶)C(O)R¹⁰⁷, —N(R¹⁰⁸)C(O)NR¹⁰⁹R¹¹⁰, —S(O)₂R¹¹¹, —S(O)₂NR¹¹²R¹¹³, and —NS(O)₂NR¹¹²R¹¹³;
wherein the C₃-C₆-cycloalkyl, C₃-C₆-cycloalkyl-C₁-C₆-alkyl, C₅-C₁₀-cycloalkenyl, aryl, aryl-C₁-C₆-alkyl, 4- to 10-membered ring heterocyclyl, 4- to 10-membered ring heterocyclyl-C₁-C₆-alkyl, 5- to 10-membered ring heteroaryl, and 5- to 10-membered ring heteroaryl-C₁-C₆-alkyl of the R¹ aryl and the R¹ 5- to 10-membered ring heteroaryl may be unsubstituted or substituted with one, two, or three substituents independently selected from the group consisting of halogen, cyano, C₁-C₆-alkyl, halo-C₁-C₆-alkyl, cyano-C₁-C₆-alkyl, and C₁-C₆-alkoxycarbonyl;
wherein R¹⁰⁴, R¹⁰⁵, R¹⁰⁷, R¹⁰⁹, R¹¹⁰, R¹¹¹, R¹¹², and R¹¹³ are independently selected from the group consisting of hydrogen, C₁-C₆-alkyl, C₁-C₆-alkoxyC₁-C₆-alkyl, halo-C₁-C₆-alkyl, cyano-C₁-C₆-alkyl, C₃-C₆-cycloalkyl, halo-C₃-C₆-cycloalkyl, 4- to 10-membered ring heterocyclyl, 4- to 10-membered ring heterocyclyl-C₁-C₆-alkyl, aryl, haloaryl, and C₁-C₆-alkylaryl; and
wherein R¹⁰⁶ and R¹⁰⁸ are independently selected from the group consisting of hydrogen and C₁-C₆-alkyl; and
the R¹ 4- to 10-membered ring heterocyclyl may be unsubstituted or substituted with one, two, or three substituents independently selected from the group consisting of halogen, OH, C₁-C₆-alkyl, halo-C₁-C₆-alkyl, C₁-C₆-alkoxycarbonyl, 4- to 10-membered ring heterocyclyl, and —S(O)₂R¹⁴; wherein R¹⁴ is selected from the group consisting of hydrogen and C₁-C₆-alkyl;
R² is selected from the group consisting of C₁-C₆-alkyl, C₂-C₆-alkenyl, C₃-C₆-cycloalkyl, aryl, 4- to 10-membered ring heterocyclyl, and 5- to 10-membered ring heteroaryl; wherein:
the R² C₁-C₆-alkyl or the C₂-C₆-alkenyl may be unsubstituted or substituted with one, two, or three substituents independently selected from the group consisting of halogen, hydroxy, cyano, C₁-C₆-alkoxy, halo-C₁-C₆-alkoxy, C₃-C₆-cycloalkyl, halo-C₃-C₆-cycloalkyl, C₃-C₆-cycloalkoxy, halo-C₃-C₆-cycloalkoxy, aryl, haloaryl, aryloxy, haloaryloxy, and C₁-C₆-alkylaryloxy;
the R² C₃-C₆-cycloalkyl may be unsubstituted or substituted with one, two, or three substituents independently selected from the group consisting of halogen, C₁-C₆-alkyl, and halo-C₁-C₆-alkyl;
the R² aryl and the R² 5- to 10-membered ring heteroaryl may be unsubstituted or substituted with one, two, or three substituents independently selected from the group consisting of halogen, hydroxy, cyano, C₁-C₆-alkyl, hydroxy-C₁-C₆-alkyl, halo-C₁-C₆-alkyl, cyano-C₁-C₆-alkyl, C₃-C₆-cycloalkyl, C₁-C₆-alkoxy, halo-C₁-C₆-alkoxy, —S(O)₂R²⁰¹, and —S(O)₂NR²⁰²R²⁰³; wherein R²⁰¹, R²⁰², and R²⁰³ are independently selected from the group consisting of hydrogen and C₁-C₆-alkyl; and
the R² 4- to 10-membered ring heterocyclyl may be unsubstituted or substituted with one, two, or three substituents independently selected from the group consisting of halogen, C₁-C₆-alkyl, and halo-C₁-C₆-alkyl;
R³ and R⁴ are independently selected from the group consisting of hydrogen, C₁-C₆-alkyl, C₁-C₆-alkoxy, C₁-C₆-alkylthio, and C₃-C₆-cycloalkyl, wherein the C₁-C₆-alkyl may be substituted with one, two, or three halogen;
R⁵ is selected from the group consisting of hydrogen, methyl, methoxy, and cyano; and
R⁶ is selected from the group consisting of hydrogen or —CH₂-phosphate.

2. The compound of claim 1, or the pharmaceutically acceptable salt thereof, wherein R¹ comprises aryl or 5- to 10-membered ring heteroaryl selected from the group consisting of phenyl, pyridinyl, pyrimidinyl, pyrazinyl, 1H-indolyl, 2H-indolyl, pyrazolyl, 1H-imidazolyl, oxazolyl, isoxazolyl, pyrazolyl, quinolinyl, isoquinolinyl, furo[3,2-b]pyridinyl, furo[4,3-b]pyridinyl, furo[5,4-b]pyridinyl, and benzo[c][1,2,5]oxadiazol-5-yl, each of which may be substituted or unsubstituted.

3. The compound of claim 1, or the pharmaceutically acceptable salt thereof, wherein R¹ comprises C₃-C₆-cycloalkyl or C₃-C₆-cycloalkenyl selected from the group consisting of cyclopropyl, cyclobutyl, cyclobutenyl, cyclopropyl, cyclopropenyl, cyclohexyl, and cyclohexenyl, each of which may be substituted or unsubstituted.

4. The compound of claim 1, or the pharmaceutically acceptable salt thereof, wherein R¹ comprises 4- to 10-membered ring heterocyclyl selected from the group consisting of 2,3-dihydrofuranyl, 2,5-dihydrofuranyl, tetrahydrofuranyl, 1,3-dioxolanyl, 1,3-dioxanyl, tetrahydro-2H-pyranyl, 3,4-dihydro-2H-pyranyl, 3,6-dihydro-2H-pyranyl, 2H-pyranyl, 4H-pyranyl, pyrrolidinyl, 2,3-dihydro-1H-pyrrolyl, 2,5-dihydro-1H-pyrrolyl, 4H-1,3-dioxinyl, 1,4-dioxanyl, 2,3-dihydro-1,4-dioxinyl, piperidinyl, 2-oxa-7-azaspiro[3.5]nonanyl, 1,2-dihydropyridinyl, 1,4-dihydropyridinyl, 2,3-dihydropyridinyl, 3,4-dihydropyridinyl, 1,2,3,6-tetrahydropyridinyl, isoxazolidinyl, oxazolidinyl, 2,3-dihydroisoxazolyl, 2,5-dihydroisoxazolyl, and morpholino, each of which may be substituted or unsubstituted.

5. The compound of claim 1, or the pharmaceutically acceptable salt thereof, wherein R¹ is selected from the group consisting of hydrogen, methyl, ethyl, propyl, isopropyl, trifluoromethyl, ethenyl, ethynyl, propenyl, propynyl, t-butyl, butenyl, butynyl, cyano, iodo, chloro, fluoro, and bromo, each of which may be substituted or unsubstituted.

6. The compound of claim 1, or the pharmaceutically acceptable salt thereof, wherein R² is selected from the group consisting of methyl, trifluoromethyl, ethyl, n-propyl, isopropyl, tert-butyl, neopentyl, cyclopropylethyl, cyclopropyl, cyclobutyl, cyclopentyl, cyclohexyl, difluorocyclohexyl, cyclopropylvinyl, dimethylbutenyl, fluorostyryl.

7. The compound of claim 1, or the pharmaceutically acceptable salt thereof, wherein R² is selected from the group consisting of phenyl, trifluoromethoxyphenyl, 4-fluoro-2-methoxyphenyl, para-fluorophenethyl, para-fluorophenoxyethyl, toylyoxymethyl, meta-trifluoromethylpyridinyl, para-trifluoromethylpyridinyl, para-fluoropyridinyl, para-trifluoromethoxypyridinyl, para-difluoromethylpyridinyl.

8. The compound of claim 1, or the pharmaceutically acceptable salt thereof, wherein R⁵ and R⁶ are hydrogen.

9. The compound of claim 8, or the pharmaceutically acceptable salt thereof, of Formula (II-A): 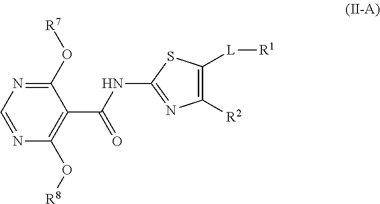
or the pharmaceutically acceptable salt thereof,
wherein L, R¹, and R² are as defined in claim 1; and
R⁷ and R⁸ are independently selected from the group consisting of hydrogen, C₁-C₆-alkyl, and C₃-C₆-cycloalkyl.

10. The compound of claim 8, or the pharmaceutically acceptable salt thereof, of Formula (II-B): 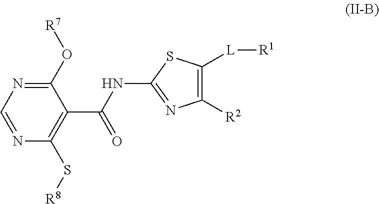
or the pharmaceutically acceptable salt thereof,
wherein L, R¹, and R² are as defined in claim 1; and
R⁷ and R⁸ are independently selected from the group consisting of hydrogen, C₁-C₆-alkyl, and C₃-C₆-cycloalkyl.

11. The compound of claim 1, or the pharmaceutically acceptable salt thereof, of Formula (III): 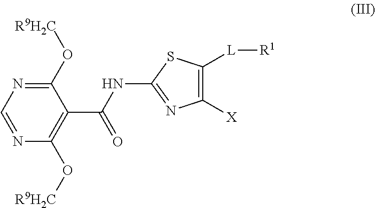
or the pharmaceutically acceptable salt thereof,
wherein L and R¹ are as defined in claim 1;
X is either C(R¹⁰)₃, pyridine-C(R¹⁰)₃, or pyridine-O—C(R¹⁰)₃;
each R⁹ is independently —H or —CH₃; and
each R¹⁰ is independently —H, —CH₃, or —F.

12. The compound of claim 11, or the pharmaceutically acceptable salt thereof, wherein each R⁹ is —H or —CH₃ and X is selected from the group consisting of —CF₃, pyridine-CF₃, and —C(CH₃)₃.

13. The compound of claim 11, or the pharmaceutically acceptable salt thereof, wherein each R⁹ is —H, X is —CF₃, L is a bond, and R¹ is halo.

14. The compound of claim 11, or the pharmaceutically acceptable salt thereof, wherein each R⁹ is —H or —CH₃, X is —CF₃, L is a bond, and R¹ is C₁-C₁₀ alkyl, which may be substituted or unsubstituted.

15. The compound of claim 14, or the pharmaceutically acceptable salt thereof, wherein R¹ is C₁-C₁₀ alkyl substituted with OR¹⁰¹.

16. The compound of claim 11, or the pharmaceutically acceptable salt thereof, wherein each R⁹ is —H, X is —CF₃, L is a bond, and R¹ is methyl, substituted with OR¹⁰¹, and further wherein R¹⁰¹ is methyl.

17. The compound of claim 14, or the pharmaceutically acceptable salt thereof, wherein R¹ is C₁-C₁₀ alkyl substituted with amino.

18. The compound of claim 14, or the pharmaceutically acceptable salt thereof, wherein R¹ is C₁-C₁₀ alkyl substituted with 4- to 10-membered ring heterocyclyl.

19. The compound of claim 14, or the pharmaceutically acceptable salt thereof, wherein R¹ is C₁-C₁₀ alkyl substituted with aryl.

20. The compound of claim 11, or the pharmaceutically acceptable salt thereof, wherein each R⁹ is —H, X is —CF₃, L is a bond, and R¹ is C₂-C₁₀-alkenyl, which may be substituted or unsubstituted.

21. The compound of claim 20, or the pharmaceutically acceptable salt thereof, wherein R¹ is C₂-C₁₀-alkenyl substituted with C₁-C₆ alkoxy.

22. The compound of claim 11, or the pharmaceutically acceptable salt thereof, wherein each R⁹ is —H, X is —CF₃, L is a bond, and R¹ is C₂-C₁₀-alkynyl, which may be substituted or unsubstituted.

23. The compound of claim 22, or the pharmaceutically acceptable salt thereof, wherein R¹ is C₂-C₁₀-alkynyl substituted with C₃-C₆ cycloalkyl.

24. The compound of claim 22, or the pharmaceutically acceptable salt thereof, wherein R¹ is C₂-C₁₀-alkynyl substituted with C₁-C₆ alkoxy.

25. The compound of claim 11, or the pharmaceutically acceptable salt thereof, wherein each R⁹ is —H, X is —CF₃, L is a bond, and R¹ is aryl, which may be unsubstituted.

26. The compound of claim 11, or the pharmaceutically acceptable salt thereof, wherein R¹ is phenyl, substituted with one or more substituents independently selected from cyano, halogen, —OR¹⁰⁴, —S(O)₂NR¹¹²R¹¹³ or 5- to 10-membered ring heteroaryl-C₁-C₆-alkyl.

27. The compound of claim 11, or the pharmaceutically acceptable salt thereof, wherein each R⁹ is —H, X is —CF₃, L is a bond, and R¹ is 5- to 10-membered ring heteroaryl, which may be substituted or unsubstituted.

28. The compound of claim 27, or the pharmaceutically acceptable salt thereof, wherein R¹ is pyridine substituted with one or more substituents independently selected from C₁-C₆-alkyl, C₁-C₄ alkoxy, cyano-C₁-C₆-alkyl and morpholine.

29. The compound of claim 27, or the pharmaceutically acceptable salt thereof, wherein R¹ is pyridine substituted with C₃-C₆-cycloalkyl or 4- to 10-membered ring heterocyclyl, and further wherein the C₃-C₆-cycloalkyl or the 4- to 10-membered ring heterocyclyl may be substituted or unsubstituted.

30. The compound of claim 27, or the pharmaceutically acceptable salt thereof, wherein R¹ is pyridine substituted with one or more substituents independently selected from C₁-C₄ alkoxy, cyano, halo, halo-C₁-C₆-alkyl, or —OR¹⁰⁴.

31. The compound of claim 27, or the pharmaceutically acceptable salt thereof, wherein R¹ is pyridine substituted with cyano and —OR¹⁰⁴.

32. The compound of claim 31, or the pharmaceutically acceptable salt thereof, wherein R¹⁰⁴ is C₁-C₆-alkyl substituted with 4- to 10-membered heterocyclyl, which may be substituted or unsubstituted.

33. The compound of claim 31, or the pharmaceutically acceptable salt thereof, wherein R¹⁰⁴ is methyl substituted with 4- to 10-membered heterocyclyl, which may be substituted or unsubstituted.

34. The compound of claim 31, or the pharmaceutically acceptable salt thereof, wherein R¹⁰⁴ is methyl substituted with oxetanyl.

35. The compound of claim 27, or the pharmaceutically acceptable salt thereof, wherein R¹ is pyrazole substituted with one or more substituents independently selected from C₁-C₆-alkyl, halo, halo-C₁-C₆-alkyl, or with C₃-C₆-cycloalkyl, wherein the C₁-C₆-alkyl or the C₃-C₆-cycloalkyl may be substituted or unsubstituted.

36. The compound of claim 27, or the pharmaceutically acceptable salt thereof, wherein R¹ is pyrimidine substituted with halo-C₁-C₆-alkyl or C₃-C₆-cycloalkyl, which C₃-C₆-cycloalkyl may be substituted or unsubstituted.

37. The compound of claim 27, or the pharmaceutically acceptable salt thereof, wherein R¹ is quinoline, isoxazole or benzo[c][1,2,5]oxadiazole.

38. The compound of claim 11, or the pharmaceutically acceptable salt thereof, wherein each R⁹ is —H, X is —CF₃, L is a bond, and R¹ is C₃-C₆-cycloalkyl.

39. The compound of claim 11, or the pharmaceutically acceptable salt thereof, wherein each R⁹ is —H, X is —CF₃, L is a bond, and R¹ is C₅-C₁₀-cycloalkenyl.

40. The compound of claim 11, or the pharmaceutically acceptable salt thereof, wherein each R⁹ is —H, X is —CF₃, L is a bond, and R¹ is 4- to 10-membered ring heterocyclyl, which may be substituted or unsubstituted or R¹ is substituted imino.

41. The compound of claim 40, or the pharmaceutically acceptable salt thereof, wherein R¹ is pyridine substituted by cyano-C₁-C₆-alkyl.

42. The compound of claim 11, or the pharmaceutically acceptable salt thereof, wherein each R⁹ is —H, X is —CF₃, L is —C(O)— or —C(O)O—, and R¹ is C₁-C₁₀ alkyl.

43. The compound of claim 11, or the pharmaceutically acceptable salt thereof, wherein each R⁹ is —H, X is —CF₃, L is —C(O)—, and R¹ is 5- to 10-membered ring heteroaryl, which may be substituted or unsubstituted.

44. The compound of claim 11, or the pharmaceutically acceptable salt thereof, wherein each R⁹ is —H, X is pyridine-CF₃, L is a bond, and R¹ is hydrogen or R¹ is C₁-C₁₀ alkyl, which may be unsubstituted or substituted with OR¹⁰¹.

45. The compound of claim 11, or the pharmaceutically acceptable salt thereof, wherein each R⁹ is —H, X is pyridine-CF₃, L is a bond, and R¹ is substituted imino.

46. The compound of claim 11, or the pharmaceutically acceptable salt thereof, wherein each R⁹ is —H, X is —C(CH₃)₃, L is a bond, and R¹ is halo or cyano.

47. The compound of claim 11, or the pharmaceutically acceptable salt thereof, wherein each R⁹ is —H, X is —C(CH₃)₃, L is —C(O)O—, and R¹ is C₁-C₁₀ alkyl.

48. The compound of claim 1, or the pharmaceutically acceptable salt thereof, wherein the compound is selected from the group consisting of:
4,6-dimethoxy-N-[5-methyl-4-(trifluoromethyl)-1,3-thiazol-2-yl]pyrimidine-5-carboxamide;
N-[5-iodo-4-(trifluoromethyl)-1,3-thiazol-2-yl]-4,6-dimethoxypyrimidine-5-carboxamide;
4,6-dimethoxy-N-[4-(trifluoromethyl)-1,3-thiazol-2-yl]pyrimidine-5-carboxamide;
N-[5-(4-chlorophenyl)-4-(trifluoromethyl)-1,3-thiazol-2-yl]-4,6-dimethoxypyrimidine-5-carboxamide;
N-[5-(4-chlorophenyl)-4-(trifluoromethyl)-1,3-thiazol-2-yl]-4,6-diethoxypyrimidine-5-carboxamide;
N-[5-ethyl-4-(trifluoromethyl)-1,3-thiazol-2-yl]-4,6-dimethoxypyrimidine-5-carboxamide;
N-[5-cyano-4-(trifluoromethyl)-1,3-thiazol-2-yl]-4,6-diethoxypyrimidine-5-carboxamide;
N-[5-(6-fluoropyridin-3-yl)-4-(trifluoromethyl)-1,3-thiazol-2-yl]-4,6-dimethoxypyrimidine-5-carboxamide;
N-(4,5-dimethyl-1,3-thiazol-2-yl)-4,6-dimethoxypyrimidine-5-carboxamide;
4,6-dimethoxy-N-{4-(trifluoromethyl)-5-[(trimethylsilyl)ethynyl]-1,3-thiazol-2-yl}pyrimidine-5-carboxamide;
N-[5-ethynyl-4-(trifluoromethyl)-1,3-thiazol-2-yl]-4,6-dimethoxypyrimidine-5-carboxamide;
N-[5-(3-hydroxyprop-1-yn-1-yl)-4-(trifluoromethyl)-1,3-thiazol-2-yl]-4,6-dimethoxypyrimidine-5-carboxamide;
N-[5-(3-hydroxypropyl)-4-(trifluoromethyl)-1,3-thiazol-2-yl]-4,6-dimethoxypyrimidine-5-carboxamide;
N-[5-(cyclopropylethynyl)-4-(trifluoromethyl)-1,3-thiazol-2-yl]-4,6-dimethoxypyrimidine-5-carboxamide;
4,6-dimethoxy-N-[5-(3-methoxyprop-1-yn-1-yl)-4-(trifluoromethyl)-1,3-thiazol-2-yl]pyrimidine-5-carboxamide;
4,6-dimethoxy-N-[5-(3-methoxypropyl)-4-(trifluoromethyl)-1,3-thiazol-2-yl]pyrimidine-5-carboxamide;
4,6-dimethoxy-N-{5-[(1Z)-3-methoxyprop-1-en-1-yl]-4-(trifluoromethyl)-1,3-thiazol-2-yl}pyrimidine-5-carboxamide;
4,6-dimethoxy-N-{5-[(1E)-3-methoxyprop-1-en-1-yl]-4-(trifluoromethyl)-1,3-thiazol-2-yl}pyrimidine-5-carboxamide;
N-[5-(3-hydroxy-3-methylbut-1-yn-1-yl)-4-(trifluoromethyl)-1,3-thiazol-2-yl]-4,6-dimethoxypyrimidine-5-carboxamide;
N-[5-(3-hydroxy-3-methylbutyl)-4-(trifluoromethyl)-1,3-thiazol-2-yl]-4,6-dimethoxypyrimidine-5-carboxamide;
ethyl 2-{[(4,6-dimethoxypyrimidin-5-yl)carbonyl]amino}-4-(trifluoromethyl)-1,3-thiazole-5-carboxylate;
N-[5-(2-hydroxyethyl)-4-(trifluoromethyl)-1,3-thiazol-2-yl]-4,6-dimethoxypyrimidine-5-carboxamide;
4,6-dimethoxy-N-{5-[6-(morpholin-4-yl)pyridin-3-yl]-4-(trifluoromethyl)-1,3-thiazol-2-yl}pyrimidine-5-carboxamide;
N-[5-(3-cyanopyridin-4-yl)-4-(trifluoromethyl)-1,3-thiazol-2-yl]-4,6-dimethoxypyrimidine-5-carboxamide;
N-[5-(2-cyano-3-fluorophenyl)-4-(trifluoromethyl)-1,3-thiazol-2-yl]-4,6-dimethoxypyrimidine-5-carboxamide;
N-(5-ethyl-4-phenyl-1,3-thiazol-2-yl)-4,6-dimethoxypyrimidine-5-carboxamide;
N-[5-(5-fluoropyridin-3-yl)-4-(trifluoromethyl)-1,3-thiazol-2-yl]-4,6-dimethoxypyrimidine-5-carboxamide;
4,6-dimethoxy-N-[5-(pyrazin-2-yl)-4-(trifluoromethyl)-1,3-thiazol-2-yl]pyrimidine-5-carboxamide;
4,6-dimethoxy-N-{5-[5-(morpholin-4-yl)pyrazin-2-yl]-4-(trifluoromethyl)-1,3-thiazol-2-yl}pyrimidine-5-carboxamide;
4,6-dimethoxy-N-{4-(trifluoromethyl)-5-[6-(trifluoromethyl)pyridin-2-yl]-1,3-thiazol-2-yl}pyrimidine-5-carboxamide;
N-[5-(6-cyanopyridin-2-yl)-4-(trifluoromethyl)-1,3-thiazol-2-yl]-4,6-dimethoxypyrimidine-5-carboxamide;
4,6-dimethoxy-N-[5-(morpholin-4-yl)-4-(trifluoromethyl)-1,3-thiazol-2-yl]pyrimidine-5-carboxamide;
N-{5-[2-(2-cyanopropan-2-yl)pyridin-4-yl]-4-(trifluoromethyl)-1,3-thiazol-2-yl}-4,6-dimethoxypyrimidine-5-carboxamide;
4,6-dimethoxy-N-[5-propyl-4-(trifluoromethyl)-1,3-thiazol-2-yl]pyrimidine-5-carboxamide;
N-[5-(2,2-dimethyl-1,3-dioxolan-4-yl)-4-(trifluoromethyl)-1,3-thiazol-2-yl]-4,6-dimethoxypyrimidine-5-carboxamide;
4,6-dimethoxy-N-{5-[2-(morpholin-4-yl)pyrimidin-5-yl]-4-(trifluoromethyl)-1,3-thiazol-2-yl}pyrimidine-5-carboxamide;
N-[5-cyclopropyl-4-(trifluoromethyl)-1,3-thiazol-2-yl]-4,6-dimethoxypyrimidine-5-carboxamide;
N-[5-(3,6-dihydro-2H-pyran-4-yl)-4-(trifluoromethyl)-1,3-thiazol-2-yl]-4,6-dimethoxypyrimidine-5-carboxamide;
4,6-dimethoxy-N-[5-(tetrahydro-2H-pyran-4-yl)-4-(trifluoromethyl)-1,3-thiazol-2-yl]pyrimidine-5-carboxamide;
N-[5-(3-cyanophenyl)-4-(trifluoromethyl)-1,3-thiazol-2-yl]-4,6-dimethoxypyrimidine-5-carboxamide;
N-[5-(4-cyanophenyl)-4-(trifluoromethyl)-1,3-thiazol-2-yl]-4,6-dimethoxypyrimidine-5-carboxamide;
N-[5-(2-cyanopyridin-3-yl)-4-(trifluoromethyl)-1,3-thiazol-2-yl]-4,6-dimethoxypyrimidine-5-carboxamide;
N-[5-(4-cyano-2-methoxyphenyl)-4-(trifluoromethyl)-1,3-thiazol-2-yl]-4,6-dimethoxypyrimidine-5-carboxamide;
N-[5-(6-cyano-1H-indol-3-yl)-4-(trifluoromethyl)-1,3-thiazol-2-yl]-4,6-dimethoxypyrimidine-5-carboxamide;
4,6-dimethoxy-N-[5-(pyridin-2-yl)-4-(trifluoromethyl)-1,3-thiazol-2-yl]pyrimidine-5-carboxamide;
N-[5-(5-chloropyridin-2-yl)-4-(trifluoromethyl)-1,3-thiazol-2-yl]-4,6-dimethoxypyrimidine-5-carboxamide;
N-[5-formyl-4-(trifluoromethyl)-1,3-thiazol-2-yl]-4,6-dimethoxypyrimidine-5-carboxamide;
4,6-dimethoxy-N-{5-[(4-methoxypiperidin-1-yl)methyl]-4-(trifluoromethyl)-1,3-thiazol-2-yl}pyrimidine-5-carboxamide;
4,6-dimethoxy-N-[5-(pyrrolidin-1-ylmethyl)-4-(trifluoromethyl)-1,3-thiazol-2-yl]pyrimidine-5-carboxamide;
4,6-dimethoxy-N-[5-(morpholin-4-ylmethyl)-4-(trifluoromethyl)-1,3-thiazol-2-yl]pyrimidine-5-carboxamide;
N-{5-[(cyclopentylamino)methyl]-4-(trifluoromethyl)-1,3-thiazol-2-yl}-4,6-dimethoxypyrimidine-5-carboxamide;
4,6-dimethoxy-N-[5-{[(2-methylpropyl)amino]methyl}-4-(trifluoromethyl)-1,3-thiazol-2-yl]pyrimidine-5-carboxamide;
N-{5-[(4-fluoropiperidin-1-yl)methyl]-4-(trifluoromethyl)-1,3-thiazol-2-yl}-4,6-dimethoxypyrimidine-5-carboxamide;
4,6-dimethoxy-N-[5-{[methyl(2-methylpropyl)amino]methyl}-4-(trifluoromethyl)-1,3-thiazol-2-yl]pyrimidine-5-carboxamide;
N-[5-{[tert-butyl(methyl)amino]methyl}-4-(trifluoromethyl)-1,3-thiazol-2-yl]-4,6-dimethoxypyrimidine-5-carboxamide;
N-{5-[(tert-butylamino)methyl]-4-(trifluoromethyl)-1,3-thiazol-2-yl}-4,6-dimethoxypyrimidine-5-carboxamide;
N-[5-{[(3 S)-3-fluoropyrrolidin-1-yl]methyl}-4-(trifluoromethyl)-1,3-thiazol-2-yl]-4,6-dimethoxypyrimidine-5-carboxamide;
N-[5-{[(3R)-3-fluoropyrrolidin-1-yl]methyl}-4-(trifluoromethyl)-1,3-thiazol-2-yl]-4,6-dimethoxypyrimidine-5-carboxamide;
4,6-dimethoxy-N-[5-{[(3S)-3-methoxypyrrolidin-1-yl]methyl}-4-(trifluoromethyl)-1,3-thiazol-2-yl]pyrimidine-5-carboxamide;
4,6-dimethoxy-N-[5-{[(3R)-3-methoxypyrrolidin-1-yl]methyl}-4-(trifluoromethyl)-1,3-thiazol-2-yl]pyrimidine-5-carboxamide;
4,6-dimethoxy-N-[5-(2-oxa-7-azaspiro[3.5]non-7-ylmethyl)-4-(trifluoromethyl)-1,3-thiazol-2-yl]pyrimidine-5-carboxamide;
4,6-dimethoxy-N-[5-(2-oxa-6-azaspiro[3.5]non-6-ylmethyl)-4-(trifluoromethyl)-1,3-thiazol-2-yl]pyrimidine-5-carboxamide;
N-[5-chloro-4-(trifluoromethyl)-1,3-thiazol-2-yl]-4,6-dimethoxypyrimidine-5-carboxamide;
4,6-dimethoxy-N-[5-(2-methylpyridin-3-yl)-4-(trifluoromethyl)-1,3-thiazol-2-yl]pyrimidine-5-carboxamide;
4,6-dimethoxy-N-{5-[1-methyl-3-(trifluoromethyl)-1H-pyrazol-5-yl]-4-(trifluoromethyl)-1,3-thiazol-2-yl}pyrimidine-5-carboxamide;
N-[5-(2-cyanopyridin-4-yl)-4-(trifluoromethyl)-1,3-thiazol-2-yl]-4,6-dimethoxypyrimidine-5-carboxamide;
N-[5-(1,3-dimethyl-1H-pyrazol-4-yl)-4-(trifluoromethyl)-1,3-thiazol-2-yl]-4,6-dimethoxypyrimidine-5-carboxamide;
{[(4,6-dimethoxypyrimidin-5-yl)carbonyl][5-(1,3-dimethyl-1H-pyrazol-4-yl)-4-(trifluoromethyl)-1,3-thiazol-2-yl]amino}methyl dihydrogen phosphate;
N-[5-ethyl-4-(trifluoromethyl)-1,3-thiazol-2-yl]-4,6-dimethoxy-2-methylpyrimidine-5-carboxamide;
4-ethoxy-N-[5-ethyl-4-(trifluoromethyl)-1,3-thiazol-2-yl]-6-methoxypyrimidine-5-carboxamide;
4,6-diethoxy-N-[5-ethyl-4-(trifluoromethyl)-1,3-thiazol-2-yl]pyrimidine-5-carboxamide;
4,6-dimethoxy-N-(5-(pyridin-4-yloxy)-4-(trifluoromethyl)thiazol-2-yl)pyrimidine-5-carboxamide;
4,6-dimethoxy-N-{4-[5-(trifluoromethyl)pyridin-2-yl]-1,3-thiazol-2-yl}pyrimidine-5-carboxamide;
([(4,6-dimethoxypyrimidin-5-yl)carbonyl]{4-[5-(trifluoromethyl)pyridin-2-yl]-1,3-thiazol-2-yl}amino)methyl dihydrogen phosphate;
ethyl 2-{[(4,6-dimethoxypyrimidin-5-yl)carbonyl]amino}-4-[5-(trifluoromethyl)pyridin-2-yl]-1,3-thiazole-5-carboxylate;
N-{5-formyl-4-[5-(trifluoromethyl)pyridin-2-yl]-1,3-thiazol-2-yl}-4,6-dimethoxypyrimidine-5-carboxamide;
4,6-dimethoxy-N-{5-(morpholin-4-ylmethyl)-4-[5-(trifluoromethyl)pyridin-2-yl]-1,3-thiazol-2-yl}pyrimidine-5-carboxamide;
4,6-dimethoxy-N-{5-(pyrrolidin-1-ylmethy)-4-[5-(trifluoromethyl)pyridin-2-yl]-1,3-thiazol-2-yl}pyrimidine-5-carboxamide;
N-{5-(dimethylcarbamoyl)-4-[5-(trifluoromethyl)pyridin-2-yl]-1,3-thiazol-2-yl}-4-hydroxy-6-methoxypyrimidine-5-carboxamide;
N-{5-(hydroxymethyl)-4-[5-(trifluoromethyl)pyridin-2-yl]-1,3-thiazol-2-yl}-4,6-dimethoxypyrimidine-5-carboxamide;
([(4,6-dimethoxypyrimidin-5-yl)carbonyl]{5-(hydroxymethyl)-4-[5-(trifluoromethyl)pyridin-2-yl]-1,3-thiazol-2-yl}amino)methyl dihydrogen phosphate;
N-{5-(1-hydroxyethyl)-4-[4-(trifluoromethoxy)phenyl]-1,3-thiazol-2-yl}-4,6-dimethoxypyrimidine-5-carboxamide;
N-{5-[(1R*)-1-hydroxyethyl]-4-[5-(trifluoromethyl)pyridin-2-yl]-1,3-thiazol-2-yl}-4,6-dimethoxypyrimidine-5-carboxamide;
N-{5-[(1S*)-1-hydroxyethyl]-4-[5-(trifluoromethyl)pyridin-2-yl]-1,3-thiazol-2-yl}-4,6-dimethoxypyrimidine-5-carboxamide;
N-{5-(2-hydroxypropan-2-yl)-4-[5-(trifluoromethyl)pyridin-2-yl]-1,3-thiazol-2-yl}-4,6-dimethoxypyrimidine-5-carboxamide;
4,6-dimethoxy-N-{5-(methoxymethyl)-4-[5-(trifluoromethyl)pyridin-2-yl]-1,3-thiazol-2-yl}pyrimidine-5-carboxamide;
N-{5-acetyl-4-[5-(trifluoromethyl)pyridin-2-yl]-1,3-thiazol-2-yl}-4,6-dimethoxypyrimidine-5-carboxamide;
N-{5-[(1E)-N-hydroxyethanimidoyl]-4-[5-(trifluoromethyl)pyridin-2-yl]-1,3-thiazol-2-yl}-4,6-dimethoxypyrimidine-5-carboxamide;
4,6-dimethoxy-N-{5-[(1E)-N-methoxyethanimidoyl]-4-[5-(trifluoromethyl)pyridin-2-yl]-1,3-thiazol-2-yl}pyrimidine-5-carboxamide;
4,6-dimethoxy-N-{5-(2-methyl-1,3-dioxolan-2-yl)-4-[5-(trifluoromethyl)pyridin-2-yl]-1,3-thiazol-2-yl}pyrimidine-5-carboxamide;
N-[4-(5-fluoropyridin-2-yl)-1,3-thiazol-2-yl]-4,6-dimethoxypyrimidine-5-carboxamide;
4,6-dimethoxy-N-{4-[5-(trifluoromethoxy)pyridin-2-yl]-1,3-thiazol-2-yl}pyrimidine-5-carboxamide;
4,6-dimethoxy-N-{4-[4-(trifluoromethoxy)phenyl]-1,3-thiazol-2-yl}pyrimidine-5-carboxamide;
N-{4-[5-(difluoromethyl)pyridin-2-yl]-1,3-thiazol-2-yl}-4,6-dimethoxypyrimidine-5-carboxamide;
4,6-dimethoxy-N-{5-methyl-4-[5-(trifluoromethyl)pyridin-2-yl]-1,3-thiazol-2-yl}pyrimidine-5-carboxamide;
N-{5-(3-hydroxypropyl)-4-[5-(trifluoromethyl)pyridin-2-yl]-1,3-thiazol-2-yl}-4,6-dimethoxypyrimidine-5-carboxamide;
N-{5-cyano-4-[5-(trifluoromethyl)pyridin-2-yl]-1,3-thiazol-2-yl}-4,6-dimethoxypyrimidine-5-carboxamide;
N-(4-cyclopentyl-1,3-thiazol-2-yl)-4,6-dimethoxypyrimidine-5-carboxamide;
N-[4-(4,4-difluorocyclohexyl)-1,3-thiazol-2-yl]-4,6-dimethoxypyrimidine-5-carboxamide;
N-(4-tert-butyl-5-fluoro-1,3-thiazol-2-yl)-4,6-dimethoxypyrimidine-5-carboxamide;
N-(4-cyclobutyl-5-fluoro-1,3-thiazol-2-yl)-4,6-dimethoxypyrimidine-5-carboxamide;
N-(5-cyano-4-cyclobutyl-1,3-thiazol-2-yl)-4,6-dimethoxypyrimidine-5-carboxamide;
N-{4-[(E)-2-cyclopropylethenyl]-1,3-thiazol-2-yl}-4,6-dimethoxypyrimidine-5-carboxamide;
N-{4-[(1E)-3,3-dimethylbut-1-en-1-yl]-1,3-thiazol-2-yl}-4,6-dimethoxypyrimidine-5-carboxamide;
N-[4-(2-cyclopropylethyl)-1,3-thiazol-2-yl]-4,6-dimethoxypyrimidine-5-carboxamide;
N-{4-[(E)-2-(4-fluorophenyl)ethenyl]-1,3-thiazol-2-yl}-4,6-dimethoxypyrimidine-5-carboxamide;
N-{4-[2-(4-fluorophenyl)ethyl]-1,3-thiazol-2-yl}-4,6-dimethoxypyrimidine-5-carboxamide;
N-[4-(4-fluoro-2-methoxyphenyl)-1,3-thiazol-2-yl]-4,6-dimethoxypyrimidine-5-carboxamide;
4,6-dimethoxy-N-[5-(p-tolylsulfonyl)-4-[5-(trifluoromethyl)-2-pyridyl]-1,3-thiazol-2-yl]pyrimidine-5-carboxamide;
4,6-dimethoxy-N-[5-(p-tolylsulfonyl)-4-(trifluoromethyl)-1,3-thiazol-2-yl]pyrimidine-5-carboxamide;
N-[5-benzylsulfonyl-4-(trifluoromethyl)-1,3-thiazol-2-yl]-4,6-dimethoxy-pyrimidine-5-carboxamide;
N-(5-(benzylsulfinyl)-4-(trifluoromethyl)thiazol-2-yl)-4,6-dimethoxypyrimidine-5-carboxamide;
4,6-dimethoxy-N-[5-methylsulfinyl-4-[5-(trifluoromethyl)-2-pyridyl]-1,3-thiazol-2-yl]pyrimidine-5-carboxamide;
4,6-dimethoxy-N-[5-methylsulfonyl-4-[5-(trifluoromethyl)-2-pyridyl]-1,3-thiazol-2-yl]pyrimidine-5-carboxamide;
4,6-dimethoxy-N-[5-phenylsulfanyl-4-(trifluoromethyl)-1,3-thiazol-2-yl]pyrimidine-5-carboxamide;
N-[5-(2-hydroxypropan-2-yl)-4-(trifluoromethyl)-1,3-thiazol-2-yl]-4,6-dimethoxypyrimidine-5-carboxamide;
N-[5-(4-hydroxytetrahydro-2H-pyran-4-yl)-4-(trifluoromethyl)-1,3-thiazol-2-yl]-4,6-dimethoxypyrimidine-5-carboxamide;
N-[5-(4-fluorotetrahydro-2H-pyran-4-yl)-4-(trifluoromethyl)-1,3-thiazol-2-yl]-4,6-dimethoxypyrimidine-5-carboxamide;
N-[5-(1-hydroxycyclobutyl)-4-(trifluoromethyl)-1,3-thiazol-2-yl]-4,6-dimethoxypyrimidine-5-carboxamide;
N-{5-[hydroxy(phenyl)methyl]-4-(trifluoromethyl)-1,3-thiazol-2-yl}-4,6-dimethoxypyrimidine-5-carboxamide;
N-[5-benzoyl-4-(trifluoromethyl)-1,3-thiazol-2-yl]-4,6-dimethoxypyrimidine-5-carboxamide;
N-[5-(1-hydroxy-1-phenylethyl)-4-(trifluoromethyl)-1,3-thiazol-2-yl]-4,6-dimethoxypyrimidine-5-carboxamide;
N-[5-(1-hydroxy-2,2-dimethylpropyl)-4-(trifluoromethyl)-1,3-thiazol-2-yl]-4,6-dimethoxypyrimidine-5-carboxamide;
N-[5-(2,2-dimethylpropanoyl)-4-(trifluoromethyl)-1,3-thiazol-2-yl]-4,6-dimethoxypyrimidine-5-carboxamide;
4,6-dimethoxy-N-{5-[(E)-(methoxyimino)(phenyl)methyl]-4-(trifluoromethyl)-1,3-thiazol-2-yl}pyrimidine-5-carboxamide;
N-{5-[(E)-(hydroxyimino)(phenyl)methyl]-4-(trifluoromethyl)-1,3-thiazol-2-yl}-4,6-dimethoxypyrimidine-5-carboxamide;
4,6-dimethoxy-N-[5-(pyridin-2-ylcarbonyl)-4-(trifluoromethyl)-1,3-thiazol-2-yl]pyrimidine-5-carboxamide;
N-{5-[(E)-(hydroxyimino)(pyridin-2-yl)methyl]-4-(trifluoromethyl)-1,3-thiazol-2-yl}-4,6-dimethoxypyrimidine-5-carboxamide;
N-{5-[1-hydroxy-1-(pyridin-2-yl)ethyl]-4-(trifluoromethyl)-1,3-thiazol-2-yl}-4,6-dimethoxypyrimidine-5-carboxamide;
N-{5-[(5-fluoropyridin-2-yl)(hydroxy)methyl]-4-(trifluoromethyl)-1,3-thiazol-2-yl}-4,6-dimethoxypyrimidine-5-carboxamide;
N-{5-[(5-fluoropyridin-2-yl)carbonyl]-4-(trifluoromethyl)-1,3-thiazol-2-yl}-4,6-dimethoxypyrimidine-5-carboxamide;
N-{5-[(E)-(5-fluoropyridin-2-yl)(hydroxyimino)methyl]-4-(trifluoromethyl)-1,3-thiazol-2-yl}-4,6-dimethoxypyrimidine-5-carboxamide;
N-[5-acetyl-4-(trifluoromethyl)-1,3-thiazol-2-yl]-4,6-dimethoxypyrimidine-5-carboxamide;
N-{5-[(1E)-N-hydroxyethanimidoyl]-4-(trifluoromethyl)-1,3-thiazol-2-yl}-4,6-dimethoxypyrimidine-5-carboxamide;
4,6-dimethoxy-N-{5-[(1E)-N-methoxyethanimidoyl]-4-(trifluoromethyl)-1,3-thiazol-2-yl}pyrimidine-5-carboxamide;
4,6-dimethoxy-N-{5-[(1Z)—N-methoxyethanimidoyl]-4-(trifluoromethyl)-1,3-thiazol-2-yl}pyrimidine-5-carboxamide;
4,6-dimethoxy-N-[5-(2-methyl-1,3-dioxolan-2-yl)-4-(trifluoromethyl)-1,3-thiazol-2-yl]pyrimidine-5-carboxamide;
N-[5-(1,3-dioxolan-2-yl)-4-(trifluoromethyl)-1,3-thiazol-2-yl]-4,6-dimethoxypyrimidine-5-carboxamide;
N-[5-(1,3-dioxan-2-yl)-4-(trifluoromethyl)-1,3-thiazol-2-yl]-4,6-dimethoxypyrimidine-5-carboxamide;
4,6-dimethoxy-N-[5-(1,3-oxazol-5-yl)-4-(trifluoromethyl)-1,3-thiazol-2-yl]pyrimidine-5-carboxamide;
4,6-dimethoxy-N-[5-(4-methyl-1,3-oxazol-5-yl)-4-(trifluoromethyl)-1,3-thiazol-2-yl]pyrimidine-5-carboxamide;
N-[5-(1-ethyl-1H-pyrazol-5-yl)-4-(trifluoromethyl)-1,3-thiazol-2-yl]-4,6-dimethoxypyrimidine-5-carboxamide;
N-[5-(4,5-dihydrofuran-2-yl)-4-(trifluoromethyl)-1,3-thiazol-2-yl]-4,6-dimethoxypyrimidine-5-carboxamide;
4,6-dimethoxy-N-[5-(tetrahydrofuran-2-yl)-4-(trifluoromethyl)-1,3-thiazol-2-yl]pyrimidine-5-carboxamide;
N-(5-(4,5-dihydrofuran-3-yl)-4-(trifluoromethyl)thiazol-2-yl)-4,6-dimethoxypyrimidine-5-carboxamide;
4,6-dimethoxy-N-[5-(tetrahydrofuran-3-yl)-4-(trifluoromethyl)-1,3-thiazol-2-yl]pyrimidine-5-carboxamide;
N-[5-(3,4-dihydro-2H-pyran-5-yl)-4-(trifluoromethyl)-1,3-thiazol-2-yl]-4,6-dimethoxypyrimidine-5-carboxamide;
4,6-dimethoxy-N-[5-(tetrahydro-2H-pyran-3-yl)-4-(trifluoromethyl)-1,3-thiazol-2-yl]pyrimidine-5-carboxamide;
4,6-dimethoxy-N-{5-[(3S*)-tetrahydro-2H-pyran-3-yl]-4-(trifluoromethyl)-1,3-thiazol-2-yl}pyrimidine-5-carboxamide;
4,6-dimethoxy-N-{5-[(3R)-tetrahydro-2H-pyran-3-yl]-4-(trifluoromethyl)-1,3-thiazol-2-yl}pyrimidine-5-carboxamide;
N-{5-[2-(1-cyanocyclopropyl)pyridin-4-yl]-4-(trifluoromethyl)-1,3-thiazol-2-yl}-4,6-dimethoxypyrimidine-5-carboxamide;
N-{5-[2-(cyanomethyl)pyridin-4-yl]-4-(trifluoromethyl)-1,3-thiazol-2-yl}-4,6-dimethoxypyrimidine-5-carboxamide;
N-[5-(6-fluoropyridin-2-yl)-4-(trifluoromethyl)-1,3-thiazol-2-yl]-4,6-dimethoxypyrimidine-5-carboxamide;
N-{5-[6-(3-cyanooxetan-3-yl)pyridin-2-yl]-4-(trifluoromethyl)-1,3-thiazol-2-yl}-4,6-dimethoxypyrimidine-5-carboxamide;
N-{5-[6-(cyanomethyl)pyridin-2-yl]-4-(trifluoromethyl)-1,3-thiazol-2-yl}-4,6-dimethoxypyrimidine-5-carboxamide;
4,6-dimethoxy-N-{5-[5-(propan-2-yloxy)pyridin-3-yl]-4-(trifluoromethyl)-1,3-thiazol-2-yl}pyrimidine-5-carboxamide;
N-{5-[5-(1-cyanocyclopropyl)pyridin-2-yl]-4-(trifluoromethyl)-1,3-thiazol-2-yl}-4,6-dimethoxypyrimidine-5-carboxamide;
N-{5-[5-(2-cyanopropan-2-yl)pyridin-2-yl]-4-(trifluoromethyl)-1,3-thiazol-2-yl}-4,6-dimethoxypyrimidine-5-carboxamide;
N-[5-(2-cyanophenyl)-4-(trifluoromethyl)-1,3-thiazol-2-yl]-4-methoxy-6-(methylsulfanyl)pyrimidine-5-carboxamide;
N-[5-(2-cyanophenyl)-4-(trifluoromethyl)-1,3-thiazol-2-yl]-4-(ethylsulfanyl)-6-methoxypyrimidine-5-carboxamide;
2-cyano-N-[5-iodo-4-(trifluoromethyl)-1,3-thiazol-2-yl]-4-methoxy-6-(trifluoromethyl)pyrimidine-5-carboxamide;
N-[5-(2-cyanopyrimidin-5-yl)-4-(trifluoromethyl)-1,3-thiazol-2-yl]-4,6-dimethoxypyrimidine-5-carboxamide;
N-[4,5-bis(trifluoromethyl)-1,3-thiazol-2-yl]-4,6-dimethoxypyrimidine-5-carboxamide;
N-[5-(4-cyanocyclohex-1-en-1-yl)-4-(trifluoromethyl)-1,3-thiazol-2-yl]-4,6-dimethoxypyrimidine-5-carboxamide;
310 4,6-dimethoxy-N-{5-[1-(methylsulfonyl)-1,2,3,6-tetrahydropyridin-4-yl]-4-(trifluoromethyl)-1,3-thiazol-2-yl}pyrimidine-5-carboxamide;
4,6-dimethoxy-N-{4-(trifluoromethyl)-5-[2-(trifluoromethyl)pyridin-4-yl]-1,3-thiazol-2-yl}pyrimidine-5-carboxamide;
4,6-dimethoxy-N-[5-(quinolin-4-yl)-4-(trifluoromethyl)-1,3-thiazol-2-yl]pyrimidine-5-carboxamide;
N-[5-(1-cyclopropyl-H-pyrazol-4-yl)-4-(trifluoromethyl)-1,3-thiazol-2-yl]-4,6-dimethoxypyrimidine-5-carboxamide;
4,6-dimethoxy-N-[5-(1,2-oxazol-4-yl)-4-(trifluoromethyl)-1,3-thiazol-2-yl]pyrimidine-5-carboxamide;
4,6-dimethoxy-N-{5-[3-(propan-2-yl)-4,5-dihydro-1,2-oxazol-5-yl]-4-(trifluoromethyl)-1,3-thiazol-2-yl}pyrimidine-5-carboxamide;
4,6-dimethoxy-N-{5-[3-(propan-2-yl)-1,2-oxazol-5-yl]-4-(trifluoromethyl)-1,3-thiazol-2-yl}pyrimidine-5-carboxamide;
4,6-dimethoxy-N-[5-(1,2-oxazol-3-yl)-4-(trifluoromethyl)-1,3-thiazol-2-yl]pyrimidine-5-carboxamide;
N-{5-[4-(2-cyanopropan-2-yl)phenyl]-4-(trifluoromethyl)-1,3-thiazol-2-yl}-4,6-dimethoxypyrimidine-5-carboxamide;
4,6-dimethoxy-N-{5-[1-(propan-2-yl)-1H-pyrazol-4-yl]-4-(trifluoromethyl)-1,3-thiazol-2-yl}pyrimidine-5-carboxamide;
N-{5-[1-(cyclopropylmethyl)-1H-pyrazol-4-yl]-4-(trifluoromethyl)-1,3-thiazol-2-yl}-4,6-dimethoxypyrimidine-5-carboxamide;
4,6-dimethoxy-N-[5-(1-methyl-1H-pyrazol-5-yl)-4-(trifluoromethyl)-1,3-thiazol-2-yl]pyrimidine-5-carboxamide;
N-[5-(1-ethyl-1H-pyrazol-4-yl)-4-(trifluoromethyl)-1,3-thiazol-2-yl]-4,6-dimethoxypyrimidine-5-carboxamide;
N-{4-[2-(4-fluorophenoxy)ethyl]-1,3-thiazol-2-yl}-4,6-dimethoxypyrimidine-5-carboxamide;
4,6-dimethoxy-N-{4-[(2-methylphenoxy)methyl]-1,3-thiazol-2-yl}pyrimidine-5-carboxamide;
4,6-dimethoxy-N-{5-[1-(propan-2-yl)-1H-pyrazol-5-yl]-4-(trifluoromethyl)-1,3-thiazol-2-yl}pyrimidine-5-carboxamide;
4,6-dimethoxy-N-{5-[1-methyl-3-(propan-2-yl)-1H-pyrazol-4-yl]-4-(trifluoromethyl)-1,3-thiazol-2-yl}pyrimidine-5-carboxamide;
4,6-dimethoxy-N-{5-[2-(morpholin-4-yl)pyridin-4-yl]-4-(trifluoromethyl)-1,3-thiazol-2-yl}pyrimidine-5-carboxamide;
4,6-dimethoxy-N-[5-(2-methoxypyrimidin-5-yl)-4-(trifluoromethyl)-1,3-thiazol-2-yl]pyrimidine-5-carboxamide;
N-[5-(5-cyano-6-methoxypyridin-3-yl)-4-(trifluoromethyl)-1,3-thiazol-2-yl]-4,6-dimethoxypyrimidine-5-carboxamide;
4,6-dimethoxy-N-{5-[2-(morpholin-4-yl)pyridin-3-yl]-4-(trifluoromethyl)-1,3-thiazol-2-yl}pyrimidine-5-carboxamide;
4,6-dimethoxy-N-{5-[1-methyl-3-(trifluoromethyl)-1H-pyrazol-4-yl]-4-(trifluoromethyl)-1,3-thiazol-2-yl}pyrimidine-5-carboxamide;
N-[5-(furo[3,2-b]pyridin-2-yl)-4-(trifluoromethyl)-1,3-thiazol-2-yl]-4,6-dimethoxypyrimidine-5-carboxamide;
N-{5-[1-(cyclopropylmethyl)-1H-pyrazol-5-yl]-4-(trifluoromethyl)-1,3-thiazol-2-yl}-4,6-dimethoxypyrimidine-5-carboxamide;
N-(4-tert-butyl-5-cyano-1,3-thiazol-2-yl)-4,6-dimethoxypyrimidine-5-carboxamide;
N-[4-cyclopropyl-5-(4-fluorobenzyl)-1,3-thiazol-2-yl]-4,6-dimethoxypyrimidine-5-carboxamide;
N-[4-(2,2-dimethylpropyl)-1,3-thiazol-2-yl]-4,6-dimethoxypyrimidine-5-carboxamide;
N-(5-bromo-4-tert-butyl-1,3-thiazol-2-yl)-4,6-dimethoxypyrimidine-5-carboxamide;
ethyl 4-tert-butyl-2-{[(4,6-dimethoxypyrimidin-5-yl)carbonyl]amino}-1,3-thiazole-5-carboxylate;
365 4,6-dimethoxy-N-{5-[6-(2,2,2-trifluoroethoxy)pyridin-3-yl]-4-(trifluoromethyl)-1,3-thiazol-2-yl}pyrimidine-5-carboxamide;
4,6-dimethoxy-N-{5-[3-(morpholin-4-ylsulfonyl)phenyl]-4-(trifluoromethyl)-1,3-thiazol-2-yl}pyrimidine-5-carboxamide;
4,6-dimethoxy-N-{4-(trifluoromethyl)-5-[5-(trifluoromethyl)pyridin-3-yl]-1,3-thiazol-2-yl}pyrimidine-5-carboxamide;
4,6-dimethoxy-N-[5-{3-[(2-methoxyethyl)(methyl)sulfamoyl]phenyl}-4-(trifluoromethyl)-1,3-thiazol-2-yl]pyrimidine-5-carboxamide;
4,6-dimethoxy-N-{5-[4-(morpholin-4-ylmethyl)phenyl]-4-(trifluoromethyl)-1,3-thiazol-2-yl}pyrimidine-5-carboxamide;
N-[5-(2,1,3-benzoxadiazol-5-yl)-4-(trifluoromethyl)-1,3-thiazol-2-yl]-4,6-dimethoxypyrimidine-5-carboxamide;
N-[5-(1-ethyl-3-methyl-1H-pyrazol-4-yl)-4-(trifluoromethyl)-1,3-thiazol-2-yl]-4,6-dimethoxypyrimidine-5-carboxamide;
N-[5-{3-[(dimethylsulfamoyl)amino]phenyl}-4-(trifluoromethyl)-1,3-thiazol-2-yl]-4,6-dimethoxypyrimidine-5-carboxamide;
4,6-dimethoxy-N-{4-(trifluoromethyl)-5-[2-(trifluoromethyl)pyrimidin-5-yl]-1,3-thiazol-2-yl}pyrimidine-5-carboxamide;
N-[5-(2-cyclopropylpyrimidin-5-yl)-4-(trifluoromethyl)-1,3-thiazol-2-yl]-4,6-dimethoxypyrimidine-5-carboxamide;
N-{5-[5-cyano-6-(morpholin-4-yl)pyridin-3-yl]-4-(trifluoromethyl)-1,3-thiazol-2-yl}-4,6-dimethoxypyrimidine-5-carboxamide;
N-(5-(6-cyano-5-(oxetan-3-yl-methoxy)pyridine-3-yl)-4-(trifluoromethyl)thiazol-2-yl)-4,6-dimethoxypyrimidine-5-carboxamide; and
4,6-dimethoxy-N-(5-(methoxymethyl)-4-(trifluoromethyl)thiazol-2-yl)pyrimidine-5-carboxamide.

49. The compound of claim 1, or the pharmaceutically acceptable salt thereof, wherein the compound is selected from the group consisting of:
N-{5-[2-(2-cyanopropan-2-yl)pyridin-4-yl]-4-(trifluoromethyl)-1,3-thiazol-2-yl}-4,6-dimethoxypyrimidine-5-carboxamide;
4,6-dimethoxy-N-[5-(2-methylpyridin-3-yl)-4-(trifluoromethyl)-1,3-thiazol-2-yl]pyrimidine-5-carboxamide;
N-{4-[5-(difluoromethyl)pyridin-2-yl]-1,3-thiazol-2-yl}-4,6-dimethoxypyrimidine-5-carboxamide;
4,6-dimethoxy-N-{5-methyl-4-[5-(trifluoromethyl)pyridin-2-yl]-1,3-thiazol-2-yl}pyrimidine-5-carboxamide;
4,6-dimethoxy-N-[5-(4-methyl-1,3-oxazol-5-yl)-4-(trifluoromethyl)-1,3-thiazol-2-yl]pyrimidine-5-carboxamide;
4,6-dimethoxy-N-[5-(2-methoxypyrimidin-5-yl)-4-(trifluoromethyl)-1,3-thiazol-2-yl]pyrimidine-5-carboxamide;
N-[5-(1-ethyl-3-methyl-1H-pyrazol-4-yl)-4-(trifluoromethyl)-1,3-thiazol-2-yl]-4,6-dimethoxypyrimidine-5-carboxamide;
4,6-dimethoxy-N-{4-(trifluoromethyl)-5-[2-(trifluoromethyl)pyrimidin-5-yl]-1,3-thiazol-2-yl}pyrimidine-5-carboxamide;
N-(5-(6-cyano-5-(oxetan-3-yl-methoxy)pyridine-3-yl)-4-(trifluoromethyl)thiazol-2-yl)-4,6-dimethoxypyrimidine-5-carboxamide; and
4,6-dimethoxy-N-(5-(methoxymethyl)-4-(trifluoromethyl)thiazol-2-yl)pyrimidine-5-carboxamide.

50. A pharmaceutical composition comprising a therapeutically effective amount of a compound of formula (I) according to claim 1 or the pharmaceutically acceptable salt thereof, in combination with a pharmaceutically acceptable carrier.

51. A method for treating atopic dermatitis, eczema, itch, or psoriasis in a subject in need of such treatment comprising administering to the subject a therapeutically effective amount of a compound of formula (I) according to claim 1, or the pharmaceutically acceptable salt thereof.
